SEQUENCE LISTING (63 sequences)

SEQ ID NO: 1 - DNA, 1374 bp, Homo sapiens
caggtgcagctggtgcagtctggagctgaggtgaagaagcctggggcctcagtgacggtctcctgtcaggtttctggttacacccttaccagctacggtctcagttgggtgcgacaggcccctggacaagggcttgagtgggtgggctggattaacacttacgatggtcagacaaagtatgtaaagaagttccagggccgagtcaccatgaccacacacacaggcacgaacacagcctacatggaaatgaagagcctgagatctgacgacacggccgtgtattactgtgcgagagtcgaaggggttcggggagttatgggctttcattactacccaatggacgtctggggccaagggacaatggtcaccgtctcgagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgtgaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctccgcacaaccactacacgcagaagagcctctccctgtctccgggtaaatgatga

SEQ ID NO: 2 - protein, 456 aa, Homo sapiens
QVQLVQSGAEVKKPGASVTVSCQVSGYTLTSYGLSWVRQAPGQGLEWVGWINTYDGQTKYVKKFQGRVTMTTHTGTNTAYMEMKSLRSDDTAVYYCARVEGVRGVMGFHYYPMDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEAPHNHYTQKSLSLSPGK

SEQ ID NO: 3 - protein, 5 aa, Homo sapiens
SYGLS

SEQ ID NO: 4 - protein, 17 aa, Homo sapiens
WINTYDGQTKYVKKFQG

SEQ ID NO: 5 - protein, 17 aa, Homo sapiens
VEGVRGVMGFHYYPMDV

SEQ ID NO: 6 - protein, 30 aa, Homo sapiens
QVQLVQSGAEVKKPGASVTVSCQVSGYTLT

SEQ ID NO: 7 - protein, 14 aa, Homo sapiens
WVRQAPGQGLEWVG

SEQ ID NO: 8 - protein, 32 aa, Homo sapiens
RVTMTTHTGTNTAYMEMKSLRSDDTAVYYCAR

SEQ ID NO: 9 - protein, 11 aa, Homo sapiens
WGQGTMVTVSS

SEQ ID NO: 10 - protein, 5 aa, Homo sapiens
YDGQT

SEQ ID NO: 11 - protein, 4 aa, Homo sapiens
VRGV

SEQ ID NO: 12 - DNA, 660 bp, Homo sapiens
cagtctgtgttgacgcagccgccctcagtgtctggggccccagggcagagggtcaccatctcctgcactgggagcagctccaacatcggggcaggttatgctgtacactggtaccagcagcttccaggaacagcccccaaactcctcatctctggtaacagcaatcggccctcaggggtccctgaccgattctctggctccaagtctggcacctcagcctccctggccatcactgggctccaggctgaggatgaggctgattattactgccagtcctatgacagcagcctgagtggttcggtattcggcggaggaaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcggcgcgccag

SEQ ID NO: 13 - protein, 219 aa, Homo sapiens
QSVLTQPPSVSGAPGQRVTISCTGSSSNIGAGYAVHWYQQLPGTAPKLLISGNSNRPSGVPDRFSGSKSGTSASLAITGLQAEDEADYYCQSYDSSLSGSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVHEGSTVEKTVAPTECSARQ

SEQ ID NO: 14 - protein, 14 aa, Homo sapiens
TGSSSNIGAGYAVH

SEQ ID NO: 15 - protein, 7 aa, Homo sapiens
GNSNRPS

SEQ ID NO: 16 - protein, 11 aa, Homo sapiens
QSYDSSLSGSV

SEQ ID NO: 17 - protein, 22 aa, Homo sapiens
QSVLTQPPSVSGAPGQRVTISC

SEQ ID NO: 18 - protein, 15 aa, Homo sapiens
WYQQLPGTAPKLLIS

SEQ ID NO: 19 - protein, 32 aa, Homo sapiens
GVPDRFSGSKSGTSASLAITGLQAEDEADYYC

SEQ ID NO: 20 - protein, 11 aa, Homo sapiens
FGGGTKLTVLG

SEQ ID NO: 21 - protein, 221 aa, Influenza virus
GLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 22 - protein, 221 aa, Influenza virus
GIFGAIAGFIENGWEGMIDGWYGFRHQNSEGTGQAADLKSTQAAIDQINGKLNRIIEKTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDVYRDEALNNRFQIKGVELKSGYKDWILWISFAISCFLLCVVLLGFIMWACQRGNIRCNICI

SEQ ID NO: 23 - protein, 221 aa, Influenza virus
GIFGAIAGFIENGWEGMIDGWYGFRHQNSEGTGHAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 24 - protein, 221 aa, Influenza virus
GLFGAIAGFIENGWEGMIDGWYGFRHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 25 - protein, 221 aa, Influenza virus
GIFGAIAGFIENGWEGMVDGWYGFGHQNSEGTGQAADLKSTQAAIDQINGKLNRVIEKTNEKFHQIEKEFSEVEGRIQDLEKYVEDTKIDLWSYNAELLVALENQHTIDLTDSEMNKLFEKTRRQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 26 - protein, 221 aa, Influenza virus
GIFGAIAGFIENGWEGMVDGWYGFRHQNSEGTGQAADLKSTQAAIDQINGKLNRLIEKTNEKFHQIEKEFSEVEGRIQDLEKYVEDTKIDLWSYNAELLVALENQHTIDLTDSEMNKLFEKTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 27 - protein, 221 aa, Influenza virus
GIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 28 - protein, 221 aa, Influenza virus
GIFGAIAGFIENGWEGMVDGWYGFRHQNSEGTGQAADLKSTQAAINQINGKLNRLIEKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 29 - protein, 221 aa, Influenza virus
GIFGAIAGFIENGWEGMVDGWYGFRHQNSEGTGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIESIRNGTYDHDVYRDEALNNRFQIKGVELKSGYKDWILWISFAISCFLLCVALLGFIMWACQKGNIRCNICI

SEQ ID NO: 30 - protein, 221 aa, Influenza virus
GIFGAIAGFIENGWEGMVDGWYGFRHQNSEGIGQAADLKSTQAAINQINGKLNRLIGKTNEKFHQIEKEFSEVEGRIQDLEKYVEDTKIDLWSYNAELLVALENQHTIDLTDSEMNKLFERTKKQLRENAEDMGNGCFKIYHKCDNACIGSIRNGTYDHDVYRDEALNNRFQIKGVELKSGYKDWILWISFAISCFLLCVVLLGFIMWACQKGNIRCNICI

SEQ ID NO: 31 - protein, 221 aa, Influenza virus
GLFGAIAGFIENGWEGMIDGWYGFRHQNSEGTGQAADLKSTQAAIDQINGKLNRVIERTNEKFHQIEKEFSEVEGRIQDLEKYVEDTKIDLWSYNAELLVALENQHTIDLTDSEMNKLFEKTRRQLRENAEDMGNGCFKIYHKCDNACIESIRNGTYDHDIYRDEALNNRFQIKGVELKSSYKDWILWISFAISCFLLCVVLLGFIMWACQRGNIRCNICI

SEQ ID NO: 32 - protein, 221 aa, Influenza virus
GLFGAIAGFIENGWEGLIDGWYGFRHQNAQGEGTAADYKSTQSAIDQITGKLNRLIEKTNQQFELIDNEFTEVEKQIGNVINWTRDSMTEVWSYNAELLVAMENQHTIDLADSEMNKLYERVKRQLRENAEEDGTGCFEIFHKCDDDCMASIRNNTYDHSRYREEAMQNRIQIDPVKLSSGYKDVILWFSFGASCFILLAIAMGLVFICVKNGNMRCTICI

SEQ ID NO: 33 - protein, 222 aa, Influenza virus
GLFGAIAGFIEGGWTGMIDGWYGYHHQNEQGSGYAADQKSTQNAIDGITNKVNSVIEKMNTQFTAVGKEFNNLERRIENLNKKVDDGFLDIWTYNAELLVLLENERTLDFHDSNVRNLYEKVKSQLKNNAKEIGNGCFEFYHKCDDACMESVRNGTYDYPKYSEESKLNREEIDGVKLESMGVYQILAIYSTVASSLVLLVSLGAISFWMCSNGSLQCRICI

SEQ ID NO: 34 - protein, 222 aa, Influenza virus
GLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNNECMESVKNGTYDYPKYSEESKLNREKIDGVKLESMGVYQILAIYSTVASSLVLLVSLGAISFWMCSNGSLQCRICI

SEQ ID NO: 35 - protein, 222 aa, Influenza virus
GLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 36 - protein, 222 aa, Influenza virus
GLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 37 - protein, 222 aa, Influenza virus
GLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 38 - protein, 212 aa, Influenza virus
GLFGAIAGFIEGGWPGLVAGWYGFQHSNDQGVGMAADRDSTQKAIDKITSKVNNIVDKMNKQYGIIDHEFSEIETRLNMINNKIDDQIQDIWTYNAELLVLLENQKTLDEHDANVNNLYNKVKRALGSNAMEDGKGCFELYHKCDDQCMETIRNGTYNRRKYKEESKLERQKIEGIKLESEGTYKILTIYSTVASSLVIAMGFAAFLFWAMS

SEQ ID NO: 39 - protein, 329 aa, Influenza virus
QDLSGNDNSTATLCLGHHAVSNGTVVKTITDDRVEVTNATELVQSSSTGKICNNPHRILDGRDCTLIDALLGDPHCDVFQDETWDLFIERSNAFSNCYPYDVPDHASLRSLVASSGTLEFICEGFTWAGVTQNGESGACKRGPANGFFSRLNWLTKSGSTYPVLNVTMPNNDNFDKLYIWGVHHPSTNQEQTNLYVQASGRVTVSTRRSQQTIIPNIGSRPWVRGQSGRISIYWTIVKPGDVLVINSNGNLIAPRGYFKMRTGKSSIMRSDVPIDTCVSECITPNGSIPNDKPFQNVNKITYGACPKYVKQNTLKLATGMRNVPEKQTR

SEQ ID NO: 40 - protein, 331 aa, Influenza virus
ALVAIIPTNADKICLGHHAVSNGTKVNTLTERGVEVVNATETVERTNVPRICSKGKRTVDLGQCGLLGTITGPPQCDQFLEFSADLIIERREGSDVCYPGKFVNEEALRQILRESGGIDKETMGFTYSGIRTNGATSACRRSGSSFYAEMKWLLSNTDNAAFPQMTKSYKNTRKDPALIIWGIHHSGSTTEQTKLYGSGNKLITVGSSNYQQSFVPSPGARPQVNGQSGRIDFHWLMLNPNDTVTFSFNGAFIAPDRASFLRGKSMGIQSSVQVDANCEGDCYHSGGTIISNLPFQNINSRAVGKCPRYVKQESLMLATGMKNVPEIPKGR

SEQ ID NO: 41 - protein, 337 aa, Influenza virus
LLCAFAATNADTICIGYHANNSTDTVDTVLEKNVTVTHSVNLLEDSHNGKLCKLKGIAPLQLGKCNIAGWLLGNPECDLLLTASSWSYIVETSNSENGTCYPGDFIDYEELREQLSSVSSFEKFEIFPKTSSWPNHETTKGVTAACSYAGASSFYRNLLWLTKKGSSYPKLSKSYVNNKGKEVLVLWGVHHPPTGTDQQSLYQNADAYVSVGSSKYNRRFTPEIAARPKVRDQAGRMNYYWTLLEPGDTITFEATGNLIAPWYAFALNRGSGSGIITSDAPVHDCNTKCQTPHGAINSSLPFQNIHPVTIGECPKYVRSTKLRMATGLRNIPSIQSR

SEQ ID NO: 42 - protein, 337 aa, Influenza virus
LLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSR

SEQ ID NO: 43 - protein, 335 aa, Influenza virus
LILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPEIATRPKVNGLGGRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESR

SEQ ID NO: 44 - protein, 340 aa, Influenza virus
LFAIVSLVKSDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSYWSSHEASLGVSSACPYQGKSSFFRNVVWLTKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKR

SEQ ID NO: 45 - protein, 330 aa, Influenza virus
ILLVVTASNADKICIGYQSTNSTETVDTLTETNVPVTHAKELLHTEHNGMLCATNLGHPLILDTCTIEGLIYGNPSCDLLLGGREWSYIVERPSAVNGMCYPGNVENLEELRSLFSSASSYQRIQIFPDTIWNVSYSGTSKACSDSFYRSMRWLTQKNNAYPIQDAQYTNNRGKSILFMWGINHPPTDTVQTNLYTRTDTTTSVTTEDINRTFKPVIGPRPLVNGLHGRIDYYWSVLKPGQTLRVRSNGNLIAPWYGHILSGESHGRILKTDLNSGNCVVQCQTERGGLNTTLPFHNVSKYAFGNCPKYVGVKSLKLAVGLRNVPARSSR

SEQ ID NO: 46 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 47 - protein, 90 aa, Homo sapiens
QSVLTQPPSVSGAPGQRVTISCTGSSSNIGAGYDVHWYQQLPGTAPKLLIYGNSNRPSGVPDRFSGSKSGTSASLAITGLQAEDEADYYC

SEQ ID NO: 48 - protein, 329 aa, Influenza virus
QDLPGNDNSTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTR

SEQ ID NO: 49 - protein, 329 aa, Influenza virus
QDLPRNDNSTATLCLGHHAVPNGTLVKTITDDQIEVTNATELVQSSSTGKICNNPHRILDGIDCTLIDALLGDPHCDGFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTDQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLATGMRNVPEKQTR

SEQ ID NO: 50 - protein, 329 aa, Influenza virus
QDFPGNDNSTATLCLGHHAVPNGTLVKTITNDQIEVTNANELVQSSSTGKICNNPHRILDGINCTLIDALLGDPHCDGFQNETWDLFVERSKAFSNCYPYDVPDYASLRSLVASSGTLEFINEGFTWTGVTQNGGSNACKRGPDSGFFSRLNWLYKSGSTYPVLNVTMPNNDNFDKLYIWGVHHPSTDQEQTNLYVQASGRVTVSTKRSQQTIIPNIGSRPWVRGLSSRISIYWTIVKPGDILLINSNGNLIAPRGYFKMRTGKSSIMRSDAPIGTCISECITPNGSIPNDKPFQNVNKITYGACPKYVKQNTLKLATGMRNVPEKQTR

SEQ ID NO: 51 - protein, 329 aa, Influenza virus
QNLPRNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQNGGSYACKRGPDNSFFSRLNWLYESESKYPVLNVTMPNNDNFDKLYIWGVHHPSTDKEQTNLYVQASGRVTVSTKRSQQTIIPNVGSRPWVRGLSSRISIYWTIVKPGDILLINSNGNLIAPRGYFKIRTGKSSIMRSDAPIGTCSSECITPNGSIPNDKPFQNVNKITYGACPKYVKQNTLKLATGMRNVPEKQTR

SEQ ID NO: 52 - protein, 329 aa, Influenza virus
QNLPGNDNSTATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEGFNWTGVTQSGGSYTCKRGSDNSFFSRLNWLYESESKYPALNVTMPNNGNFDKLYIWGVHHPSTDKEQTKLYVRASGRVTVSTKRSQQTIIPNIGPRPWVRGLSSRISIYWTIVKPGDILLINSSGNLIAPRGYFKIRTGKSSIMRSDAPIGTCSSECITPNGSIPNDKPFQNVNRITYGACPKYVKQNTLKLATGMRNIPEKQTR

SEQ ID NO: 53 - protein, 329 aa, Influenza virus
QKLPGNDNSKATLCLGHHAVPNGTLVKTITNDQIEVTNATELVQSSSTGRICDSPHRILDGKNCTLIDALLGDPHCDGFQNEKWDLFVERSKAFSNCYPYDVPDYASLRSLVASSGTLEFINEDFNWTGVTQSGGSYACKRGSVNSFFSRLNWLHESEYKYPALNVTMPNNGKFDKLYIWGVHHPSTDKEQTKLYVRASGRVTVSTKRSQQTVIPNIGSRPWVRGLSSGISIYWTIVKPGDILLINSIGNLIAPRGYFKIRTGKSSIMRSDAPIGTCSSECITPNGSIPNDKPFQNVNKITYGACPRYVKQNTLKLATGMRNVPEKQTR

SEQ ID NO: 54 - protein, 329 aa, Influenza virus
QKLPGNDNSTATLCLGHHAVPNGTLVKTITNDQIEVTNATELVHSSSTGRICDSPHRILDGKNCTLIDALLGDPHCDGFQNKEWDLFVERSKAYSNCYPYDVPDYASLRSLVASSGTLEFINEDFNWTGVAQSGGSYACKRGSINSFFSRLNWLHESEHKYPALNVTMPNNGKFDKLYIWGVHHPITDREQTNLYVRASGRVTVSTKRSQQTVIPNIGSRPWVRGLSSRISIYWTIVKPGDILLINSTGNLIAPRGYFKIRTGKSSIMRSDAPIGTCSSECITPNGSIPNDKPFQNVNRITYGACPRYVKQNTLKLATGMRNVPEKQTR

SEQ ID NO: 55 - protein, 329 aa, Influenza virus
QKLPGNDNSTATLCLGHHAVPNGTLVKTITNDQIEVTNATELVQSSSTGRICDSPHRILDGKNCTLIDALLGDPHCDGFQNKEWDLFVERSKAYSNCYPYDVPDYASLRSLVASSGTLEFINEDFNWTGVAQDGGSYACKRGSVNSFFSRLNWLHKSEYKYPALNVSMPNNGKFDKLYIWGVHHPSTDSDQTSLYVQASGRVTVSTKRSQQTVTPNIGSRPWVRGQSSRISIYWTIVKPGDILLINSTGNLIAPRGYFKIRNGKSSIMRSDAPIGTCSFECITPNGSIPNDKPFQNVNRITYGACPRYVKQNTLKLATGMRNVPEKQTR

SEQ ID NO: 56 - protein, 329 aa, Influenza virus
QKIPGNDNSTATLCLGHHAVPNGTLVKTITNDQIEVTNATELVQSSSTGRICDSPHRILDGENCTLIDALLGDPHCDGFQNKEWDLFVERSKAYSNCYPYDVPDYASLRSLVASSGTLEFNNESFNWTGVAQNGTSYACKRSSIKSFFSRLNWLHQLKYKYPALNVTMPNNDKFDKLYIWGVHHPSTDSDQTSIYAQASGRVTVSTKRSQQTVIPNIGSRPWVRGISSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 57 - protein, 329 aa, Influenza virus
QKLPGNDNSTATLCLGHHAVSNGTLVKTITNDQIEVTNATELVQSSSTGRICDSPHQILDGENCTLIDALLGDPHCDGFQNKEWDLFVERSKAYSNCYPYDVPDYASLRSLVASSGTLEFNNESFNWTGVAQNGTSSACKRRSNKSFFSRLNWLHQLKYKYPALNVTMPNNEKFDKLYIWGVHHPSTDSDQISIYAQASGRVTVSTKRSQQTVIPNIGSSPWVRGVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 58 - protein, 329 aa, Influenza virus
QKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWAGVTQNGTSSACKRRSNKSFFSRLNWLTHLKYKYPALNVTMPNNEKFDKLYIWGVHHPGTDSDQISLYAQASGRITVSTKRSQQTVIPNIGSRPRVRDVSSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 59 - protein, 329 aa, Influenza virus
QKLPGNDNSTATLCLGHHAVPNGTIVKTITNDQIEVTNATELVQSSSTGGICDSPHQILDGENCTLIDALLGDPQCDGFQNKKWDLFVERSKAYSNCYPYDVPDYASLRSLVASSGTLEFNNESFNWTGVTQNGTSSSCKRRSNNSFFSRLNWLTHLKFKYPALNVTMPNNEKFDKLYIWGVHHPVTDNDQIRLYAQASGRITVSTKRSQQTVIPNIGSRPRVRDIPSRISIYWTIVKPGDILLINSTGNLIAPRGYFKIRSGKSSIMRSDAPIGKCNSECITPNGSIPNDKPFQNVNRITYGACPRYVKQNTLKLATGMRNVPEKQTR

SEQ ID NO: 60 - protein, 336 aa, Influenza virus
LLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTGVSASCSHNGKSSFYRNLLWLTGKNGLYPNLSKSYVNNKEKEVLVLWGVHHPPNIGNQRALYHTENAYVSVVSSHYSRRFTPEIAKRPKVRDQEGRINYYWTLLEPGDTIIFEANGNLIAPWYAFALSRGFGSGIITSNAPMDECDAKCQTPQGAINSSLPFQNVHPVTIGECPKYVRSAKLRMVTGLRNIPSIQSR

SEQ ID NO: 61 - DNA, 21 bp, Artificial
  H3HA forward primer
gcaaaagcaggggataattct

SEQ ID NO: 62 - DNA, 25 bp, Artificial
  H3HA backward primer
gtagaaacaagggtgtttttaatta

SEQ ID NO: 63 - DNA, 24 bp, Artificial
  H3HA 586 primer
tgaacgtgactatgccaaacaatg

(19) United States
(12) Patent Application Publication
(10) Pub. No.: US 2014/0086927 A1
(43) Pub. Date: Mar. 27, 2014

(54) NOVEL EPITOPE AND MECHANISM OF ANTIGEN-ANTIBODY INTERACTION IN AN INFLUENZA VIRUS

(71) Applicants: Fujita Health University, Toyoake-shi (JP); The Research Foundation for Microbial Diseases of Osaka University, Osaka (JP); Osaka University, Osaka (JP)

(72) Inventors: Yoshikazu Kurosawa, Toyoake-shi (JP); Yoshitaka Iba, Toyoake-shi (JP); Nobuko Ohshima, Toyoake-shi (JP); Shigeyuki Yokoyama, Yokohama-shi (JP); Mikako Shirouzu, Yokohama-shi (JP); Yoshifumi Fujii, Yokohama-shi (JP); Tomomi Sumida, Yokohama-shi (JP); Kazuyoshi Ikuta, Suita-shi (JP); Shota Nakamura, Suita-shi (JP); Norihito Kawashita, Suita-shi (JP); Mitsuhiro Nishimura, Suita-shi (JP); Akifumi Yamashita, Suita-shi (JP); Yoshinobu Okuno, Kanonji-shi (JP); Ritsuko Kubota-Koketsu, Kanonji-shi (JP); Masahiro Okubo, Kanonji-shi (JP)

(73) Assignees: Fujita Health University, Toyoake-shi (JP); The Research Foundation for Microbial Diseases of Osaka University, Osaka (JP); Osaka University, Osaka (JP)

(21) Appl. No.: 14/035,495

(22) Filed: Sep. 24, 2013

Related U.S. Application Data
(63) Continuation-in-part of application No. 13/832,818, filed on Mar. 15, 2013.
(60) Provisional application No. 61/705,504, filed on Sep. 25, 2012.
(60) Provisional application No. 61/705,504, filed on Sep. 25, 2012.

(51) Int. Cl. C07K 16/10; G01N 33/569

(52) U.S. Cl. CPC C07K 16/1018; G01N 33/56983
USPC 424/139.1; 530/387.9; 435/5; 530/350; 436/501; 506/9

(57) ABSTRACT

Antibodies (Abs) play roles in protection against influenza. Neutralizing Abs either inhibit the binding of hemagglutinin (HA) to cellular receptors or prevent the conformational change of HA induced by low pH. The former Ab binds to the regions near the sialic acid-binding pocket on the globular head formed by HA1 and generally shows narrow strain specificity. The latter Ab binds to the stem region formed mainly by HA2 and shows broad strain specificity. We isolated a broadly neutralizing Ab against H3N2 viruses. X-ray analysis of the HA/Ab complex indicated that the Ab binds to the valley formed by two neighboring HA monomers at the side of the globular head. The Ab shows neutralizing activity by preventing the conformational change of HA induced at low pH.

### CROSS-REFERENCE TO RELATED APPLICATIONS

This application claims priority under 35 U.S.C. 119(e) to U.S. Provisional Application No. 61/705,504, filed Sep. 25, 2012; and is a continuation-in-part of U.S. application Ser. No. 13/832,818, filed on Mar. 15, 2013 (now pending), which claims priority under 35 U.S.C. 119(e) to U.S. Provisional Application No. 61/705,504, filed Sep. 25, 2012; this application is also a continuation-in-part of U.S. application Ser. No. ______ (previously Provisional Application No. 61/787,399, filed Mar. 15, 2013, which application has been converted to a nonprovisional application on Sep. 24, 2013, with serial number to follow); all of these applications are incorporated herein by reference in their entireties.

### BACKGROUND OF THE INVENTION

1. Field of the Invention

The present invention relates to novel antigen-antibody interaction of hemagglutinin trimer of influenza virus, and use thereof for screening novel types of vaccines or neutralizing antibodies. In particular, the present invention is related to a new conserved neutralizing epitope at the globular head of hemagglutinin in H3N2 influenza viruses. A broadly neutralizing antibody bridging two neighboring head regions of H3 influenza hemagglutinins at novel epitopes are also discussed.

2. Description of Background Art

Influenza A viruses are subclassified by two surface proteins, HA and neuraminidase (NA). There are 16 HA subtypes (H1-16) and 9 NA subtypes (N1-9). Based on the similarity of amino acid sequences, the 16 HA subtypes are further classified into group 1 and group 2. Last century, H1, H2 and H3 subtype viruses infected human and caused three major pandemics. In 2009, a novel H1N1 virus originating in swine caused the first pandemic influenza in the 21st century. The highly pathogenic avian influenza (HPAI) H5N1 virus remains as the most serious threat because of its potential to cause future pandemic. H1, H2 and H5 belong to group 1 and H3 belongs to group 2. Since Abs play important roles in protection against and recovery from influenza virus infection, passive immunization with neutralizing monoclonal Abs (MAbs) is considered to be a prophylactic and therapeutic strategy to combat the pandemic caused by HPAI virus. However, since HA is the main target for virus-neutralizing Abs and mutations are easily introduced into the epitope on HA, it is impossible to predict with 100% fidelity the exact antigenic structure of a putative virus which will cause pandemic in future. Thus, human mAbs showing a broad neutralizing activity, for instance, against all H5 subtype viruses, all group 1 viruses, all group 2 viruses, and all influenza A viruses are predicted as potential Ab drugs against the future pandemic.

HA mediates virus entry into the cells in two different steps. Firstly, HA binds to the cell receptor, particularly the sialic acid thereon. After internalization of viruses by endocytosis, HA undergoes a drastic conformational change induced by low pH, resulting in fusion between the virus envelop and the cell membrane. It has been shown that neutralizing Abs have one of the following two activities: the prevention of the binding reaction between HA and the sialic acid and the prevention of the conformational change of HA. However, the dominant immune response against influenza HA is thought to be the first type, and the epitopes are located at defined sites near the sialic acid-binding pocket. Since Abs against these sites are very potent and mutations can be introduced into these sites without losing the receptor-binding activity, variant viruses that have acquired resistance to these Abs become dominant and cause annual epidemics.

In 1993, Okuno et al. described mouse mAb C179, which has a broad neutralizing activity against H1N1, H2N2 and H5N1 in group 1. Based on the results that C179 recognizes conserved sequences 318-322 of HA1 and 47-58 of HA2, which are located at the middle of the stem region of HA, it was suggested that C179 shows neutralizing activity by inhibiting hemagglutinin-mediated membrane fusion. Fifteen years later, three groups independently isolated human mAbs that broadly neutralize group 1 viruses. They screened combinatorial Ab libraries constructed from human B cells and isolated clones that were similar to one another. The Abs neutralized both H5N1 and H1N1 viruses and utilized the VH1-69 gene. The X-ray structural analyses of HA/Ab complexes of two clones, CR6261 and F10, further indicated that the Abs block infection by inserting their heavy (H) chain into a conserved pocket in the stem region, thus preventing membrane fusion. The light (L) chains were not directly involved in the interactions with HA. Since the publication of these results, many papers have described the isolation of broadly neutralizing Abs and identification of their epitopes.

### SUMMARY OF INVENTION

Antibodies (Abs) play roles in protection against influenza, and hemagglutinin (HA) is the target for neutralizing Abs. While HA mediates virus entry into the cells, neutralizing Abs either inhibit the binding of HA to a cellular receptor, or prevent the conformational change of HA induced by low pH. The former Ab binds to the regions near the sialic acid-binding pocket on the globular head formed by HAL and shows narrow strain specificity. The latter Ab binds to the stem region formed mainly by HA2, and shows broad strain specificity. We isolated a broadly neutralizing Ab against H3N2 viruses. X-ray analysis of the HA/Ab complex indicated that it binds to the side of the globular head of HA. The Ab prevents the conformational change of HA induced at low pH.

As such, the present invention provides the following:

### Item Z1

An isolated antibody directed to hemagglutinin (HA) trimer of an influenza virus, wherein said antibody comprises:

(i) the sequence of CDR1 (SEQ ID NO: 3) of F005-126 antibody heavy chain (SEQ ID NO: 2), or a functionally equivalent sequence thereof;

(ii) the sequence of CDR2 (SEQ ID NO: 4) of F005-126 antibody heavy chain (SEQ ID NO: 2), or a functionally equivalent sequence thereof;

(iii) the sequence of CDR1 (SEQ ID NO: 5) of F005-126 antibody heavy chain (SEQ ID NO: 2), or a functionally equivalent sequence thereof; and

(iv) the sequence of FR3 (SEQ ID NO: 8) of F005-126 antibody heavy chain (SEQ ID NO: 2), or a functionally equivalent sequence thereof.

### Item Z2A

The antibody according to item Z1, which further comprises

(v) the sequence of FR1 (SEQ ID NO: 6) of F005-126 antibody heavy chain (SEQ ID NO: 2), or a functionally equivalent sequence thereof

(vi) the sequence of FR2 (SEQ ID NO: 7) of F005-126 antibody heavy chain (SEQ ID NO: 2), or a functionally equivalent sequence thereof and

(vii) the sequence of FR4 (SEQ ID NO: 9) of F005-126 antibody heavy chain (SEQ ID NO: 2), or a functionally equivalent sequence thereof.

### Item Z2B

The antibody according to item Z1 or Z2A, which further comprises:

the sequence of F005-126 antibody light chain (SEQ ID NO: 13), or a functionally equivalent sequence thereof.

### Item Z3

The antibody according to item Z1, Z2A or Z2B, wherein said antibody comprises at least one of the properties selected from the group consisting of:

(1) having broad strain specificity against H3;

(2) binds to HA1 head region but does not inhibit binding to cell;

(3) inhibits structural change of HA;

(4) said CDR1, CDR3 and FR3 bind to HA by van der Waals contact;

(5) said CDR2 binds to N285 sugar chain (according to the Kabat's numbering shown in FIG. 5-2) which is conserved in HA;

(6) binds to the HA trimer across two HA subunits thereof which are adjacent to each other;

(7) intra- and inter-subunit interactions between HA1 and HA2 by salt bridges are located on the amino acid sequence of the molecular surface in the vicinity of the portion which maintains structure of the HA trimer;

(8) comprising hydrogen bonds.

### Item Z4

The antibody according to item Z3, wherein the antibody has a property of binding to the HA trimer across two HA subunits thereof which are adjacent to each other.

### Item Z5

The antibody according to item Z1, Z2A, Z2B, Z3, or Z4 which is a neutralizing antibody.

### Item Z6

The antibody according to item Z1, Z2A, Z2B, Z3, Z4 or Z5 which is an antibody neutralizing H3.

### Item Z7

The antibody according to item Z1, Z2A, Z2B, Z3, Z4, Z5 or Z6, wherein the antibody comprises (a) the sequence set forth in SEQ ID NO: 2 (the full sequence), or (b) a sequence derived from the sequence of (a) comprising one or more amino acid substitution(s), deletion(s) and/or addition(s).

### Item Z8

The antibody according to item Z1, Z2A, Z2B, Z3, Z4, Z5, Z6 or Z7, wherein the antibody comprises:

(a) the sequence set forth in SEQ ID NO: 2 (the full sequence), or (b) a sequence derived from the sequence of (a) comprising one or more amino acid substitution(s), deletion(s) and/or addition(s) at positions other than the binding site with HA of CDR1 sequence of F005-126 antibody heavy chain (amino acid No. 31 (Ser) of SEQ ID NO. 2), the binding site with HA of CDR2 sequence of F005-126 antibody heavy chain (SEQ ID NO: 10 (amino acids No. 54-58 (Tyr Asn Gly Asn Thr) of SEQ ID NO. 2)), the binding site with HA of CDR3 sequence of F005-126 antibody heavy chain (amino acids No. 74-76 (Thr Ser Thr) of SEQ ID NO. 2), and the binding site with HA of FR3 sequence of F005-126 antibody heavy chain (SEQ ID NO: 11 (amino acids No. 102-105 (Val Arg Gly Val) of SEQ ID NO. 2)), wherein the sequence maintains the binding activity with the HA trimer.

### Item Z9

The antibody according to item Z1, Z2A, Z2B, Z3, Z4, Z5, Z6, Z7 or Z8, wherein the antibody comprises:

(a) the sequence set forth in SEQ ID NO: 2 (the full sequence), or

(b) a sequence derived from the sequence of (a) comprising one or more amino acid substitution(s), deletion(s) and/or addition(s) at positions other than the CDR1 sequence of F005-126 antibody heavy chain (SEQ ID NO: 3), the CDR2 sequence of F005-126 antibody heavy chain (SEQ ID NO: 4), the CDR3 sequence of F005-126 antibody heavy chain (SEQ ID NO: 5), and the FR3 sequence of F005-126 antibody heavy chain (SEQ ID NO: 8), wherein the sequence maintains the binding activity with the HA trimer.

### Item Z10

The antibody according to item Z1, Z2A, Z2B, Z3, Z4, Z5, Z6, Z7, Z8 or Z9, which consists of the sequence set forth in SEQ ID NO: 2 (the full sequence of the heavy chain) and SEQ ID NO: 13 (the full sequence of the light chain).

### Item Z11

A screening kit for an antibody against hemagglutinin (HA) trimer of an influenza virus, comprising the antibody according to item Z1, Z2A, Z2B, Z3, Z4, Z5, Z6, Z7, Z8, Z9 or Z10.

### Item Z12

The kit according to item Z11, further comprising a protein or protein complex comprising the sequence of concave region of the HA trimer (SEQ ID NOs: 48 and 21).

### Item Z13

An influenza virus passive immunotherapy agent comprising the antibody according to item Z1, Z2A, Z2B, Z3, Z4, Z5, Z6, Z7, Z8, Z9 or Z10.

### Item Z14

A method of influenza virus passive immunotherapy comprising the step of administering the antibody according to item Z1, Z2A, Z2B, Z3, Z4, Z5, Z6, Z7, Z8, Z9 or Z10 to a patient in need thereof.

### Item Y1

A kit for paratope analysis of an influenza neutralizing antibody comprising a protein or protein complex comprising the sequence of concave region of a HA trimer (e.g. SEQ ID NOs: 48 and 21).

### Item Y2

The kit according to item Y1, wherein the protein or protein complex is (A) the full length sequence of the HA trimer (e.g. SEQ ID NO: 48 and 21); or (B) a sequence derived from the full length sequence of (B) comprising one or more amino acid substitution(s), deletion(s) and/or addition(s) at the positions other than the sequence of concave region of the HA trimer, wherein the sequence maintains the binding activity with F005-126 antibody.

### Item Y3

The kit according to item Y1 or Y2, wherein the protein or protein complex consists of (A) the full length sequence of the HA trimer (e.g. SEQ ID NO: 48 and 21).

### Item Y4

The kit according to item Y1, Y2 or Y3, wherein the paratope is related to an antibody against Group 2 hemagglutinin.

### Item Y5

The kit according to item Y1, Y2, Y3 or Y4 wherein the paratope is related to an antibody against hemagglutinin H3.

### Item Y6

The kit according to item Y1, Y2, Y3, Y4 or Y5 wherein the paratope is related to an antibody against hemagglutinin H3, whose strain is selected from the group consisting of Aic68 (SEQ ID NO: 48 and 21), Fuk70 (SEQ ID NO: 48 and 22), Tok73 (SEQ ID NO: 50 and 23), Yam77 (SEQ ID NO: 51 and 24), Nii81 (SEQ ID NO: 52 and 25), Fuk85 (SEQ ID NO: 53 and 26), Gui89 (SEQ ID NO: 54), Kit93 (SEQ ID NO: 55), Syd97 (SEQ ID NO: 56 and 27), Pan99 (SEQ ID NO: 57 and 28), Wyo03 (SEQ ID NO: 58 and 29) and NY04 (SEQ ID NO: 59 and 30).

### Modeling Inventions

### Item X1

A method for identifying a binding substance to a hemagglutinin (HA) trimer of an influenza virus, the method comprising the steps of:

(A) providing a 3D structural representation of the HA trimer, wherein the 3D structural representation of the HA trimer comprises the atomic co-ordinates relating to a 3D structural representation of the amino acid residues contained in the HA of Table 1 which is described at the bottom of the specification:

(B) providing a 3D structural representation of a candidate substance of the binding substance;

(C) using a computer to dock the 3D structural representation of the candidate substance with the 3D structural representation of the HA trimer, wherein a candidate substance that docks with the HA trimer at the site comprising the amino acid residues contained in the HA of the Table 1, is identified as the binding substance of the HA trimer;

(D) contacting the candidate substance identified in step (C) with HA trimer or a fragment thereof containing the 3D structure of the amino acid residues contained in the HA of the Table 1; and

(E) assaying the interaction between the candidate substance and the HA trimer or the fragment thereof, to determine whether the binding substance identified in step (C) is a binding substance for the HA trimer.

### Item X2

The method according to item X1, wherein the 3D structural representation comprises at least one interaction selected from the group consisting of van der Waals contacts, electrostatic interactions, and hydrogen bonding.

### Item X3

The method according to item X1 or X2, wherein the 3D structural representation comprises van der Waals contacts, electrostatic interactions, and hydrogen bonding.

### Item X4

The method according to item X1, X2 or X3, wherein the 3D structural representation of the amino acid residues contained in the HA of the following Table 1 comprises

(A) the atomic co-ordinates set forth in Table 2 consisting of Tables 2-1, 2-2, 2-3 and 2-4 which is described at the bottom of the specification:

or

(B) variant atomic co-ordinates of (A), in which the r.m.s. deviation of the x, y and z co-ordinates for all heavy atoms is less than 2.5 Angstroms or 4.0 Angstroms.

### Item X5

The method according to item X1, X2, X3 or X4, wherein the 3D structural representation of the amino acid residues contained in the HA of Table 1 comprises the entire atomic co-ordinates set forth in PDB1, PDB2, PDB3 and/or PDB4.

### Item X6

The method according to item X1, X2, X3 or X4, wherein said step of docking comprises geometric matching or minimizing the energy of interaction between the candidate substance and the amino acid residues of the HA trimer contained in the HA of the Table 1.

### Item X7

The method according to item X1, X2, X3, X4 or X5, wherein the candidate substance comprises a library of antibodies.

### Item X8

The method according to item X1, X2, X3, X4, X5 or X6 wherein the binding substance is a (fusogenic) conformational change inhibitor of HA trimer.

### Item X9

The method according to item X1, X2, X3, X4, X5, X6 or X7, wherein the step of docking comprises referring to the 3D structural representation of the antibody set forth in Table 1.

### Antigen Series A

### Item A1

A conformational epitope formed by an antibody-antigen complex of antibody F005-126, antigen HA-a+HA-b and/or water molecule, wherein the HA-a and HA-b are HA1 selected from the group consisting of SEQ ID NOs: 48-60 and 39-45 and HA2 selected from the group consisting of SEQ ID NOs: 21-38, wherein the conformational epitope comprises:

the following amino acid residues of the amino acid sequences of HA1 of H3N2 Aic 68 (SEQ ID NO: 48), or corresponding amino acid residues thereto:

a Site L epitope element comprising amino acid residues N171, D172, N173, P239 and G240;

a Site R epitope element comprising amino acid residues S91, K92, S270, D271, A272, P273, P284 and N285;

a Site R epitope element comprising sugar chains NAG (N-acetyl-D-glucosamine)1, NAG2 BMA(beta-D-mannose)3, MAN(alpha-D-mannose)4, MAN5, MAN6 and MAN7, linked to amino acid residue N285, wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

### Item A2

The epitope according to item A1, wherein said crystal has the atomic co-ordinates set forth in PDB1, PDB2, PDB3 or PDB4.

### Item A3

An antigen comprising the epitope according to item A1 or A2.

### Item A4

A vaccine comprising the antigen according to item A3.

### Item A5

A screening method of a neutralizing antibody using the antigen according to item A3.

### B Series: Antibody

### Item B1

A paratope of antibody F005-126 in an antibody-antigen complex of antibody F005-126, antigen HA-a+HA-b and water molecule, wherein the HA-a and HA-b are HA1 selected from the group consisting of SEQ ID NOs: 48-60 and 39-45 and HA2 selected from the group consisting of SEQ ID NOs: 21-38, wherein the paratope comprises:

the following amino acid residues of F005-126 heavy chain (SEQ ID NO:2), or corresponding amino acid residues thereto:

a paratope element comprising amino acid residues T73, G74, and T75 (according to the Kabat's numbering shown in FIG. 5-2);

a paratope element comprising amino acid residue S31 (according to the Kabat's numbering shown in FIG. 5-2);

a paratope element comprising amino acid residues Y53, D54, G55, Q56 and H57 (according to the Kabat's numbering shown in FIG. 5-2); and

a paratope element comprising V98, R99, G100, and V100a (according to the Kabat's numbering shown in FIG. 5-2);

wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

### Item B2

The paratope according to item B1, wherein said crystal has the atomic co-ordinates set forth in PDB1, PDB2, PDB3 or PDB4.

### Item B3

A neutralizing antibody comprising the paratope according to item B1 or B2.

### Item B4

A passive immune therapy agent comprising an antibody comprising the paratope according to item B1.

### Item B5

A screening method of a vaccine using the paratope according to item B1 or B2.

### Series C: Screening:

### Item C1

A partial or full complex of a conformational epitope and paratope formed by an antibody-antigen complex of antibody F005-126, antigen HA-a+HA-b and water molecule, wherein the HA-a and HA-b are HA1 selected from the group consisting of SEQ ID NOs: 48-60 and 39-45 and HA2 selected from the group consisting of SEQ ID NOs: 21-38, wherein the conformational epitope comprises:

the following amino acid residues of the amino acid sequences of HA1 of H3N2 Aic 68 (SEQ ID NO: 48), or corresponding amino acid residues thereto:
a Site L epitope element comprising amino acid residues N171, D172, P239 and G240
a Site R epitope element comprising amino acid residues S270, D271, A272, P273, P284 and N285;
a Site R epitope element comprising sugar chains NAG(N-acetyl-D-glucosamine)1, NAG2 BMA(beta-D-mannose)3, MAN(alpha-D-mannose)4, MAN5, MAN6 and MAN7, linked to amino acid residue N285,

wherein the paratope comprises:
the following amino acid residues of F005-126 heavy chain (SEQ ID NO:2), or corresponding amino acid residues thereto:
a paratope element comprising amino acid residues T73, G74, and T75 (according to the Kabat's numbering shown in FIG. 5-2)
a paratope element comprising amino acid residue S31 (according to the Kabat's numbering shown in FIG. 5-2);
a paratope element comprising amino acid residues Y53, D54, G55, Q56 and H57 (according to the Kabat's numbering shown in FIG. 5-2)
a paratope element comprising V98, R99, G100, and V100a (according to the Kabat's numbering shown in FIG. 5-2);

wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

### Item C2

A complex comprising the partial complex according to item C1.

### Item C3

A screening method for a neutralizing antibody or a vaccine using the partial complex according to item C1 or a complex according to C2.

To reveal the repertoire of neutralizing antibodies (Abs) against influenza viruses in humans, we collected large numbers of B lymphocytes by apheresis from three healthy donors and constructed Ab libraries by using phage-display technology. The libraries were screened with virus particles of 12 kinds of H3N2 influenza vaccine strains from 1968 to 2004. Clones that bound to virus particles were isolated, and their binding and neutralizing activities against the 12 H3N2 virus strains were examined. We previously reported the results obtained from the library of a donor born in 1974. The collection of neutralizing Abs contained two types of clones that showed broad strain specificity. The first type of clones neutralized not only H3N2 but also H1N1, H2N2, and H5N1 viruses, although the activities were largely varied. The epitope recognized by this type of clone, F045-092, was described in a previous paper. The second type of clones neutralized all 12 H3N2 viruses but not group 1 viruses. As shown in FIG. 1A, the IgG type of F005-126 neutralized 12 kinds of H3N2 viruses with various activities ranging from 0.1 to 100 nM (50% inhibitory concentration [IC50]). In the present study, we analyzed the epitope recognized by F005-126. The amino acid sequences of the VH and VL fragments of F005-126 are shown in FIG. 5.

The present invention also provides the following:

### Item D1

A method for screening an active agent for hemagglutinin comprising:
(a) constructing a 3D structure model of hemagglutinin using any one of PDB1, PDB2, PDB3 and PDB4;
(b) identifying a dock site;
(c) carrying out docking simulations for a first library of compounds as an initial screen;
(d) selecting hits from the initial screen; and
(e) performing a secondary screen using a combined library of the hits from the initial screen and a second library thereby determining the active agents.

### Item D2

A method for estimating variations within subtypes of Influenza A viruses, comprising:
(a) providing amino acid sequences of Influenza A virus;
(b) extracting complete Hemagglutinin sequences from the amino acid sequences of step (a);
(c) aligning the sequences extracted in step (b) and identifying the epitope regions according to the positions shown in FIG. 8.; and
(d) estimating the variation of each subtype by computing Shannon index of each site, by counting the number of different kind of sequences and by making sequence logos.

### Item D3

A method for screening active agent for regulating influenza virus or influenza virus hemagglutinin comprising:
(a) constructing a 3D structure model of hemagglutinin using any one of PDB1, PDB2, PDB3 and PDB4;
(b) identifying a dock site;
(c) carrying out docking simulations for a first library of compounds as an initial screen;
(d) selecting hits from the initial screen; and
(e) performing a biological assay with the candidate compound to confirm that the compound has the regulating activity.

In all these aspects, it is understood that each embodiment described as used herein can be applied to other aspects, as long as it is applicable.

### EFFECTS OF INVENTION

The present invention provides new mechanism of HA-trimer inhibition and thus provides new model of screening methods for antibody drugs and vaccines.

### BRIEF DESCRIPTION OF THE DRAWINGS

This patent application file contains at least one drawing executed in color. Copies of this patent application publication with color drawings will be provided by the Office upon request and payment of the necessary fee.

FIG. 1. Neutralizing activity of F005-126 and the binding activity of various Abs to HAs artificially expressed on cells. (A) The neutralizing activity of F005-126 IgG against the 12 H3N2 strain viruses was examined by focus reduction assay. (B) The binding of various Abs to HA artificially expressed on cells was examined by FCM. Green line: HA of H3N2 (Aic68); Red line: HA of H1N1 (NC99); Gray filled: mock transfection. F045-092 binds to both H3-type and H1-type HAs. F49 binds to an epitope on HA2 commonly present in H3 HA. Anti-V5 Ab binds to the V5 tag located at the membrane-proximal end of HA. (C) The binding of Abs to HA (green) and HA1 domain (red) of H3N2 (Fuk85) was analyzed by FCM. F019-102 binds to site E on HA1. (D) The binding of Abs to HA (green) and truncated HAs, HA39-319 (pink) and HA44-309 (blue), of H3N2 (Fuk85) was analyzed by FCM. Anti-myc Ab binds to the myc tag located at the membrane-proximal end of HA.

FIG. 2. Competition for binding to HA between F005-126 and four mAbs that bind to site C1/C2, E and B1 on HA1. Formalin-inactivated H3N2 (Yam77) virus particles were coated onto a MaxiSoap immunoplate. Competitive ELISA was performed by using Fab-PP for detection of binding activity and Fab-cp3 [cp3 denotes coat protein 3] as a competitor. Fab-cp3 molecules in the supernatant of the E. coli culture were concentrated 20-fold. Fifty ml of Fab-PP (P denotes a single Fc-binding domain of protein A) at an optimized concentration was mixed with 50 ml of the 20-fold-concentrated Fab-cp3 or with PBS (Fab-cp3−) and added to the virus-coated immunoplate. Then, peroxidase-conjugated rabbit Ab was added. Finally OPD was added, and the OD at 492 nm was measured. The antigenic sites recognized by the four mAbs are indicated in parentheses above the graph.

FIGS. 3-1, 3-2 and 3-3. Crystal structures of Aic68 HA in complex with F005-126. (A) Side view of the crystal structure. The trimeric HA is depicted by surface representation, and 2 HA subunits, HA-R and HA-L, are shown in navy blue and in green, respectively. F005-126 Fabs are shown as ribbons. One Fab is colored. The H chain is in red and the L chain is in yellow. Glycans are depicted as spheres. (B) Close-up view of site R and site L. HA-R and HA-L are depicted by surface representation. Glycans at Asn165 in HA1-L and Asn285 in HA1-R are depicted as yellow (upper) and dark blue (lower) spheres, respectively. CDRs 1, 2 and 3 and FR3 of the H chain are depicted by wireframe in white, purple, green, and orange, respectively. Sites R and L are colored red. (C) Interactions between site L and FR3 of the H chain are shown. (D) Interactions among site R, the glycan at HA1-R:Asn285, HCDR2, and HCDR3 are shown. N-acetyl-D-glucosamine, a-D-mannose, and b-D-mannose are abbreviated as NAG, MAN, and BMA, respectively. (E) The interactions in FIG. 3D are schematically depicted.

FIG. 4. Inhibition of the low pH-induced conformational change by binding of F005-126 to HA1 s and the binding sites close to salt bridges keeping the B loop in the prefusion state at neutral pH. (A) HA was digested by trypsin after incubation at low pH (lane 3), but F005-126 prevented the low pH-induced conformational change and thus rendered the HA susceptible to protease (lane 7). (B) The amino acid residues involved in salt bridges between HA1 and the B loop in HA2 are depicted as spheres: K109 and K269 in HA1-R (blue) and E67 in HA2-R (purple); K238 in HA1-L (green) and E72 in HA2-R (purple). The following residues in sites R and L are shown as spheres: 91, 92 and 270-273 in HA1-R (cyan), 284 and 285 in HA1-R (magenta), and 171-173, 239, and 240 in HA1-L (red). The illustration was constructed by using the structure of H3 HA (PDB 1HGD) according to molecular graphic viewer Rasmol 2.7.5.

FIGS. 5-1 and 5-2. Amino acid sequences of F005-126 VH and VL (SEQ ID NOs: 2 and 62, respectively). The amino acid numbers are according to the Kabat numbering system. Framework regions (FR) 1, 2, 3, and 4 and complementary determining regions (CDR) 1, 2, and 3 are shown. Comparison of amino acid sequences of F005-126 VH and VL to germline sequences was performed according to IgBLAST (http://www.ncbi.nlm.nih.gov/igblast/). Identity (%) of the amino acid sequences of the VH and the VL with the germline sequences (GHV1-18*01 (SEQ ID NO: 46, and IGLV1-40*01 (SEQ ID NO: 47)) is shown. From X-ray analysis, the amino acid sequences colored blue (denoted with “#”), red (denoted with “+”), and green (denoted with “*”) were bound to site R, site L, and a glycan at HA-R: Asn285, respectively. The amino acid numbers according to Kabat's numbering were shown in FIG. 5-2. As used herein, when referring to paratope numbers or binding region, Kabat's numbering may be used.

FIG. 6. Binding of F005-126 to a mutant Aic68HA/N285Y. FCM analyses of the cells expressing Aic68HA/Wild and a mutant Aic68HA/N285Y were performed. FCM signals for mock-transfection (gray filled), wild HA-expressing cells (green), and the mutant HA-expressing cells (pink) are shown. F49 Ab binds to an epitope on HA2 that is commonly present on H3 subtype viruses. F045-092, F003-137 and F035-015 bind to the HA head region. The reactivity of F005-126 to Aic68HA/N285Y was weaker than that of wild HA.

FIG. 7 Possible recognition site of F005-126. A possible epitope recognized by F005-126 is shown in the 3D structure of HA. The illustration was constructed by displaying the structure of H3 HA (PDB 1HA0) using molecular graphic viewer Rasmol 2.7.5. Residues 50-57 and 275-279 in antigenic site C are shown in yellow. Residues 62-83 in antigenic site E are shown in violet. Residues 39-43, 310-316, and 285 in HA1 are shown in green, cyan, and red, respectively. Residues 9-38 and 317-329 in HA1 are shown in orange. Residues 44-309 except the residues above mentioned are shown in blue. HA2 domain is colored grey. Glycans are colored white.

FIGS. 8-1 and 8-2 Comparison of amino acid sequences of HAs from various strains. The sequences of HA1 (A) and HA2 (B) are shown. The sequence of Aichi/68 H3N2 HA is used as a standard for comparison. The bars indicate the same amino acid as in the standard. Amino acid sequences in site A, B, C, D, and E are boxed. Residues 91, 92, 270-273, 284, and 285 in site R (cyan; columns above denotation “τ”) and residues 171-173, 239, and 240 in site L (red; columns above denotation “*”) are indicated in bold. HA1:238 (green; column above denotation “#”), HA1:109, 269, 299, and 300 (only H7N3) (blue; columns above denotation “+”; except H7N3 for residue 299; only H7N3 for residue 300), HA2:67, 69, and 72 (purple; columns above denotation “φ”) are indicated in bold. Structural data of the following HAs are present in Protein Data Bank: H1N1/SC1918 (1RUZ), H1N1/Cal09pdm (3LZG), H2N2/JPN57 (2WRD), H3N2/Aic68 (1HGD), H5N1/Viet04 (2FK0), H7N3/aviIta02 (1TI8), and H9N2/swHK98 (1JSD). The following amino acids make salt bridges: HA1:109-HA2:69 in H1N1, H2N2, and H5N1; HA1:109-HA2:67, HA1:269-HA2:67, HA1:299-HA2:69, and HA1-L:238-HA2-R:72 in H3N2; HA1:300-HA2:69 in H7N3; HA1:109-HA2:69 and HA1:269-HA2:69 in H9N2.

FIG. 9-1 Salt-bridges between HA1 and B-loop in HA2. Residues involved in the salt-bridges are depicted in spheres: 109, 269, and 300 in HA1-R (blue), and 69 in HA2-R (purple). It seems that thre is no salt-briges between HA1 and B-loop in H5N1 HA according to crystal structure (PDB: 2FK0). Following residues are shown in spheres: 91, 92 and 270-273 in HA1-R (cyan), 284 and 285 in HA1-R (magenta), and 171-173, 239, and 240 in HA1-L (red). Numbering of residues is based on that of H3N2 HA as shown in FIGS. 8-1 and 8-2. FIG. 9-2 shows the amino acid sequences corresponding to site L and R.

FIG. 10 Comparison of crystal structures of HA/Ab complexes. (A) F005-126 Fab: the complex with two HA monomers in an Aic68 HA trimer is depicted. HA-L, HA-R, H chain, and L chain are in green, blue, red, and yellow, respectively. (B) HC45 Fab: the complex with A/X-31(H3N2) HA monomer is depicted (32, PDB 1QFU). HA corresponds to HA-R in (A) and is shown in blue. Upper: the view from the H chain side is shown. Lower: the view from the L chain side is shown. In the lower figures L chains are depicted in ribbons. Residues 171-173, 239 and 240 in site L are in orange. Residues 91, 92, 270-273 in site R are in cyan, and Pro284 and Asn285 are shown in pink. HA1-R: Ser47 is white-colored as a positional marker. The illustrations were constructed using molecular graphic viewer Rasmol 2.7.5.

FIG. 11 Electron density map of X-ray crystallography. The figure shows a 2Fo-Fc electron density map covering the helix of H3 (in blue) from residues 408-423 (79-94 in HA2). The electron density is contoured at 1.5σ. The electron density is a 12-fold non-crystallographically averaged map. In this regard, it is understood that side chains cannot be usually observed at four (4) Angstrom resolution. However, in the subject test, NCS averaging & B-factor sharpening have been conducted and thus side chains have been observed. This is an exceptional case where the minimum unit of the crystal to be tested fall within 6-24 molecules and the like, and such can be averaged to drastically improve the electron density, which is the subject case. The electron density to prove such are shown herein. This Figure shows the case of 12 units (12Fab-4HA (Trimer)).

FIG. 12 X-ray crystallographic analysis of complex: 12Fab-4HA (Trimer) Red: F05126-VH, Yellow:F05126-VL Blue, Light Yellow, Green:HA, ASU [Assymmetric unit] (12Fab-4HA Trimer/ASU), Refine lowers

FIG. 13 X-ray crystallographic analysis of complex: 3Fab-HA (Trimer) Red: F05126-VH, Yellow:F05126-VL Blue, Light Yellow, Green:HA, JKL-90

FIG. 14 The concave region in Aichi/68 H3N2 strain. The concave region's amino acid sequences from various strains were shown in FIGS. 8-1 and 8-2.

FIG. 15 (a-e) Superimposed models of H7(H1,H2,H3,H5,H9)-Fab model by program coot and energy-minimized by CNS. HA docksite, the results are shown in FIGS. 15-a and 15-b. FIG. 15-b is a magnified figure of FIG. 15-a. Docking site has been set with a slightly broad width as shown in the Figure, which is shown in small red spheres and light pink spheres in FIG. 15-c, FIG. 15-d. FIG. 15-e is a binding pocket region when that was carried out in MOE except MOE dock.

FIG. 16 Dock-Sites and dock-results of H3N2 (Aic68)

FIG. 17 Sequence variation and accumulation rates at the Site L and the site R sequences in H1N1, H3N2 and H5N1 derived from different hosts.

FIG. 18. Sequence variation and accumulation rates at the sites L and R in H1N1, H3N2 and H5N1 derived from different hosts. The sequences and the accumulated rates of top 10 sequences at the site L, R, upper and lower epitope regions in H1N1 derived from human and swine as well as H1N1 (pdm); H3N2 derived from human and swine; and H5N1 derived from human and avian are shown in this graph. The most abundant sequence was used as consensus and serially laid out the next rate sequences. (•) indicates the same amino acid residue. Shannon indexes of the sequence variation are also shown.

FIG. 19. Sequence logo of the site L, site R in influenza A virus H1N1, H3N2 and H5N1 derived from different hosts. The Sites L and R were extracted from the Influenza Virus Resource at the National Center for Biotechnology Information: human H1N1, swine H1N1, and H1N1 (pdm); human H3N2 and swine H3N2; and human H5N1 and avian H5N1. All of the sequences in individual subtypes with different host origin were used for this sequence logo analysis.

FIG. 20 depicts selected binding cavity demonstrated in Experiment-6.

FIG. 21 depicts docking pose of Bacitracin.

FIG. 22 depicts docking pose of Colistimethate sodium.

FIG. 23 depicts docking pose of Polymyxin B sulfate.

### MODE FOR CARRYING OUT THE INVENTION

With respect to the present invention, various embodiments will be described below. It should be understood that, throughout the present specification, singular expressions (e.g., “a”, “an”, “the” etc. in the case of English, corresponding articles, adjectives etc. in other languages) also include concepts of its plural, unless otherwise is indicated. In addition, it should be understood that the terms used herein are used in a sense normally used in the art, unless otherwise indicated. Therefore, all specialized terminology and scientific and technical terminology used herein have the same meanings as those generally understood by a person skilled in the field to which the present invention belongs, unless defined differently. When there are some inconsistencies, the present specification (including definitions) prevails.

### DEFINITION OF TERMINOLOGY

Hereinafter, particular terms used herein are provided.

As used herein the term “influenza hemagglutinin (HA)” or “hemagglutinin (HA)”, “influenza hemagglutinin (HA) trimer” or “hemagglutinin (HA) trimer” interchangeably used to refer to a type of hemagglutinin found on the surface of the influenza viruses. Hemagglutinin is known as a substance that causes red blood cells to agglutinate. The HA trimer consists of three monomer of HA molecules. Influenza hemagglutinin is an antigenic glycoprotein. Influenza hemagglutinin is responsible for binding the virus to the cell that is being infected. HA proteins bind to cells with sialic acid on the membranes, such as cells in the upper respiratory tract or erythrocytes. There are at least 17 different HA antigens. These subtypes are named H1 through H17. H16 was discovered only in 2004 on influenza A viruses isolated from black-headed gulls from Sweden and Norway. The most recent H17 was discovered in 2012 in fruit bats, therefore H1-H16 are the main subjects for discussion during analysis. The first three hemagglutinins, H1, H2, and H3, are found in human influenza viruses. Viral neuraminidase (NA) is another protein found on the surface of influenza. Influenza viruses are characterized by the type of HA and NA that they carry; hence H1N1, H3N2, H4N6, H5N2 etc.

HA is a homotrimeric integral membrane glycoprotein. It is shaped like a cylinder, and is approximately 13.5 nanometres long. The three identical monomers that constitute HA are constructed into a central α helix coil; three spherical heads contain the sialic acid binding sites. HA monomers are synthesized as precursors that are then glycosylated and cleaved into two smaller polypeptides: the HA1 and HA2 subunits. Each HA monomer consists of a long, helical chain anchored in the membrane by HA2 and topped by a large HA1 globule. As the HA trimer consists of three identical monomers, when two or more different monomers are referred, such monomers may be represented as “HA-a”, “HA-b” and/or “HA-c”. As such, the term “HA-a+HA-b” refer to dimeric portion of HA trimer or dimer itself.

Since hemagglutinin is the major surface protein of the influenza A virus and is essential to the entry process, it is the primary target of neutralizing antibodies. Neutralizing antibodies against flu have been found to act by two different mechanisms, mirroring the dual functions of hemagglutinin: Inhibition of attachment to target cells, and Inhibition of membrane fusion (entry).

Most commonly, conventional antibodies against hemagglutinin act by inhibiting attachment. This is because these conventional antibodies bind near the top of the hemagglutinin “head” and physically block the interaction with sialic acid receptors on target cells. In contrast, some conventional antibodies have been found to have no effect on attachment. Instead, this latter group of conventional antibodies acts by preventing membrane fusion. Most of these conventional antibodies recognize sites in the stem/stalk region, far away from the receptor binding site.

As used herein the term “a functionally equivalent sequence (or variant)” refers to an entity such as antibody or hemagglutinin which may vary in terms of structure (sequence or variant) but is the same or similar to the original protein or gene product or the like. Functionally equivalent sequence or proteins or peptides may be created via the application of recombinant DNA technology, in which changes in the protein structure may be engineered, based on considerations of the properties of the amino acids being exchanged. Skilled in the art may introduce designed changes through the application of site-directed mutagenesis techniques or mutations may be introduced randomly and screened later for the desired function, as described below.

As used herein “functionally equivalent sequence” of the “sequence of CDR1” (of a heavy chain or light chain of an antibody) refers to a sequence derived from an original CDR1 sequence such as SEQ ID NO: 3 in the case of antibody F05126 heavy chain, but with variations such as substitution(s), addition(s) and/or deletion(s), while maintaining at least one of the functions possessed by the original CDR1, such as antigen binding activity and/or specificity and the like.

As used herein “functionally equivalent sequence” of the “sequence of CDR2” (of a heavy chain or light chain of an antibody) refers to a sequence derived from an original CDR2 sequence such as SEQ ID NO: 4 in the case of antibody F05126 heavy chain, but with variations such as substitution(s), addition(s) and/or deletion(s), while maintaining at least one of the functions possessed by the original CDR2, such as antigen binding activity and/or specificity and the like.

As used herein “functionally equivalent sequence” of the “sequence of CDR3” (of a heavy chain or light chain of an antibody) refers to a sequence derived from an original CDR3 sequence such as SEQ ID NO: 5 in the case of antibody F05126 heavy chain, but with variations such as substitution(s), addition(s) and/or deletion(s), while maintaining at least one of the functions possessed by the original CDR3, such as antigen binding activity and/or specificity and the like.

As used herein “functionally equivalent sequence” of the “sequence of FR1” (of a heavy chain or light chain of an antibody) refers to a sequence derived from an original FR1 sequence such as SEQ ID NO: 6 in the case of antibody F05126 heavy chain, but with variations such as substitution(s), addition(s) and/or deletion(s), while maintaining at least one of the functions possessed by the original FR1, such as antigen binding activity and/or specificity and the like.

As used herein “functionally equivalent sequence” of the “sequence of FR2” (of a heavy chain or light chain of an antibody) refers to a sequence derived from an original FR2 sequence such as SEQ ID NO: 7 in the case of antibody F05126 heavy chain, but with variations such as substitution(s), addition(s) and/or deletion(s), while maintaining at least one of the functions possessed by the original FR2, such as antigen binding activity and/or specificity and the like.

As used herein “functionally equivalent sequence” of the “sequence of FR3” (of a heavy chain or light chain of an antibody) refers to a sequence derived from an original FR3 sequence such as SEQ ID NO: 8 in the case of antibody F05126 heavy chain, but with variations such as substitution(s), addition(s) and/or deletion(s), while maintaining at least one of the functions possessed by the original FR3, such as antigen binding activity and/or specificity and the like.

As used herein “functionally equivalent sequence” of the “sequence of FR4” (of a heavy chain or light chain of an antibody) refers to a sequence derived from an original FR4 sequence such as SEQ ID NO: 9 in the case of antibody F05126 heavy chain, but with variations such as substitution(s), addition(s) and/or deletion(s), while maintaining at least one of the functions possessed by the original FR4, such as antigen binding activity and/or specificity and the like.

As used herein the term “having broad strain specificity against H3” refers to a specificity of an antibody which can bind to at least two, three, four, five, ten, twenty, fifty, a hundred, one thousand, and the like of the strains within H3, and preferably all strains falling within H3. It has been known that all H3 variants that the 285th amino acid has a carbohydrate chain. In addition to human and pig origin H3, the antibody (F005-126) may have specificity to bird origin H3N8. A side chain of FR3 of an antibody couples to the main chain of the HA by van der Waals forces. Thus, it is thought that specificity is not affected by variation therein.

As used herein the term “binds to HA1 head region but does not inhibit binding to cell” refers to the property of an antibody, particularly a neutralizing antibody, and particularly refers that such an antibody binds to HA1 head region, but has no or substantially no HI activity. As used herein the HA1 head region refers to amino acid positions 39 to 319 of HA1 in the case of SEQ ID NO: 48. (residues 39-319) As used herein “HI (hemagglutination inhibition)” activity refers to the HA assay used to measure flu-specific antibody levels in blood serum. This property is a new inhibition mode for an antibody. An antibody usually binds to an epitope of an HA antigen in (1) HA1 or (2) HA2. If the epitope is located on HA1, such an antibody usually has HI activity. On the other hand, if the epitope is located on HA2, such an antibody has an activity of membrane fusion inhibition. The antibodies of the invention, having “binds to HA1 head region but does not inhibit binding to cell,” will bind to HA1, but do not have HI activity. It is believed that such an antibody binds to HA from the side, has a new neutralization mode in addition to conventional inhibition modes such as (1) HA1-HI+ and (2) HA2-Fusion inhibition.

As used herein the term “inhibits structural change of HA” refers to inhibiting initial conformational change of HA1 and the ensuing drastic conformational change of HA2 induced by low pH which is required for membrane fusion in the process of infection of influenza viruses. As used herein, “fusogenic conformational change” refers to a type of conformational change, which facilitating fusion of cells.

As used herein the term “bind to . . . by van der Waals contact” refers to a mode of binding reaction wherein such a binding reaction is achieved by van der Waals contact. Such a van der Waals contact may be measured by crystallography known in the art as exemplified herein.

As used herein the term “binds to the HA trimer across two HA subunits thereof which are adjacent to each other” refers to Site R, Site L covers up two Salt bridge in a liaison part of HA1 and HA2. As a result, even if pH is in an acidic range (4.5-5.5), a head domain of HA1 does not cause a substantial structural change.

As used herein the term “intra- and inter-subunit interactions between HA1 and HA2 by salt bridges” refers to interactions which are located on the amino acid sequence of the molecular surface in the vicinity of the portion which maintains structure of the HA trimer and avoids disassembling from a trimer to a monomer. It is known that cell fusion is the mechanism which is indespensable to the growth of an influenza virus, virus multiplication can be inhibited by disfunctioning the mechanism switch which is effected by the intra- and/or inter salt bridges.

As used herein the term “comprising hydrogen bond” refers to an interaction between two or more entities such as molecules, wherein the interaction comprises hydrogen bond. Such hydrogen bond may be measured by crystallography known in the art as exemplified herein.

As used herein the term “positions other than the binding site with HA of CDR1 sequence of F005-126 antibody (amino acid No. 31 (Ser) of SEQ ID NO. 2), the binding site with HA of CDR2 sequence of F005-126 antibody (SEQ ID NO: 10 (amino acids No. 54-58 (Tyr Asn Gly Asn Thr) of SEQ ID NO. 2)), the binding site with HA of CDR3 sequence of F005-126 antibody (amino acids No. 74-76 (Thr Ser Thr) of SEQ ID NO. 2), and the binding site with HA of FR3 sequence of F005-126 antibody (SEQ ID NO: 11 (amino acids No. 102-105 (Val Arg Gly Val) of SEQ ID NO. 2))” refers to positions in the sequences of CDR1, CDR2, CDR3 and FR3 wherein the positions do not affect the function of the original antibody, such as interaction with HA trimer, particularly within the concave region.

As used herein the term “a sequence maintains the binding activity with the HA trimer” refers to a variant sequence which has variations such as a substitution, addition and/or deletion or the like derived from an original sequence (such as F005-126 antibody, HA trimer and the liker), while maintaining the antigen-antibody binding activity between the original antibody and the original antigen.

As used herein the term “paratope” refers to the part of an antibody which recognizes an antigen, the antigen-binding site of an antibody. It is a small region (of e.g. 15-22 amino acids) of the antibody's Fv region and contains parts of the antibody's heavy and light chains. The part of the antigen to which the paratope binds is called an epitope. This can be mimicked by a mimotope. The figure given on the right hand side depicts the antibody commonly found on a B leukocyte. The engraved inner portions of idiotype are the paratopes where the epitope of the antigen binds.

As used herein the term “paratope analysis” refers to any analysis for finding or screening analysis from candidate entities, and/or any study for detailed characteristics for known paratopes.

As used herein the term “concave region” of HA trimer refers to the region of the HA trimer, particularly type H3 HA trimer but not limited thereto, wherein the region specifically interacts with the antibody F005-126 or equivalent thereof, which forms concave-cap structure. Such a concave region is not formed in the monomer state. However, when the monomers form a trimer, such concave regions are formed between the two monomers of the trimer. As such, there are three concave regions in the trimer in total. Specifically, such a concave region is represented by the case of H3N2 Aic68 (SEQ ID NOs: 48 and 21, the following amino acid residues refer to those in SEQ ID NO: 48 for HA1 and SEQ ID NO: 21 for HA2):

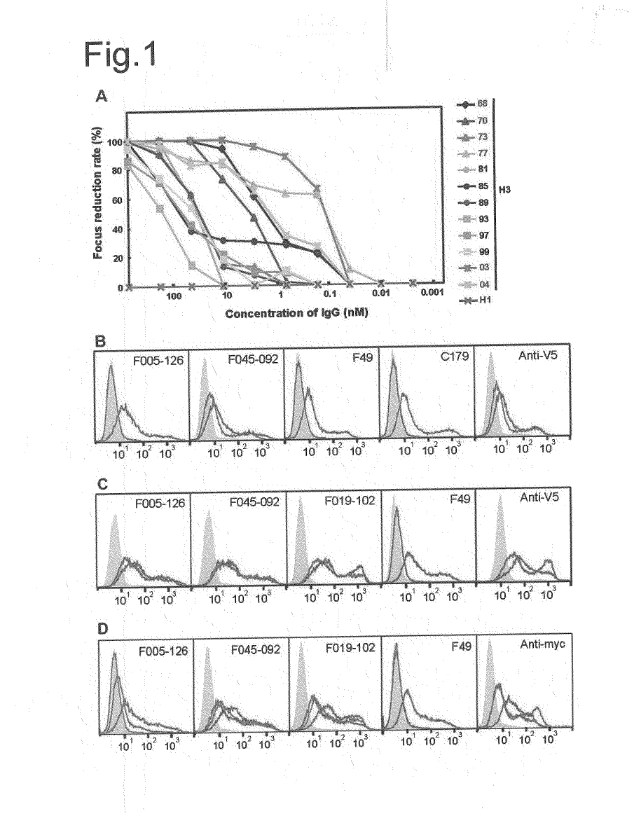

HA1-Site R (shown in brown in FIGS. 8-1 and 8-2)

Ser47 Thr48 Gly49 Lys50 Asp60 Ile62 Asp63 Cys64
Thr65 Asp68 Asp73 His75 Glu89 Arg90 Ala93 Phe94
Ser95 Asn96 Arg109 Pro221 Trp222 Val223 Arg224
Gly225 Arg269 Ile274 Asp275 Asn296 Val297 Asn298
Lys299 Ile300 Tyr308 val309 Lys310

HA2-Site R (shown in brown in FIGS. 8-1 and 8-2)

Glu67 Lys68 Glu69 Phe70 Ser71 Glu72 Asp86 Ile89
Asp90

HA1-Site L (shown in pink in FIGS. 8-1 and 8-2)

Ser114 Ser115 Glu123 Thr167 Met168 Pro169 Asn170
Phe174 Asp175 Lys176 Tyr178 Arg207 Arg208 Lys238
Asp241 Val242 Val244 Tyr257 Lys259 Met260 Arg261
Thr262 Gly263 Lys264 Ser265

HA2-Site L (shown in pink in FIGS. 8-1 and 8-2)

Glu61 Lys62 Phe63

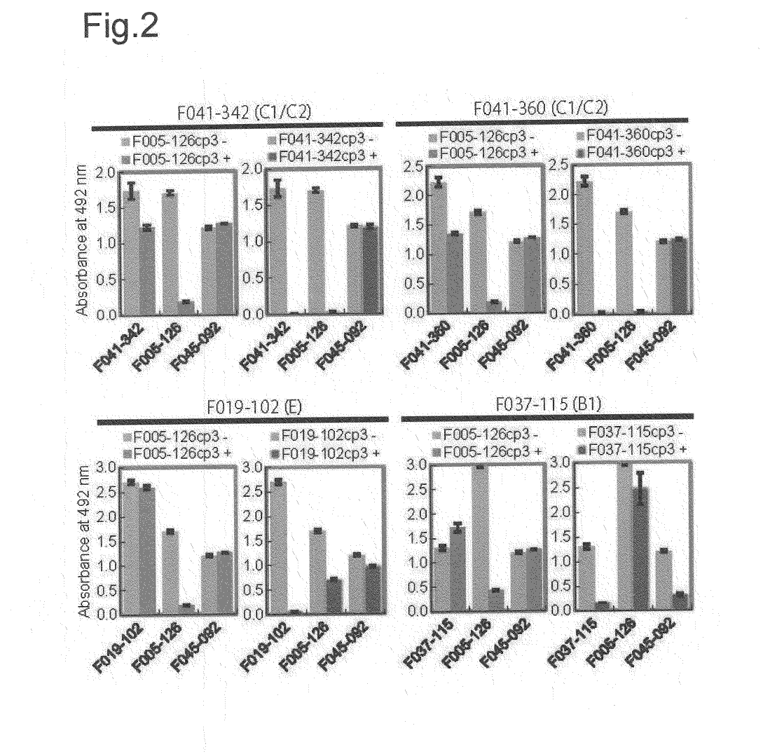

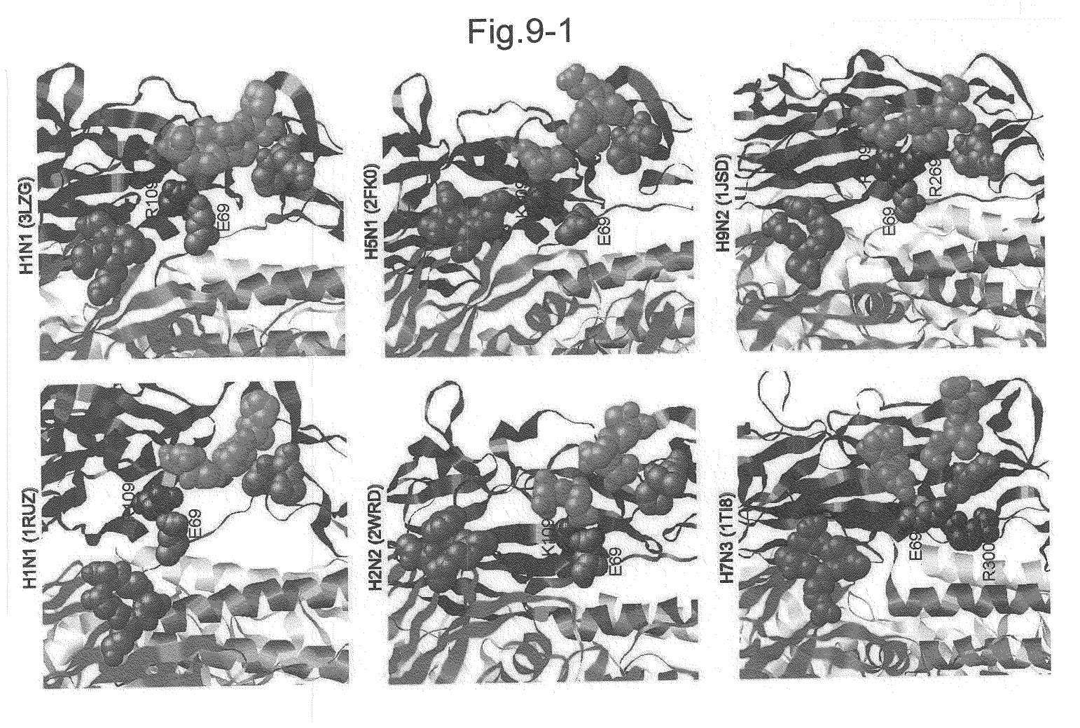

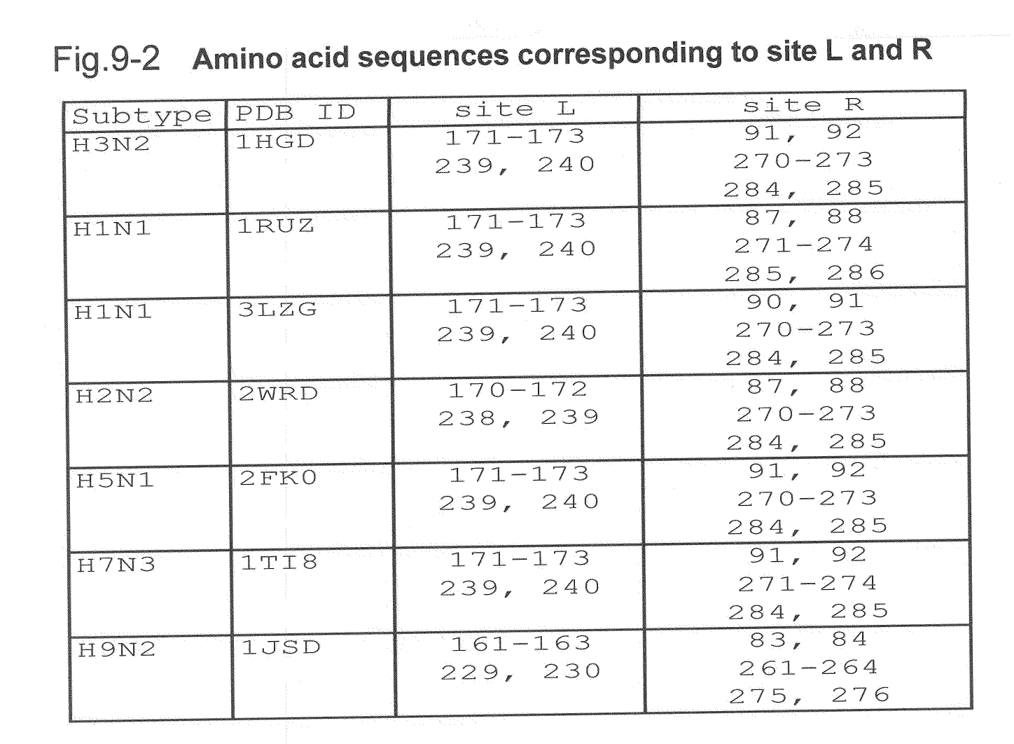

but the concave region is not limited to the above identified residues, and may vary depending on the strain of interest, but the skilled in the art may appropriately determine the corresponding amino acid residues which define the concave region, in reference to FIG. 9-2, as necessary. Furthermore, the actual amino acid residues may vary depending on the software or application that is actually employed.

As used herein the term “positions other than the sequence of concave region” refers to positions of HA trimer except for the concave region as defined herein above. The positions other than the sequence of concave region should likely be free of negative effects on the interaction of the HA trimer and F005-126 antibody or equivalent thereof.

As used herein the term “Group 2 hemagglutinin” refers to a group of hemagglutinin, to which type H3 and the like belongs. Hemagglutinin subtypes divided into two main groups: group 1 and group 2. Phylogenetically, there are two groups of HAs: group 1 contains H1, H2, H5, H6, H8, H9, H11, H12, H13, and H16, and group 2 contains H3, H4, H7, H10, H14, and H15 (14, 15). Newly found H17 is closedly related to Group 1.

As used herein the term “hemagglutinin H3” refers to a type of hemagglutinin, which infects human. Influenza hemagglutinin (H3 serotype) was the first glycoprotein structure to be solved at atomic resolution, by Ian Wilson, John Skehel and Don Wiley in 1981. The collaboration between the Skehel and Wiley labs provided great insight into hemagglutinin function, and it remains the prototype for understanding receptor recognition, antigenic variation, and the extraordinary conformational changes associated with target membrane insertion and ultimately fusion of viral with cell membrane to allow the viral genome to enter the cell and replicate.

As used herein the term “hemagglutinin H3 strain” includes strains Aic68, Fuk70, Tok73, Yam77, Nii81, Fuk85, Gui89, Kit93, Syd97, Pan99, Wyo03 and NY04 and the like, and are not limited thereto. Therefore, any hemagglutinin H3 strains may be used for the present invention.

As used herein the term “a candidate substance” refers to a substance which is a candidate for an object of an assay, such as a binding substance when the substance is concerned with binding assay.

As used herein the term “a binding substance” refers to a substance which has a binding activity against to a target.

As used herein the term “dock” refers to aligning the 3D structures of two or more molecules to predict the conformation of a complex formed from the molecules as exemplified and described herein detail.

As used herein the term “epitope element” refers to a portion of element which forms an epitope together with one or more different epitope element(s). Usually such an epitope formed by a plurality of epitope elements is a conformational epitope.

As used herein the term “paratope element” refers to a portion of element which forms a paratope together with one or more different paratope element(s).

As used herein the term “Site L epitope element” refers to a part of HA trimer, a Site L epitope element comprising amino acid residues N171, D172 and N173 (herein also refers to HA-a-L1) and P239 and G240 (herein also refers to HA-a-L2). The term Site L may herein also be referred to simply “L.”

As used herein the term “Site R epitope element” refers to a part of HA trimer, a Site R epitope element comprising amino acid residues S91 and K92 (herein also refers to HA-b-R1), S270, D271, A272, and P273 (herein also refers to HA-b-R2) and P284 and N285 (herein also refers to HA-b-R3). The term Site R may herein also be referred to simply “R”.

A partial or full complex of a conformational epitope and paratope formed by an antibody-antigen complex of antibody F005-126, antigen HA-a+HA-b and water molecule, wherein the HA-a and HA-b are HA1 selected from the group consisting of SEQ ID NOs: 48-60 and 39-45 and HA2 selected from the group consisting of SEQ ID NOs: 21-38, wherein the conformational epitope comprises:

the following amino acid residues of the amino acid sequences of HA of H3N2 Aic 68 (SEQ ID NO: 48), or corresponding amino acid residues thereto:

Site L epitope element comprising amino acid residues N171, D172 and N173 (HA-a-L1) and P239 and G240 (HA-a-L2): HA-a-L1 and HA-a-L2 form a site which is herein referred to as Site L. The positions that form salt bridges are located on K238 and E72, and the salt bridges are formed by HA-a-(HA1)-K238 and HA-b-(HA2)-E72 which are referred to Site-LS. (S was named in connection with S of Salt bridge.)

Site R epitope element comprising amino acid residues S91 and K92 (HA-b-R1), S270, D271, A272 and P273 (HA-b-R2), and P284 and N285 (HA-b-R3). HA-b-R1, HA-b-R2, and HA-b-R3 form a site which is herein referred to as Site R. The positions that form salt bridges are located on R109 and 8269 on HA1, and E67, and the salt bridges are formed by HA-b-(HA1)-R109 and HA-b-(HA2)-E67, HA-b-(HA1)-R269 and HA-b-(HA2)-E67 which are referred to Site-RS. (S stands for salt bridge(s).)

A Site R epitope element comprising sugar chains NAG(N-acetyl-D-glucosamine)1, NAG2, BMA(beta-D-mannose)3, MAN(alpha-D-mannose)4, MAN5, MAN6 and MAN7, linked to amino acid residue N285 (the sugar chains ding to 285N. This binding moiety is referred as HA-b-G).

HA-b-G, HA-b-R1, HA-b-R2, and HA-b-R3 form a site which is herein referred to as Site RG.

Site-RS and HA-b-G form a site which is herein referred to as Site RSG.

As used herein, the paratope of the invention comprises:

the following amino acid residues of F005-126 heavy chain (SEQ ID NO:2), or corresponding amino acid residues thereto:

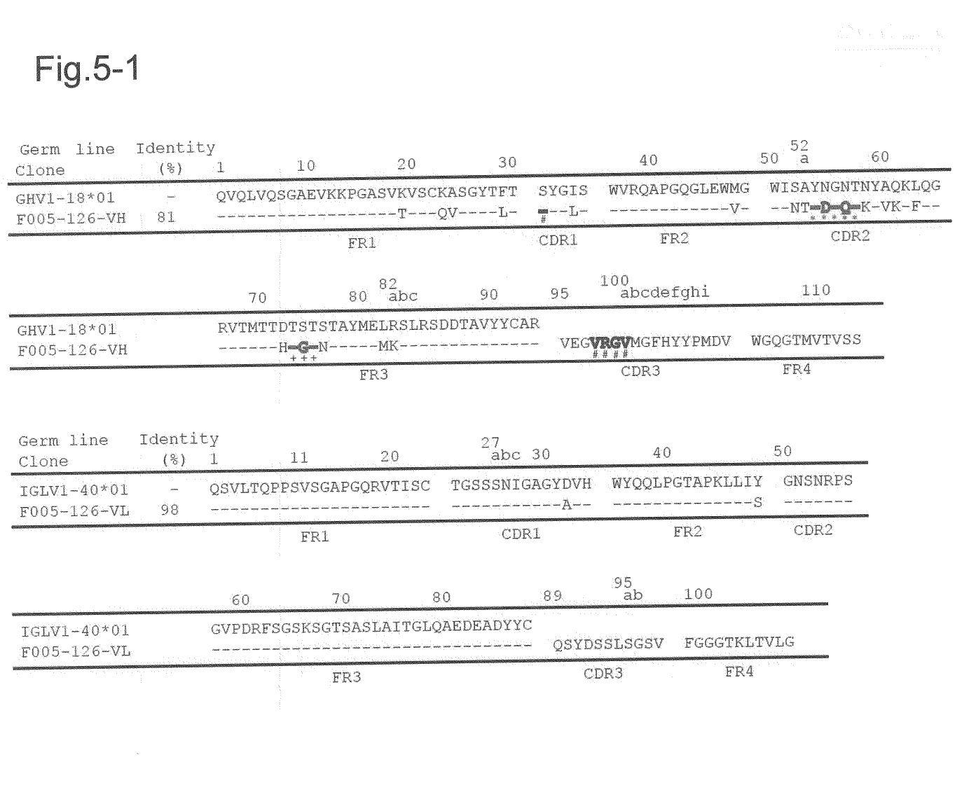

a paratope element comprising amino acid residues T73, G74, and T75 (according to the Kabat's numbering shown in FIG. 5-2) (named as P-Site-L); as used herein paratope binding Site L is called P-Site-L. (in FR3)

a paratope element comprising amino acid residues S31 (according to the Kabat's numbering shown in FIG. 5-2) (named as P-Site-R-1) (in CDR1);

a paratope element comprising amino acid residues Y53, D54, G55, Q56 and H57 (according to the Kabat's numbering shown in FIG. 5-2) (named as P-Site-R-2) (in CDR2);

a paratope element comprising V98, R99, G100, and V100a (according to the Kabat's numbering shown in FIG. 5-2) (named as P-Site-R-3) (in CDR3);

P-Site-R-1, P-Site-R-2 and P-Site-R-3 form “P-Site-R”. Site-LS and FR3 (P-Site-L) form “EP-L” (epitope-paratope-L). Site RSG and CDR1, CDR2 and CDR3 (P-Site-R) form “EP-R”. Binding region of antibody F005-126 (VH) to the HA trimer spans between EP-L and EP-R. EP-L and EP-R form EP-LR.

As used herein the term “a partial complex” refers to a part of a complex. Depending on the context, such a partial complex may encompass the entire complex.

As used herein, “protein”, “polypeptide”, “oligopeptide” and “peptide” refer to a polymer of amino acids of any length. This polymer may be linear, branched, or cyclic. The amino acid may be naturally-occurring, non-naturally-occurring, or may be an altered amino acid. This term can also include an assembly of a plurality of polypeptide chains into a complex. This term also includes natural or artificially altered amino acid polymers. Such alteration includes disulfide bond formation, glycosylation, lipidation, acetylation, phosphorylation or any other manipulation or alteration (e.g. conversion into a bound body with a labeling component). This definition also includes, for example, polypeptides including one or two or more analogs of amino acids (e.g. including non-naturally-occurring amino acids), peptide-like compounds (e.g. peptoids) and other alterations known in the art.

As used herein, an “amino acid” may be naturally-occurring or non-naturally-occurring, as far as the object of the present invention is satisfied.

As used herein, “nucleic acid” can also be used interchangeably with a gene, cDNA, mRNA, an oligonucleotide, and a polynucleotide. A particular nucleic acid sequence also includes “splice variants”. Similarly, a particular protein encoded by a nucleic acid implicitly includes any protein encoded by a splice variant of the nucleic acid. As suggested by its name, a “splice variant” is a product of alternative splicing of a gene. After transcription, a first nucleic acid transcript can be spliced so that a different (another) nucleic acid splice product encodes a different polypeptide. The mechanism of producing the splice variant includes alternative splicing of an exon as well as other means. A different polypeptide derived from the same nucleic acid sequence by transcription readthrough is also included in this definition. Any product of a splicing reaction (including a recombinant splice product) is also included in this definition. An allele variant also falls into this range.

As used herein, “polynucleotide”, “oligonucleotide” and “nucleic acid” are used in the same sense, and refer to a polymer having a nucleotide of any length. This term also includes “oligonucleotide derivative” or “polynucleotide derivative”. “Oligonucleotide derivative” or “polynucleotide derivative” includes a derivative of a nucleotide, or refers to an oligonucleotide or a polynucleotide in which binding between nucleotides is different from normal binding; these are interchangeably used. Examples of such oligonucleotide specifically include 2′-O-methyl-ribonucleotide, an oligonucleotide derivative in which a phosphate diester bond in an oligonucleotide was converted into a phosphorothioate bond, an oligonucleotide derivative in which a phosphate diester bond in an oligonucleotide was converted into an N3′-P5′ phosphoroamidate bond, an oligonucleotide derivative in which ribose and a phosphate diester bond in an oligonucleotide were converted into a peptide nucleic acid bond, an oligonucleotide derivative in which uracil in an oligonucleotide was substituted with C-5 propynyluracil, an oligonucleotide derivative in which uracil in an oligonucleotide was substituted with C-5 thiazoleuracil, an oligonucleotide derivative in which cytosine in an oligonucleotide was substituted with C-5 propynylcytosine, an oligonucleotide derivative in which cytosine in an oligonucleotide was substituted with phenoxazine-modified cytosine, an oligonucleotide derivative in which ribose in DNA was substituted with 2′-O-propylribose and an oligonucleotide derivative in which ribose in an oligonucleotide was substituted with 2′-methoxyethoxyribose. It is intended that a particular nucleic acid sequence also includes a variant thereof which was conservatively altered (e.g. a degenerate codon substituted body) and a complementary sequence thereof, like an explicitly shown sequence, unless otherwise indicated. Specifically, the degenerate codon substituted body can be attained by making a sequence in which the third position of one or more selected (or all) codons is substituted with a mixed base and/or deoxyinosine residue (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)).

As used herein, “nucleotide” may be naturally-occurring or non-naturally-occurring, as far as the objective function is retained.

An amino acid can be referred to herein, by either of the generally known three letters symbol thereof, or one letter symbol recommended by IUPAC-IUB Biochemical Nomenclature Commission. A nucleotide can be similarly referred by the generally recognized one letter code.

As used herein, “sugar chain” refers to a compound produced by connection of one or more sugar units (monosaccharide and/or derivative thereof). When two or more sugar units are connected, respective sugar units are bound by dehydration condensation with a glycoside bond. Examples of such a sugar chain include, but are not limited to, in addition to polysaccharides (glucose, galactose, mannose, fucose, xylose, N-acetylglucosamine, N-acetylgalactosamine, sialic acid as well as complexes and derivatives thereof), a variety of sugar chains degraded or derived from complex biomolecules such as degraded polysaccharides, glycoproteins, proteoglycans, glycosaminoglycans, and glycolipids. Therefore, as used herein, the sugar chain can be used interchangeably with “sugar”, “polysaccharide”, “glucide”, and “carbohydrate”. In addition, when not particularly referred, as used herein, “sugar chain” includes both of a sugar chain and a sugar chain-containing substance. Representatively, the sugar chain is a substance in which about 20 kinds of monosaccharides (glucose, galactose, mannose, fucose, xylose, N-acetylglucosamine, N-acetylgalactosamine, sialic acid as well as complexes and derivatives thereof) are connected in chains, and is attached to proteins or lipids inside or outside the cells of living bodies. The sugar chain is different in function depending on a sequence of a monosaccharide, and is usually complexly branched; it is predicted that there are several hundred kinds or more of sugar chains having various structures in the human body and, further, it is thought that there are several tens of thousands or more types of structures useful in the human body. It is believed that the sugar chain is involved in the high order function served by proteins or lipids in living bodies, such as molecule/cell recognizing function between cells, but the majority of the mechanism is unknown. Sugar chains are studied in current life science as a third biological polymer next to nucleic acids and proteins. Inter alia, the function of the sugar chain as a ligand in cellular recognition (information molecules) is expected, and application thereof to the development of high-functional materials has been studied.

As used herein, “sugar chain group” is a name given when the sugar chain binds to another group. The sugar chain group refers to monovalent or divalent groups depending on the case. Examples of the sugar chain group include a sialyl Lewis X Group, an N-acetyllactosamine group, and an α1-6 mannobiose group. Among abbreviations of the sugars used as used herein, MAN is mannose, Neu5Ac is N-acetylneuraminic acid, Gal is galactose, GlcNAc or NAG is N-acetylglucosamine, GalNAc is N-acetylgalactosamine, and R is a non-sugar part (e.g., peptide, protein, lipid etc.) and BMA refers to b eta-D-mannose.

In the present specification, “isolated” substance (e.g., a biological factor, such as a nucleic acid or a protein) refers to a substance substantially isolated or purified from other substances (preferably, biological factors) in the environment in which such a substance is naturally-occurring (e.g., in the cells of an organism) (for example, the “isolated” substance means that, in the case of the nucleic acid, factors other than nucleic acids and nucleic acids containing nucleic acid sequences other than that of the nucleic acid of interest; and, in the case of the protein, factors other than proteins and proteins containing amino acid sequences other than that of the protein of interest). The term “isolated” nucleic acid and protein encompasses nucleic acids and proteins purified by standard purification techniques. Therefore, the isolated nucleic acid and protein encompasses chemically synthesized nucleic acids and proteins.

In the present specification, “purified” substance (e.g., a biological factor such as a nucleic acid or a protein) refers to one from which at least a portion of naturally accompanying factors has been removed. Therefore, ordinarily, the purity of a purified substance is higher than that of a substance in a normal state (i.e., concentrated).

In the present specification, “purified” and “isolated” mean that the same type of a substance is present preferably at least 75% by weight, more preferably at least 85% by weight, even more preferably at least 95% by weight, and most preferably at least 98% by weight.

As used herein, “homology” of a gene refers to a degree of identity of 2 or more gene sequences to each other. Therefore, as homology of 2 genes is higher, identity or similarity of those sequences is higher. Whether two kinds of genes have homology or not can be investigated by direct comparison of sequences, or in the case of a nucleic acid, by a hybridization method under the stringent conditions. When two gene sequences are directly compared, in the case where a DNA sequence is representatively at least 80% identical, preferably at least 90% identical, and more preferably at least 95% identical between the gene sequences, those genes have homology.

In the present invention, comparisons of similarity, identity and homology of amino acid sequences and nucleotide sequences are calculated using default parameters employing the BLAST tool for sequence analysis. Retrieval of identity can be performed, for example, using BLAST 2.2.9 of NCBI (issued on May 12, 2004). The value of identity as used herein usually refers to a value when alignment is performed under the default conditions using the BLAST, provided that when a higher value is obtained by change in a parameter, the highest value is adopted as a value of identity. When identity is assessed in a plurality of regions, the highest value among values is adopted as the value of identity.

As used herein, a “corresponding” gene refers to a gene which has, or is predicted to have the same action as that of a predetermined gene in a species or strain such as type H3 HA as a standard of comparison, in a certain species and, when there are a plurality of genes having such an action, refers to a gene having the evolutionally same origin. Therefore, a gene corresponding to a certain gene (e.g. HA) can be the orthologue of the gene. Therefore, a gene corresponding to a type H3 influenza virus gene can be also found in other species or strain of the virus (e.g. Groups 1 and, H1, H2, H4, H5 and the like). Such a corresponding gene can be identified using techniques well known in the art. Therefore, for example, a corresponding gene in a certain strain or species can be found by retrieving a sequence database of the virus (e.g. Groups 1 and, H1, H2, H4, H5 and the like) and using a sequence of a gene as a standard for the corresponding gene as a query sequence.

As used herein, the “corresponding” amino acid (residue) means an amino acid (residue) in a protein or polypeptide molecule that has or expected to have an action, such as a site of interaction including antigen-antibody binding, neutralizing activity, similar to that of a predetermined amino acid (residue) in the protein or polypeptide to be compared. Corresponding amino acid residues may be analyzed by using sequence alignment and the like, e.g. HA sequences and antibody CDR1, CDR2, CDR3 and FR3 sequences and the like

As used herein, “fragment” refers to a polypeptide or a polynucleotide having a sequence length of 1 to n−1 relative to a full length polypeptide or polynucleotide (length is n). The length of the fragment can be appropriately changed depending on the purpose thereof, and examples of a lower limit of the length, in the case of the polypeptide, includes 3, 4, 5, 6, 7, 8, 9, 10, 15, 20, 25, 30, 40, 50 and more amino acids, and any length represented by an integer not specifically listed herein (e.g. 11 etc.) can be also suitable as the lower limit. In the case of the polynucleotide, examples of a lower limit of the length includes 5, 6, 7, 8, 9, 10, 15, 20, 25, 30, 40, 50, 75, 100 and more nucleotides, and any length represented by an integer not specifically listed herein (e.g. 11 etc.) can be also suitable as the lower limit. As used herein, the lengths of the polypeptide and the polynucleotide can be represented by the number of amino acids or nucleic acids, respectively, as described above, but the aforementioned number is not absolute, and it is intended that the aforementioned number as the upper limit or the lower limit also includes numbers which are a few more or less (or e.g. 10% more or less) than the number, as far as the same function is possessed. In order to express such an intention, as used herein, the intention is expressed by adding “about” before the number, in some cases. However, as used herein, it should be understood that the presence or absence of “about” does not influence the interpretation of the numerical value. As used herein, the length of a useful fragment can be determined as whether at least one function among the functions of a full length protein as a standard of the fragment is retained or not.

As used herein, “variant”, “variant sequence” or “analog” refers to one in which a part is changed relative to the original substance such as a polypeptide or a polynucleotide. Examples of such a variant include a substitution variant, an addition variant, a deletion variant, a truncated variant, and an allelic variant. The allele refers to genetic variants which belong to the same locus, and are discriminated from each other. Therefore, “allele variant” refers to a variant which is in a relationship of an allele relative to a certain gene. “Homolog” refers to one having homology (preferably, 80% or more homology, more preferably, 90% or more homology) with a certain gene at an amino acid level or a nucleotide level, in a certain species. A method of obtaining such a homolog is apparent from the description of the present specification.

In the present invention, in order to make a functionally equivalent polypeptide, addition, deletion or modification of an amino acid in addition to substitution of an amino acid can be also performed. Substitution of an amino acid refers to substitution of the original peptide with 1 or more, for example, 1 to 10, preferably 1 to 5, more preferably 1 to 3 amino acids. Addition of an amino acid refers to addition of 1 or more, for example, 1 to 10, preferably 1 to 5, more preferably 1 to 3 amino acids to the original peptide chain. Deletion of an amino acid refers to deletion of 1 or more, for example, 1 to 10, preferably 1 to 5, more preferably 1 to 3 amino acids from the original peptide. Amino acid modification includes, but is not limited to, amidation, carboxylation, sulfation, halogenation, alkylation, phosphorylation, hydroxylation, and acylation (e.g. acetylation). An amino acid to be substituted or added may be a naturally-occurring amino acid, a non-naturally-occurring amino acid, or an amino acid analog, and a naturally-occurring amino acid is preferable.

A nucleic acid encoding such a polypeptide can be obtained by a well-known PCR method, or can be chemically synthesized. These methods may be combined, for example, with a site-specific mutagenesis method, or a hybridization method.

As used herein, “substitution, addition and/or deletion” of a polypeptide or a polynucleotide refers to substitution, addition, or removal of an amino acid or a substitute thereof, or a nucleotide or a substitute thereof, respectively, relative to the original polypeptide or polynucleotide. The technique of such substitution, addition, and/or deletion is well-known in the art, and examples of such techniques include a site-specific mutagenesis technique. These changes in a nucleic acid molecule or a polypeptide as a standard can be generated at a 5′ end or a 3′ end of this nucleic acid molecule, or can be generated at an amino terminal or a carboxy terminal of an amino acid sequence indicating this polypeptide, or can be generated anywhere between those end sites, and can be individually scattered between residues in a standard sequence, as far as the objective function (e.g. binding to HA in an anti-HA antibody, an antigen-binding fragment thereof or a HA-binding molecule) is retained. Substitution, addition, or deletion may be any number as far as the number is 1 or more, and such a number is not limited, as far as the objective function (e.g. binding to HA) is retained in a variant having the substitution, the addition or the deletion. For example, such a number can be 1 or a few, preferably within 5% of the full-length, or 25 or less.

As used herein, “similar amino acid” refers to an amino acid in a relationship of conservative substitution, and the following amino acids correspond thereto. It is understood that variants in which the following substitution was performed also fall within the scope of the present invention, from the particular sequence (e.g. 1B2) of the present invention.

### A: G, I, V, L

C: M (S-containing amino acid)

### D: N, Q or E

### E: N, Q or D

### F: Y, A etc.

### G: A

### H: W etc.

### I: A, L, V, (G)

### K: R

### L: A, I, V, (G)

### M: S etc.

### N: E, D or Q

### P: HyP

### Q: N, E or D

### R: K

### S: T, Y

### T: S, Y

### V: I, L, A, (G)

### W: H

### Y: F, S, T

Substitution between these amino acids is also referred to as “conservative substitution.”

(Antibody)

As used herein, “antibody” collectively refers to a protein which is produced in a living body by stimulation with an antigen and specifically binds to or reacts with an antigen, in an immune reaction, or proteins having the same sequence thereto, which were produced by chemical synthesis, etc. The antibody is actually an immunoglobulin, and is also referred to as Ab.

F005-126 antibody (herein also referred to as “F5126” (antibody), “F05126” (antibody), which are interchangeably used herein) is a type of IgG, and as shown in FIG. 1A and consists of SEQ ID NOs: 2 and 62. F005-126 antibody neutralizes 12 strains of H3N2 viruses with various activities ranging from 0.1 to 100 nM. In the present invention, we analyzed the epitope recognized by F005-126, and it has been shown that the binding regions are located in the Site L and Site R, which is referred to as “concave region”, which is defined elsewhere herein.

As used herein, “antigen-binding fragment” of an antibody refers to, regarding a certain antibody, a fragment having a binding property to the same antigen as an antigen of the antibody. Whether the antibody falls into the scope of such “antigen-binding fragment” or not can be assessed by an affinity assay described as used herein. As used herein, such affinity can be indicated using a concentration at which a binding amount of a labeled HA molecule to an antibody is 50% inhibited (IC₅₀ value) as an index, and the IC₅₀ value can be calculated, for example, by a regression model based on a logistic curve (Rodbard et al., Symposium on RIA and related procedures in medicine, P165, Int. Atomic Energy Agency, 1974).

As used herein, “anti-HA antibody” refers to an antibody which was raised against HA, or has a binding ability equivalent thereto.

As used herein, “interaction” with a certain antigen refers to an influence on each other without the need of binding.

As used herein, immunoglobulin “heavy chain variable domain (VH)” and “light chain variable (VL) domain” are used in the sense usually used in the art. Immunoglobulin is such that two L chains (light chains) and two H chains (heavy chains) having the same fundamental structure are connected with an S—S bond, the H chains are connected so that two fragments of Fc (crystallizable fragment) on a C terminal side and Fab (antigen binding fragment) on an N terminal side are bent at the hinge part, and a Y letter form is taken as a whole. In both of the L chain and the H chain, a sequence of about 110 amino acids (about half the length of the L chain) from the N terminal is a sequence which is partially different depending on antigen specificity. This part is called a variable part (variable region, V part), both variable parts of the L chain and the H chain (VL, VH) are involved in determination of the antigen specificity. A part other than the variable part is almost constant for each class or subclass, and is called a constant part (constant region, C part). The constant part is such that the number of a polypeptide unit comprising about 110 amino acids (homologous unit) is one in the case of the L chain (CL), three in IgG, IgA, and IgD (CH1, CH2, CH3), and four in IgM and IgE in the case of the H chain, and each unit, or a region generated by binding with an opposite site is called a domain.

### (Method of Expressing an Antibody Molecule, an Antigen-Binding Fragment, or a Binding Molecule)

As used herein, unless otherwise are indicated, any polypeptide chain of an antibody, etc. is described as having an amino acid sequence beginning at an N-terminal extremity and ending at a C-terminal extremity. When an antigen-binding site includes both of V_{H} and V_{L} domains, these can be positioned on the same polypeptide molecule; preferably, each domain can be positioned at a separate chain and, in this case, the V_{H} domain is a part of a heavy chain of immunoglobulin, that is, an antibody or a fragment thereof, and V_{L} is a part of a light chain of immunoglobulin, that is, an antibody or a fragment thereof.

Examples of “antibody or antigen-binding fragment” used as used herein include an antibody and a chimeric antibody produced by a B cell or a hybridoma, a CDR transplantation antibody or a human antibody or any fragment thereof, for example, F(ab′)₂ and a Fab fragment, a single chain antibody and a single domain antibody. Therefore, it is understood that examples of “HA-binding molecule” as used herein include these antibodies and chimeric antibodies produced by a B cell or a hybridoma, a CDR transplantation antibody or a human antibody or any fragment thereof, for example, F(ab′)₂ and a Fab fragment, and a single chain antibody and a single domain antibody bound with other molecules.

The single chain antibody comprises variable domains of a heavy chain and a light chain of an antibody which covalently bind with a peptide linker comprising 10 to 30 amino acids, preferably 15 to 25 amino acids. For this reason, it is thought that the structure thereof does not include constant parts of a heavy chain and a light chain, and a small peptide spacer has lower antigenecity than that of a whole constant part. “Chimeric antibody” means an antibody in which the constant region(s) of a heavy chain or a light chain or both of them is (are) derived from human, while the variable domains of both of a heavy chain and a light chain are derived from non-human (e.g. mouse), or derived from human, but are derived from another human antibody. “CDR transplantation antibody” means an antibody in which a hypervariable site region (CDR) is derived from a donor antibody such as a non-human (e.g. mouse) antibody or another human antibody, while all or substantially all other parts of the immunoglobulin, for example, a constant region and a highly conserved part of a variable domain, that is, a framework region is derived from an accepter antibody, for example, a human-derived antibody. However, the CDR transplantation antibody includes a few amino acids of a donor sequence in the framework region, for example, in a part of the framework region adjacent to a hypervariable region. “Humanized antibody” means an antibody in which all of constant and variable regions of both of a heavy chain and a light chain are derived from human, or substantially the same as a human-derived sequence, but are not necessarily derived from the same antibody, and include an antibody produced by a mouse in which genes of a mouse immunoglobulin variable part and a mouse immunoglobulin constant part are substituted with human counterparts, for example, those described in general terminology in EP Patent 0546073B1, U.S. Pat. No. 5,545,806 etc.

As used herein, “titer” refers to an amount of an antibody binding to an antigen, which is contained in a unit volume of anti-serum, in a serum reaction. Actual measurement is performed by adding a constant amount of an antigen to a dilution series of anti-serum, and a measured value is expressed by a dilution multiple number at an end point at which a reaction is generated.

As used herein, “affinity” refers to a binding force between an antibody and its recognition substance. As used herein, affinity (K_{D}) is indicated using a dissociation constant of an antibody and its recognition substance such as an antigen as an index. A method of measuring affinity (K_{D}) is well-known to a person skilled in the art, and affinity can be also obtained, for example, using a sensor chip.

The framework can include any kind of a framework region, and is preferably a human-derived framework. A suitable framework region can be selected by referring to the literature of Kabat E. A. et al. A preferable heavy chain framework is a human heavy chain framework and, for example, is a framework of an anti-HA antibody shown in SEQ ID NO.:SEQ ID NOs.: 6-9. It can be determined from a sequence shown in SEQ ID NO.: 6-9 by referring to the literature, and comprises sequences of FR1, FR2, FR3 and FR4 regions. By a similar method, an anti-HA light chain framework can be determined from a sequence shown in SEQ ID NO.: 6-9 by referring to the literature, and comprises sequences of FR1′, FR2′, FR3′ and FR4′ regions. In the present invention, portions of CDR1, CDR2, CDR3 and FR3 may be necessary for interaction, and thus the remaining portions may be varied without substantively losing the interaction activity.

Monoclonal antibodies generated to all proteins which are naturally seen in human can be typically produced in a non-human system, for example, a mouse. As a direct result, when administered to a human, a heterogeneous antibody as produced by a hybridoma elicits an undesirable immunological response which is predominantly mediated with a constant part of heterogeneous immunoglobulin. This can limit the use of an antibody which cannot be administered over a long period of time. Therefore, use of a single chain, a single domain, a chimera, CDR transplantation, or particularly a human antibody which is predicted not to exhibit substantial allergy response when administered to human is particularly preferable.

As is well known, a minor change such as deletion, addition, insertion or substitution of one amino acid or a plurality of amino acids makes it possible to produce a protein corresponding to the original protein having substantial identity.

A constant part of a human heavy chain can be γ₁, γ₂, γ₃, γ₄, μ, α₁, α₂, δ or ε type, preferably γ type, more preferably γ₁ type, while a constant part of a human light chain can be κ or λ, type (including λ₁, λ₂ and λ₃ subtypes), preferably κ type. Amino acid sequences of all these constant parts are provided by Kabat et al.

(Production of Antibody)

The antibody or a funtionally equivalent variant of the present invention can be produced using any method well known in the art. Examples of such a method are described in the examples, but are not limited thereto. Immunization of an animal using an antigen allows production of an antibody.

Herein, such an antigen, e.g., a part of HA or a glycosylated peptide thereof, may be prepared by a recombinant DNA method or chemical synthesis. Such a method is exemplified in the examples. The resulting type 3 HA is mixed with an adjuvant, and is used as an antigen. Examples of the adjuvant include Freund complete adjuvant, and Freund incomplete adjuvant, and any of them may be mixed.

Regarding a monoclonal antibody, the spleen or a lymph node is collected from a mammal, and an antibody-producing cell obtained therefrom is fused with a myeloma cell, and thus a monoclonal antibody-producing hybridoma can be obtained. A method of cell fusion can be performed by a known method, and the hybridoma can be made, for example, according to the method of Koehler & Milstein (Nature, 256, 495-497 (1975)). In order to make a specific antibody recognizing the objective protein, the objective animal (e.g. mouse) is immunized according to the aforementioned method. A sufficient increase in blood titer is confirmed, and blood is taken, or a spleen cell is separated. A hybridoma producing a monoclonal antibody, particularly, a monoclonal antibody recognizing a C-terminal or a loop of the protein can be made by fusing the thus separated spleen cell and a myeloma cell. The spleen cell is derived from the immunized animal, preferably a mouse. The myeloma cell is derived from a mammal, and is preferably a mouse myeloma cell. In fusion of cells, polyethylene glycol, etc. can be used. A desired hybridoma can be selected by screening and cloning the hybridoma obtained by fusion. In order to make a monoclonal antibody, the resulting hybridoma is cultured in vitro or in vivo. Preferably, the hybridoma is cultured in vivo. For example, in order to produce ascites containing mouse monoclonal, the hybridoma is administered into the abdominal cavity of a mouse. The monoclonal antibody can be easily purified from the produced ascites by a method known to a person skilled in the art. It is preferable to collect spleen cells from the immunized animal 3 to 10 days after final immunization, but is not limited thereto.

In order to obtain a hybridoma from the resulting immunized cell, a plasmacytoma cell and an immune cell producing an antibody are fused, for example, in the presence of Sendai virus and polyethylene glycol for the purpose of allowing cells to be subcultured, by the method described, for example, in “Experimental Manual for Molecular Cell Biology” (Nankodo Co., Ltd., Takeichi Horie et al., 1994) etc., and thus a hybridoma can be obtained.

The hybridoma is selected by HAT medium (hypoxanthine, aminopterin, thymidine-added medium) and, at a stage of confirmation of a colony, binding of an antibody secreted in the culture supernatant and an antigen is investigated (screened), and thus a hybridoma producing the objective antibody can be obtained.

Examples of the screening method include various methods generally used in detecting an antibody, such as a spot method, an agglutination reaction method, a Western blot method, and an ELISA method and, preferably, the screening method is implemented according to the ELISA method using reactivity with a HA glycopeptide as an index, regarding the culture supernatant of the hybridoma, for example, as exemplified in the examples.

Cloning of the objective antibody-producing strain obtained as the result of screening can be implemented by a normal limiting dilution method or a soft agar method. The cloned hybridoma can be cultured at a large scale in a serum medium or a serum-free medium, if necessary. According to this culturing, a desired antibody having a comparatively high purity can be obtained as the culture supernatant. Alternatively, the hybridoma is inoculated into the abdominal cavity of a mammal having compatibility with the hybridoma, for example, a mouse, and a desired antibody can be also recovered as mouse ascites at a large amount. The culture supernatant of the antibody-producing hybridoma of the present invention and the ascites of a mouse, etc. can be used as a crude antibody liquid as they are. In addition, these can be purified by subjecting to ammonium sulfate fractionation, salting out, a gel filtration method, ion exchange chromatography, or an affinity chromatography method according to the common method.

A polyclonal antibody is obtained by collecting blood, for example, from a mammal immunized with an immunogen. In the method, as the mammal to be immunized with an immunogen, rabbit, goat, sheep, mouse and rat are generally used.

An immunizing method can be performed, for example, by administering an immunogen to a mammal by intravenous, intracutaneous, subcutaneous, or intraperitoneal injection according to a general method. More specifically, for example, an immunogen is diluted with a physiological saline-containing phosphate buffer (PBS) or physiological saline to a suitable concentration, and the mixture is administered to a test animal a few times at a 2 to 3 week interval, optionally together with a normal adjuvant. When a mouse is used, single dose is around 50 to 100 μg per animal. Herein, the adjuvant refers to a substance which non-specifically enhances an immune reaction to an antigen when administered with an antigen. Examples of the adjuvant which is usually used include pertussis vaccine, and Freund's adjuvant. Collection of the blood of a mammal 3 to 10 days after final immunization makes it possible to obtain the anti-serum. The anti-serum can be used as it is, or it can be purified, and also used as a polyclonal antibody.

Examples of a method of purifying a polyclonal antibody include a non-specific purification method and a specific purification method. The non-specific purification method is mainly for the purpose of obtaining an immunoglobulin fraction by a salting out method or an ion exchange chromatography method. Examples of the specific purification method include an affinity chromatography method using an immobilized antigen.

As used herein, “immunogen” used upon production of an antibody, represents a substance having the ability to generate an immune response, or to cause an immune response in an organism. The immunogen used in production of the antibody or a functionally equivalent variant of the present invention can be made using an activated hapten and a carrier protein by an active ester method described in Antibodies: A Laboratory Manual, (1989) (Cold Spring Harbor Laboratory Press), etc. Alternatively, the antigen can also be made by other methods described in Antibodies: A Laboratory Manual, (1989) (Cold Spring Harbor Laboratory Press), etc., for example, a carbodiimide method, a glutaraldehyde method or a diazo method.

(Immunological Measurement Method)

As a single specific antibody to be used in the present immunological measurement method, a monoclonal antibody which can be stably supplied is desirable, but the single specific antibody is not limited thereto, and any molecule can be used. Hereinafter, the method is exemplified using the monoclonal antibody. A sandwich immunological measurement method including the steps of immobilizing an antibody (first monoclonal antibody) on a solid phase, and incubating the antibody with a sample containing an antigen; further adding a labeled second monoclonal antibody, and incubating the resulting mixture; and detecting a labeled antigen antibody complex produced in the mixture is exemplified. Alternatively, in the immunological measurement method of the present invention, a sample, a solid phased first monoclonal antibody and a labeled second monoclonal antibody may be incubated at the same time. As the sandwich immunological measurement method, all sandwich immunological measurement methods such as a sandwich radiation immunological measurement method (RIA method), a sandwich enzyme immunological measurement method (EIA method), a sandwich fluorescent immunological measurement method (FIA method), a sandwich light emitting immunological measurement method (CLIA method), a sandwich light emitting enzyme immunological measurement method (CLEIA method), an immunological chromatograph method based on a sandwich method, etc. can be applied. For quantitation, the RIA method and the EIA method are preferable.

As used herein, “cross reactivity” refers to immunological cross reactivity. When an antibody obtained by immunization with a certain antigen also exhibits a binding reaction with another antigen (associated antigen), this reaction is referred to as a cross reaction. When a reaction amount between the objective antigen and its antibody is used as a standard, a degree of a reaction amount between the associated antigen and its antibody can be indicated as cross reactivity. As used herein, representatively, when indicated by a relative value (%) of affinity of 1%, 2%, 3%, or 0.5%, 0.2%, or 0.1% etc., cross reactivity can be said to be low. As the value is lower, cross reactivity is lower, and it is indicated that the objective antigen possesses specficity. In many cases, cross reactivity occurs mainly due to high similarity between structures of the objective antigen and an associated antigen.

An antibody of the present invention, an antigen binding fragment thereof or a HA-binding molecule can be solid-phased on carriers such as microtiter plates, beads, tubes, membranes, filter paper, and plastic cups and, particularly, polyethylene beads are used. A sample to be measured can be a sample containing human HA such as human plasma, serum, blood and urine. The anti-HA antibody of the present invention, an antigen-binding fragment thereof or a HA-binding molecule can be labeled with a radioactive isotopic element, an enzyme, a fluorescent substance, a light emitting substance, or in a visual-determinable simple measurement method, with a gold colloid or a coloring latex etc. Examples of the radioactive isotopic element used in labeling include ¹⁴C, ³H, ³²P, ¹²⁵I, and ¹³¹I and, particularly, ¹²⁵I is suitably used. These can be bound to a monoclonal antibody by a chloramine T method, a peroxidase method, an Iodogen method, or a Volt Hunter method etc. Examples of the enzyme which can be used in labeling include β-galactosidase (βGAL), alkaline phosphatase (ALP), and horseradish peroxidase (HRP). These can be bound to a monoclonal antibody by a periodic acid crosslinking method (Nakane method), or a method of Ishikawa et al. (Igaku-Shoin Ltd.; Enzyme Immunological Measurement Method, third edition, 75-127 (1987)), etc. Examples of the fluorescent substance used in labeling include fluorescein, fluorescamine, fluorescein isothiocyanate, and tetramethylrhodamine isothiocyanate. Examples of the light emitting substance used in labeling include luciferin, a luminol derivative, and an acridinium ester. In a simple measurement method etc., a gold colloid or a coloring latex may be used.

According to a preferred embodiment, a sandwich RIA method can be performed. In the sandwich RIA method, specifically, a bead solid-phased with a first monoclonal antibody is added to a standard solution or a sample, and they are mixed, and incubated at 4° C. to 45° C. preferably 25° C. to 37° C. for 1 to 4 hours, preferably 2 hours (first reaction). After washing, a solution containing a second monoclonal antibody labeled, for example, with ¹²⁵I added, and the mixture is incubated at 4° C. to 45° C., preferably 25° C. to 37° C. for 1 to 4 hours, preferably 2 hours to form an antibody/antibody complex on the bead (second reaction). After washing, radioactivity of the antigen antibody complex bound to the bead is detected with a gamma counter etc., and thus an amount can be measured. According to another preferable embodiment, a sandwich EIA method can be performed. In the sandwich EIA method, specifically, a bead on which a first monoclonal antibody is immobilized and is added to a standard solution or a sample, and they are mixed, and incubated at 4° C. to 45° C., preferably 25° C. to 37° C. for 1 to 4 hours, preferably 2 hours (first reaction). After washing, a solution containing a second monoclonal antibody labeled with an enzyme, for example, horseradish peroxidase (HRP) is added, and the mixture is incubated at 4° C. to 45° C., preferably 25° C. to 37° C. for 1 to 4 hours, preferably 2 hours to form an immunological complex comprising antibody-antibody on a bead (second reaction). The enzyme activity on the bead is measured by a colorimetric method via a substrate specific for an enzyme, for example, tetramethylbenzidine (TMB) when a labeling enzyme is HRP, and thus a captured amount on the bead can be measured. Colorimetric quantitation is performed with a normal spectral photometer.

Neutralization activity can be measured using the antibody-dependent cytotoxicity as an index.

The antibody-dependent cytotoxicity can be measured as follows. That is, the antibody-dependent cytotoxicity can be analyzed by a chromium release test. Human peripheral mononuclear cell (PBMC) is separated from peripheral blood of a healthy subject using Ficoll-paque PLUS (manufactured by GE Healthcare) according to a package insert. DMEM containing 10% FCS is added so that the separated PBMC becomes 4×10⁶/ml.

According to the technical level in the art, a person skilled in the art can make a humanized antibody, for example, by a CDR grafting method (e.g. EP 239400).

(Medicament)

Although the compound of the present invention or a pharmaceutically acceptable salt thereof can be administered alone, it is usually preferably provided as various pharmaceutical preparations. In addition, those pharmaceutical preparations are used in animals and human.

As an administration route, it is preferable to use an administration route which is most effective upon treatment, and examples thereof include oral route, and a parenteral route such as rectal, intraoral, subcutaneous, intramuscular, intravenous, etc. As a dosage form, it can be formulated into capsules, tablets, granules, powders, syrups, emulsions, suppositories, injectables, etc. Liquid preparations such as emulsions and syrups which are suitable for oral administration can be produced using water, sugars such as sucrose, sorbit, and fructose, glycols such as polyethylene glycol, and propylene glycol, oils such as sesame oil, olive oil, and soybean oil, antiseptics such as p-hydroxybenzoic acid esters, flavors such as strawberry flavor, and peppermint, etc. In addition, capsules, tablets, powders, granules, etc. can be produced using excipients such as lactose, glucose, sucrose, and mannitol, disintegrating agents such as sodium alginate, lubricants such as magnesium stearate, and talc, binders such as polyvinyl alcohol, hydroxypropylcellulose, and gelatin, surfactants such as a fatty acid ester, plasticizers such as glycerin, etc.

A preparation suitable for parenteral administration preferably comprises a sterilized aqueous preparation including an active compound which is isotonic with blood of a recipient. For example, in the case of injectables, solutions for injection are prepared using carriers comprising salt solutions, glucose solutions or a mixture of aqueous salt and glucose solutions, etc.

Local preparations are prepared by dissolving or suspending an active compound in one or more media, for example, mineral oils, petroleums, polyhydric alcohols etc., or other bases used in local pharmaceutical preparations. Preparations for intestinal administration are prepared using normal carriers, for example, cacao butter, hydrogenated fat, hydrogenated fat carboxylic acid etc. The present invention may be provided as suppositories.

In the present invention, also in parenteral agents, one or more kinds of auxiliary components selected from glycols, oils, flavors, antiseptics (including antioxidants), excipients, disintegrating agents, lubricants, binders, surfactants, plasticizers etc. exemplified in oral agents may be added.

An effective dose and an administration time of the compound of the present invention or a pharmaceutical acceptable salt thereof are different depending on an administration form, the age and weight of a patient, the nature or severity of symptoms to be treated etc., and a dose is usually 0.01 to 1000 μg/person, preferably 5 to 500 μg/person per day, and it is preferable that regarding the administration time, the compound or a salt thereof is administered once a day or by division.

(Structure-Based Drug Design Techniques)

Structure-based drug design techniques can be applied to the structural representation of the HA trimer or antibody in order to identify compound, antigen or antibody that interacts with antibody/antigen to block antigen/antibody binding. A variety of suitable techniques [e.g. Further details: Rational drug design: novel methodology and practical applications, ACS Symposium Series vol. 719 (Parrill & Reddy eds., 1991).] are available to the skilled person.

Software packages for implementing molecular modeling techniques for use in structure-based drug design include SYBYL [Available from Tripos Inc (http://www.tripos.com)], AMBER [Available from Oxford Molecular (http://www.oxmol.co.uk/)], CERIUS² [Available from Molecular Simulations Inc], INSIGHT II [Available from Molecular Simulations Inc], CATALYST [Available from Molecular Simulations Inc], HYPERCHEM [Available from Hypercube Inc (http://www.hyper.com/).], CHEMSITE [Available from Pyramid Learning (http://www.chemsite.org/)] etc.

These softwares can be used to determine binding surfaces of an antibody and/or antigen (e.g. HA trimer) in order to reveal features such as van der Waals contacts, electrostatic interactions, and/or hydrogen bonding opportunities. These binding surfaces may be used as follows:

(Docking)

Docking aligns the 3D structures of two or more molecules to predict the conformation of a complex formed from the molecules [e.g. Blaney & Dixon (1993) Perspectives in Drug Discovery and Design 1:301]. According to the present invention, molecules, such as HA trimer or an antibody, are docked with the structure to assess their ability to interact with a different molecule such as an antibody (e.g. F005-126 antibody) or HA trimer.

Docking can be accomplished by either geometric matching of the antigen or ligand and its partner such as antibody or receptor or by minimizing the energy of interaction. Geometric matching algorithms are preferred because of their relative speed.

Suitable docking algorithms include, but are not limited to:

DOCK [Kuntz et al. (1982) J. Mol. Biol. 161:269-288); available from UCSF], the prototypical program for structure-based drug design.

AUTODOCK [Goodsell & Olson (1990) Proteins: Structure, Function and Genetics 8:195-202, Available from Oxford Molecular (http://www.oxmol.co.uk/)], which docks ligands/antigens/antibodies in a flexible manner to receptors using grid-based Monte Carlo simulated annealing. The flexible nature of the AUTODOCK procedure helps to avoid bias (e.g. in orientation and conformation of the ligand in the active site) introduced by the user [Meyer et al. (1995) Persp. Drug Disc. Des. 3:168-195], because while the starting conformation in a rigid docking is normally biased towards a minimum energy conformation of the ligand, the binding conformation may be of relatively high conformational energy [Nicklaus et al. (1995) Bioorganic & Medicinal Chemistry 3:411].

MOE-DOCK [Available from Chemical Computing Group Inc. (http://www.chemcomp.com/)], in which a simulated annealing search algorithm is used to flexibly dock ligands/antigens/antibodies. A grid-based energy evaluation is used to score docked conformations.

FLExX [Available from Tripos Inc (http://www.tripos.com)], which docks conformationally flexible ligands/antigens/antibodies into a binding site using an incremental construction algorithm that builds the ligand/antigen/antibody in the site; Docked conformations are scored based on the strength of ligand-receptor interactions.

GOLD [Jones et al. (1997) J. Mol. Biol. 267:727-748], a genetic algorithm for flexible ligand/antigen/antibody docking, with full ligand/antigen/antibody and partial protein flexibility. Energy functions are partly based on conformation and non-bonded contact information.

AFFINITY [Available from Molecular Simulations Inc], which uses a two-step process to dock ligands/antigens/antibodies. First, initial placements of the ligand within the receptor are made using a Monte Carlo type procedure to search both conformational and Cartesian spaces. Second, a simulated annealing phase optimises the location of each ligand placement. During this phase, AFFINITY holds the ‘bulk’ of the receptor (atoms not in the binding site) rigid, while the binding site atoms and ligand atoms are movable.

C² LigandFit, which uses the energy of the ligand-receptor complex to automatically find best binding modes. Stochastic conformational search techniques are used, and the best results from the conformational sampling are retained. A grid method is used to evaluate non-bonded interactions between the rigid receptor/antibody/antigen and the flexible ligand/antigen/antibody atoms.

Preferably, the docking algorithm is used, in a ‘high throughput’ mode, in which members of large structural libraries of potential ligands/antigens/antibodies are screened against the receptor/antibody/antigen structure [Martin (1992) J. Med. Chem. 35:2145-54].

Suitable structural libraries include antibodies, is not limited thereto and other low molecular weight molecules and the like may also be used such as the ACD (Available Chemical Directory, from MDL Inc), AsInEx, Bionet, ComGenex, the Derwent World Drug Index (WDI), the Contact Service Company database, LaboTest, ChemBridge Express Pick, ChemStar, BioByteMasterFile, Orion, SALOR, TRIAD, ILIAD, the National Cancer Institute database (NCI), and the Aldrich, Fluka, Sigman and Maybridge catalogs. These are commercially available (e.g. the HTS Chemicals collections from Oxford Molecular or the LeadQuest™ files from Tripos).

(Pharmacophore Hypotheses)

A pharmacophore (i.e. a collection of chemical features and 3D constraints that expresses specific characteristics responsible for activity) can be defined for the HA trimer and/or F005-126 antibody. The pharmacophore preferably includes surface-accessible features, more preferably including hydrogen bond donors and acceptors, charged/ionisable groups, and/or hydrophobic patches. These may be weighted depending on their relative importance in conferring activity [also Computer-Assisted Lead Finding and Optimization (eds. Testra & Folkers, 1997)].

Pharmacophores can be determined using software such as CATALYST (including HypoGen or HipHop) [Available from Molecular Simulations Inc (http://www.msi.com/)], CERIUS2, or constructed by hand from a known conformation of a lead compound. The pharmacophore can be used to screen structural libraries, using a program such as CATALYST [Available from Molecular Simulations Inc]. The CLIX program [Davic & Lawrence (1992) Proteins 12:31-41] can also be used, which searches for orientations of candidate molecules in structural databases that yield maximum spatial coincidence with chemical groups which interact with the receptor/antibody/antigen.

(De Novo Compound Design)

The binding surface or pharmacophore of the antigen such as HA trimer and/or an antibody such as F005-126 can be used to map favorable interaction positions for functional groups (e.g. protons, hydroxyl groups, amine groups, hydrophobic groups and/or divalent cations) or small molecule fragments. Compounds can then be designed de novo in which the relevant functional groups are located in the correct spatial relationship to interact with the corresponding partner such as HA trimer and/or F005-126.

Once functional groups or small molecule fragments which can interact with specific sites in the surface of an antigen such as HA trimer and/or antibody such as F005-126 have been identified, they can be linked in a single compound using either bridging fragments with the correct size and geometry or frameworks which can support the functional groups at favorable orientations, thereby providing a compound according to the invention. While linking of functional groups in this way can be done manually, perhaps with the help of software such as QUANTA or SYBYL, automated or semi-automated de novo design approaches are also available:

MCDLNG [Gehlhaar et al. (1995) J. Med. Chem. 38:466-72], which fills a receptor binding site with a close-packed array of generic atoms and uses a Monte Carlo procedure to randomly vary atom types, positions, bonding arrangements and other properties.

MCSS/HOOK [Caflish et al. (1993) J. Med. Chem. 36:2142-67, Eisen et al. (1994) Proteins: Str. Funct. Genet. 19:199-221, Available from Molecular Simulations Inc], which links multiple functional groups with molecular templates taken from a database.

LUDI [Bohm (1992) J. Comp. Aided Molec. Design 6:61-78, Available from Molecular Simulations Inc, which computes the points of interaction that would ideally be fulfilled by a ligand/antigen/antibody, places fragments in the binding site based on their ability to interact with the receptor/antibody/antigen, and then connects them to produce a ligand/antigen/antibody.

GROW [Moon & Howe (1991) Proteins: Str. Funct. Genet. 11:314-328], which starts with an initial ‘seed’ fragment (placed manually or automatically) and grows the ligand outwards.

SPROUT suite [Available from http://www.simbiosys.com/sprout/index.html] which includes modules to: identify favourable hydrogen bonding and hydrophobic regions within a binding pocket (HIPPO module); select functional groups and position them at target sites to form starting fragments for structure generation (EleFAnT); generate skeletons that satisfy the steric constraints of the binding pocket by growing spacer fragments onto the start fragments and then connecting the resulting part skeletons (SPIDeR); substitute hetero atoms into the skeletons to generate molecules with the electrostatic properties that are complementary to those of the receptor site (MARABOU). The solutions can be clustered and scored using the ALLigaTOR module.

LEAPFROG [Available from Tripos Inc (http://www.tripos.com)], which evaluates ligands by making small stepwise structural changes and rapidly evaluating the binding energy of the new compound. Changes are kept or discarded based on the altered binding energy, and structures evolve to increase the interaction energy with the receptor.

GROUPBUILD [Rotstein et al. (1993) J. Med. Client. 36:1700], which uses a library of common organic templates and a complete empirical force field description of the non-bonding interactions between a ligand and receptor to construct ligands that have chemically reasonable structures and have steric and electrostatic properties complimentary to the receptor binding site.

CAVEAT [Lauri & Bartlett (1994) Comp. Aided Mol. Design 8:51-66], which designs linking units to constrain acyclic molecules.

RASSE [Lai (1996) J. Chem. Inf. Comput. Sci. 36:1187-1194]

(The HA Trimer/F005-126 Binding Site)

To simplify computational complexity, algorithms for docking and ligand design will typically focus only on the binding site of a receptor. It is pointless to attempt to dock a ligand/antigen/antibody with a region in the receptor/antibody/antigen which is known not to be involved. Binding site identification is included in some algorithms (e.g. C²LigandFit, the ‘Binding Site Analysis’ module of INSIGHT II, the SPHGEN routine of DOCK). Some manual guidance may be required (e.g. AFFINITY).

Where a binding site has to be defined for the HA timer-F005-126 antibody, this should include one or more of

the following amino acid residues of the amino acid sequences of HA of H3N2 Aic 68 (SEQ ID NO: 48), or corresponding amino acid residues thereto:
a Site L epitope element comprising amino acid residues N171, D172, P239 and G240 (Need to be verified)
a Site R epitope element comprising amino acid residues S270, D271, A272, P273, P284 and N285;
a Site R epitope element comprising sugar chains NAG(N-acetyl-D-glucosamine)1, NAG2 BMA(beta-D-mannose)3, MAN(alpha-D-mannose)4, MAN5, MAN6 and MAN7, linked to amino acid residue N285; and/or

the following amino acid residues of F005-126 (SEQ ID NO: 2):
a paratope element comprising amino acid residues T73, G74, and T75 (according to the Kabat's numbering shown in FIG. 5-2);
a paratope element comprising amino acid residue S31 (according to the Kabat's numbering shown in FIG. 5-2);
a paratope element comprising amino acid residues Y53, D54, G55, Q56 and H57 (according to the Kabat's numbering shown in FIG. 5-2);
a paratope element comprising V98, R99, G100, and V100a (according to the Kabat's numbering shown in FIG. 5-2).

The binding sites may include antibody F005-126 (SEQ ID NO: 2), antigen HA-a+HA-b (e.g. SEQ ID NO: 48 and 21) and/or water molecule.

(The Structural Representation)

The invention involves the use of a 3D structural representation of the antibody F005-126 and/or antigen HA-a+HA-b in the antibody F005-126-antigen HA-a+HA-b complex state. This may be a representation of (a) a conformational epitope formed by an antibody-antigen complex of antibody F005-126, antigen HA-a+HA-b and/or water molecule; (b) a paratope of antibody F005-126 in an antibody-antigen complex of antibody F005-126, antigen HA-a+HA-b and water molecule; or (c) a partial or full complex of a conformational epitope and paratope formed by an antibody-antigen complex of antibody F005-126, antigen HA-a+HA-b and/or water molecule. Such domains play an important role in initiating neutralizing activity.

The structural representation is preferably based on or derived from the atomic co-ordinates PDB1 [Refine lowres] and PDB2 [JKL-90] or PDB3 [refine_{—}0101-deposit (p2445)] and PDB4 [JKL-90-0101] [all of which are shown in U.S. non-provisional patent application Ser. No. 13/832,818, U.S. provisional patent application Ser. No. 61/787,399 and U.S. provisional patent application Ser. No. 61/705,504 which are incorporated by reference], which represents the complex of antibody F-5125 and two elements of the HA trimer (HA-a and HA-b) and water. Suitable structural representations include 3D models and molecular surfaces derived from these atomic co-ordinates.

The 3D structural data of PDB1, PDB2, PDB 3 and PDB4 are also provided at the following database:

http://www.fujita-hu.ac.jp/˜ibayoshi/crystal.html; and
https://skydrive.live.com/?cid=34819606A7A8444D&id=34819606A7A8444D %21105.
Further, PDB3 is deposited as 3WHE in the Protein Data Bank, which is found at http://www.rcsb.org/pdb/search/structidSearch.do?structureId=3WHE.

Variants of data contained in PDB 1 and PDB2 or PDB3 and PDB4 can also be used for the invention, such as variants in which the r.m.s. deviation of the x, y and z co-ordinates for all heavy (i.e. not hydrogen) atoms are all less than 4.0 Angstroms or 2.5 Angstrroms (e.g. less than 2 Angstroms, preferably less than 1 Angstroms, and more preferably less than 0.5 Angstroms or less than 0.1 Angstroms) compared with PDB 1 and PDB2 or PDB3 and PDB4. Co-ordinate transformations which retain the 3D spatial relationships of atoms may also be used to give suitable variants.

Preferred fragments of the data contained in PDB1 and PDB2 or PDB3 and PDB4 whose coordinates can be used in the invention include the following amino acid residues:

the following amino acid residues of the amino acid sequences of HA of H3N2 Aic 68 (SEQ ID NO: 48), or corresponding amino acid residues thereto:
a Site L epitope element comprising amino acid residues N171, D172, N173, P239 and G240
a Site R epitope element comprising amino acid residues S270, D271, A272, P273, P284 and N285;
a Site R epitope element comprising sugar chains NAG(N-acetyl-D-glucosamine)1, NAG2 BMA(beta-D-mannose)3, MAN(alpha-D-mannose)4, MAN5, MAN6 and MAN7, linked to amino acid residue N285; and/or

the following amino acid residues of F005-126 (SEQ ID NO: 2):
a paratope element comprising amino acid residues T73, G74, and T75 (according to the Kabat's numbering shown in FIG. 5-2);
a paratope element comprising amino acid residues S31 (according to the Kabat's numbering shown in FIG. 5-2);
a paratope element comprising amino acid residues Y53, D54, G55, Q56 and H57 (according to the Kabat's numbering shown in FIG. 5-2);
a paratope element comprising G97, V98, R99, G100, and V100a (according to the Kabat's numbering shown in FIG. 5-2).

It is preferred that the methods of the invention use only a portion of PDB 1, PDB2, PDB3 and/or PDB4.

The water molecules in PDB1, PDB2, PDB3 and/or PDB4 can optionally be omitted when performing the methods of the invention.

The atomic co-ordinates given herein can also be used as the basis of models of further protein structures. For example, a homology model could be based on the structure of a conformational epitope formed by an antibody-antigen complex of antibody F005-126, antigen HA-a+HA-b and water molecule, a paratope of antibody F005-126 in an antibody-antigen complex of antibody F005-126, antigen HA-a+HA-b and water molecule or a partial or full complex of a conformational epitope and paratope formed by an antibody-antigen complex of antibody F005-126, antigen HA-a+HA-b and water molecule of the present invention. The co-ordinates can also be used in the solution or refinement of further crystal structures of a complex of a conformational epitope and paratope formed by an antibody-antigen complex of antibody F005-126, antigen HA-a+HA-b and water molecule.

(The Storage Medium)

The storage medium in which a conformational epitope formed by an antibody-antigen complex of antibody F005-126, antigen HA-a+HA-b and/or water molecule, a paratope of antibody F005-126 in an antibody-antigen complex of antibody F005-126, antigen HA-a+HA-b and water molecule or a partial or full complex of a conformational epitope and paratope formed by an antibody-antigen complex of antibody F005-126, antigen HA-a+HA-b and water molecule of the present invention is provided, is preferably random-access memory (RAM), but may also be read-only memory (ROM e.g. CDROM), or a diskette. The storage medium may be local to the computer, or may be remote (e.g. a networked storage medium, including the internet).

The invention also provides a computer-readable medium for a computer, characterized in that the medium contains atomic co-ordinates and/or a 3D structural representation of a conformational epitope formed by an antibody-antigen complex of antibody F005-126, antigen HA-a+HA-b and water molecule, a paratope of antibody F005-126 in an antibody-antigen complex of antibody F005-126, antigen HA-a+HA-b and water molecule or a partial or full complex of a conformational epitope and paratope formed by an antibody-antigen complex of antibody F005-126, antigen HA-a+HA-b and water molecule of the present invention. The atomic co-ordinates are preferably PDB1, PDB2, PDB3 and/or PDB4 or variants thereof.

Any suitable computer can be used in the present invention.

(Testing Compounds, Antibodies or Antigens)

The methods may comprise further steps of: providing a compound, antigen or antibody identified by said structure-based drug design techniques; and contacting said compound, antigen or antibody with HA trimer or F005-126 antibody or equivalent thereof preferably containing or binding to the concave region thereof and assaying the interaction between them, and optionally assaying whether neutralizing activity is raised. As used herein the concave region which may be used in the present methods is, in the case of H3N2 Aic68 (SEQ ID NOs:48 and 21, the following amino acid residues refer to those in SEQ ID NO: 48 for HA1 and SEQ ID NO: 21 for HA2):

HA1-SiteL (shown in pink in FIGS. 8-1 and 8-2)

Ser114 Ser115 Glu123 Thr167 Met168 Pro169 Asn170
Phe174 Asp175 Lys176 Tyr178 Arg207 Arg208 Lys238
Asp241 Val242 Val244 Tyr257 Lys259 Met260 Arg261
Thr262 Gly263 Lys264 Ser265

HA2-SiteL (shown in pink in FIGS. 8-1 and 8-2)

Glu61 Lys62 Phe63

HA1-SiteR (shown in brown in FIGS. 8-1 and 8-2)

Ser47 Thr48 Gly49 Lys50 Asp60 Ile62 Asp63 Cys64
Thr65 Asp68 Asp73 His75 Glu89 Arg90 Ala93 Phe94
Ser95 Asn96 Arg109 Pro221 Trp222 Val223 Arg224
Gly225 Arg269 Ile274 Asp275 Asn296 Val297 Asn298
Lys299 Ile300 Tyr308 val309 Lys310

HA2-SiteR (shown in brown in FIGS. 8-1 and 8-2)

Glu67 Lys68 Glu69 Phe70 Ser71 Glu72 Asp86 Ile89
Asp90

The concave region is automatically assigned by a docking software used in the invention such as those employed in the Examples e.g., AutoDock and the like. Depending on the software or application that is actually used, types recognized as a concave region may vary. However, those skilled in the art will understand even if such a variation exists, the present invention may be practiced using the data considering such a variation (e.g. 0.5-2.0 Angstrohms (0.7-1.7 Angstrohms)

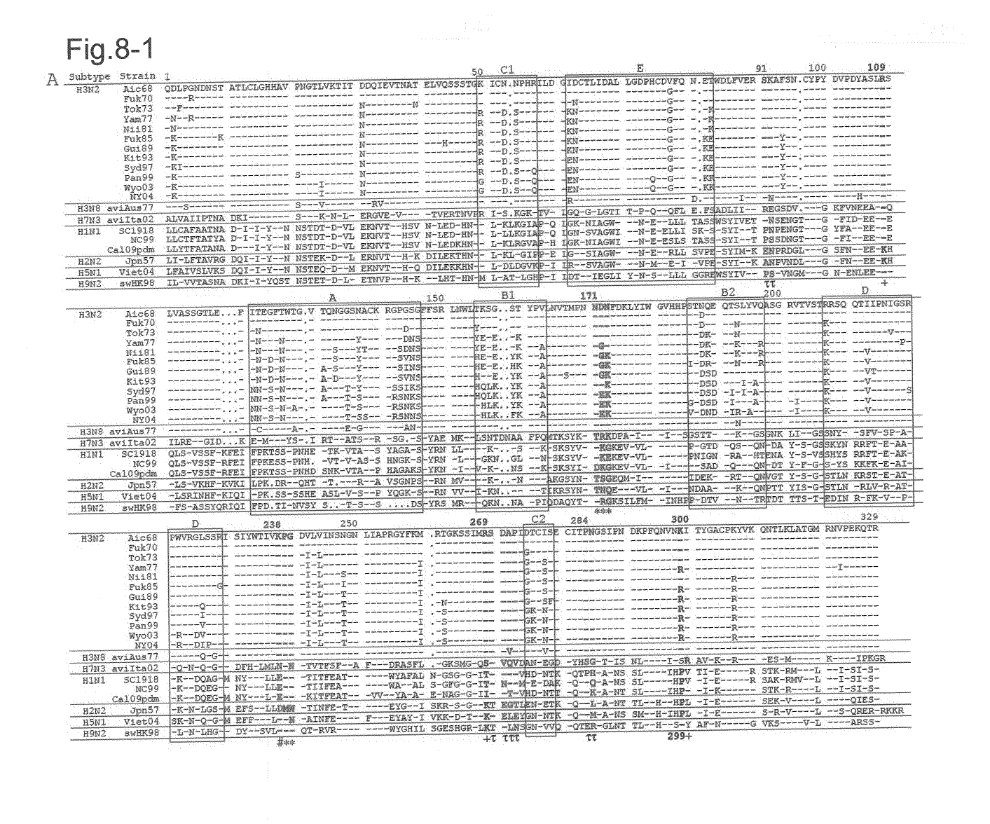

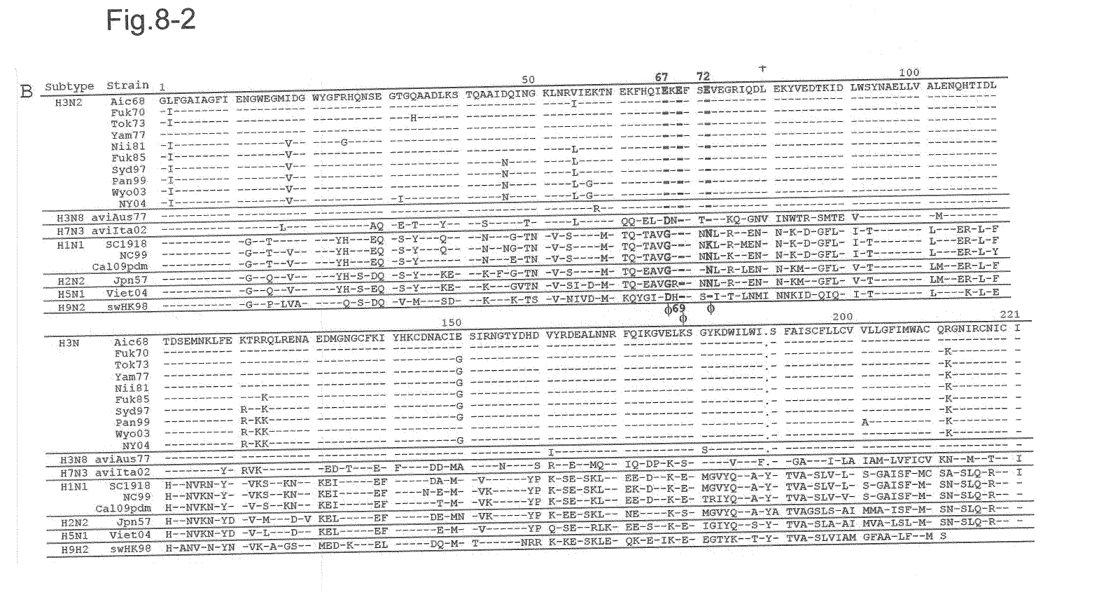

In the present invention, HA trimer may be of any type from the influenza virus, but preferably of Group II, and more preferably of H3 types. A number of H3 subyptes are described in FIG. 8 (FIG. 8-1 and FIG. 8-2). Except for H3 subtypes, when a variant does not own a carbohydrate chain in a domain corresponding to Site L and Site R of H3, a variant is not limited in particular in the docking model.

The assay may be of a competitive nature. For example, the assay may include F005-126 antibody (either purified, or in the context of the influenza virus antibody), such that HA trimer or equivalent thereof and the compound, antigen or antibody compete for binding to F005-126 antibody or vice versa.

(Compounds, Antigens, and Antibodies and their Uses)

The methods of the invention identify compounds, antigens and/or antibodies that can interact with HA trimer of influenza virus or F005-126 antibodies. These compounds may be designed de novo, may be known compounds, antigens and/or antibodies, or may be based on known compounds, antigens and/or antibodies. The compounds may be useful pharmaceuticals themselves, or may be prototypes which can be used for further pharmaceutical refinement (i.e. lead compounds, antigens and/or antibodies) in order to improve binding affinity or other pharmacologically important features (e.g. bio-availability, toxicology, metabolism, pharmacokinetics etc.).

The invention thus provides: (i) a compound, antigen and/or antibody identified using the methods of the invention; (ii) a compound, antigen and/or antibody identified using the methods of the invention for use as a pharmaceutical; (iii) the use of a compound, antigen and/or antibody identified using the methods of the invention in the manufacture of a medicament for treating influenza virus infection; and (iv) a method of treating a patient with influenza virus infection, comprising administering an effective amount of a compound, antigen and/or antibody identified using the methods of the invention.

These compounds, antigens, and/or antibodies preferably interact with HA timer, and/or competes with F005-126 antibody with a binding constant in the micromolar or, more preferably, nanomolar range or stronger.

As well as being useful compounds, antigens, and/or antibodies individually, ligands identified in silico by the structure-based design techniques can also be used to suggest libraries of compounds, antigens, and/or antibodies for ‘traditional’ in vitro or in vivo screening methods. Important pharmaceutical motifs in the ligands, antigens and/or antibodies can be identified and mimicked in compound, antigen, and/or antibody libraries (e.g. combinatorial libraries) for screening for HA trimer-influenza and/or neutralizing activity.

(Crystals)

The invention also provides a composition comprising an antibody-antigen binding region (e.g. the concave region of the HA timer of the present invention) of HA trimer complex with an antibody such as F005-126 antibody or equivalent in crystalline form. The crystal can be used for diffraction studies e.g. X-ray or neutron diffraction.

The crystal is preferably in a form wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

In some embodiments, the composition may include ligands, antigens and/or antibodies which are co-crystallised with the HA trimer or antibody F005-126; in other embodiments the composition may be essentially pure protein.

(Production of HA Trimer)

The ectodomain of hemagglutinin (e.g. amino acid residues 17-520:HA SEQ ID NO: 2) may be derived from any strain such as those including but not limited to A/Aich/2/1968(H3N2) and those listed in FIG. 8 (FIGS. 8-1 and 8-2), and such may be cloned into an suitable expression vector such as pBAC-3, e.g. as C-terminal fusions with a thrombin protease cleavage site, a trimerization ‘foldon’ sequence and His-tag (James Stevens, et al Science 2004, 303, 1866). Such fusion proteins may be synthesized by a baculovirus expression system or any other known means for production.

The incubation of the system may be carried out at an appropriate temperature for an appropriate length of time, e.g. at 27° C. for 48 hours, HA protein may be secreted into the culture medium, which may be subsequently used. Cell debris may be removed by an appropriate means such as by centrifugation e.g. at 3500×g for 20 min, and supernatant may be concentrated with an appropriate system, e.g. QuickStand System (GE Healthcare). The concentrated culture supernatant may then be loaded on a suitable column, such as HisTrap column (5 ml; GE Healthcare) pre-equilibrated with buffer A (10 mM Tris-HCl (pH 8.0) containing 500 mM NaCl, and 20 mM imidazole). Such a column may then be washed with an appropriate elution buffer, e.g. 50 ml of buffer A, and HA may then be eluted with an appropriate elution buffer, e.g. 10 mM Tris-HCl (pH 8.0) containing 500 mM NaCl, and 500 mM imidazole.

Optionally, the fractions may be pooled and dialyzed against a suitable buffer e.g. 20 mM Tris-HCl (pH 8.0) and 20 mM NaCl containing thrombin protease to cleave the His-tag. Optionally, to separate the His-tag and uncleaved protein, the protein may be loaded on a suitable column, such as HisTrap column, and the flow-through fractions may be collected. The protein may further be purified by a suitable means such as ion exchange on a HiTrap Q column (5 ml; GE Healthcare) and size-exclusion chromatography on a HiLoad 16/60 Superdex 200 pg column (GE Healthcare), in a final buffer containing 20 mM Tris-HCl (pH 8.0), 150 mM NaCl.

(Processing of F005-126 Antibody)

IgG antibody (F005-126) may be incubated with an appropriate carrier protein such as immobilized papain (Pierce), and Fab fragments generated by papain digestion may be separated from undigested IgG and Fc fragment by an appropriate means such as chromatography on a HiTrap rProteinA FF column (GE Healthcare). For the crystallization of the HA-Fab complex, HA and Fab may be mixed in an appropriate ratio, e.g., in a 1:1.2 molar ratio and incubated for an appropriate length of time, e.g. overnight at 4° C. The HA-Fab complex, formed by incubating the proteins together, may be separated from the uncomplexed proteins by chromatography on a suitable column, e.g. HiLoad 16/60 Superdex 200 pg column, preequilibrated with a suitable buffer such as 20 mM Tris-HCl (pH 8.0), 150 mM NaCl. The fractions containing the complex proteins may be pooled and concentrated to an appropriate level, e.g. 10 mg/ml with e.g. an Amicon-15 filter (Millipore).

(HA Trimer-Antibody Crystallization)

The initial screening of crystallization conditions may be conducted using commercially available screening kits such as those available from Hampton Research. The HA-Fab complex crystals may be obtained by an appropriate means, e.g. in some crystallization conditions with the sitting-drop vapor diffusion method using e.g. PEG as precipitants. Crystals may be obtained under an appropriate condition, e.g. a few days by mixing e.g. 1 μl sample solution and e.g. 1 μl reservoir solution containing of e.g., 12% PEG8000, 0.2 M KCl, 0.1 M Mg(CH₃COO)₂ and Na-citrate buffer pH 5.5. For the diffraction data collection, the crystals may be gradually soaked in an appropriate solution e.g. reservoir solution containing 20% glycerol. The data sets may be collected at an appropriate apparataus, e.g. BL41XU beam line (SPring-8).

(Data Collection, Structure Determination and Refinement)

Diffraction data were collected at 100K on the BL41XU beamline at the SPring-8 (Hyogo, Japan). Diffraction images were processed with XDS (Kabsch, 1993) and HKL2000 (Otwinowski et al., 1997). The structure was solved at 4.0 Å resolution by molecular replacement with PHASER using the structures of H3 (PDB 1HA0) and Fab (PDB 1EO8) (Chen et al., 2000, Fleury et al, 2000) as starting models. The asymmetric unit contains four HA trimers and twelve F005-126 molecules and was refined using CNS with tight restrains and manually rebuilt with Coot (Emsley et al., 1994). Positive density for N-linked glycosylation was observed at 5 of the 6 predicted sites on HA, and a total of 20 sugar residues were built. Hydrogen bonds and van der Waals contacts between F005-126 and H3 HA were calculated using HBPLUS and CONTACSYM, respectively (McDonald et al., 1994, Sheriff et al., 1987). Surface area buried upon Fab binding was calculated with MS (Connolly et al., 1983). PyMol (DeLano Scientific) was used to render structure figures and for general manipulations. Numbers of amino acids in F5126 H or L chain are shown as serial numbers of the amino acids in the H or L chain in the atomic coordinates. Kabat numbering is applied to the amino acids in Table 1 and 4, and Figures, and documents in the present patent using the AbNum server (Abhinandan et al., 2008). Final refinement statistics are summarized in Table 3, which is located at the bottom of the specification.

Diffraction data may be collected under an appropriate condution, e.g. 100K on the BL41XU beamline at the SPring-8 (Hyogo, Japan). Diffraction images may be processed with an appropriate software such as XDS (Kabsch, 1993) and HKL2000 (Otwinowski et al., 1997). The structure may be solved at e.g. 4.0-Å resolution (this may be further improved at 3.0-Å resolution, 2.5-Å resolution, 2.0-Å resolution, 1.5-Å resolution, 1.0-Å resolution, 0.5-Å resolution and the like, as appropriate) by molecular replacement with an appropriate program, e.g. PHASER using the structures of H3 (PDB 1HA0) and Fab (PDB 1EO8) (Chen et al., 2000, Fleury et al, 2000) as starting models. Generally, the asymmetric unit contains four HA trimers and twelve F005-126 molecules and may be refined using CNS with tight restrains and manual rebuilding with an appropriate software, e.g. Coot (Emsley et al., 1994). Positive density for N-linked glycosylation may be observed at 5 of the 6 predicted sites on HA, and a total of 20 sugar residues may be built. Hydrogen bonds and van der Waals contacts between F005-126 and H3 HA or HA from other strain may be calculated using an appropriate program, e.g., HBPLUS and CONTACSYM, respectively (McDonald et al., 1994, Sheriff et al., 1987). Surface area buried upon Fab binding may be calculated with an appropriate program, e.g., MS (Connolly et al., 1983). PyMol (DeLano Scientific) or any other suitable programs may be used to render structure figures and for general manipulations. Kabat numbering may be applied to the coordinates using an appropriate program, e.g., the AbNum server (Abhinandan et al., 2008). Exemplary final refinement statistics are summarized in Refien Lower, JKL-90_{—}120608.

Furthermore, those regions involved in the interaction between F005-126 and HA trimer are summarized in Table 1 and 2. Table 1 shows the relevant amino acid residues and/or saccharide residues involved in the interaction. Hydrogen bond and van der Waals contacts are also shown in Table 1 described at the bottom of the specification. Tables 2A and 2B show the atomic coordinates of H3 which are particularly relevant to the binding (CDR1, CDR2, FR3 and CDR3 of the heavy chain). Table 2A shows the atomic coordinates of H3 which are particularly relevant to the binding (JKL-90_{—}120608.pdb). Table 2B shows the atomic coordinates of H3 which are particularly relevant to the binding (JKL-90_{—}0101.pdb). The effective number of decimal place is first decimal place, in notation of the atomic coordinate.

(Diffraction and Structure Solving)

Native and derivative diffraction data may be collected in house or using commercially available service. Additional native data (e.g. at 4.0 Angstroms or higher resolution) may be collected on e.g. beamline ID14 (ESRF, Grenoble, France), on a MAR CCD detector. All data may be processed using DENZO and SCALEPACK [Otwinowski & Minor (1996) Methods Enzymol. 276:307] and merged using the CCP4 program suite [CCP4, Acta Crystallogr. D50, 760 (1994)]. Crystallographic phases were calculated with CCP4 programs and refined using SHARP [Fortelle &. Bricogne (1997) Methods Enzymol. B 472] and SOLOMON [Abrahams & Leslie (1996) Acta Crystallogr. D52, 30.]. The resulting electron density maps may allow about 80% of the two independent molecules to be traced. Model building and inspection may be based on the O suite [Jones et al. (1991) Acta Crystallogr. A47, 110]. The structure may be refined using CNS [Brunget et al. (1998) Acta Crystallogr. D 54, 905] and REFMAC [Murshudov et al. (1997) Acta Crystallogr. D 53, 240], 0.5% of the unique data may be used to monitor the free R-factor. The Ramachandran plot may be used for further analysis.

The interaction between the target molecule such as HA trimer or antibody or the like and its complementary binding molecule (antibody or antigen or the like) is specific to the functional binding site and this means that when the target molecule is bound to at least part of the substrate, the functional binding site must be orientated in such a way as to be available for subsequent interaction with its complementary binding molecule. This has implications with respect to the relative position of the concave region of HA trimer and functional binding site on the antibody.

In addition to the data disclosed herein, additional experimental 3-D structure of the target molecule such as HA trimer and/or antibody obtained by x-ray diffraction or NMR spectroscopy techniques is possibly the preferable source of information for the modelling of the present invention. Both published and proprietary databases may be used in this regard. For instance, the Protein Data Bank (PDB) is the largest worldwide repository for the processing and distribution of 3-D structure data of large molecules such as proteins. In the absence of such experimental structure, homology modelling may generate a software-based 3-D model of the target molecule. For example, for a target protein this may be done using its amino acid sequence and relating that to the structures of known proteins.

It may also be appropriate to undertake a bioinformatic search of relevant databases to search for the presence of potential anchoring sites. For example, public or proprietary databases of protein motifs or domain such as NCBI Dart, Smart, Pfam, Prosite, Interpro or Blocks may provide data and tools to identify which domains are present within the target molecule such as HA trimer and/or antibody (Marchler-Bauer et al., CDD: a database of conserved domain alignments with links to domain three-dimensional structures. Nucleic Acids research 30 281-283 (2002)). Analysis of protein-protein interaction screening data experimentally generated, for example, using yeast two-hybrid screens, may also provide information on which anchoring sites are present within the target molecule.

Alternative concave region in a HA trimer, which may be used in the present invention, may also be identified by computer modelling of the 3-D structure of a given target molecule such as a variant of HA trimer. One skilled in the art would be familiar with sources of such information and with the kind of computer hardware/software that may be employed. However, while ligand active sites can be identified, for example, by using the Grid, MCSS, superstar, Q-fit programs or the Sphgen module from the Dock computer programs suite, identifying binding sites on a protein surface is recognized as being a difficult task. Indeed, it has been shown that a binding site present at the surface of a protein may be practically indistinguishable from other patches on the protein surface. Palma et al., (BiGGER: a new (soft) docking algorithm for predicting protein interactions; Proteins, 2000 Jun. 1; 39(4):372-84) describe the use of BiGGER, a soft docking algorithm for predicting protein interactions based on the three-dimensional structures of unbound molecules. Recently, Ma et al., (Protein-protein interactions: Structurally conserved residues distinguish between binding sites and exposed protein surfaces, PNAS 2003 100: 5772-5777) have demonstrated that the use of polar residue hot spots can be used to determine potential binding regions.

Not all possible antibody identified to specifically bind to the concave region of HA trimer in the present invention may ultimately be useful for binding the target molecule to the substrate surface or antigen-antibody interaction or neutralizing activity and it is therefore usually necessary to identify a number of different locations on the antibody or HA trimer. This also affords design flexibility. Thus, any substances binding to concave region that are identified as candidate ligands or binding substance for binding of the concave region of the HA trimer but that would also result in non-inhibition of binding at the concave region of the HA trimer may be dismissed from further consideration.

Subsequent to identifying a suitably positioned concave region and other interaction sites on the target molecule such as HA trimer and/or antibody, the method of the invention involves generating a pharmacophore model for that concave region. In the context of the present invention the pharmacophore model is a set of spatially distributed properties or feature types that are likely to be responsible for the ability of a binding site (in this case the anchor site) to undergo some form of binding interaction. The pharmacophore model involves molecular features that relate to any form of interaction through which a binding site has binding potentials, for example, hydrophobic, electrostatic and hydrogen-bonding interactions. The pharmacophore model characterises a particular binding site by reference to such molecular features.

The pharmacophore model is a 3-D representation of molecular features and, as such, must be defined by reference to at least four centres (spatially distributed properties). It may aid flexibility of design to use pharmacophore models that are characterised by more than four centres as this brings a greater number of candidate concave region binding antibodies which may interact with the concave region as required.

The pharmacophore model can be generated by reference to the molecular features of the binding site itself and/or by reference to the molecular features of a set of one or more ligands, antibodies and/or antigens already known to bind to the concave region of interest or bindong region of F005-126 antibody. One skilled in the art would be aware of sources of information concerning complementary ligands, antibodies, and/or antigens for a given concave region of a target molecule, such as HA trimer. For example, a number of online resources are available for protein-protein interactions. The Biomolecular Interaction Network Database (BIND) stores descriptions of interactions and molecular complexes such as between proteins, nucleic acids and small molecules. The Dictionary of Interfaces in Proteins (DIP) is another resource on interacting protein surfaces.

Numerous techniques for generating a pharmacophore model are known in the art and the invention does not reside in the selection of any particular technique. By way of example, the following methodology and/or software systems may be mentioned: Catalyst; Ludi, DISCO; HipHop; GASP, Chem-X, Think and HypoGen. One skilled in the art would have no difficulty in using any of the known techniques in the context of the present invention.

Once a pharmacophore model has been generated for a concave region, the method of the invention involves using the pharmacophore model to identify an antibody or equivalent thereof binding to the concave region. The intention here is to identify an antibody or equivalent thereof which maps or fits the pharmacophore model to some extent and which therefore has potential to bind to the anchor site. Previously cited programs and others available in the art can be used to perform the virtual screening. An important aspect of the present invention is that the ligand, antibody, and/or antigen, or equivalents thereof do not have to match precisely the full pharmacophore model to be considered as a “hit” if the model is defined by reference to a large number of centers. At the very least, the ligand, antibody, and/or antigen, or equivalent thereof must match the pharmacophore model with respect to at least four centres thereof in order to have a potential to bind to an anchor site characterised by the model. Thus, if the pharmacophore model has been defined by reference to a large number of centers, it will be appreciated that the number of potentially useful ligands, antibodies, and/or antigens, or equivalent thereof that may be identified against the model will be increased. It will also be appreciated that if the pharmacophore model is defined by reference to a large number of centres, it may be possible to rank the likelihood of ligands exhibiting the necessary binding interaction based on the number of centres to which the ligand, antibody, and/or antigen, or equivalent thereof matches. A ligand which matches a pharmacophore model with respect to a large number of centers is likely to be more suitable than a ligand, antibody, and/or antigen, or equivalent thereof which matches the model in a more limited way.

With respect to the screening method of the present invention it may be useful to resort to compound/antibody databases which generally correspond to a corporate collection of physically available compounds/antibodies or compounds/antibodies available externally from chemical compound suppliers. In this latter case, two types of libraries can be used. The first type originates from molecules that can be bought on a one-at-the-time basis. Individual supplier catalogue of compounds can be used or compilations such as the MDL's ACD (Available Chemicals Directory) or CambridgeSoft's ChemACX might be a more comprehensive source. The second type of library is a screening library from screening compound collection suppliers where the full library or part of it can be acquired. Compilations of screening libraries are also available like the MDL Screening Compounds Directory or CambridgeSoft's ChemACX-SC. Another source of information might be a virtual library corresponding to compounds generated by computer software (CombiLibMaker, Legion) from a list of reagent and a given chemistry.

Molecular modelling software and techniques known in the art may also be used to translate a particular pharmacophore model into suitable ligand/antibody/antigen structures. Ludi is an example of a program that offers a de novo technique that has been recently extended to work with larger databases of flexible molecules. Techniques known in the art for performing this particular step are well suited for designing relatively small ligands (molecules) and they cannot readily be extended to the design of surface biomimetics. The main reason for this is the nature of the binding interactions involved in the binding event for a given binding site. For proteins, at least, the average contact area is 800 angstroms and molecules that could complement such a large surface area are generally rare. Furthermore, molecules in the high range of surface area generally have a large number (e.g. in excess of 15) of rotatable bonds (excluding terminal groups) and it is either not possible or not practical to use current pharmacophore methodologies for processing the vast array of possible configurations. Thus, the anchor site binding ligands generated in this step are relatively small and simple molecules. In practice it is expected that other surface components will contribute to the total binding energy that results in the binding of a target molecule.

In reality it is not guaranteed that an antibody or other compound binding to the concave region identified in accordance with the present invention will bind as desired to an anchor site. For instance, part of the antibody or other compound may collide with residues of the concave region or one or more structural features in the candidate antibody or other compound may be incompatible with one or more functional groups of the concave region. The technique which is adopted generates candidate antibody or other compound and the method of the invention preferably also includes a docking step to ensure binding efficacy of a concave region/antibody or other compound pair. This also allows antibody or other compound to be ranked according to binding affinity for the concave region.

Docking may be performed by various techniques known in the art such as Dock, FlexX, Slide, Fred, Gold, Glide, AutoDock, LigandFit, ICM, QXP, as exemplified elsewhere herein. It is to be noted that the optimized complex may no longer fit the pharmacophore centres that were initially used to position the antibody or any other ligand. The result may be that an antibody or any other compound binding to the concave region is predicted to bind to the concave region.

In the method of the present invention, candidate antibody or any other compounds or substances may be provided onto a surface of a substrate. The antibody or any other compounds or substances must be immobilized on the surface of the substrate so that the HA trimer or any other substance comprising the concave region may itself be immobilized.

The fact that the antibodies or any other compounds or substances, e.g., antigen-binding antibody fragments, are small molecule compounds greatly increases the likelihood of being able to provide them with the correct spatial distribution on the substrate surface. In the prior art, low affinity ligands identified through experimental means have been tethered together through flexible linkers to form higher affinity ligands (D. J. Maly, et al. Combinatorial target guided ligand assembly: Identification of potent subtype-selective c-Src inhibitors., Proc. Natl. Acad. Sci., 97, 2000, 2419-2424; S. B. Shuker, et al., Discovering high-affinity ligands for proteins: SAR by NMR, Science, 274, 1996, 1531-1534.). However, the focus of such work was to develop small molecule drug candidates and not polymeric coatings. Also, Lacroix et al (Lacroix, M., Dionne, G., Zrein, M., Dwyer, R. J. and Chalifour, R. J. “The use of synthetic peptides as solid phase antigens” Chapter 16 in CRC Immunochemistry of Solid Phase Immunoassays, J. E. Butler, Ed., 1991), describe that the use of synthetic peptide antigens that ideally represent only the minimal size necessary to mimic a given antigenic determinant resulted in an increase in the density of epitope which could be coated on a solid phase. One advantage of this high epitope density was that it leads to bivalent attachment of antibodies, a condition that could result in a 1000-fold increase in functional affinity (avidity) relative to monovalent antibody attachment.

The substrate may be formed of any material conventionally used in the intended field of application. For example, the substrate may be glass, silica or plastic. Suitable plastics materials include: nitrocellulose; polyolefins such as polyethylene, polypropylene and polymethylpentene; polystyrene or substituted polystyrenes; fluorinated polymers such as poly(tetrafluoroethylene) and polyvinylidene difluoride; polysulfones such as polysulfone and polyethersulfone; polyesters such as polyethylene terephthalate and polybutylene terephthalate; polyacrylates and polycarbonates; and vinyl polymers such as polyvinylchloride and polyacrylonitriles.

The substrate may take any form. In biological applications the substrate will usually be in the form of beads, membranes, multi-well plates, slides, capillary columns or any other format that is used for biological assays, affinity separations, diagnostics or other applications where biological molecules are immobilised on some insoluble material (solid support).

Depending upon the chemical functionality available to attach the antibody or any other compounds or substances to the substrate, it may be appropriate to functionalize the substrate to facilitate suitable coupling of the antibody or any other compounds or substances. Obviously, the latter must be attached to the substrate in such a way that its ability to undergo a suitable binding interaction with a concave region of a HA trimer is preserved. By way of example, if the antibody or any other compounds or substances includes a carboxylic acid functionality available for coupling the antibody or any other compounds or substances to the substrate, it may be appropriate to derivatize or modify the surface of the substrate in some way to enable coupling of the antibody or any other compounds or substances through this carboxylic acid functionality. This may be achieved by coating of the substrate with a material that is reactive towards the carboxylic acid functionality of the antibody or any other compounds or substances. It is of course necessary to assess the effect of such coating on the intended binding interaction between the antibody or any other compounds or substances and a concave region of a HA trimer, and this may be done experimentally, as described herein. By way of illustration, when the antibody or any other compounds or substances includes a carboxylic acid functionality available for coupling of the ligand to the substrate, the substrate may be coated with polyethyleneimine, the amino groups of which are able to react with the carboxylic acid functionality of the antibody or any other compounds or substances.

Where specific compounds are referred to above as being the antibody or any other compounds or substances, it will be appreciated that the compound must be coupled to a substrate prior to use. It is envisaged that this coupling will rely on a functional group present in the compound. This is described above in relation to compounds including carboxylic acid functionality.

### DESCRIPTION OF PREFERRED EMBODIMENTS

It should be understood that while description of preferred embodiments is described below, the embodiments are illustrative of the present invention and the scope of the present invention is not limited to such preferred embodiments. It should be also understood that those skilled in the art can readily carry out modification, alternation or the like within the scope of the present invention with reference to the preferred embodiments below.

(Antibody F005-126 and Use Thereof)

In an aspect, the present invention provides an isolated antibody directed to hemagglutinin (HA) trimer of an influenza virus. The antibody or a funtionally equivalent variant of the present invention comprises:

(i) the sequence of CDR1 (SEQ ID NO: 3) of F005-126 antibody heavy chain (SEQ ID NO: 2), or a functionally equivalent sequence thereof;

(ii) the sequence of CDR2 (SEQ ID NO: 4) of F005-126 antibody heavy chain (SEQ ID NO: 2), or a functionally equivalent sequence thereof;

(iii) the sequence of CDR1 (SEQ ID NO: 5) of F005-126 antibody heavy chain (SEQ ID NO: 2), or a functionally equivalent sequence thereof; and

(iv) the sequence of FR3 (SEQ ID NO: 8) of F005-126 antibody heavy chain (SEQ ID NO: 2), or a functionally equivalent sequence thereof. In the present invention, the inventors found that the specific portions of CDR1, CDR2, CDR3 and FR3 of F005-126 antibody heavy chain play an important role in binding to a HA trimer. Therefore, it should be understood that as long as such specific portions are conserved, any functionally equivalent sequence thereof may be used.

In a specific embodiment, the antibody or a funtionally equivalent variant of the present invention further comprises

(v) the sequence of FR1 (SEQ ID NO: 6) of F005-126 antibody heavy chain (SEQ ID NO: 2), or a functionally equivalent sequence thereof

(vi) the sequence of FR2 (SEQ ID NO: 7) of F005-126 antibody heavy chain (SEQ ID NO: 2), or a functionally equivalent sequence thereof; and

(vii) the sequence of FR4 (SEQ ID NO: 9) of F005-126 antibody heavy chain (SEQ ID NO: 2), or a functionally equivalent sequence thereof. It should be undestood that such specific FR1, FR2 and FR4 are optional and thus are not always necessary, since these FR regions are not directly involved in the binding of the antibody to the HA trimer.

In another embodiment, said antibody comprises at least one of the properties selected from the group consisting of: (1) having broad strain specificity against H3; (2) binds to HA1 head region but does not inhibit binding to cell; (3) inhibits structural change of HA; (4) said CDR1, CDR3 and FR3 bind to HA by van der Waals contact; (5) said CDR2 binds to N285 sugar chain which is conserved in HA; (6) binds to the HA trimer across two HA subunits thereof which are adjacent to each other; (7) intra- and inter-subunit interactions between HA1 and HA2 by salt bridges are located on the amino acid sequence of the molecular surface in the vicinity of the portion which maintains structure of the HA trimer; and (8) comprising hydrogen bonds. It should be understood that such features are not known in the conventional anti-HA/influenza virus antibodies. As such, the antibodies of the present invention provide signficant effects associated with these binding features, and provide novel aspects of manners of assaying or screening for a novel anitbody which may be used for passive immunotherapy for influenza virus and/or vaccines therefor.

In a preferably embodiment, the antibody has a property of binding to the HA trimer across two HA subunits thereof which are adjacent to each other. Such a feature of “bridging” the two monomers by an antibody is not known or suggested. Although not wishing to be bound to any theory, such “bridging” feature provides a stabilizing action of HA trimers by an antibody, which may result in improvement of raising neutralizing activity. Therefore, it may be possible that the bridging feature results in improvement in the therapeutic activity for influenza virus infection.

In another embodiment, the antibody or a funtionally equivalent variant of the present invention is a neutralizing antibody.

In a particular embodiment, the antibody or a funtionally equivalent variant of the present invention is a neutralizing antibody which neutralizes at least H3. However, it should be noted that the antibody or a funtionally equivalent variant of the present invention may cross react with the other type of influenza including, but not limited to, H1, H2 and H5 and the like.

In an additional embodiment, the antibody comprises (a) the sequence set forth in SEQ ID NO: 2 (the full sequence), or (b) a sequence derived from the sequence of (a) comprising one or more amino acid substitution(s), deletion(s) and/or addition(s). Such an amino acid substitution may preferaly be a conservative substitution.

In an further embodiment, the antibody comprises: (a) the sequence set forth in SEQ ID NO: 2 (the full sequence), or (b) a sequence derived from the sequence of (a) comprising one or more amino acid substitution(s), deletion(s) and/or addition(s) at positions other than the binding site with HA of CDR1 sequence of F005-126 antibody (amino acid No. 31 (Ser) of SEQ ID NO. 2), the binding site with HA of CDR2 sequence of F005-126 antibody (SEQ ID NO: 10 (amino acids No. 54-58 (Tyr Asn Gly Asn Thr) of SEQ ID NO. 2)), the binding site with HA of CDR3 sequence of F005-126 antibody (amino acids No. 74-76 (Thr Ser Thr) of SEQ ID NO. 2), and the binding site with HA of FR3 sequence of F005-126 antibody (SEQ ID NO: 11 (amino acids No. 102-105 (Val Arg Gly Val) of SEQ ID NO. 2)), wherein the sequence maintains the binding activity with the HA trimer.

In a furthermore embodiment, the antibody comprises: (a) the sequence set forth in SEQ ID NO: 2 (the full sequence), or (b) a sequence derived from the sequence of (a) comprising one or more amino acid substitution(s), deletion(s) and/or addition(s) at the positions other than the CDR1 sequence of F005-126 antibody (SEQ ID NO: 3), the CDR2 sequence of F005-126 antibody (SEQ ID NO: 4), the CDR3 sequence of F005-126 antibody (SEQ ID NO: 5), and the FR3 sequence of F005-126 antibody (SEQ ID NO: 8), wherein the sequence maintains the binding activity with the HA trimer. Such substitution is preferably a conservative substitution.

In a specific embodiment, the antibody of the present invention consists of the sequence set forth in SEQ ID NO: 2 (the full sequence).

In another aspect, the present invention provides a screening kit for an antibody against hemagglutinin (HA) trimer of an influenza virus, comprising the antibody or a funtionally equivalent variant of the present invention as disclosed herein. Such an antibody may be of any embodiment described herein as long as the object of the kit, e.g., screening for an antibody against HA trimer is achieved. In an embodiment, the kit comprises the antibody of the invention as an internal standard, for, e.g. competitive assay.

In a certain embodiment, the kit of the present invention further comprises a protein or protein complex comprising the sequence of concave region of the HA trimer (e.g. SEQ ID NO: 48 and 21). The concave regions employed herein are defined herein elsewhere. The concave region may be included in a single protein comprising e.g., two monomers expressed as a single fusion protein or a chimeric protein, or a protein complex comprising e.g. two monomers or equivalent thereof.

In another aspect, the present invention provides an influenza virus passive immunotherapy agent comprising the antibody or a funtionally equivalent variant of the present invention. The antibodies of the present invention interact with the concave region, which has now been clarified to play an imporant role in raising neutralizing activity, the antibody which interacts with the concave region may be able to raise neutralizing activity, and therefore may be used as an influenza virus passive immunotherapy agent.

In another aspect, the present invention provides a method of influenza virus passive immunotherapy comprising the step of administering the antibody or a funtionally equivalent variant of the present invention to a patient in need thereof. In the method of the passive immunotherapy, any type antibody or a funtionally equivalent variant of the present invention disclosed herein may be used.

(Concave Region)

In a different aspect, the present invention provides a kit for paratope analysis of an influenza neutralizing antibody comprising a protein or protein complex comprising the sequence of concave region of the HA trimer (e.g. SEQ ID NO: 48 and 21). The concave region of the present invention is useful for analyzing and screening a paratope of an antibody, and subsequently be used for development and improvement of an antibody, thereby be used for immunotherapy. The kit of the present invention may be used in any assay as long as it is appropriately used. Exemplary assay methods are described elsewhere herein, including any immunochemical assays such as ELISA or Western blot and the like.

In certain embodiments, the protein or protein complex is (A) the full length sequence of the HA trimer (e.g. SEQ ID NO: 48 and 21); or (B) a sequence derived from the full length sequence of (B) comprising one or more amino acid substitution(s), deletion(s) and/or addition(s) at the positions other than the sequence of concave region, wherein the sequence maintains the binding activity with F005-126 antibody.

In another embodiment, the protein or protein complex consists of (A) the full length sequence of the HA trimer (e.g. SEQ ID NO: 48 and 21), in a single protein form or a complex form.

In another embodiment, the paratope is related to an antibody against Group 2 hemagglutinin, however the present invention is not limited thereto, and may also be used for Group 1 hemagglutinin.

In another embodiment, the paratope is related to an antibody against hemagglutinin H3, however the present invention is not limited thereto, and may also be used for other subtypes of hemagglutinin including H1, H2, H5 and the like.

In another embodiment, the paratope is related to an antibody against hemagglutinin H3, whose strain is selected from the group consisting of (e.g. see FIG. 8-1 which includes H3N2 and H3N8)

(Modeling Inventions)

In another aspect, the present invention provides a method for identifying a binding substance to a hemagglutinin (HA) trimer of an influenza virus, the method comprising the steps of:

(A) providing a 3D structural representation of the HA trimer, wherein the 3D structural representation of the HA trimer comprises the atomic co-ordinates relating to a 3D structural representation of the amino acid residue position contained in the HA of Table 1 which is described at the bottom of the specification;

(B) providing a 3D structural representation of a candidate substance of the binding substance;

(C) using a computer to dock the 3D structural representation of the candidate substance with the 3D structural representation of the HA trimer, wherein a candidate substance that docks with the HA trimer at the site comprising the amino acid residue positions contained in the HA of the Table 1, is identified as the binding substance of the HA trimer;

(D) contacting the candidate substance identified in step (C) with HA trimer or a fragment thereof containing the 3D structure of the amino acid residues contained in the HA of the Table 1; and

(E) assaying the interaction between the candidate substance and the HA trimer or the fragment thereof, to determine whether the binding substance identified in step (C) is a binding substance for the HA trimer.

The method of the present invention may be performed using any program used in the bioinformatics or molecular modelling art, some of which are exemplified and described elsewhere herein. Assaying the interaction may be conducted by any methods such as immunochemical assays such as ELISA, Western blotting and the like, which are also described elsewhere herein in detail.

In certain embodiments, the 3D structural representation comprises at least one interaction selected from the group consisting of van der Waals contacts, electrostatic interactions, and hydrogen bonding. Methods using such interactions may be performed using any program used in the bioinformatics or molecular modelling art, some of which are exemplified and described elsewhere herein. Preferably, the 3D structural representation comprises van der Waals contacts, electrostatic interactions, and hydrogen bonding.

In certain embodiments, the 3D structural representation of the amino acid residues contained in the HA of the following Table 1 comprises

(A) the atomic co-ordinates set forth in Table 2 [Tables 2-1 to 2-4] which is located at the bottom of the specification
or
(B) variant atomic co-ordinates of (A), in which the r.m.s. deviation of the x, y and z co-ordinates for all heavy atoms is less than 4.0 (or 2.5) Angstroms.

In certain embodiments, the 3D structural representation of the amino acid residues contained in the HA of the following Table 1 comprises the entire atomic co-ordinates set forth in PDB1, PDB2, PDB3 and/or PDB4.

In certain embodiments, said step of docking comprises geometric mathcing or minimizing the energy of interaction between the candidate substance and the HA trimer of the amino acid residue position contained in the HA of the Table 1.

In certain embodiments, the candidate substance comprises a library of antibodies. Such a library may be prepared using conventional technology, which are also described elsewhere herein, or obtained from known source.

In certain embodiments, the binding substance is an inhibitor for HA trimer. Such an inhibitory activity may be assayed using a conventional technology which are exemplified and described elsewhere herein.

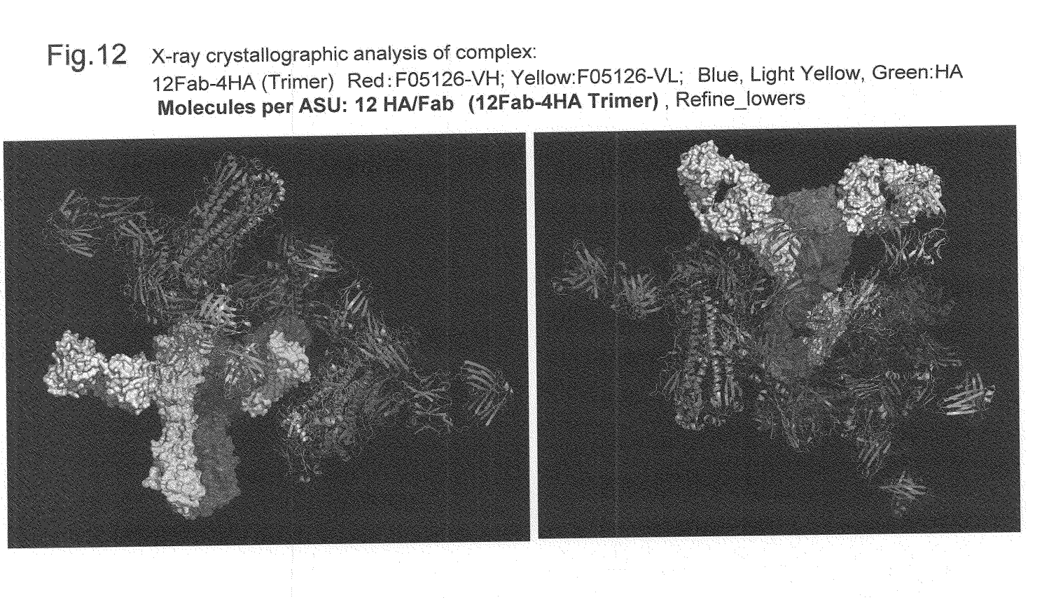

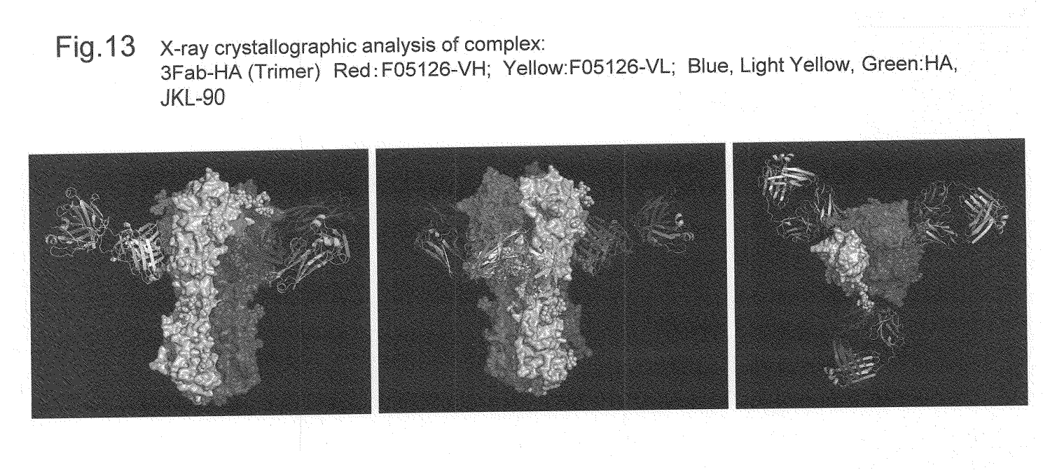

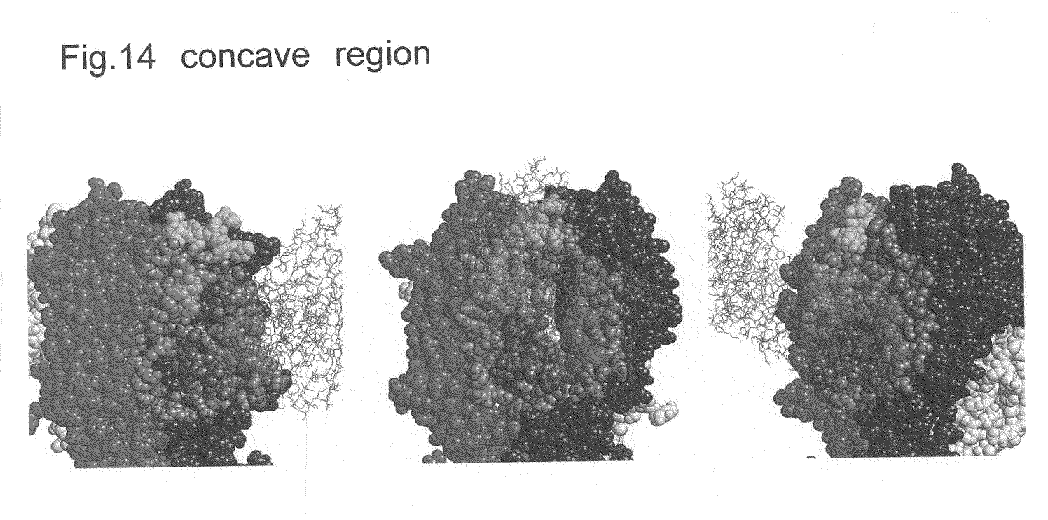

In certain embodiments, the step of docking comprises referring to the 3D structural representation of the antibody set forth in PDB1, PDB2, PDB3 and/or PDB4 and FIGS. 12 and 13, preferably PDB2 and/or PDB4 and FIG. 13. Methods using such interaction may be performed using any program used in the bioinformatics or molecular modelling art, some of which are exemplified and described elsewhere herein.

(Antigen in the Complex)

In another aspect, the present invention provides a conformational epitope formed by an antibody-antigen complex of antibody F005-126, antigen HA-a+HA-b and water molecule, wherein the HA-a and HA-b HA1 selected from the group consisting of SEQ ID NOs: 48-60 and 39-45 and HA2 selected from the group consisting of SEQ ID NOs: 21-38, wherein the conformational epitope comprises:

the following amino acid residues of the amino acid sequences of HA of H3N2 Aic 68 (SEQ ID NO: 48), or corresponding amino acid residues thereto (See FIGS. 8-1 and 8-2):

a Site L epitope element comprising amino acid residues N171, D172, N173, P239 and G240;

a Site R epitope element comprising amino acid residues S91, K92, S270, D271, A272, P273, P284 and N285;

a Site R epitope element comprising sugar chains NAG(N-acetyl-D-glucosamine)1, NAG2 BMA(beta-D-mannose)3, MAN(alpha-D-mannose)4, MAN5, MAN6 and MAN7, linked to amino acid residue N285,

wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

In certain embodiments, said crystal has the atomic co-ordinates set forth in PDB1, PDB2, PDB3 or PDB4.

In certain aspects, the present invention provides an antigen comprising the epitope of the present invention. It should be noted that such an antigen may be used for therapeutic or preventive use, or as a reagent for screening or any other analysis methods, which are also described and exemplified elsewhere herein.

In certain aspects, the present invention provides a vaccine comprising the antigen of the present invention. It should be noted that such a vaccine may be used for therapeutic or preventive use.

In certain aspects, the present invention provides a screening method of a neutralizing antibody using the antigen of the present invention. It should be noted that such a neutralizing antibody may be used for therapeutic or preventive use. Vaccines and neutralizing antibodies may be used in a medicament form and such a medicament is exemplified and described elsewhere herein.

(Antibody in the Complex)

In another aspect, the present invention provides a paratope of antibody F005-126 in an antibody-antigen complex of antibody F005-126, antigen HA-a+HA-b and water molecule, wherein the HA-a and HA-b are selected from the group consisting of SEQ ID NOs: 48-60, wherein the paratope comprises: the following amino acid residues of F005-126 heavy chain (SEQ ID NO: 2):
a paratope element comprising amino acid residues T73, G74, and T75 (according to the Kabat's numbering shown in FIG. 5-2);
paratope element comprising amino acid residue S31 (according to the Kabat's numbering shown in FIG. 5-2);
a paratope element comprising amino acid residues Y53, D54, G55, Q56 and H57 (according to the Kabat's numbering shown in FIG. 5-2); and
a paratope element comprising V98, R99, G100, and V100a (according to the Kabat's numbering shown in FIG. 5-2);

wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

In certain embodiments, said crystal has the atomic co-ordinates set forth in PDB1, PDB2, PDB3 and/or PDB4.

In certain aspects, the present invention provides a neutralizing antibody comprising the paratope of the present invention. It should be noted that such an antibody may be used for therapeutic or preventive use, or as a reagent for screening or any other analysis methods, which are also described and exemplified elsewhere herein.

In certain aspects, the present invention provides a passive immune therapy agent comprising an antibody comprising the paratope of the present invention. It should be noted that such an immune therapy agent may be used for therapeutic or preventive use.

In certain aspects, the present invention provides a screening method of a vaccine using the paratope of the present invention. It should be noted that such a vaccine may be used for therapeutic or preventive use. Vaccines and immune therapy agents may be used in a medicament form and such a medicament is exemplified and described elsewhere herein.

(Screening)

In another aspect, the present invention provides a partial complex or full complex of a conformational epitope and paratope formed by an antibody-antigen complex of antibody F005-126, antigen HA-a+HA-b and water molecule, wherein the HA-a and HA-b are selected from the group consisting of SEQ ID NOs: 48-60, wherein the conformational epitope comprises:

the following amino acid residues of the amino acid sequences of HA of H3N2 Aic 68 (SEQ ID NO: 48), or corresponding amino acid residues thereto:

a Site L epitope element comprising amino acid residues N171, D172, N173, P239 and G240

a Site R epitope element comprising amino acid residues S91, K92, S270, D271, A272, P273, P284 and N285;

a Site R epitope element comprising sugar chains NAG(N-acetyl-D-glucosamine)1, NAG2 BMA(beta-D-mannose)3, MAN(alpha-D-mannose)4, MAN5, MAN6 and MAN7, linked to amino acid residue N285,

wherein the paratope comprises:

the following amino acid residues of F005-126 (SEQ ID NO:2):
a paratope element comprising amino acid residues T73, G74, and T75 (according to the Kabat's numbering shown in FIG. 5-2);
a paratope element comprising amino acid residues S31 (according to the Kabat's numbering shown in FIG. 5-2) (named as P-Site-R-1) (in CDR1);
a paratope element comprising amino acid residues Y53, D54, G55, Q56 and H5 (according to the Kabat's numbering shown in FIG. 5-2) 7;
a paratope element comprising V98, R99, G100, and V100a (according to the Kabat's numbering shown in FIG. 5-2);

wherein the space group of the crystal formed by the complex is C2, and the lattice constant thereof is |a|=391.037±5.0 Angstroms, |b|=241.173±5.0 Angstroms, |c|=223.214±5.0 Angstroms, α=γ=90°, β=123.62°, which is an orthorhombic system.

In certain aspects, the present invention provides a complex comprising the partial or full complex of the present invention. It should be noted that such a complex may be used as a reagent for screening or any other analysis methods, which are also described and exemplified elsewhere herein.

In certain aspects, the present invention provides a screening method for a neutralizing antibody or a vaccine using the partial or full complex of the present invention. It should be noted that such a neutralizing antibody and/or vaccine may be used for therapeutic or preventive use. Vaccines and neutralizing antibodies may be used in a medicament form and such a medicament is exemplified and described elsewhere herein.

In certain aspects, the present invention provides a method for screening an active agent for hemagglutinin for hemagglutinin comprising: (a) constructing a 3D structure model of hemagglutinin using any one of PDB1, PDB2, PDB3 or PDB4; (b) identifying a dock site; (c) carrying out docking simulations for a first library of compounds as an initial screen; (d) selecting hits from the initial screen; and (e) performing a secondary screen using a combined library of the hits from the initial screen and a second library. It should be noted that such an active agent for hemagglutinin may be used for therapeutic or preventive use. Such an active agent for hemagglutinin may be used in a medicament form and such a medicament is exemplified and described elsewhere herein.

In certain aspects, the present invention provides a method for estimating variations within subtypes of Influenza A viruses, comprising: (a) providing amino acid sequences of Influenza A virus; (b) extracting complete Hemagglutinin sequences from the amino acid sequences of step (a); (c) aligning the sequences extracted in step (b) and identifying the epitope regions according to the positions shown in FIG. 8.; and (d) estimating the variation of each subtype by computing Shannon index of each site, by counting the number of different kind of sequences and by making sequence logos. It should be noted that such variations within subtypes of Influenza A viruses may be used for diagnostic, therapeutic or preventive use. Such variations within subtypes of Influenza A viruses may be used in preparing a medicament and such a medicament is exemplified and described elsewhere herein.

In certain aspects, the present invention provides a method for screening an active agent for hemagglutinin for hemagglutinin comprising: (a) constructing a 3D structure model of hemagglutinin using any one of PDB1, PDB2, PDB3 or PDB4; (b) identifying a dock site; (c) carrying out docking simulations for a first library of compounds as an initial screen; (d) selecting hits from the initial screen; and (e) performing a secondary screen using a combined library of the hits from the initial screen and a second library. It should be noted that such an active agent for hemagglutinin may be used for therapeutic or preventive use. Such an active agent for hemagglutinin may be used in a medicament form and such a medicament is exemplified and described elsewhere herein.

In certain aspects, the present invention provides a method for estimating variations within subtypes of Influenza A viruses, comprising: (a) providing amino acid sequences of Influenza A virus; (b) extracting complete Hemagglutinin sequences from the amino acid sequences of step (a); (c) aligning the sequences extracted in step (b) and identifying the epitope regions according to the positions shown in FIG. 8.; and (d) estimating the variation of each subtype by computing Shannon index of each site, by counting the number of different kind of sequences and by making sequence logos. It should be noted that such variations within subtypes of Influenza A viruses may be used for diagnostic, therapeutic or preventive use. Such variations within subtypes of Influenza A viruses may be used in preparing a medicament and such a medicament is exemplified and described elsewhere herein.

In certain aspects, the present invention provides a method for screening active agent for regulating influenza virus or influenza virus hemagglutinin comprising: (a) constructing a 3D structure model of hemagglutinin using any one of PDB1, PDB2, PDB3 and PDB4; (b) identifying a dock site; (c) carrying out docking simulations for a first library of compounds as an initial screen; (d) selecting hits from the initial screen; and (e) performing a biological assay with the candidate compound to confirm that the compound has the regulating activity. It should be noted that such an active agent for influenza virus or hemagglutinin may be used for therapeutic or preventive use. Such an active agent for hemagglutinin may be used in a medicament form and such a medicament is exemplified and described elsewhere herein. As used herein, a biological assay may be any assay which allows judgment of the regulating activity of influenza virus or hemagglutinin, such as trypsin assay as in Experiment 6 of the present specification.

When the subject screening method of the invention is performed, an agent to be screened includes, but not limited to compounds such as low molecular weight compounds; biological molecules such as proteins, polypeptides, peptides, nucleic acids and the like; antagonists, agonists and the like.

As used herein, the term “agent” may be any substance or other entity (e.g., energy, such as light, radiation, heat, electricity, or the like) as long as the intended purpose can be achieved. Examples of such a substance include but are not limited to, proteins, polypeptides, oligopeptides, peptides, polynucleotides, oligonucleotides, nucleotides, nucleic acids (e.g., DNA such as cDNA, genomic DNA, or the like, and RNA such as mRNA), polysaccharides, oligosaccharides, lipids, low molecular weight organic molecules (e.g., hormones, ligands, information transfer substances, molecules synthesized by combinatorial chemistry, low molecular weight molecules (e.g., pharmaceutically acceptable low molecular weight ligands and the like) and the like) and the combinations of these molecules. Examples of an agent specific to a polynucleotide include, but are not limited to, representatively, a polynucleotide having a sequence complementarily to the sequence of the polynucleotide with a predetermined sequence homology (e.g., 70% or more sequence identity), a polypeptide such as a transcriptional agent binding to a promoter region and the like. Examples of an agent specific to a polypeptide include, but are not limited to, representatively, an antibody specifically directed to the polypeptide or derivatives or analogs thereof (e.g., single chain antibody), a specific ligand or receptor when the polypeptide is a receptor or ligand, a substrate when the polypeptide is an enzyme and the like.

As used herein, the term “compound” refers to any identifiable chemical substance or molecule, including but not limited to, a low molecular weight molecule, a peptide, a protein, a sugar, a nucleotide or a nucleic acid. Such a compound may be a naturally-occurring product or a synthetic product.

As used herein, the term “agent regulating” a certain target such as influenza virus or influenza virus hemagglutinin, such as a nucleic acid molecule or polypeptide refers to an agent which has a level of regulation such as suppression or activation of the target equal to or higher than that of the normal status. Such an agent may also be referred to as an “active agent. Examples of such an agent include, but are not limited to, when a target is a polypeptide, an antibody, a single chain antibody, an antigen fragment thereof, either of a pair of a receptor and a ligand, either of a pair of an enzyme and a substrate, and the like.

As used herein, the term “agonist” refers to an agent which binds to the receptor of a certain biologically acting substance (e.g., ligand, etc.), and has the same or similar function as the function of the substance.

As used herein, the term “antagonist” refers to a factor which competitively binds to the receptor of a certain biologically acting substance (ligand), and does not produce physiological action via the receptor. Antagonists include antagonist drugs, blockers, inhibitors and the like.

As used herein, the term “low molecular weight organic molecule” refers to an organic molecule having a relatively small molecular weight. Usually, the low molecular weight of an organic molecule refers to a molecular weight of about 1,000 or less, or alternatively may refer to a molecular weight of more than 1,000. Low molecular weight organic molecules can be ordinarily synthesized by methods known in the art or combinations thereof. These low molecular weight organic molecules may be produced by organisms. Examples of the low molecular weight organic molecules include, but are not limited to, hormones, ligands, information transfer substances, synthesized by combinatorial chemistry, pharmaceutically acceptable low molecular weight molecules (e.g., low molecular weight ligands and the like) and the like.

As used herein, the term “biological molecule” refers to molecules, or aggregates of molecules, relating to an organism and aggregates of organisms. As used herein, the term “biological” or “organism” refers to a biological organism, including but being not limited to, an animal, a plant, a fungus, a virus and the like. Biological molecules include molecules extracted from an organism and aggregations thereof, though the present invention is not limited to this. Any molecules or aggregates of molecules relating to an organism and aggregates of organisms fall within the definition of a biological molecule. Therefore, low molecular weight molecules (e.g., low molecular weight molecule ligands, etc.) capable of being used as medicaments fall within the definition of a biological molecule as long as an effect on an organism is intended. Examples of such a biological molecule include, but are not limited to, proteins, polypeptides, oligopeptides, peptides, polynucleotides, oligonucleotides, nucleotides, nucleic acids (e.g., DNA such as cDNA and genomic DNA; RNA such as mRNA), polysaccharides, oligosaccharides, lipids, low molecular weight molecules (e.g., hormones, ligands, information transmitting substances, low molecular weight organic molecules, etc.), and composite molecules thereof and aggregations thereof (e.g., glycolipids, glycoproteins, lipoproteins, etc.) and the like. A biological molecule may include a cell itself or a portion of tissue as long as it is intended to regulate the influenza virus and/or the hemagglutinin thereof. Typically, a biological molecule may be a nucleic acid, a protein, a lipid, a sugar, a proteolipid, a lipoprotein, a glycoprotein, a proteoglycan or the like. Preferably, a biological molecule may include a nucleic acid (DNA or RNA) or a protein. In an embodiment, a biological molecule is a nucleic acid (e.g., genomic DNA or cDNA, or DNA synthesized by PCR or the like). In another embodiment, a biological molecule may be a protein. Such a biological molecule may be a hormone or a cytokine.

The reference documents such as scientific documents, patents and patent applications cited as used herein are incorporated by reference as used herein to the same degree that entirety thereof is specifically described, respectively.

The present invention will be described below based on examples, but the following examples are provided only for the purpose of exemplification. Therefore, the scope of the present invention is not limited to the aforementioned embodiments or the following example, but is limited only by the attached claims.

### EXAMPLES

The present invention will be described in more detail below by way of examples, but the technical scope of the present invention is not limited by the examples, etc. Reagents, resins, etc. used in the following examples can be obtained from Wako Pure Chemical Industries, Ltd., Sigma-Aldrich, etc. unless otherwise indicated.

Abbreviations used in the present examples have the following meanings

HA: hemagglutinin
Ab: antibody
Ag: antigen
The abbreviations used in Figs:
Fab-cp3: fragment, antigen binding-coat protein 3
Fab-pp: fragment, antigen binding-P denotes a single Fc-binding domain of protein A
HI activity: haemagglutinin-inhibition activity

### Materials and Methods

### Viruses

The following influenza virus strains were used for experiments and analyses. A/H1N1 strains: A/New Calcdonia/20/1999 (NC99). A/H3N2 strains: A/Aichi/2/1968 (Aic68), A/Fukuoka/1/1970 (Fuk70), A/Tokyo/6/1973 (Tok73), A/Yamanashi/2/1977 (Yam77), A/Niigata/102/1981 (Nii81), A/Fukuoka/C29/1985 (Fuk85), A/Guizhou/54/1989 (Gui89), A/Kitakyushu/159/1993 (Kit93), A/Sydney/5/1997 (Syd97), A/Panama/2007/1999 (Pan99), A/Wyoming/3/2003 (Wyo03), and A/New York/55/2004 (NY04). In addition to these strains, the amino acid sequences and the 3D structural data of HA of the following strains were used for analyses. A/H1N1 strains: A/South Carolina/1/1918 (SC1918) and A/California/04/2009 (Cal09pdm). A/H2N2 strain: A/Japan/305+/1957 (Jpn57). A/H3N8 strain: A/wedge-tailed shearwater/Western Australia/405/1977 (aviAus77). A/H5N1 strain: A/Viet Nam/1203/2004 (Viet04). A/H7N3 strain: A/Turkey/Italy/214845/2002 (aviIta02). A/H9N2 strain: A/Swine/Hong Kong/9/98 (swHK98). Abbreviations for the strains are shown in the parentheses. The A/H3N2, and A/H1N1 strains listed above have been used for influenza vaccines in Japan.

### Construction of an Ab Library

A large combinatorial Ab library was constructed by using the phage-display method as previously described (Marks et al., 1991, Ohshima et al., 2011). In brief, 1.3×10⁹ B lymphocytes from a healthy donor born in 1974 were collected by apheresis from the equivalent of 3 L of blood. From the B lymphocytes, heavy (H) and light (L) chain libraries were constructed, which contained 3.7×10⁹ and 1.6×10⁸ clones, respectively. Finally H and L chain were combinatorially assembled. The resulting Ab library contained 2.9×10¹⁰ clones.

### Screening of the Ab Library

Phages bound to virus particles were selected by a panning method as described previously (Ohshima et al., 2011). In brief, formalin-treated virus particles were used as antigens (Ags) in the screenings. After three time pannings, the phages eluted were infected to E. coli (DH12S) and spread onto the LB plates containing 100 μg/ml ampicillin and 0.5% glucose. Colonies were picked up, and the E. coli colonies harboring phagemid were grown in 2×YT medium containing 100 μg/ml ampicillin, 0.1% glucose and 1 mM isopropyl-β-D-thiogalactopyranoside at 30° C. overnight. The Fab-cp3 form of MAb was secreted into the medium (Iba et al., 1997). The culture supernatants containing Fab-cp3 molecules were subjected to ELISA against 12 kinds of H3 strains and an H1 strain of influenza viruses. Clones that bound to H3 but not to H1 were selected and subjected to sequencing for classification. The amino acid positions of VH and VL were coordinated according to Kabat numbering using the AbNum server (Abhinandan et al., 2008).

Preparation of mAbs

Fab-cp3 Abs were purified with an anti-cp3 mAb-conjugated column. The Fab-PP Abs (Ito et al., 1993) (P denotes a single Fc-binding domain of protein A) were purified with IgG sepharose (GE Healthcare). IgG was prepared from a high expression vector and purified with Protein A Sepharose (GE Healthcare).

### ELISA

Inactivated virus particles were coated onto Maxisorp immunoplates (Nunc). The plates were incubated with human IgG Ab, and then peroxidase-conjugated goat anti-human IgG (H+L chain; MBL) was added. When Fab-cp3 Ab in the supernatant of E. coli culture was added to the virus-coated plate, rabbit anti cp3 Ab and peroxidase-conjugated goat anti-rabbit IgG (H+L chain; MBL) were used as the 2nd and 3rd Abs, respectively. Finally, HRP substrate (OPD; Wako) was added, and the OD at 492 nm was measured.

### Western Blot

Formalin-inactivated virus particles were separated by SDS-PAGE under non-reducing conditions and transferred to Immobilon-P membrane (Millipore). The membranes were incubated with the Fab-cp3 Abs. For detection, rabbit anti-cp3 Ab (MBL) was used as the primary Ab, and peroxidase-conjugated goat anti-rabbit IgG (H+L chain; MBL) was used as the secondary Ab. Then, the immunoreactive bands were visualized by using ECL Plus Western blotting Detection Regents (GE Healthcare).

### Neutralization Test

Neutralizing activity was measured by the focus reduction assay as described previously (Okuno et al., 1990). A series of dilutions of F005-126 IgG (50 μl) was mixed with 100 FFU of influenza viruses (50 μl) and then applied to monolayers of MDCK cells in 96-well microplates. After culturing for 6 hours, the cells were fixed with ethanol and incubated with mouse anti-influenza NP antibody C43, and then rabbit anti-mouse IgG antibody (Cappel) followed by goat anti-rabbit IgG antibody. Finally, the cells were incubated with peroxidase and rabbit anti-peroxidase (PAP) complex (Cappel). The infected cells were stained with 3,3′-diaminobenzidine tetrahydrochloride. The data were expressed as the percentage of reduction rate of infected cells.

### Isolation of Escape Mutants

Escape mutants were isolated by incubating Aic68 strain with IgG1 Ab with a modification of the method previously described (Nakagawa et al., 2003). Briefly, 1×10⁵ focus forming units/ml of virus was incubated for 1 h at 37° C. in the presence of 5 μg of Ab. The virus-Ab mixture was inoculated to MDCK cells in 24-well plates and incubated at 35° C. for 3 days. The neutralization tests on each well were performed separately with Abs in order to identify the escape mutants. The nucleotide sequence of the escape mutants was determined by using the following primers: H3HA forward, 5′-GCAAAAGCAGGGGATAATTCT (SEQ ID NO.:61); H3HA backward, 5′-GTAGAAACAAGGGTGTTTTTAATTA (SEQ ID NO.:62); H3HA 568, 5′-TGAACGTGACTATGCCAAACAATG (SEQ ID NO.:63).

### Construction of Plasmid DNAs for Cell Surface Expression of HA and HA1 Domain

DNAs encoding Fuk85 HA ectodomain (the residues 1-513; corresponding to SEQ ID NO.:53 and a.a. 1-184 of SEQ ID NO.:26), Fuk85 HA1 domain (the residues 1-329; SEQ ID NO.:53), and NC99 ectodomain (the residues 1-513; corresponding to SEQ ID NO.:60 and a.a. 1-180 of SEQ ID NO.:34) were amplified by PCR and inserted into the KpnI-ApaI sites of pYD1 (Invitrogen Inc.), resulting in the HA and the derivatives connected with a V5 epitope tag. DNAs encoding HA and the derivatives with a V5 tag were amplified by PCR again and inserted into the SfiI-SalI sites of pDisplay (Invitrogen Inc.). The resultant plasmid DNAs encode a V5 tag between the transmembrane region (a transmembrane domain of PDGFR) and the extracellular domain of HA and the derivatives. The resultant plasmid DNAs encode a V5 tag between the HA ectodomain or HA1 domain of and a PDGFR transmembrane domain.

### Flow Cytometry (FCM) Analysis for 293T Cells Expressing HA on the Cell Surface

293T cells in a 150-mm dish were transiently transfected with 24 μg of the plasmid DNA for expressing HA or no DNA (mock-transfection) mixed with 60 μl of Lipofectamine LTX (Invitrogen Inc.). The cells were recovered after culture in D-MEM (Wako) at 37° C. for 24 h and blocked with 2.5% BSA in PBS on ice. Then the cells were incubated with 5 μg/ml of IgG Ab, 10 μg/ml of Fab-PP Ab, 1 μg/ml of mouse anti-H3 MAb F49 (Ueda et al., 1998), or 1 μg/ml of rabbit anti-V5 antibody (Millipore). Finally, the cells were incubated with Alexa Fluor 488 anti-human IgG, Alexa Fluor 488 anti-mouse IgG, or Alexa Fluor 488 anti-rabbit IgG (Molecular Probes), respectively, and subjected to FCM analysis using FACS Calibour (Becton Dickinson).

### FCM Analysis for 293T Cells Expressing 2 Kinds of Truncated HA

DNAs encoding Fuk85 HA regions corresponding to the residues 44-309 and 39-319 were amplified by PCR and inserted into the ApaI-SalI sites of pDisplay (Invitrogen Inc.). The resultant plasmid DNAs encode truncated Fuk85 HAs with a myc tag between the truncated HA and a PDGFR transmembrane domain. Theses truncated HAs were expressed on 293T cells and subjected to FCM analyses. Expression of the truncated HAs was verified by detecting the HAs on the cells by mouse anti-myc tag antibody (MBL).

### Epitope Mapping Through Analysis Chimeras

Previously we developed a method for epitope mapping through analysis chimeras (EMAC method) (Okada et al., 2011).

### Competitive ELISA

Competitive ELISA was performed by using Fab-PP to detect binding to virus particles and Fab-cp3 as a competitor. Fab-cp3 molecules in the supernatant of E. coli culture were concentrated 20-fold. Fab-PP of F005-126, F045-092, F041-342, F041-360, F019-102, and F037-115 were purified. Formalin-inactivated virus particles were coated onto a MaxiSorp immunoplate. A total of 50 μl of Fab-PP at an optimized concentration was mixed with 50 μl of 20-fold concentrated Fab-cp3 and added to a virus-coated immunoplate. Then, peroxidase-conjugated rabbit Ab was added. Finally OPD was added, and the OD at 492 nm was measured.

### Protease Susceptibility Assay

A protease susceptibility assay was performed as previously described (34) with modifications. In brief, 0.63 μg of purified Aic68HA and the Fab-PP form of F005-126 (2.3 Fabs per HA protomer) were mixed in 50 mM Tris-HCl (pH 8.0) containing 1% dodecylmaltoside. The pH was lowered to 4.5 by adding 0.1 M citrate Na (pH 2.5) to all samples except controls. Reaction mixtures were incubated at 25° C. for 20 minutes, and then the pH was neutralized by the addition of 1 M Tris-HCl (pH 8.9). Trypsin was added to all samples (except controls) at a final ratio of 1:200 by mass, and the samples were incubated for 40 minutes at 25° C. To verify that F005-126 itself did not prevent tryptic digestion, Fab-PP was mixed with HA that had been treated at pH 4.5 and then neutralized in the same way, and then the sample was incubated for 1 hour at 25° C. followed by tryptic digestion for 40 minutes at 25° C. Non-reducing SDS buffer was added to each reaction and boiled for 5 minutes. The samples were subjected to SDS-PAGE. The gel was stained with Coomassie Brilliant Blue.

### Protein Expression and Purification

The ectodomain of hemagglutinin (amino acid residues 17-520:HA) from A/Aich/2/1968(H3N2) was cloned into the expression vector pBAC-3, as C-terminal fusions with a thrombin protease cleavage site, a trimerization ‘foldon’ sequence and His-tag (James Stevens, et al Science 2004, 303, 1866). The fusion proteins were synthesized by the baculovirus expression systems. During incubation at 27° C. for 48 h, HA protein was secreted into the culture medium. Cell debris was removed by centrifugation at 3500×g for 20 min, and supernatant was concentrated with QuickStand System (GE Healthcare). The concentrated culture supernatant was then loaded on a HisTrap column (5 ml; GE Healthcare) pre-equilibrated with buffer A (10 mM Tris-HCl (pH 8.0) containing 500 mM NaCl, and 20 mM imidazole). The column was washed with 50 ml of buffer A, and HA was eluted with 10 mM Tris-HCl (pH 8.0) containing 500 mM NaCl, and 500 mM imidazole. The fractions were pooled and dialyzed against 20 mM Tris-HCl (pH 8.0) and 20 mM NaCl containing thrombin protease to cleave the His-tag. To separate the His-tag and uncleaved protein, the protein was loaded on a HisTrap column, and the flow-through fractions were collected. The protein was further purified by ion exchange on a HiTrap Q column (5 ml; GE Healthcare) and size-exclusion chromatography on a HiLoad 16/60 Superdex 200 pg column (GE Healthcare), in a final buffer containing 20 mM Tris-HCl (pH 8.0), 150 mM NaCl.

IgG antibody (F005-126) was incubated with immobilized papain (Pierce), and Fab fragments generated by papain digestion were separated from undigested IgG and Fc fragment by chromatography on a HiTrap rProteinA FF column (GE Healthcare).

For the crystallization of the HA-Fab complex, HA and Fab were mixed in a 1:1.2 molar ratio and incubated overnight at 4° C. The HA-Fab complex, formed by incubating the proteins together, was separated from the uncomplexed proteins by chromatography on a HiLoad 16/60 Superdex 200 pg column, preequilibrated with 20 mM Tris-HCl (pH 8.0), 150 mM NaCl. The fractions containing the complex proteins were pooled and concentrated to 10 mg/ml with an Amicon-15 filter (Millipore).

### Crystallization and X-Ray Data Collection

The initial screening of crystallization conditions was conducted using commercially available screening kits (Hampton Research). The HA-Fab complex crystals were obtained in some crystallization conditions with the sitting-drop vapor diffusion method using PEG as precipitants. Crystals were obtained in a few days by mixing 1 μl sample solution and 1 μl reservoir solution containing of 12% PEG8000, 0.2 M KCl, 0.1 M Mg(CH₃COO)₂ and Na-citrate buffer pH 5.5. For the diffraction data collection, the crystals were gradually soaked in reservoir solution containing 20% glycerol. The data sets were collected at the BL41XU beam line (SPring-8).

### Data Collection, Structure Determination and Refinement

Diffraction data were collected at 100K on the BL41XU beamline at the SPring-8 (Hyogo, Japan). Diffraction images were processed with XDS (Kabsch, 1993) and HKL2000 (Otwinowski et al., 1997). The structure was solved at 4.0 Å resolution by molecular replacement with PHASER using the structures of H3 (PDB 1HA0) and Fab (PDB 1EO8) (Chen et al., 2000, Fleury et al, 2000) as starting models. The asymmetric unit contains four HA trimers and twelve F005-126 molecules and was refined using CNS with tight restrains and manual rebuilding with Coot (Emsley et al., 1994). Positive density for N-linked glycosylation was observed at 5 of the 6 predicted sites on HA, and a total of 20 sugar residues were built. Hydrogen bonds and van der Waals contacts between F005-126 and H3 HA were calculated using HBPLUS and CONTACSYM, respectively (McDonald et al., 1994, Sheriff et al., 1987). Surface area buried upon Fab binding was calculated with MS (Connolly et al., 1983). PyMol (DeLano Scientific) was used to render structure figures and for general manipulations. Kabat numbering was applied to the coordinates using the AbNum server (Abhinandan et al., 2008). Final refinement statistics are summarized in Table 3, which is located at the bottom of the specification.

### Experiment-1

When HA of H3N2 (Aic68) and HA of H1N1 (NC99) were artificially expressed on cells, F005-126 bound only to the cells expressing HA of H3N2 (FIG. 1B). Next, when HA and HA1 of H3N2 (Aic68) were expressed on cells, F005-126 bound equally to HA and HA1 (FIG. 1C). The HA1 domain contains 329 amino acid residues (e.g. SEQ ID NO. 48 for the strain Aic68) and is structurally divided into the globular head region (residues 39-319) and the stem region (residues 1-38 and 320-329). The regions consisting of residues 39-43 and 310-319 are closely associated in the 3D structure, and they are located in a junction between the head and the stem regions. Two kinds of truncated HA, Fuk85HA39-319 and Fuk85HA44-309 which corresponded to residues 39-319 and 44-309, respectively, were expressed on the cells and the binding of F005-126 to them was examined. FIG. 1D showed that F045-092 and F019-102 bound well not only to intact HA but also to the truncated HAs, indicating that the 3D structure near the sialic acid-binding pocket is properly formed by both truncated HAs. It also showed that F005-126 bound to Fuk85HA39-319 although weakly but not to Fuk85HA44-309. These results indicated that F005-126 binds to the globular head of HA1 but not to the region near the sialic acid-binding pocket. Since F005-126 did not show any HI activity (data not shown), it is likely that the epitope recognized by F005-126 is located far from the pocket.

Previously, we reported the characteristics of mAbs that had been isolated from the library of the donor born in 1960. The epitopes recognized by them were successfully assigned to one of the five sites on the globular head that had been well characterized as neutralizing epitopes. To determine the epitope recognized by F005-126, we performed competition experiments with F005-126 and four clones whose epitopes are known. As indicated in FIG. 2, presence of a large excess of F041-342 and F041-360 that bind to site C1/C2 completely inhibited the binding of F005-126 to the Yam77 virus particle. This suggested that the epitope recognized by F041-342 and F041-360 should overlap with the epitope recognized by F005-126. Although presence of a large excess of F005-126 only partly inhibited the binding of F041-342 and F041-360, the weak inhibition may be caused by the difference in the binding activity among these clones. The presence of F019-102, which binds to site E, appeared to partly inhibit the binding of F005-126. F037-115, which binds to site B1, did not compete with F005-126 in the binding reaction to the virus particle. These results suggested that the epitope recognized by F005-126 could be located at a region that is close to or partly overlaps with site C and that is near site E. We previously reported that the Abs recognizing site C did not show any HI activity and that the Abs recognizing site E showed weak HI activity.

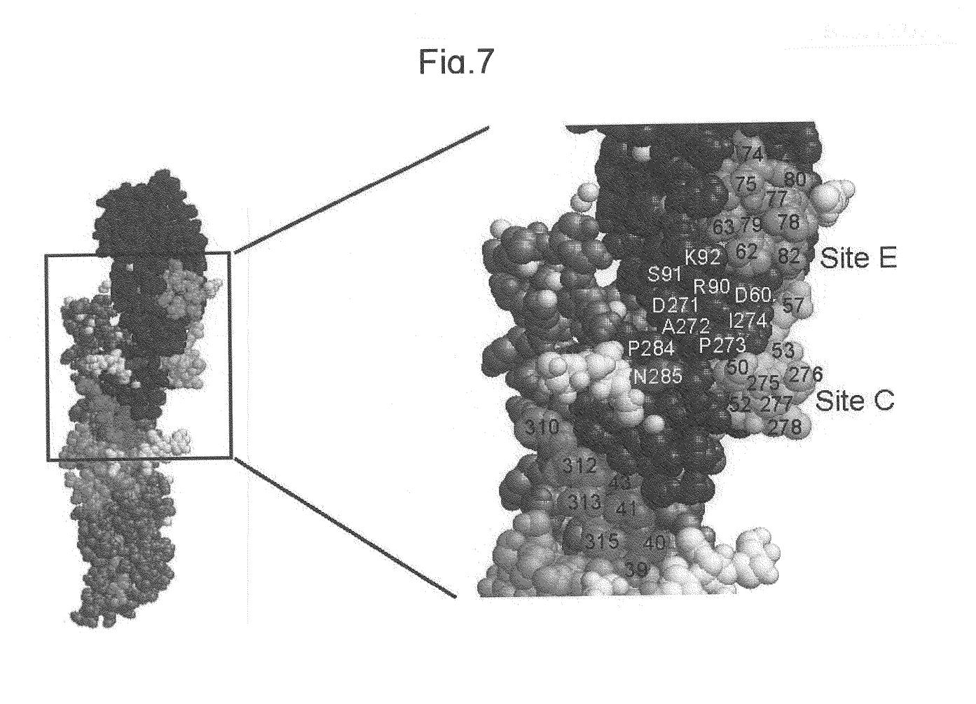

To further examine the epitope, we tried to isolate escape mutants under the presence of F005-126. Although we did not find a completely resistant variant, a partly resistant variant was isolated from Aic68 viruses. When the 50% inhibitory concentration by IgG type of F005-126 was compared between Aic68 and the variant, the variant showed around 100 times-stronger resistance. In the variant, asparagine 285 was mutated to tyrosine. Since the asparagine at this position is glycosylated, this mutation should result in deglycosylation. Involvement of the glycoside linked to Asn285 in the interaction between F005-126 and Aic68 HA was further supported by analyzing the mutant N285Y, as shown in FIG. 7. Residue 285 is located close to site C, which corresponds to residues 50-57 and 275-279. The relative positions of site C, site E and residue 285 on the 3D structure of HA is shown in FIG. 7.

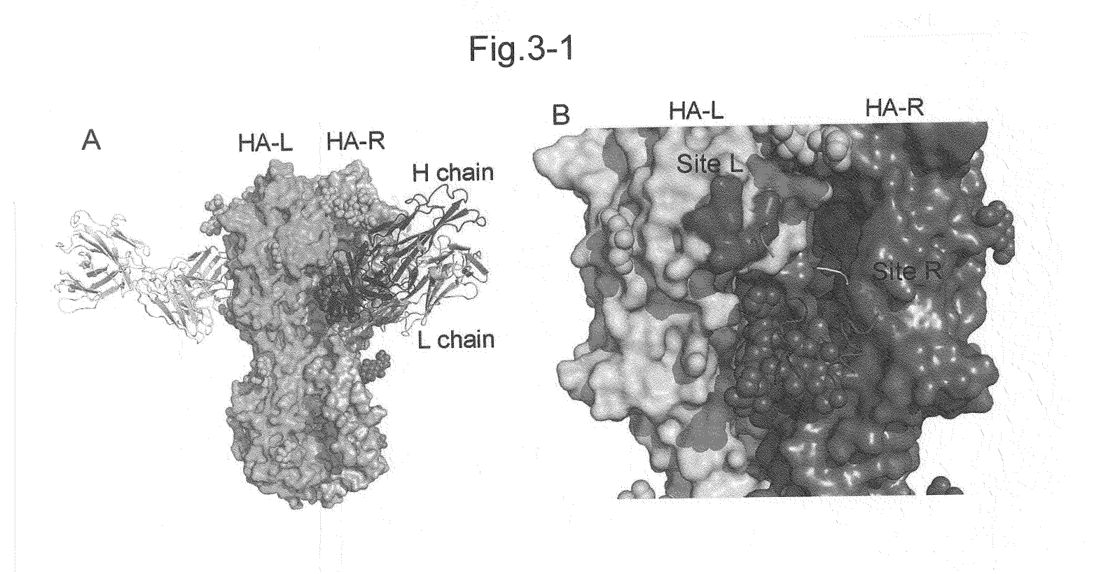

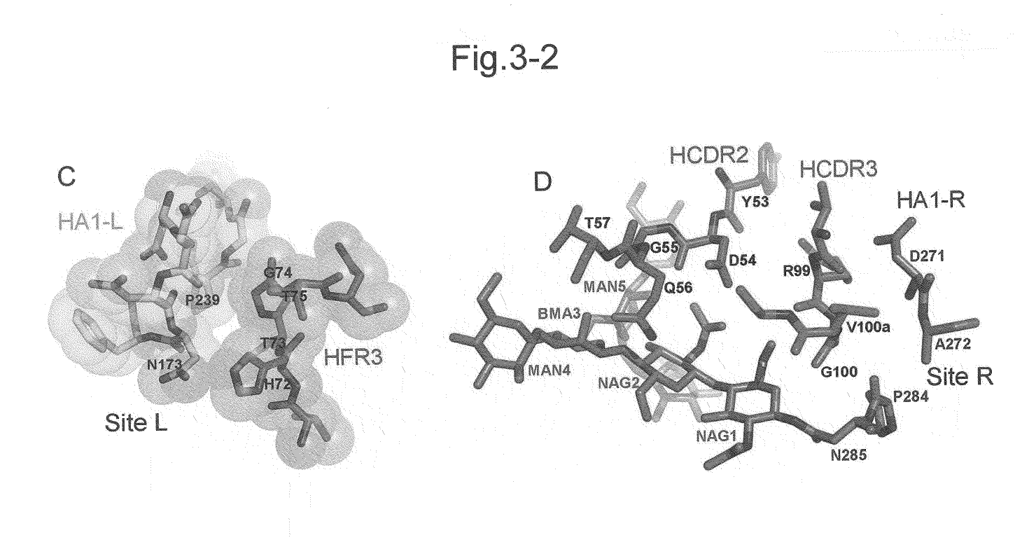

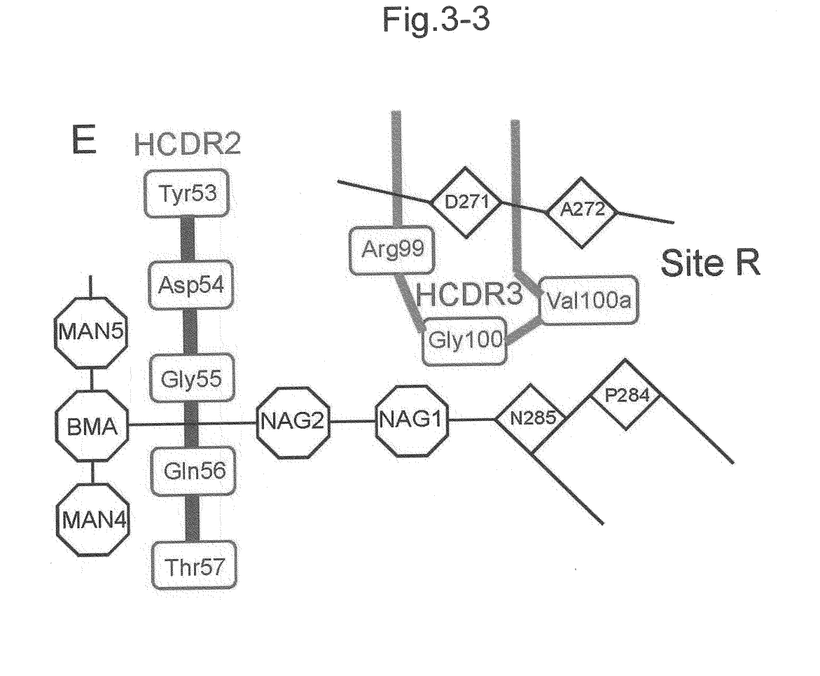

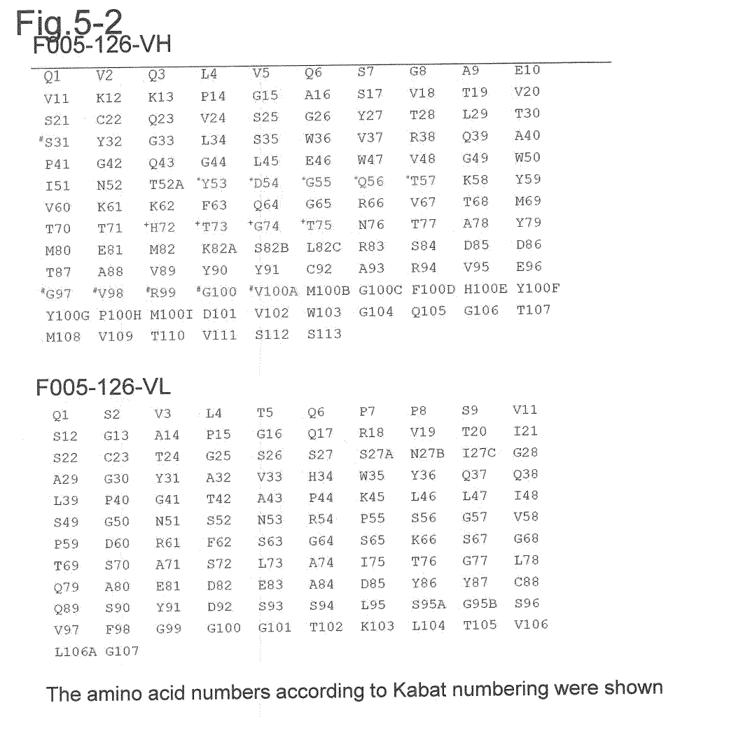

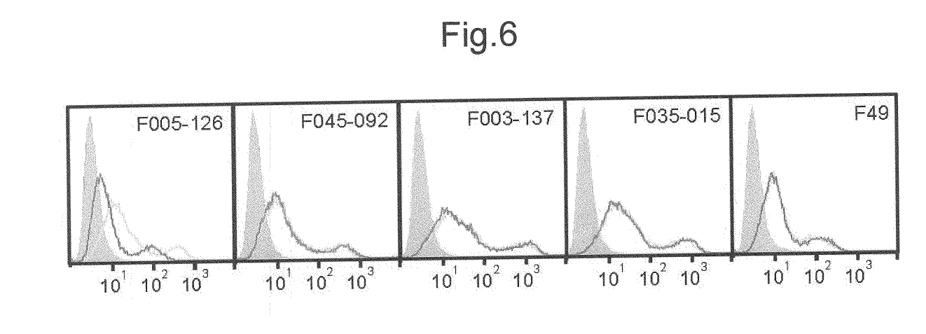

To directly examine the epitope recognized by F005-126, we determined a crystal structure of Aic68 HA in complex with F005-126 Fab at 4.0 Å resolution. The overall structure of Aic68 HA in the complex is similar to that of the H3 starting model [Protein Data Bank (PDB) accession code 1HA0]. As shown in FIG. 3-1, the Fab binds to HA at the valley formed by two neighboring HA monomers, and three Fabs bind to the HA trimer in the same manner. The contact region on HA is sandwiched by two glycosides linked to HA1-L: Asn¹⁶⁵ and HA1-R: Asn²⁸⁵. The direct interactions between the Ab and HA were observed in three main components, as indicated in FIG. 3-2. The participation of the V_{H} domain including all three complementarity-determining regions (CDRs) and one framework region (FR) in binding was identified, but the contribution of the V_{L} domain to the binding was unclear. The first component is site L in HA-L (FIG. 3-2) in which the loop containing Thr⁷³, Gly⁷⁴, and Thr⁷⁵ in FR3 is inserted into a narrow groove formed by two hairpin loops of HA1-L (residues 170-176 and 238-241) and makes van der Waals contacts (FIG. 3-3). The second component is site R in HA-R (FIG. 3-2) in which Val⁹⁸, Arg⁹⁹, Gly¹⁰⁰, and Val^{100a} in CDR3 are inserted into a narrow space formed by two loops of HA1 (residues 269-273 and 283-286) and glycoside linked to Asn²⁸⁵; this interaction makes van der Waals contacts. Furthermore, two hydrogen bonds are formed between C═O of Val⁹⁸ and HA1-R: Asp²⁷¹ as well as between the main chain NH of Arg⁹⁹ and HA1-R: Asp²⁷¹ (FIG. 3D). The C═O of Ser³¹ in CDR1 makes a hydrogen bond with the side chain of HA1-R: Ser⁹¹. For the third component (FIG. 3-2), Tyr⁵³, Asp⁵⁴, Gly⁵⁵, Gln⁵⁶, and Thr⁵⁷ in CDR2 and Arg⁹⁹, Gly¹⁰⁰, and Val^{100a} in CDR3 make long range van der Waals contacts with the glycoside linked to HA1-R:Asn²⁸⁵ (FIG. 3-3). A total area of 1084 Å² is buried at the contact surface. Sixty percent of the area arises from the peptide portion, and 40% of the area arises from the glycosides. The ratio of surface areas covered by the above three components is 17:43:40. The details of the data were summarized in FIGS. 5 and 6.

The amino acids involved in the interaction between F005-126 and HA are marked in color in FIGS. 5, 8-1 and 8-2, respectively. The amino acids at residues 172 and 173 in HA1 are variable among the 12 H3 strains. Nevertheless, F005-126 is able to neutralize all 12 H3 strains since the van der Waals contacts in this portion are formed between the side chain of the peptide in Ab and the main chain of the peptide in HA. The epitope suggested by the competition experiments is site R, and the epitope suggested by analysis of the escape mutant is the glycoside at residue 285. The presence of site L in the epitope was revealed only by the X-ray analysis of the HA/Ab complex.

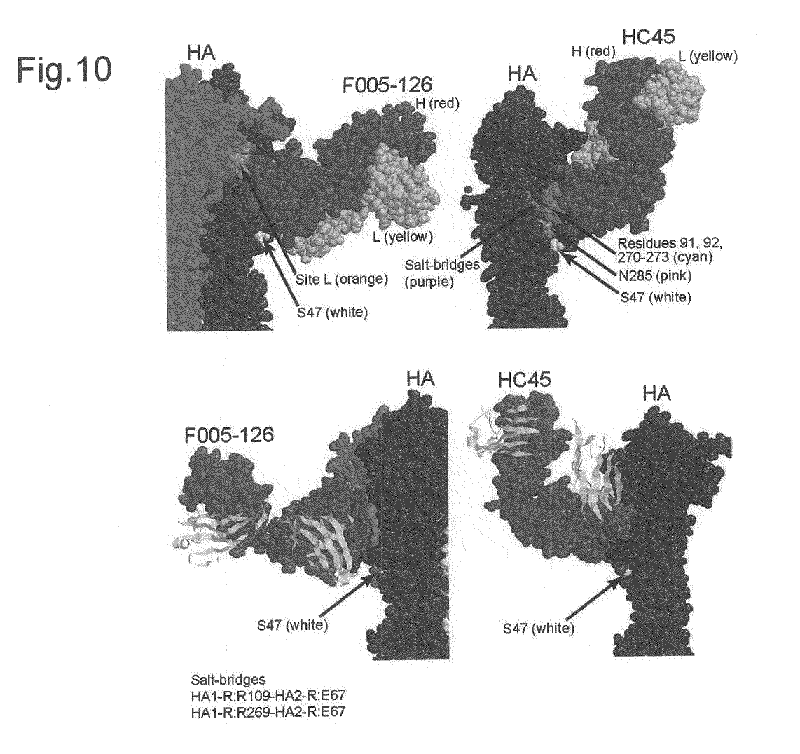

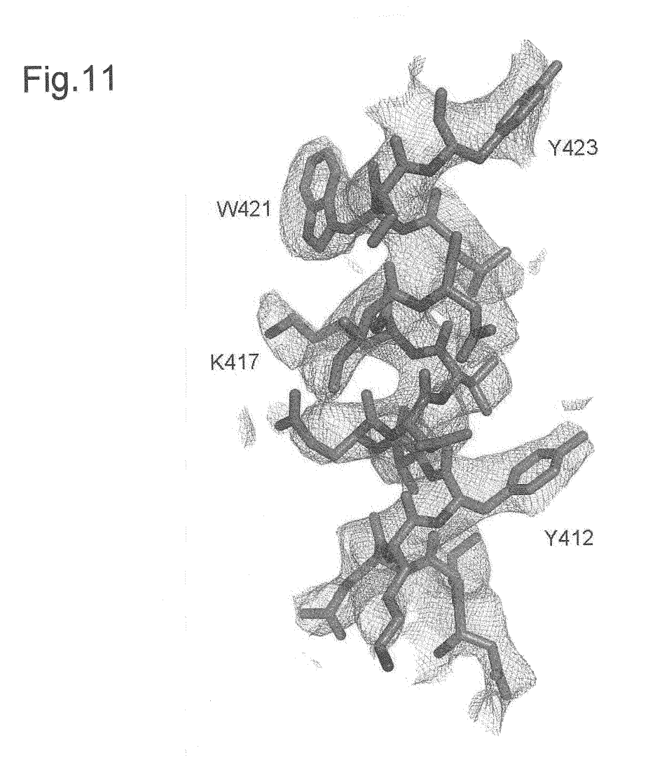

The epitope recognized by F005-126 was compared with the epitopes that have previously been revealed by X-ray analysis of the HA/Ab complex. Most of the epitopes already reported are categorized into two groups, epitopes located close to or inside of the sialic acid-binding pocket and epitopes located at the HA stalk that are directly involved in the conformational change induced by low pH. While HC45 and BH151 recognized the same epitope, it appeared to overlap with the epitope recognized by F005-126. The residues in HA1 that make contact with HC45 and BH151 are located in four peptide stretches, residues 59-63, 78-79, 90-94 and 271-273. Among them, residue 91 and the region covered by residues 271-273 are also recognized by F005-126. As indicated in FIG. 9-1, however, the distributions of contact residues on HA in the HA/Ab complexes were different between F005-126 and HC45. Moreover, whereas HC45 inhibits the binding of viruses to cells, F005-126 does not inhibit interactions between HA and sialic acid at all. This difference might be explained by the following observations. HC45 and BH151 bind to site E, and their C_{H}1 and C_{L} domains extended toward the membrane distal orientation, that is, in the direction of cells targeted by HA (FIG. 10). On the other hand, F005-126 binds to HA nearly orthogonally to the axis of HA (FIG. 10). Thus, HC45 and BH151 could sterically interfere with the interaction between HA and sialic acid but F005-126 could not. Thus, we concluded that the epitope recognized by F005-126 is new and has not yet been reported.

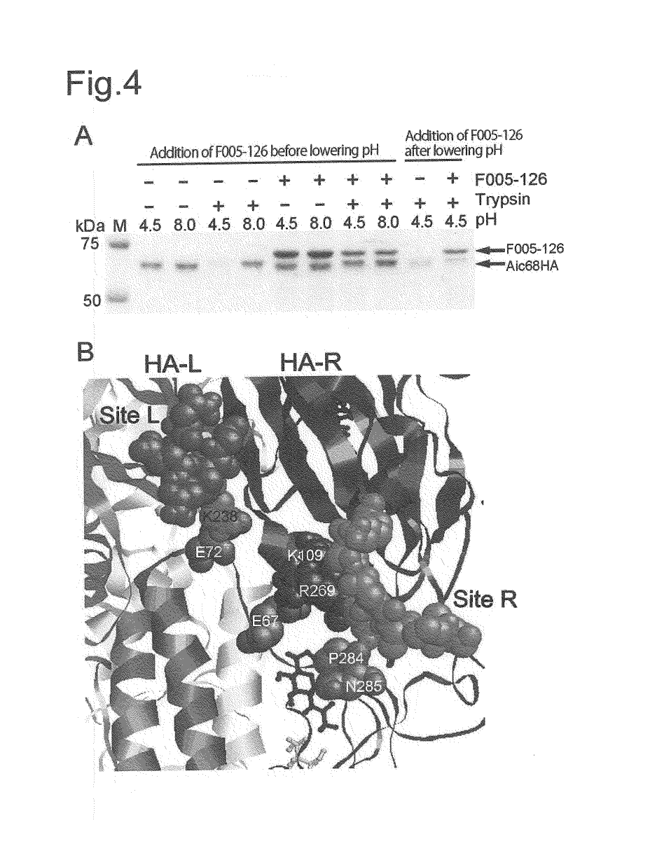

How can F005-126 show virus-neutralizing activity? Xu and Wilson reported the characteristics of HA at the early intermediate stage of membrane fusion. The large-scale conformational rearrangement of HA at low pH is triggered by a loop-to-helix transition of an interhelical loop (B loop). Broadly neutralizing Abs that bind to the stem region of HA directly inhibit this conformational change. According to Xu and Wilson, the HA1 subunit acts as a clamp to keep the B loop in its metastable prefusion state at neutral pH. The ionic interactions at the HA1-HA2 interface are reorganized at acidic pH, and the HA1 membrane-distal domain is deformed. If there is an Ab that can prevent deformation of the HA1, it could indirectly prevent a loop-to-helix transition of the B loop. Thus, we examined the ability of F005-126 to prevent the conformational change of HA at acidic pH by using the traditional method developed by Skehel et al. As indicated in FIG. 4 A, F005-126 Fab prevents conversion of HA from the protease-resistant to protease-susceptible form at pH 4.5.

There was an example where an Ab (HC63) that was bound to the globular head prevented the conformational change of HA induced by low pH. Since the epitope recognized by HC63 comprises residues from two HA subunits of one trimer, it can function as a clamp to keep the trimer structure of the globular head. Moreover, HC63 also prevents the HA/Ab interaction because the epitope recognized by the Ab is located close to the sialic acid-binding pocket. Since F005-126 binds to HA at the valley formed by two neighboring HA monomers and the epitope is distributed in two HA monomers, it would be possible that F005-126 can function as paste for stabilization of the HA trimer. However, there could be another possibility. Huang et al. suggested that dissociation of HA1 heads may be caused by their enhanced protonation leading to an increase in the positive net charge of HA1. Sivaramakrishna et al. directly showed that distinct salt bridges between HA1 and HA2 in a HA monomer (intramonomer) and between HA monomers (intermonomer) could play an essential role for the pH-dependent stability of HA. The location of salt bridges at the HA1-HA2 interface of the hinge region that may undergo a loop-to-α-helix transition at low pH was mapped to residues 85-90, 104-115 and 265-279 in HA1 and residues 67-72 in HA2. R268E and R269G mutations in HA1 resulted in a higher threshold of pH dependence for the conformational change of HA measured by proteolysis. When the pH decreases, the breakage of salt bridges at the HA1-HA2 interface that results in the deformation of HA1 could occur as the initial event before the occurrence of a loop-to-helix transition of loop B. FIG. 4B indicates the position of amino acids that form a salt bridge in the vicinities of site L and site R in our present analysis. Thus, it might be possible that F005-126 shows virus-neutralizing activity by preventing this deformation of HA1.

Thus, we believe the epitope described in this study is a new conserved neutralizing epitope.

It has been thought that neutralizing Abs should have one of the following two functions: prevention of the binding reaction between HA and sialic acids, and the prevention of the structural change of HA.

### Experiment-2

### Docking Simulation Using US Approved Drug

In the subject Example, docking simulation employing the model of the present invention was performed using US approved drug.

Interaction between an antibody (F005-126) and HA; the binding site spans two HA chains and as a domain of interaction, A Site L and/or Site R and/or carbohydrate chain.

### <Materials and Methods>

JKL-90 was used as a structure of H3 (the atomic coordinates of H3 called JKL-90; see: PDB2, see also FIG. 13). With respect to this structure, a Site Finder module of MOE (Chemical Computing Group Inc (Quebec Canada)) was used to identify binding sites thereof. Except for the parameter Connection Distance, which was changed to 3.5 Angstroms, the experiments were performed using default parameter.

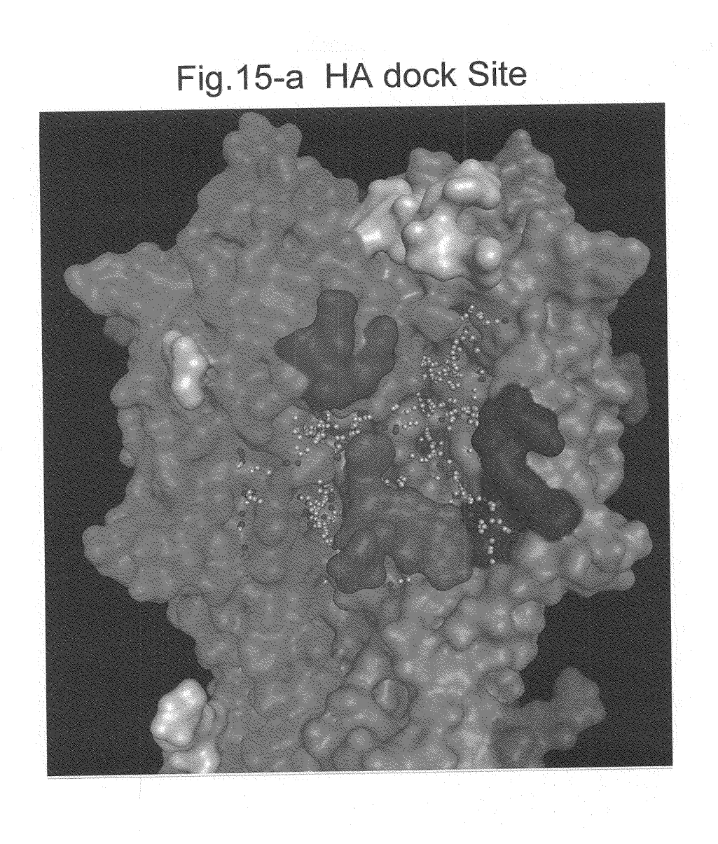

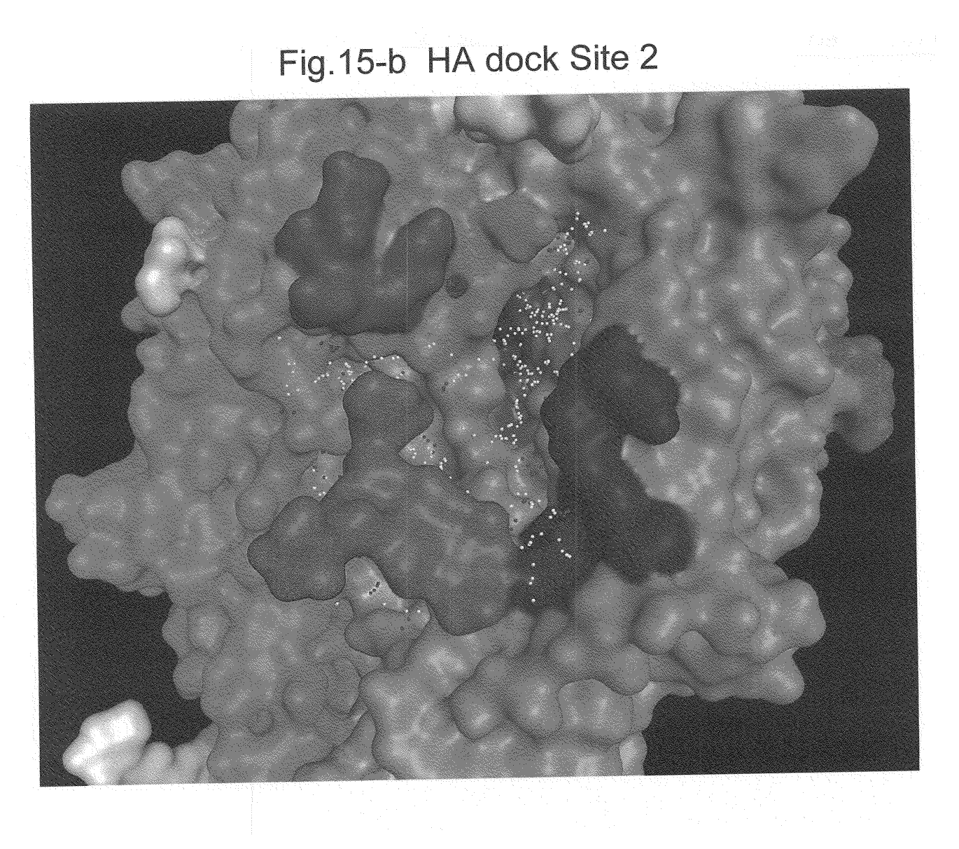

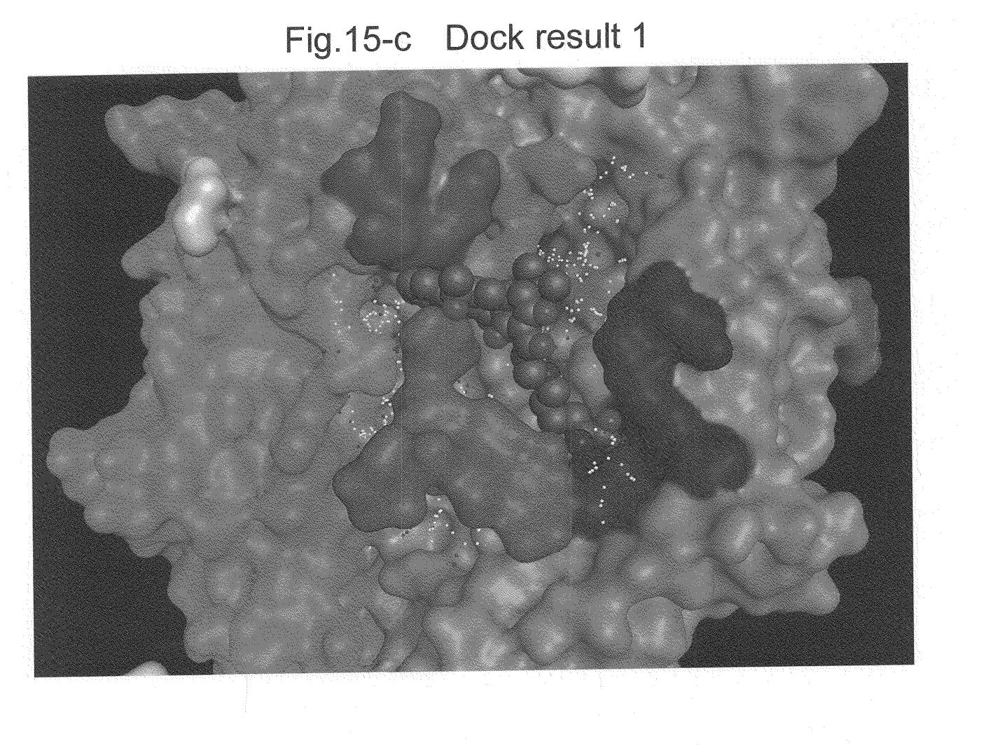

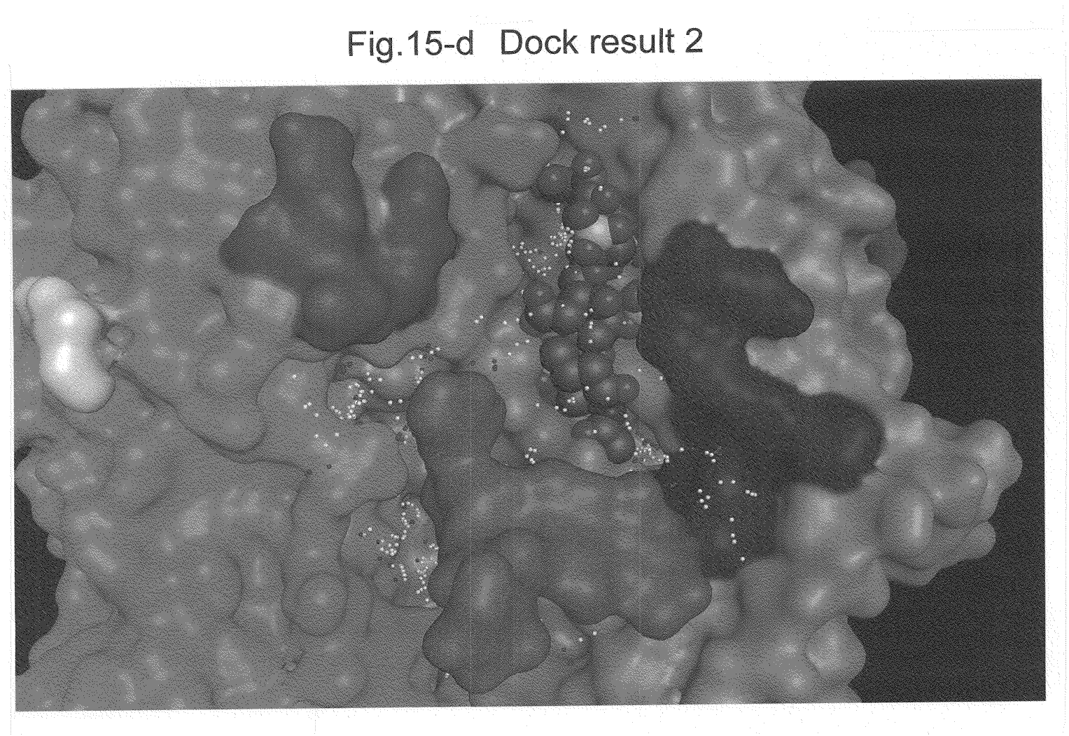

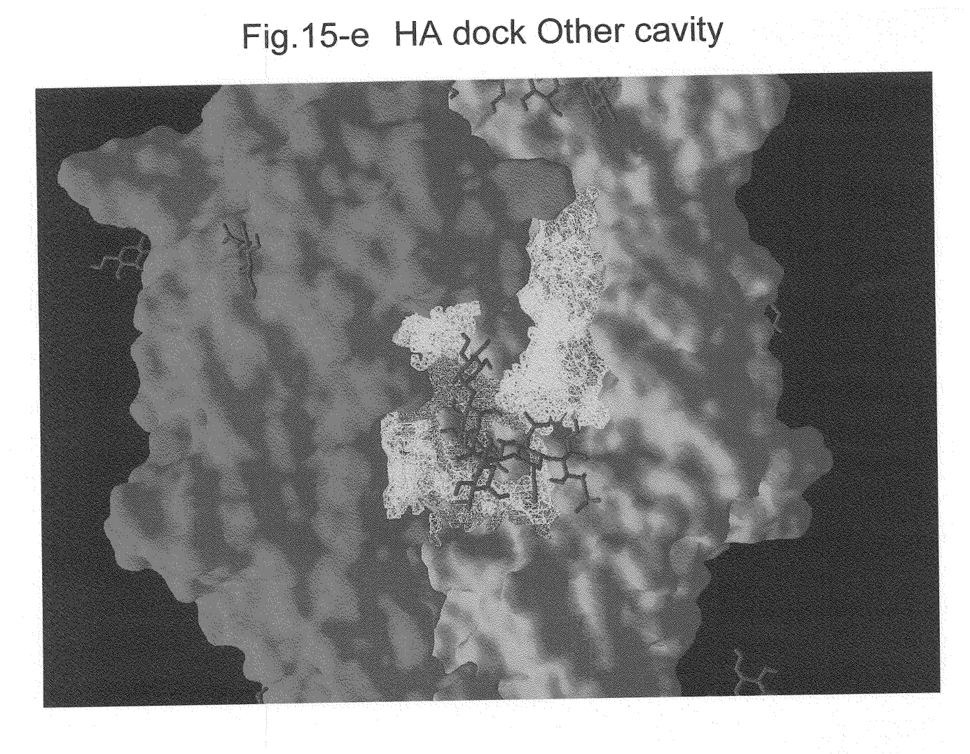

Binding pocket regions investigated herein are shown in red and white points (see FIG. 15-a). Docking simulation was performed on the sites shown in FIG. 15-a, by means of MOE dock or MOE software, which allows docking simulation.

The Approved US Drug Database, a drug bank, was used as a compound database for the analysis. Default setting of the virtual screening mode of MOE dock was used as the parameter of docking (this examination: 1411 compounds)

Database containing millions of compounds may be used for the subject experiments.

### <Result>

The Figures show molecular surface images and orange, light blue, green show HA, and purple and light pink show sugar chains. Site L is shown in red and Site R is shown in blue. An orange, light blue, green are HA, and purplish red, light pink are carbohydrate chains. Site L presents red, Site R with blue. Docking site has been set with a slightly broad width as shown in the Figure, which is shown in small red spheres and light pink spheres in FIG. 15-c, FIG. 15-d.

As for HA docksite, the results are shown in FIGS. 15-a and 15-b. FIG. 15-b is a magnified figure of FIG. 15-a. FIG. 15-c and FIG. 15-d only show coarse (i.e. with lower accuracy, or not best scores) results of screening of known inhibitors.

FIG. 15-c shows the location where the binding domain spans Site L and Site R. FIG. 15-d shows the location where a pocket is recognized, which pocket is composed of HA dimer at a deeper position than that between Site L and Site R.

The compounds of FIG. 15-c, FIG. 15-d are limecycline of an antimicrobial, ardeparin of anticoagulant respectively.

In addition, FIG. 15-e is a binding pocket region when that was carried out in MOE except MOE dock.

### Experiment-3

### Docking of HA with Hemagglutinin Other than the Particular H3

In the present Example, docking of HA with hemagglutinin other than the particular H3 used in the above Experiment is performed.

The following H1-Fab, H2-Fab, H5-Fab, H7-Fab and H9-Fab models were used: H1N1/Cal09pdm (1RUZ)-F005-126Fab
H1N1/SC1918 (3LZG)-F005-126Fab
H2N2/JPN57 (2WRD)-F005-126Fab
H5N1/Viet04 (2FK0)-F005-126Fab
H7N3/aviIta02(1TI8)-F005-126Fab
H9N2/swHK98(1JSD)-F005-126Fab

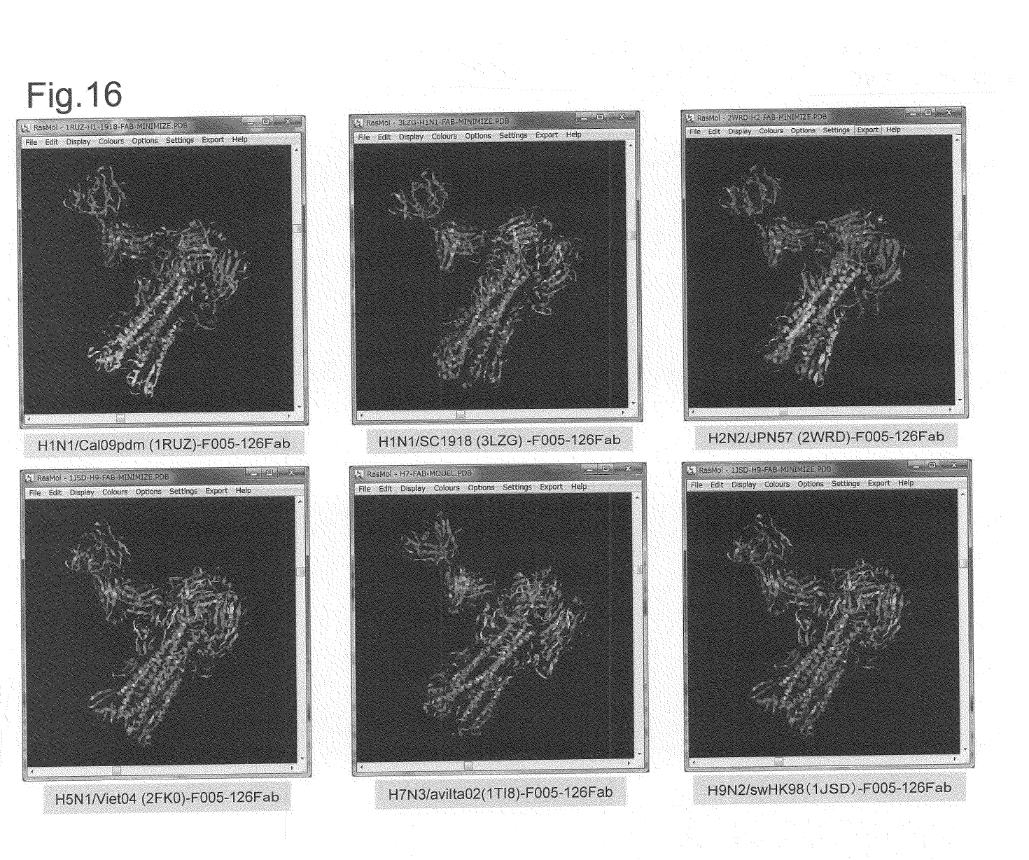

All models were superimposed by program COOT (The source 1) and energy-minimized by CNS (The source 2).
(The source 1) Emsley, P. and Cowtan, K., Acta. Crystallogr. Sect. D Biol. Crystallogr. 60, 2126-2132 (2004)
(The source 2) Brunger, A. T., Adams, P. D., Clore, G. M., DeLano, W. L., Gros, P., Grosse-Kunstleve, R. W., Jiang, J. S., Kuszewski, J., Nilges, M., Pannu, N. S., Read, R. J., Rice, L. M., Simonson, T., and Warren, G. L., Acta. Crystallogr. Sect. D Biol. Crystallogr. 54, 905-921 (1998)
   As one example, the experimental result is shown in FIG. 16. Each HA-Fab model picture has atomic coordinate data. Thus, the Appropriateness of a model can be estimated as an atomic coordinate of corresponding HA of a PDB database by comparing.

### Experiment-4

### Homology Analysis of Amino Acid Sequence Corresponding to the Region of Site L and Site R of HA Other than H3

Collection of the influenza A HA sequences and extraction of HA1 region as well as the epitope regions recognized by F005-126 are performed. Full-length protein sequences of HA of human-, swine- and avian-derived influenza A viruses H1N1 (including pandemic 2009), H3N2 and H5N1 were obtained from the Influenza Virus Resource at the National Center for Biotechnology Information (http://www.ncbi.nlm.nih.gov/genomes/FLU/FLU.html) (Source 3: Y. Bao, P. Bolotov, D. Dernovoy, B. Kiryutin, L. Zaslaysky, T. Tatusova, J. Ostell, D. Lipman, The Influenza Virus Resource at the National Center for Biotechnology Information, J. Virol. 82 (2008) 596.601.)

The HA sequences were then aligned using mafft v6.705b. (Source 4: K. Katoh, H. Toh, Recent developments in the MAFFT multiple sequence alignment program, Brief Bioinform. 9 (2008) 286-298.) Previously we reported sequence variation and accumulation in H1N1, H3N2 and H5N1 derived from different hosts. (Yamashita et al., 2010, source 5: Akifumi Yamashita, Norihito Kawashita, Ritsuko Kubota-Koketsu, Yuji Inoue, Yohei Watanabe, Madiha S. Ibrahim, Shoji Ideno, Mikihiro Yunoki, Yoshinobu Okuno, Tatsuya Takagi, Teruo Yasunag, Kazuyoshi Ikuta, Biochemical and Biophysical Research Communications 393 (2010) 614-618.

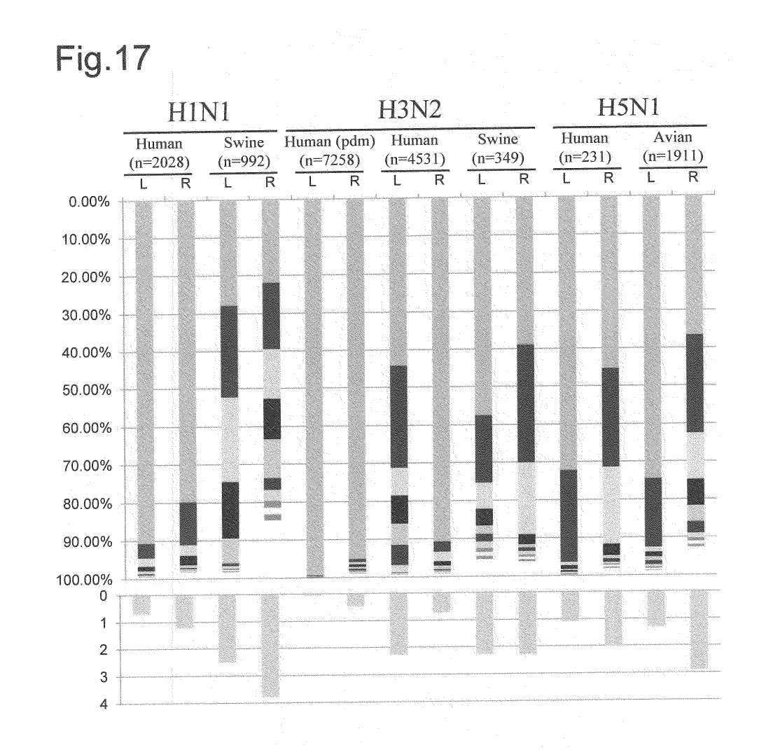

As concerns Site L and Site R, the experimental result is shown in FIG. 17 as one example.

### Experiment-5

In this Experiment, we counted the number of variations of the epitope region to estimate its variation within subtype using as much Influenza sequences downloaded from NCBI GenBank database as possible.

### <Materials and Methods>

We downloaded amino acid sequence of Influenza A virus from Influenza Virus Sequence Database at the National Center for Biotechnology Information (NCBI) on Sep. 18, 2012, and extracted complete Hemagglutinin sequence of human H1N1, H3N2, and H5N1, and swine H1N1 and H3N2, and avian H5N1, respectively. For human H1N1 sequences, we separated the pandemic H1N1 (2009) from the seasonal H1N1 as follows: we extracted hemagglutinin sequences of human H1N1 sequences obtained in 2009, and collected 7 most frequently occurred amino acid sequences, which have more than 1% occupation, as representatives of the pandemic sequences. We extracted hemagglutinin sequences of human H1N1 sequences obtained before 2009 as representatives of the seasonal H1N1 sequences. Then, we performed blastp search (Altschul, Stephen F., Thomas L. Madden, Alejandro A. Schaffer, Jinghui Zhang, Zheng Zhang, Webb Miller, and David J. Lipman, 1997. Gapped BLAST and PSI-BLAST: a new generation of protein database search programs. Nucleic Acids Res. 25:3389-3402.) of all the human H1N1 sequences against these pandemic and seasonal sequences. If a sequence is most homologous to a sequence of pandemic H1N1, the sequence was classified as a pandemic sequence, if the sequence is most homologous to a sequence of seasonal H1N1, the sequence was classified as a seasonal sequence. Subsequently, these sequences were aligned altogether using MAFFT v6.705b program (Katoh K, and Toh H., 2008. Recent developments in the MAFFT multiple sequence alignment program. Briefings in Bioinformatics. 9(4):286-298; and Katoh K, Misawa K, Kuma K, Miyata T., 2002. MAFFT: a novel method for rapid multiple sequence alignment based on fast Fourier transform. Nucleic Acids Res. 15; 30(14):3059-66.), and then, the epitope regions were extracted according to the positions shown in FIG. 8. The variation of each subtype was estimated by computing Shannon index of each site (Shannon C E., 1948. A Mathematical Theory of Communication. The Bell System Technical Journal 27: 379-423, 623-656) and by counting the number of all kind of sequences as described in Yamashita et al. (Yamashita, A., Kawashita, N., Kubota-Koketsu, R., Inoue, Y., Watanabe, Y., Ibrahim, M S., Ideno, S., Yunoki, M., Okuno, Y., Takagi, T., Yasunaga, T., Ikuta, K., 2010. Highly conserved sequences for human neutralization epitope on hemagglutinin of influenza A viruses H3N2, H1N1 and H5N1: Implication for human monoclonal antibody recognition. BBRC 393(4), 614-618.), and by making sequence logos (Gavin E. Crooks, Gary Hon, John-Marc Chandonia and Steven E. Brenner, 2004. WebLogo: A Sequence Logo Generator. Genome Research, 14:1188-1190.).

### <Results and Discussions>

The types of the epitope sequences used in this study are shown in Table 5. For human H3N2 influenza strains, newer sequences seem to be more popular in this study. Although, this result may reflect the bias that influenza sequence nowadays are more frequently sequenced and deposited to the database, this result clearly showed that in human H3N2 strains, the Site L is very easy to change while the Site R is not in human H3N2 strains. For human H1N1, both site L and R of SC1918 and Site R of pandemic strain Cal109pdm are very rare type in the database. This result may be the result of adaptation to humans.

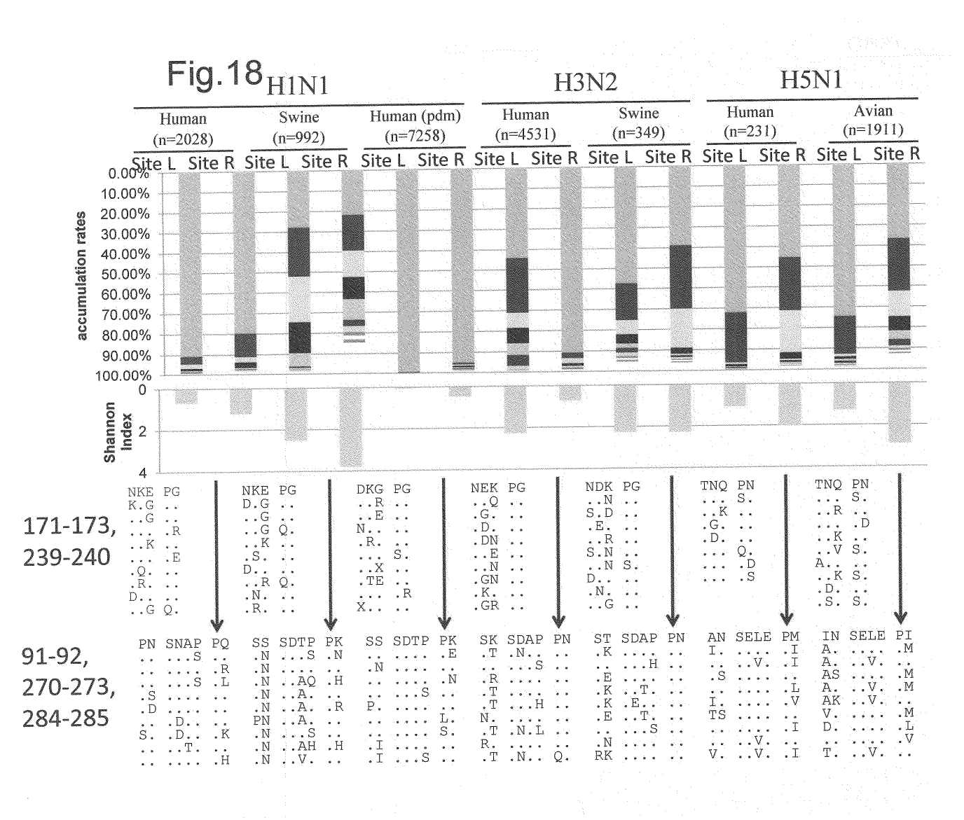

Sites L and R in human H1N1 pdm are the most conserved among subtypes analyzed this time. Those in seasonal human H1N1 are fairly conserved as well. Those in H3N2 and H5N1 have rather higher variation. Those sites in swine tend to vary more be more variant than in human, while those in avian seem to have similar variation variety with those in human in H5N1 (FIG. 18). These results are consistent with Yamashita et al. (2010, id.). In most of the cases, Site L is less variable than Site R. However, Site L shows higher variation than Site R only in human H3N2, due to low variation variety in Site R, or higher variation variety in Site L in human H3N2.

The dominant sequence in human H1N1 pdm is the same as the most frequent sequence of site R and the second most frequent sequence of Site L in swine H1N1. This result is consistent with one the other studies that hemagglutinin of human H1N1 pdm came from swine flu (Smith G J, Vijaykrishna D, Bahl J, Lycett S J, Worobey M, Pybus O G, Ma S K, Cheung C L, Raghwani J, Bhatt S, Peiris J S, Guan Y, Rambaut A., 2009. Origins and evolutionary genomics of the 2009 swine-origin H1N1 influenza A epidemic. Nature 25; 459(7250):1122-5.). The dominant sequences of Site L in human seasonal and swine H1N1 are the same. However, for Site R, only one minor sequence (PN SDAP) is the same between human and swine. This indicates that there is no significant relation between seasonal human and swine. For human and avian H5N1 sequences, the top two sequences in Site L (TNQ PN and TNQ SN) are the same, and although the order of the sequences are different, the top three sequences in Site R (AN SELE PM, 1N SELE PI, and AN SEVE PI) are also the same. This result supports the idea that so far, H5N1 mainly comes from avian, and human-to-human transmission of H5N1 Influenza virus is rare (Rabinowitz P M, Galusha D, Vegso S, Michalove J, Rinne S, Scotch M, Kane M., 2012. Comparison of Human and Animal Surveillance Data for H5N1 Influenza A in Egypt 2006-2011. PLoS One. 7(9):e43851). Similar sequence variation in human and avian H5N1 also suggests that Sites R or L does not affect the efficiency of infection into human.

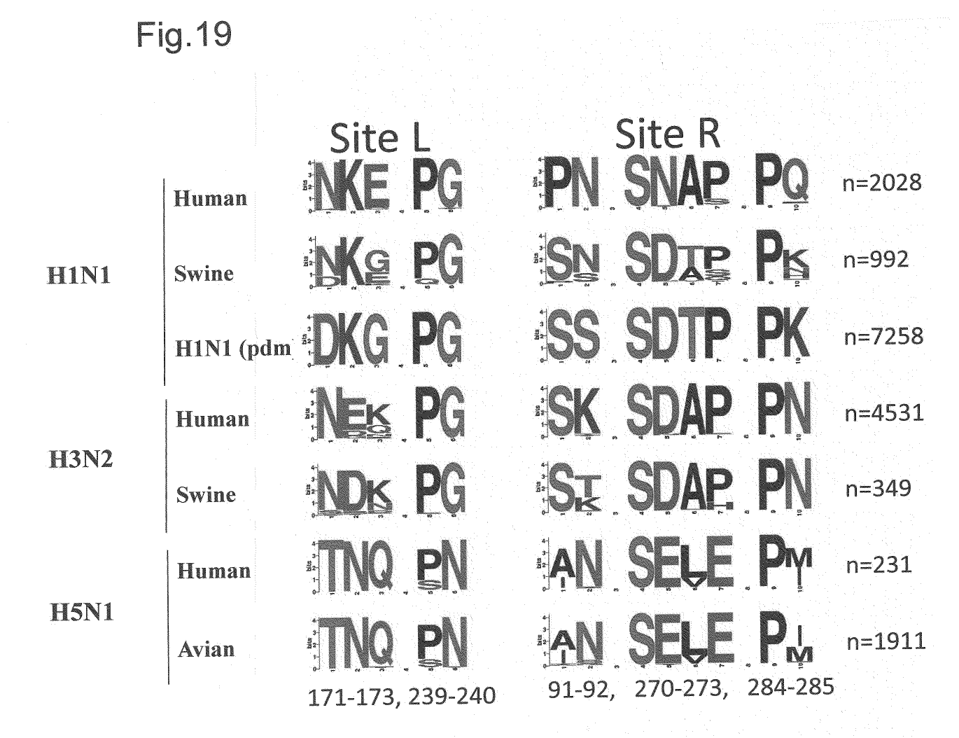

Sequence Logo analysis showed that in H1N1 and H3N2, the consensus sequences of Sites L and R are different between hosts even if the subtypes are the same. As for human H1N1, consensus sequences of Site L and R are different even between pdm and seasonal groups. This result also suggests that origin of pdm and seasonal human H1N1 flu came from different origins. Sequence Logo of Sites L and R in human and avian H5N1 are similar to each other (FIG. 19).

### Experiment-6

In the present Experiment, an additional 3D structure model of hemagglutinin was constructed, and the second round of screening was conducted.

### <Materials and Method>

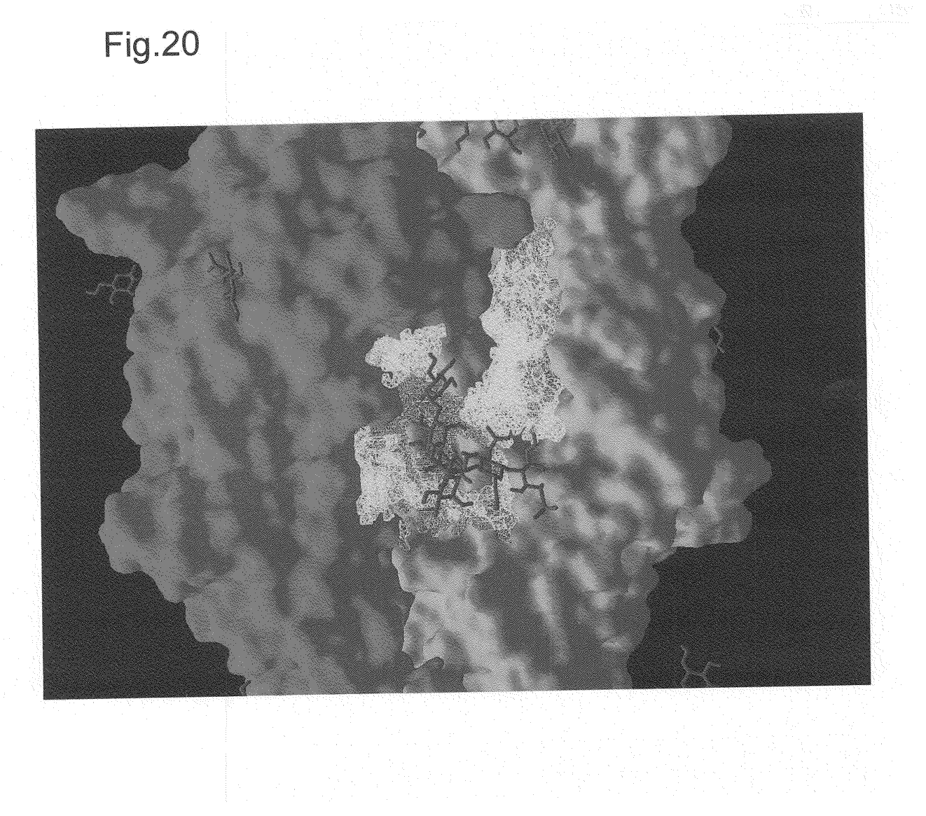

The 3D structure model of hemagglutinin was constructed using JKL-90-0101.pdb<PDB3>. For this structure, we identified its docking site by MOE 2011.10 (available from CCG.inc) Site Finder module. The only parameter changed was the connection distance (from 2.0 Å to 3.5 Å), and the other parameters were used as default. A lot of docking sites were identified but the largest one was selected and was used for the docking simulation. The selected binding site was shown in FIG. 20. The docking simulation was carried out by MOE dock and the parameter of the first screening was the virtual screening mode default. MOE leadlike database which has 653,214 compounds was used for the first screening. From the results, we selected 21,063 compounds which had S score of less than −13 and used the compounds for the second screening. In addition, Yakuri Database from Namiki Shoji Co. Ltd. (3,565 compounds) was also used for the second screening. The parameter of second screening was the induce fit mode default.

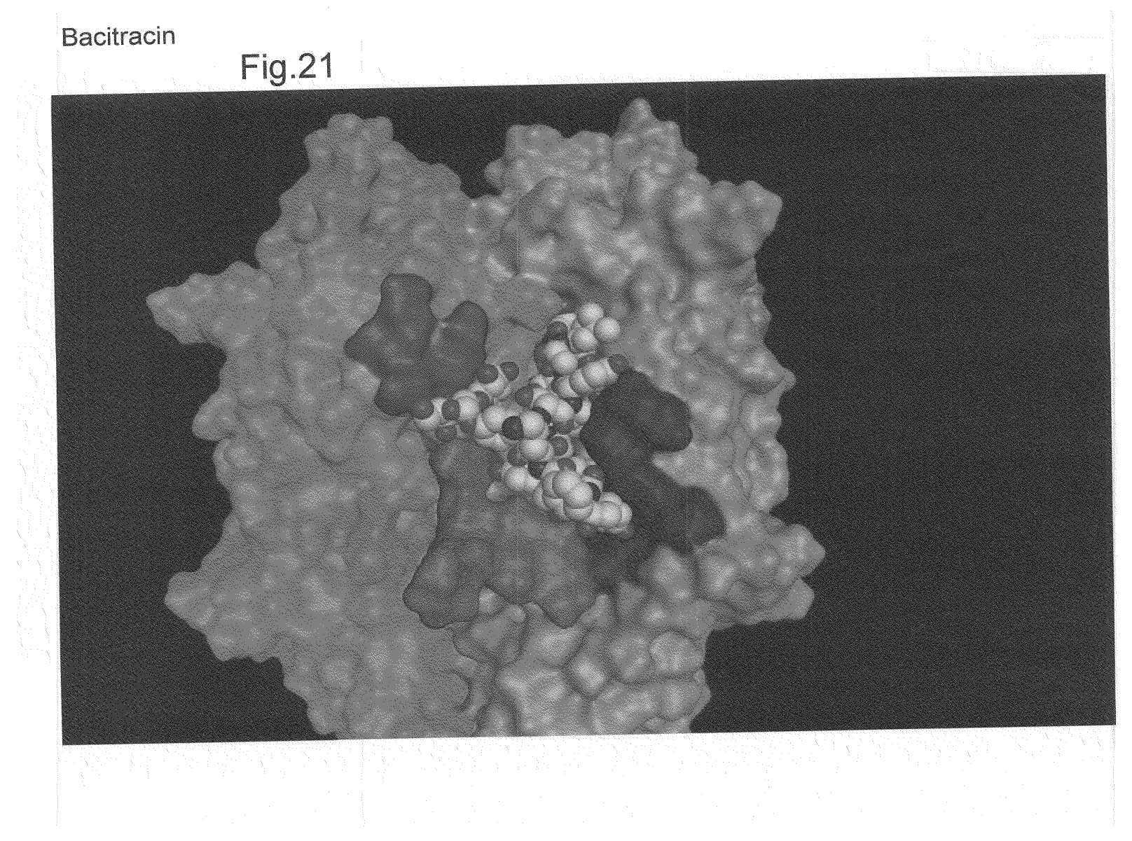

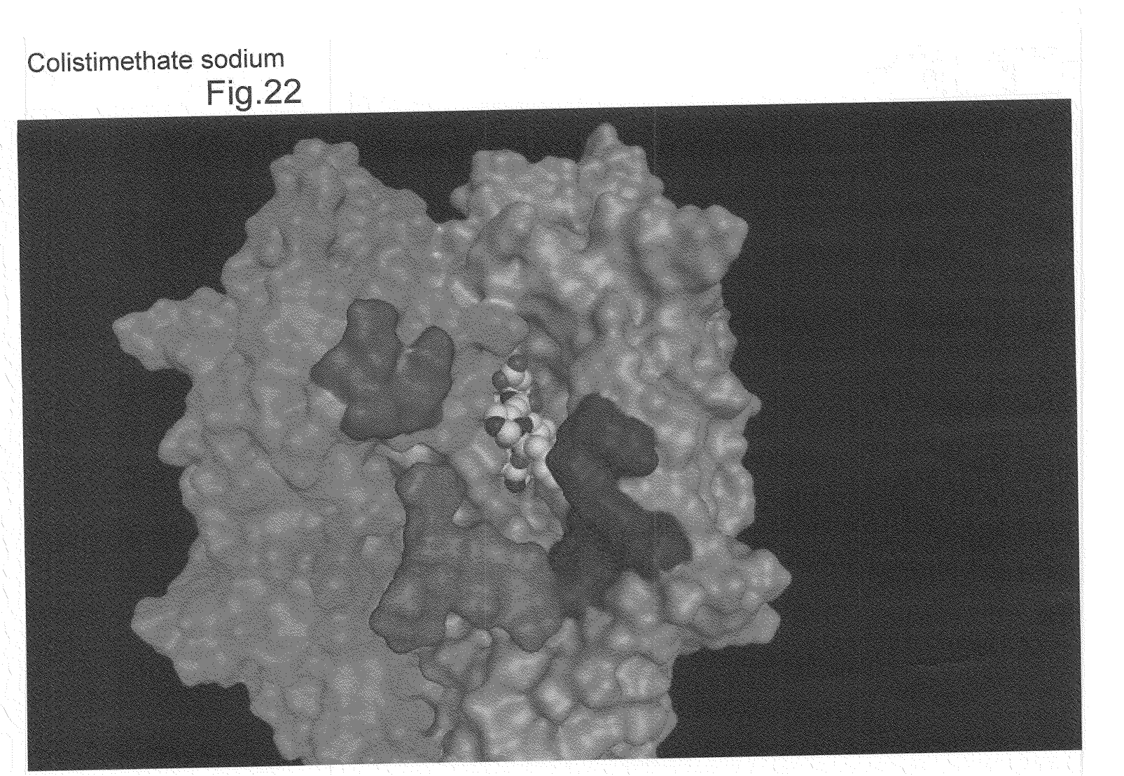

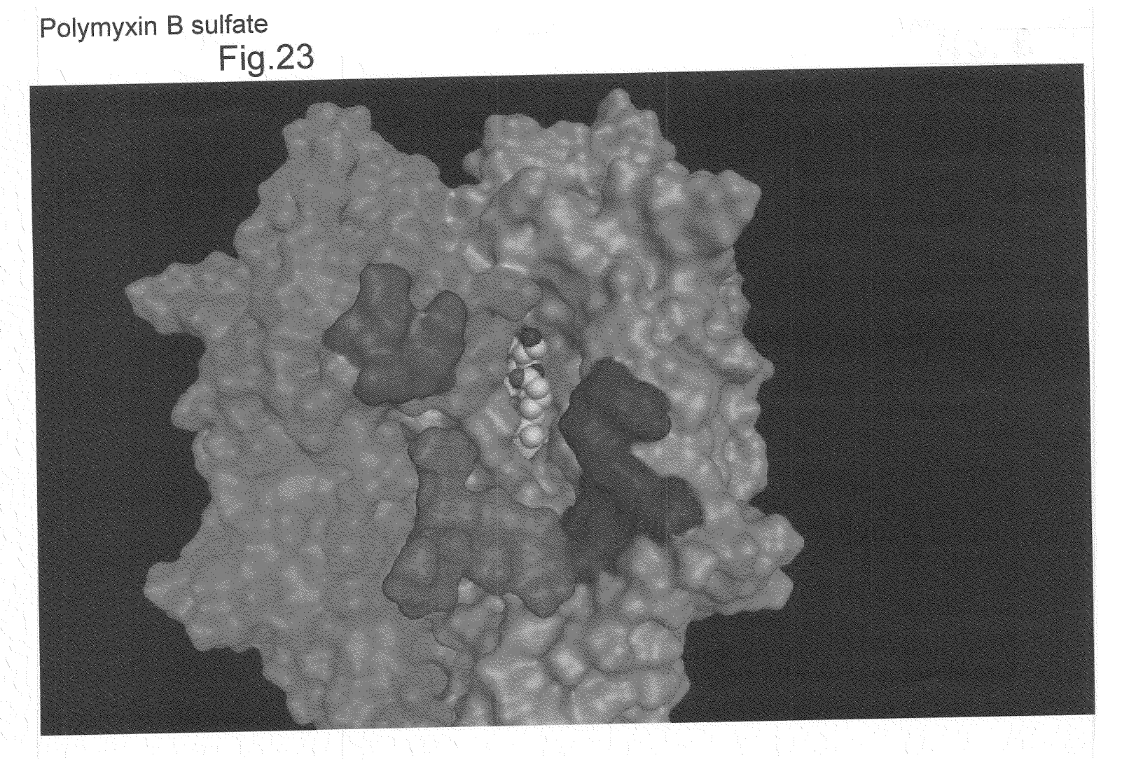

Representative best hit compounds, or possible active agents, are shown in Table 6 and FIGS. 21-23.

### <Protease Susceptibility Assay (1)>

Purified Aic68HA and bacitracin (bacitracin per HA protomer) were mixed in 50 mM Tris-HCl (pH 8.0) containing 1% dodecylmaltoside. The pH was lowered to 5.0 by adding 0.1 M citrate Na (pH 2.5) to all samples except controls.

Reaction mixtures were incubated at 25° C. for 20 minutes, and then the pH was neutralized by the addition of 1 M Tris-HCl (pH 8.9).

Trypsin was added to all samples (except controls) at a final ratio of 1:100 by mass, and the samples were digested for 40 minutes at 25° C.

To verify that F005-126 itself did not prevent tryptic digestion,

Fab-PP was mixed with HA that had been treated at pH 5.0 and then neutralized in the same way, and then the sample was incubated for 1 hour at 25° C.

Non-reducing SDS buffer was added to each reaction and each reaction mixture was boiled for 5 minutes. Samples were subjected to SDS-PAGE and 0.63 g of HA was applied to each lane. The gel was stained with Coomassie Brilliant Blue. The results are analyzed for assessment of the subject assays.

### <Protease Susceptibility Assay (2)>

Purified Aic68HA and compounds predicted to be inhibitors for HA from docking study were mixed in 50 mM Tris-HCl (pH 8.0) containing 1% dodecylmaltoside. The pH was lowered to 4.9-5.2 by adding 0.1 M citrate Na (pH 2.5) to all samples except controls. Reaction mixtures were incubated at 25° C. for 40 minutes, and then the pH was neutralized by adding 0.5 M Tris-HCl (pH 8.9). Trypsin was added to all samples (except a control) at a final ratio of 1:50 (trypsin:HA) by mass, and the samples were digested at 25° C. for 40 minutes. To verify that the compounds themselves did not prevent tryptic digestion, the compounds were mixed with HA that had been treated at pH 4.9-5.2 and then neutralized in the same way. Non-reducing SDS buffer was added to each reaction and each reaction mixture was boiled for 5 minutes. Samples were subjected to SDS-PAGE and 0.63 μg of HA was applied to each lane. The gel was stained with Coomassie Brilliant Blue.

The results are analyzed for assessment of the subject assays.

As described above, the present invention has been exemplified using preferred embodiments of the present invention, but it should not be construed that the present invention is limited to the embodiments. It is understood that the scope of the present invention should be construed only by the claims. It is understood that a person skilled in the art can carry out an equivalent scope based on the description of the present invention and the technical common knowledge, from the description of the preferred embodiments of the present invention. It is understood that the contents of patents, patent applications and documents cited as used herein are incorporated by reference as the contents thereof are specifically described herein.

### PRIOR ART DOCUMENTS

1. R. G. Webster, W. J. Bean, 0. T. Gorman, T. M. Chambers, Y. Kawaoka, Microbiol. Rev. 56, 152 (1992).
2. P. Palese, Nat. Med. 10, S82 (2004).
3. R. J. Garten et al., Science 325, 197 (2009).
4. C. P. Simmons et al., PLoS Med. 4, 928 (2007).
5. D. C. Ekiert, I. A. Wilson, Cur. Opin. Virol. 2, 134 (2012).
6. J. J. Skehel, D. C. Wiley, Annu. Rev. Biochem. 69, 531 (2000).
7. M. Knossow et al., Virology 302, 294 (2002).
8. D. C. Ekiert et al., Science 324, 246 (2009).

### 9. D. C. Wiley, I. A. Wilson, J. J. Skehel, Nature 289, 373 (1981).

10. P. A. Underwood, J. Gen. Virol. 62, 153 (1982).
11. Y. Okuno, Y. Isegawa, F. Sasao, S. Ueda, J. Virol. 67, 2552 (1993).
12. Y. A. Smimov et al., Acta Virol. 43, 237 (1999).
13. A. K. Kashyap et al., Proc. Natl. Acad. Sci. U.S.A. 105, 5986 (2008).
14. M. Throsby et al., PLoS ONE 3, e3942 (2008).
15. J. Sui et al., Nat. Struct. Mol. Biol. 16, 265 (2009).
16. T. T. Wang et al., PLoS Pathog. 6, 1 (2010).
17. D. Corti et al., J. Clin. Invest. 20, 1663 (2010).
18. C. J. Wei et al., Science 329, 1060 (2010).
19. J. Wrammert et al., J. Exp. Med. 208, 181 (2010).
20. D. C. Ekiert et al., Science 333, 843 (2011).
21. D. Corti et al., Science 333, 850 (2011).
22. J. R. Whittle et al., Proc. Natl. Acad. Sci. U.S.A. 108, 14216 (2011).
23. J. C. Krause et al., J. Virol. 85, 10905 (2011).
24. H. Hu et al., J. Virol. 86, 2978 (2012).
25. G S. Tan et al., J. Virol. 86, 6179 (2012).
26. J. C. Krause et al., J. Virol. 86, 6334 (2012).
27. J. Okada et al., Virology 397, 322 (2010).
28. N. Ohshima et al., J. Virol. 85, 11048 (2011).
29.1. A. Wilson, J. J. Skehel, D. C. Wiley, Nature 289, 366 (1981).
30. J. Okada et al., J. Gen. Virol. 92, 326 (2011).
31. T. Bizebard et al., Nature 376, 92 (1995).
32. D. Fleury et al., Nat. Struct. Biol. 6, 530 (1999).
33. D. Fleury, R. S. Daniels, J. J. Skehel, M. Knossow, T. Bizebard, Proteins 40, 572 (2000).
34. C. Barbey-Martin et al., Virology 294, 70 (2002).
35. R. Xu et al., Science 328, 357 (2010).
36. R. Xu, I. A. Wilson, J. Virol. 85, 5172 (2011).
37. J. J. Skehel et al., Proc. Natl. Acad. Sci. U.S.A. 79, 968 (1982).
38. Q. Huang, R. Opitz, E. W. Knapp, A. Herrmann, Biophys. J. 82, 1050 (2002).
39. P. Sivaramakrishna et al., FASEB J. 21, 995 (2007).
40. M. Ueda et al., J. Clin. Microbiol. 36, 340 (1998).
41. J. D. Marks et al., J. Mol. Biol. 222, 581 (1991).
42. Y. Iba, W. Ito, Y. Kurosawa, Gene 194, 35 (1997).
43. K. R. Abhinandan, A. C. Martin, Mol. Immunol. 45, 3832 (2008).
44. W. Ito, Y. Kurosawa, J. Biol. Chem. 268, 20668 (1993).
45. Y. Okuno et al., J. Clin. Microbiol. 28, 1308 (1990).
46. N. Nakagawa, R. Kubota, T. Nakagawa, Y. Okuno, J. Gen. Virol. 82, 2169 (2001).
47. W. Kabsch, J. Appl. Cryst. 26, 795 (1993).
48. Z. O. Otwinowski, W. Minor, Methods Enzymol. 276, 307 (1997).
49. J. Chen et al., Cell 95, 409 (1998).
50. P. Emsley, K. Cowtan, Acta Crystallogr. D Biol. Crystallogr. 60, 2126 (2004).
51. V. B. Chen, Acta Crystallogr. D. Biol. Crystallogr. 66, 12 (2010).
52. M. L. Connolly, J. Appl. Crystallogr. 221, 709 (1983).

SEQUENCE LISTING DESCRIPTION

SEQ ID NO.: 1: Nucleic acid sequence of F005-126 antibody
CAGGTGCAGC TGGTGCAGTC TGGAGCTGAG GTGAAGAAGC CTGGGGCCTC
AGTGACGGTC TCCTGTCAGG TTTCTGGTTA CACCCTTACC AGCTACGGTC
TCAGTTGGGT GCGACAGGCC CCTGGACAAG GGCTTGAGTG GGTGGGCTGG
ATTAACACTT ACGATGGTCA GACAAAGTAT GTAAAGAAGT TCCAGGGCCG
AGTCACCATG ACCACACACA CAGGCACGAA CACAGCCTAC ATGGAAATGA
AGAGCCTGAG ATCTGACGAC ACGGCCGTGT ATTACTGTGC GAGAGTCGAA
GGGGTTCGGG GAGTTATGGG CTTTCATTAC TACCCAATGG ACGTCTGGGG
CCAAGGGACA ATGGTCACCG TCTCGAGCGC CTCCACCAAG GGCCCATCGG
TCTTCCCCCT GGCACCCTCC TCCAAGAGCA CCTCTGGGGG CACAGCGGCC
CTGGGCTGCC TGGTCAAGGA CTACTTCCCC GAACCGGTGA CGGTGTCGTG
GAACTCAGGC GCCCTGACCA GCGGCGTGCA CACCTTCCCG GCTGTCCTAC
AGTCCTCAGG ACTCTACTCC CTCAGCAGCG TGGTGACCGT GCCCTCCAGC
AGCTTGGGCA CCCAGACCTA CATCTGCAAC GTGAATCACA AGCCCAGCAA
CACCAAGGTG GACAAGAAAG TTGAGCCCAA ATCTTGTGAC AAAACTCACA
CATGCCCACC GTGCCCAGCA CCTGAACTCC TGGGGGGACC GTCAGTCTTC
CTCTTCCCCC CAAAACCCAA GGACACCCTC ATGATCTCCC GGACCCCTGA
GGTCACATGC GTGGTGGTGG ACGTGAGCCA CGAAGACCCT GAGGTCAAGT
TCAACTGGTA CGTGGACGGC GTGGAGGTGC ATAATGCCAA GACAAAGCCG
CGTGAGGAGC AGTACAACAG CACGTACCGT GTGGTCAGCG TCCTCACCGT
CCTGCACCAG GACTGGCTGA ATGGCAAGGA GTACAAGTGC AAGGTCTCCA
ACAAAGCCCT CCCAGCCCCC ATCGAGAAAA CCATCTCCAA AGCCAAAGGG
CAGCCCCGAG AACCACAGGT GTACACCCTG CCCCCATCCC GGGATGAGCT
GACCAAGAAC CAGGTCAGCC TGACCTGCCT GGTCAAAGGC TTCTATCCCA
GCGACATCGC CGTGGAGTGG GAGAGCAATG GGCAGCCGGA GAACAACTAC
AAGACCACGC CTCCCGTGCT GGACTCCGAC GGCTCCTTCT TCCTCTACAG
CAAGCTCACC GTGGACAAGA GCAGGTGGCA GCAGGGGAAC GTCTTCTCAT
GCTCCGTGAT GCATGAGGCT CCGCACAACC ACTACACGCA GAAGAGCCTC
TCCCTGTCTC CGGGTAAATG ATGA
SEQ ID NO.: 2: Amino acid sequence of F005-126 antibody
QVQLVQSGAE VKKPGASVTV SCQVSGYTLT SYGLSWVRQA PGQGLEWVGW
INTYDGQTKY VKKFQGRVTM TTHTGTNTAY MEMKSLRSDD TAVYYCARVE
GVRGVMGFHY YPMDVWGQGT MVTVSSASTK GPSVFPLAPS SKSTSGGTAA
LGCLVKDYFP EPVTVSWNSG ALTSGVHTFP AVLQSSGLYS LSSVVTVPSS
SLGTQTYICN VNHKPSNTKV DKKVEPKSCD KTHTCPPCPA PELLGGPSVF
LFPPKPKDTL MISRTPEVTC VVVDVSHEDP EVKFNWYVDG VEVHNAKTKP
REEQYNSTYR VVSVLTVLHQ DWLNGKEYKC KVSNKALPAP IEKTISKAKG
QPREPQVYTL PPSRDELTKN QVSLTCLVKG FYPSDIAVEW ESNGQPENNY
KTTPPVLDSD GSFFLYSKLT VDKSRWQQGN VFSCSVMHEA PHNHYTQKSL
SLSPGK
SEQ ID NO.: 3: Amino acid sequence of CDR1 of F005-126 antibody
SYGLS
SEQ ID NO.: 4: Amino acid sequence of CDR2 of F005-126 antibody
WINTYDGQTKYVKKFQG
SEQ ID NO.: 5: Amino acid sequence of CDR3 of F005-126 antibody
VEGVRGVMGFHYYPMDV
SEQ ID NO.: 6: Amino acid sequence of FR1 of F005-126 antibody
QVQLVQSGAEVKKPGASVTVSCQVSGYTLT
SEQ ID NO.: 7: Amino acid sequence of FR2 of F005-126 antibody
WVRQAPGQGLEWVG
SEQ ID NO.: 8: Amino acid sequence of FR3 of F005-126 antibody
RVTMTTHTGTNTAYMEMKSLRSDDTAVYYCAR
SEQ ID NO.: 9: Amino acid sequence of FR4 of F005-126 antibody
WGQGTMVTVSS
SEQ ID NO.: 10: the binding site with HA of CDR2 sequence of
F005-126 antibody
SEQ ID NO.: 11: the binding site with HA of CDR3 sequence of
F005-126 antibody
SEQ ID NO.: 12: F005-126 Light chain(F005-126L) nucleic acid
sequence, full nelngth
CAGTCTGTGT TGACGCAGCC GCCCTCAGTG TCTGGGGCCC CAGGGCAGAG
GGTCACCATC TCCTGCACTG GGAGCAGCTC CAACATCGGG GCAGGTTATG
CTGTACACTG GTACCAGCAG CTTCCAGGAA CAGCCCCCAA ACTCCTCATC
TCTGGTAACA GCAATCGGCC CTCAGGGGTC CCTGACCGAT TCTCTGGCTC
CAAGTCTGGC ACCTCAGCCT CCCTGGCCAT CACTGGGCTC CAGGCTGAGG
ATGAGGCTGA TTATTACTGC CAGTCCTATG ACAGCAGCCT GAGTGGTTCG
GTATTCGGCG GAGGAACCAA GCTGACCGTC CTAGGTCAGC CCAAGGCTGC
CCCCTCGGTC ACTCTGTTCC CGCCCTCCTC TGAGGAGCTT CAAGCCAACA
AGGCCACACT GGTGTGTCTC ATAAGTGACT TCTACCCGGG AGCCGTGACA
GTGGCCTGGA AGGCAGATAG CAGCCCCGTC AAGGCGGGAG TGGAGACCAC
CACACCCTCC AAACAAAGCA ACAACAAGTA CGCGGCCAGC AGCTATCTGA
GCCTGACGCC TGAGCAGTGG AAGTCCCACA GAAGCTACAG CTGCCAGGTC
ACGCATGAAG GGAGCACCGT GGAGAAGACA GTGGCCCCTA CAGAATGTTC
GGCGCGCCAG
SEQ ID NO.: 13: F005-126 Light chain(F005-126L) amino acid
sequence, full nelngth
QSVLTQPPSV SGAPGQRVTI SCTGSSSNIG AGYAVHWYQQ LPGTAPKLLI
SGNSNRPSGV PDRFSGSKSG TSASLAITGL QAEDEADYYC QSYDSSLSGS
VFGGGTKLTV LGQPKAAPSV TLFPPSSEEL QANKATLVCL ISDFYPGAVT
VAWKADSSPV KAGVETTTPS KQSNNKYAAS SYLSLTPEQW KSHRSYSCQV
HEGSTVEKT VAPTECSARQ
SEQ ID NO.: 14: F005-126 (Homo sapiens) Light chain(F005-126L)
amino acid sequence, CDR1
TGSSSNIGAGYAVH
SEQ ID NO.: 15: F005-126 Light chain(F005-126L) amino acid
sequence, CDR2
GNSNRPS
SEQ ID NO.: 16: F005-126 Light chain(F005-126L) amino acid
sequence, CDR3
QSYDSSLSGSV
SEQ ID NO.: 17: F005-126 Light chain(F005-126L) amino acid
sequence, FR1
QSVLTQPPSVSGAPGQRVTISC
SEQ ID NO.: 18: F005-126 Light chain(F005-126L) amino acid
sequence, FR2
WYQQLPGTAPKLLIS
SEQ ID NO.: 19: F005-126 Light chain(F005-126L) amino acid
sequence, FR3
GVPDRFSGSKSGTSASLAITGLQAEDEADYYC
SEQ ID NO.: 20: F005-126 Light chain(F005-126L) amino acid
sequence, FR4
FGGGTKLTVLG
SEQ ID NO.: 21: Amino acid sequence of Influenza virus
Hemagglutinin HA1, type H3N2, Strain Aic68
GLFGAIAGFI ENGWEGMIDG WYGFRHQNSE GTGQAADLKS TQAAIDQING
KLNRVIEKTN EKFHQIEKEF SEVEGRIQDL EKYVEDTKID LWSYNAELLV
ALENQHTIDL TDSEMNKLFE
KTRRQLRENA EDMGNGCFKI YHKCDNACIE SIRNGTYDHD VYRDEALNNR
FQIKGVELKS GYKDWILWIS FAISCFLLCV VLLGFIMWAC QRGNIRCNIC I
[HA2]
SEQ ID NO.: 22: Amino acid sequence of Influenza virus
Hemagglutinin HA2, type H3N2, Strain Fuk70 full sequence
GIFGAIAGFI ENGWEGMIDG WYGFRHQNSE GTGQAADLKS TQAAIDQING
KLNRIIEKTN EKFHQIEKEF SEVEGRIQDL EKYVEDTKID LWSYNAELLV
ALENQHTIDL TDSEMNKLFE
KTRRQLRENA EDMGNGCFKI YHKCDNACIE SIRNGTYDHD VYRDEALNNR
FQIKGVELKS GYKDWILWIS FAISCFLLCV VLLGFIMWAC QRGNIRCNIC I
[HA2]
SEQ ID NO.: 23: Amino acid sequence of Influenza virus
Hemagglutinin HA2, type H3N2, Strain Tok73 full sequence
GIFGAIAGFI ENGWEGMIDG WYGFRHQNSE GTGHAADLKS TQAAIDQING
KLNRVIEKTN EKFHQIEKEF SEVEGRIQDL EKYVEDTKID LWSYNAELLV
ALENQHTIDL TDSEMNKLFE
KTRRQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC I
[HA2]
SEQ ID NO.: 24: Amino acid sequence of Influenza virus
Hemagglutinin HA2, type H3N2, Strain Yam77 full sequence
GLFGAIAGFI ENGWEGMIDG WYGFRHQNSE GTGQAADLKS TQAAIDQING
KLNRVIEKTN EKFHQIEKEF SEVEGRIQDL EKYVEDTKID LWSYNAELLV
ALENQHTIDL TDSEMNKLFE
KTRRQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC I
[HA2]
SEQ ID NO.: 25: Amino acid sequence of Influenza virus
Hemagglutinin HA2, type H3N2, Strain Nii81 full sequence
GIFGAIAGFI ENGWEGMVDG WYGFGHQNSE GTGQAADLKS TQAAIDQING
KLNRVIEKTN EKFHQIEKEF SEVEGRIQDL EKYVEDTKID LWSYNAELLV
ALENQHTIDL TDSEMNKLFE
KTRRQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC [HA2]
SEQ ID NO.: 26: Amino acid sequence of Influenza virus
Hemagglutinin HA2, type H3N2, Strain Fuk85 full sequence
GIFGAIAGFI ENGWEGMVDG WYGFRHQNSE GTGQAADLKS TQAAIDQING
KLNRLIEKTN EKFHQIEKEF SEVEGRIQDL EKYVEDTKID LWSYNAELLV
ALENQHTIDL TDSEMNKLFE
KTRKQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC I
[HA2]
SEQ ID NO.: 27: Amino acid sequence of Influenza virus
Hemagglutinin HA2, type H3N2, Strain Syd97 full sequence
GIFGAIAGFI ENGWEGMVDG WYGFRHQNSE GTGQAADLKS TQAAINQING
KLNRLIEKTN EKFHQIEKEF SEVEGRIQDL EKYVEDTKID LWSYNAELLV
ALENQHTIDL TDSEMNKLFE
RTRKQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC I
[HA2]
SEQ ID NO.: 28: Amino acid sequence of Influenza virus
Hemagglutinin HA2, type H3N2, Strain Pan99 full sequence
GIFGAIAGFI ENGWEGMVDG WYGFRHQNSE GTGQAADLKS TQAAINQING
KLNRLIEKTN EKFHQIEKEF SEVEGRIQDL EKYVEDTKID LWSYNAELLV
ALENQHTIDL TDSEMNKLFE
RTKKQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC I
[HA2]
SEQ ID NO.: 29: Amino acid sequence of Influenza virus
Hemagglutinin HA2, type H3N2, Strain Wyo03 full sequence
GIFGAIAGFI ENGWEGMVDG WYGFRHQNSE GTGQAADLKS TQAAINQING
KLNRLIGKTN EKFHQIEKEF SEVEGRIQDL EKYVEDTKID LWSYNAELLV
ALENQHTIDL TDSEMNKLFE
RTKKQLRENA EDMGNGCFKI YHKCDNACIE SIRNGTYDHD VYRDEALNNR
FQIKGVELKS GYKDWILWIS FAISCFLLCV ALLGFIMWAC QKGNIRCNIC I
[HA2]
SEQ ID NO.: 30: Amino acid sequence of Influenza virus
Hemagglutinin HA2, type H3N2, Strain NY04 full sequence
GIFGAIAGFI ENGWEGMVDG WYGFRHQNSE GIGQAADLKS TQAAINQING
KLNRLIGKTN EKFHQIEKEF SEVEGRIQDL EKYVEDTKID LWSYNAELLV
ALENQHTIDL TDSEMNKLFE
RTKKQLRENA EDMGNGCFKI YHKCDNACIG SIRNGTYDHD VYRDEALNNR
FQIKGVELKS GYKDWILWIS FAISCFLLCV VLLGFIMWAC QKGNIRCNIC I [HA2]
SEQ ID NO.: 31: Amino acid sequence of Influenza virus
Hemagglutinin HA2, type H3N8, aviAus77HA2 full sequence
H3N8 aviAus77HA2
GLFGAIAGFI ENGWEGMIDG WYGFRHQNSE GTGQAADLKS TQAAIDQING
KLNRVIERTN EKFHQIEKEF SEVEGRIQDL EKYVEDTKID LWSYNAELLV
ALENQHTIDL TDSEMNKLFE
KTRRQLRENA EDMGNGCFKI YHKCDNACIE SIRNGTYDHD IYRDEALNNR
FQIKGVELKS SYKDWILWIS FAISCFLLCV VLLGFIMWAC QRGNIRCNIC I
SEQ ID NO.: 32: Amino acid sequence of Influenza virus
Hemagglutinin HA2, type H7N3, aviIta02HA2 (PDB 1TI8) full
sequence
G LFGAIAGFIE NGWEGLIDGW
YGFRHQNAQG EGTAADYKST QSAIDQITGK LNRLIEKTNQ QFELIDNEFT
EVEKQIGNVI
NWTRDSMTEV WSYNAELLVA MENQHTIDLA DSEMNKLYER VKRQLRENAE
EDGTGCFEIF
HKCDDDCMAS IRNNTYDHSR YREEAMQNRI QIDPVKLSSG YKDVILWFSF
GASCFILLAI
AMGLVFICVK NGNMRCTICI
SEQ ID NO.: 33: Amino acid sequence of Influenza virus
Hemagglutinin HA2, type H1N1, HA2 SC1918 (PDB 1RUZ) full
sequence
MIDGWYGYHH QNEQGSGYAA DQKSTQNAID GITNKVNSVI EKMNTQFTAV
GKEFNNLERR
IENLNKKVDD GFLDIWTYNA ELLVLLENER TLDFHDSNVR NLYEKVKSQL
KNNAKEIGNG
CFEFYHKCDD ACMESVRNGT YDYPKYSEES KLNREEIDGV KLESMGVYQI
LAIYSTVASS
LVLLVSLGAI SFWMCSNGSL QCRICI
SEQ ID NO.: 34: Amino acid sequence of Influenza virus
Hemagglutinin HA2, type H1N1, Strain NC99 full sequence
GLFGAIAGFI EGGWTGMVDG WYGYHHQNEQ GSGYAADQKS TQNAINGITN
KVNSVIEKMN TQFTAVGKEF NKLERRMENL NKKVDDGFLD IWTYNAELLV
LLENERTLDF HDSNVKNLYE
KVKSQLKNNA KEIGNGCFEF YHKCNNECME SVKNGTYDYP KYSEESKLNR
EKIDGVKLES MGVYQILAIYS TVASSLVLLV SLGAISFWMC SNGSLQCRIC I [HA2]
SEQ ID NO.: 35: Amino acid sequence of Influenza virus
Hemagglutinin HA2, type H1N1 Cal09pdmHA2 (PDB 3LZG) full
sequence
GLFGAI AGFIEGGWTG
MVDGWYGYHH QNEQGSGYAA DLKSTQNAID EITNKVNSVI EKMNTQFTAV
GKEFNHLEKR
IENLNKKVDD GFLDIWTYNA ELLVLLENER TLDYHDSNVK NLYEKVRSQL
KNNAKEIGNG
CFEFYHKCDN TCMESVKNGT YDYPKYSEEA KLNREEIDGV KLESTRIYQI
LAIYSTVASS
LVLVVSLGAI SFWMCSNGSL QCRICI
SEQ ID NO.: 36: Amino acid sequence of Influenza virus
Hemagglutinin HA2, type H2N2 Jpn57HA2 (PDB 2WRD) full
sequence
GLFGAIAGFI EGGWQGMVDG
WYGYHHSNDQ GSGYAADKES TQKAFDGITN KVNSVIEKMN TQFEAVGKEF
SNLERRLENL
NKKMEDGFLD VWTYNAELLV LMENERTLDF HDSNVKNLYD KVRMQLRDNV
KELGNGCFEF
YHKCDDECMN SVKNGTYDYP KYEEESKLNR NEIKGVKLSS MGVYQILAIY
ATVAGSLSLA
IMMAGISFWM CSNGSLQCRI CI
SEQ ID NO.: 37: Amino acid sequence of Influenza virus
Hemagglutinin HA2, type H5N1 Viet04HA2 (PDB 2FK0) full
sequence
GLFGAIAG FIEGGWQGMV
DGWYGYHHSN EQGSGYAADK ESTQKAIDGV TNKVNSIIDK MNTQFEAVGR
EFNNLERRIE
NLNKKMEDGF LDVWTYNAEL LVLMENERTL DFHDSNVKNL YDKVRLQLRD
NAKELGNGCF
EFYHKCDNEC MESVRNGTYD YPQYSEEARL KREEISGVKL ESIGIYQILS
IYSTVASSLA
LAIMVAGLSL WMCSNGSLQC RICI
SEQ ID NO.: 38: Amino acid sequence of Influenza virus
Hemagglutinin HA2, type H9N2 swHK98HA2 (PDB 1JSD) full
sequence
GL FGAIAGFIEG GWPGLVAGWY
GFQHSNDQGV GMAADRDSTQ KAIDKITSKV NNIVDKMNKQ YGIIDHEFSE
IETRLNMINN
KIDDQIQDIW TYNAELLVLL ENQKTLDEHD ANVNNLYNKV KRALGSNAME
DGKGCFELYH
KCDDQCMETI RNGTYNRRKY KEESKLERQK IEGIKLESEG TYKILTIYST
VASSLVIAMG
FAAFLFWAMS
SEQ ID NO.: 39: Amino acid sequence of Influenza virus
Hemagglutinin HA1, type H3N8, aviAus77HA1 full sequence
H3N8 aviAus77HA1
QDLSGNDNST ATLCLGHHAV SNGTVVKTIT DDRVEVTNAT ELVQSSSTGK
ICNNPHRILD GRDCTLIDAL LGDPHCDVFQ DETWDLFIER SNAFSNCYPY
DVPDHASLRS LVASSGTLEF ICEGFTWAGV TQNGESGACK RGPANGFFSR
LNWLTKSGST YPVLNVTMPN NDNFDKLYIW GVHHPSTNQE QTNLYVQASG
RVTVSTRRSQ QTIIPNIGSR PWVRGQSGRI SIYWTIVKPG DVLVINSNGN
LIAPRGYFKM RTGKSSIMRS DVPIDTCVSE CITPNGSIPN DKPFQNVNKI
TYGACPKYVK QNTLKLATGM RNVPEKQT R
SEQ ID NO.: 40: Amino acid sequence of Influenza virus
Hemagglutinin HA1, type H7N3, aviIta02HA1 (PDB 1TI8) full
sequence
AL VAIIPTNADK ICLGHHAVSN GTKVNTLTER GVEVVNATET VERTNVPRIC
SKGKRTVDLG QCGLLGTITG PPQCDQFLEF SADLIIERRE GSDVCYPGKF
VNEEALRQIL
RESGGIDKET MGFTYSGIRT NGATSACRRS GSSFYAEMKW LLSNTDNAAF
PQMTKSYKNT
RKDPALIIWG IHHSGSTTEQ TKLYGSGNKL ITVGSSNYQQ SFVPSPGARP
QVNGQSGRID
FHWLMLNPND TVTFSFNGAF IAPDRASFLR GKSMGIQSSV QVDANCEGDC
YHSGGTIISN
LPFQNINSRA VGKCPRYVKQ ESLMLATGMK NVPEIPKGR
SEQ ID NO.: 41: Amino acid sequence of Influenza virus
Hemagglutinin HA1, type H1N1, HA1 SC1918 (PDB 1RUZ) full
sequence
LLC AFAATNADTI CIGYHANNST DTVDTVLEKN VTVTHSVNLL EDSHNGKLCK
LKGIAPLQLG KCNIAGWLLG NPECDLLLTA SSWSYIVETS NSENGTCYPG
DFIDYEELRE
QLSSVSSFEK FEIFPKTSSW PNHETTKGVT AACSYAGASS FYRNLLWLTK
KGSSYPKLSK
SYVNNKGKEV LVLWGVHHPP TGTDQQSLYQ NADAYVSVGS SKYNRRFTPE
IAARPKVRDQ
AGRMNYYWTL LEPGDTITFE ATGNLIAPWY AFALNRGSGS GIITSDAPVH
DCNTKCQTPH
GAINSSLPFQ NIHPVTIGEC PKYVRSTKLR MATGLRNIPS IQSR
SEQ ID NO.: 42: Amino acid sequence of Influenza virus
Hemagglutinin HA1, type H1N1 Cal09pdmHA1 (PDB 3LZG) full
sequence
LLY TFATANADTL CIGYHANNST DTVDTVLEKN VTVTHSVNLL EDKHNGKLCK
LRGVAPLHLG KCNIAGWILG NPECESLSTA SSWSYIVETP SSDNGTCYPG
DFIDYEELRE
QLSSVSSFER FEIFPKTSSW PNHDSNKGVT AACPHAGAKS FYKNLIWLVK
KGNSYPKLSK
SYINDKGKEV LVLWGIHHPS TSADQQSLYQ NADTYVFVGS SRYSKKFKPE
IAIRPKVRDQ
EGRMNYYWTL VEPGDKITFE ATGNLVVPRY AFAMERNAGS GIIISDTPVH
DCNTTCQTPK
GAINTSLPFQ NIHPITIGKC PKYVKSTKLR LATGLRNIPS IQSR
SEQ ID NO.: 43: Amino acid sequence of Influenza virus
Hemagglutinin HA1, type H2N2 Jpn57HA1 (PDB 2WRD) full
sequence
LILLF TAVRGDQICI GYHANNSTEK VDTILERNVT VTHAKDILEK THNGKLCKLN
GIPPLELGDC SIAGWLLGNP ECDRLLSVPE WSYIMEKENP RDGLCYPGSF
NDYEELKHLL
SSVKHFEKVK ILPKDRWTQH TTTGGSRACA VSGNPSFFRN MVWLTKKGSN
YPVAKGSYNN
TSGEQMLIIW GVHHPNDETE QRTLYQNVGT YVSVGTSTLN KRSTPEIATR
PKVNGLGGRM
EFSWTLLDMW DTINFESTGN LIAPEYGFKI SKRGSSGIMK TEGTLENCET
KCQTPLGAIN
TTLPFHNVHP LTIGECPKYV KSEKLVLATG LRNVPQIESR
SEQ ID NO.: 44: Amino acid sequence of Influenza virus
Hemagglutinin HAQ, type H5N1 Viet04HAQ (PDB 2FK0) full
sequence
LFAI VSLVKSDQIC IGYHANNSTE QVDTIMEKNV TVTHAQDILE KKHNGKLCDL
DGVKPLILRD CSVAGWLLGN PMCDEFINVP EWSYIVEKAN PVNDLCYPGD
FNDYEELKHL
LSRINHFEKI QIIPKSYWSS HEASLGVSSA CPYQGKSSFF RNVVWLTKKN
STYPTIKRSY
NNTNQEDLLV LWGIHHPNDA AEQTKLYQNP TTYISVGTST LNQRLVPRIA
TRSKVNGQSG
RMEFFWTILK PNDAINFESN GNFIAPEYAY KIVKKGDSTI MKSELEYGNC
NTKCQTPMGA
INSSMPFHNI HPLTIGECPK YVKSNRLVLA TGLRNSPQRE RRRKKR
SEQ ID NO.: 45: Amino acid sequence of Influenza virus
Hemagglutinin HA1, type H9N2 swHK98HA1 (PDB 1JSD) full
sequence
IL LVVTASNADK ICIGYQSTNS TETVDTLTET NVPVTHAKEL LHTEHNGMLC
ATNLGHPLIL DTCTIEGLIY GNPSCDLLLG GREWSYIVER PSAVNGMCYP
GNVENLEELR
SLFSSASSYQ RIQIFPDTIW NVSYSGTSKA CSDSFYRSMR WLTQKNNAYP
IQDAQYTNNR
GKSILFMWGI NHPPTDTVQT NLYTRTDTTT SVTTEDINRT FKPVIGPRPL
VNGLHGRIDY
YWSVLKPGQT LRVRSNGNLI APWYGHILSG ESHGRILKTD LNSGNCVVQC
QTERGGLNTT
LPFHNVSKYA FGNCPKYVGV KSLKLAVGLR NVPARSSR
SEQ ID NO.: 46: partial sequence of germline GHV1-18*01
SEQ ID NO.: 47: partial sequence of germline IGLV1-40*01
SEQ ID NO.: 48: Amino acid sequence of Influenza virus
Hemagglutinin HA1, type H3N2, Strain Aic68
QDLPGNDNST ATLCLGHHAV PNGTLVKTIT DDQIEVTNAT ELVQSSSTGK
ICNNPHRILD GIDCTLIDAL LGDPHCDVFQ NETWDLFVER SKAFSNCYPY
DVPDYASLRS LVASSGTLEF ITEGFTWTGV TQNGGSNACK RGPGSGFFSR
LNWLTKSGST YPVLNVTMPN NDNFDKLYIW GVHHPSTNQE QTSLYVQASG
RVTVSTRRSQ QTIIPNIGSR PWVRGLSSRI SIYWTIVKPG DVLVINSNGN
LIAPRGYFKM RTGKSSIMRS DAPIDTCISE CITPNGSIPN DKPFQNVNKI
TYGACPKYVK QNTLKLATGM RNVPEKQT R [HA1]
SEQ ID NO.: 49: Amino acid sequence of Influenza virus
Hemagglutinin HA1, type H3N2, Strain Fuk70 full sequence
QDLPRNDNST ATLCLGHHAV PNGTLVKTIT DDQIEVTNAT ELVQSSSTGK
ICNNPHRILD GIDCTLIDAL LGDPHCDGFQ NETWDLFVER SKAFSNCYPY
DVPDYASLRS LVASSGTLEF ITEGFTWTGV TQNGGSNACK RGPGSGFFSR
LNWLTKSGST YPVLNVTMPN NDNFDKLYIW GVHHPSTDQE QTSLYVQASG
RVTVSTRRSQ QTIIPNIGSR PWVRGLSSRI SIYWTIVKPG DVLVINSNGN
LIAPRGYFKM RTGKSSIMRS DAPIDTCISE CITPNGSIPN DKPFQNVNKI
TYGACPKYVK QNTLKLATGM RNVPEKQT R [HA1]
SEQ ID NO.: 50: Amino acid sequence of Influenza virus
Hemagglutinin HA1, type H3N2, Strain Tok73 full sequence
QDFPGNDNST ATLCLGHHAV PNGTLVKTIT NDQIEVTNAN ELVQSSSTGK
ICNNPHRILD GINCTLIDAL LGDPHCDGFQ NETWDLFVER SKAFSNCYPY
DVPDYASLRS LVASSGTLEF INEGFTWTGV TQNGGSNACK RGPDSGFFSR
LNWLYKSGST YPVLNVTMPN NDNFDKLYIW GVHHPSTDQE QTNLYVQASG
RVTVSTKRSQ QTIIPNIGSR PWVRGLSSRI SIYWTIVKPG DILLINSNGN
LIAPRGYFKM RTGKSSIMRS DAPIGTCISE CITPNGSIPN DKPFQNVNKI
TYGACPKYVK QNTLKLATGM RNVPEKQT R [HA1]
SEQ ID NO.: 51: Amino acid sequence of Influenza virus
Hemagglutinin HA1, type H3N2, Strain Yam77 full sequence
QNLPRNDNST ATLCLGHHAV PNGTLVKTIT NDQIEVTNAT ELVQSSSTGR
ICDSPHRILD GKNCTLIDAL LGDPHCDGFQ NEKWDLFVER SKAFSNCYPY
DVPDYASLRS LVASSGTLEF INEGFNWTGV TQNGGSYACK RGPDNSFFSR
LNWLYESESK YPVLNVTMPN NDNFDKLYIW GVHHPSTDKE QTNLYVQASG
RVTVSTKRSQ QTIIPNVGSR PWVRGLSSRI SIYWTIVKPG DILLINSNGN
LIAPRGYFKI RTGKSSIMRS DAPIGTCSSE CITPNGSIPN DKPFQNVNKI
TYGACPKYVK QNTLKLATGM RNVPEKQT R [HA1]
SEQ ID NO.: 52: Amino acid sequence of Influenza virus
Hemagglutinin HA1, type H3N2, Strain Nii81 full sequence
QNLPGNDNST ATLCLGHHAV PNGTLVKTIT NDQIEVTNAT ELVQSSSTGR
ICDSPHRILD GKNCTLIDAL LGDPHCDGFQ NEKWDLFVER SKAFSNCYPY
DVPDYASLRS LVASSGTLEF INEGFNWTGV TQSGGSYTCK RGSDNSFFSR
LNWLYESESK YPALNVTMPN NGNFDKLYIW GVHHPSTDKE QTKLYVRASG
RVTVSTKRSQ QTIIPNIGPR PWVRGLSSRI SIYWTIVKPG DILLINSSGN
LIAPRGYFKI RTGKSSIMRS DAPIGTCSSE CITPNGSIPN DKPFQNVNRI
TYGACPKYVK QNTLKLATGM RNIPEKQT R [HA1]
SEQ ID NO.: 53: Amino acid sequence of Influenza virus
Hemagglutinin HA1, type H3N2, Strain Fuk85 full sequence
QKLPGNDNSK ATLCLGHHAV PNGTLVKTIT NDQIEVTNAT ELVQSSSTGR
ICDSPHRILD GKNCTLIDAL LGDPHCDGFQ NEKWDLFVER SKAFSNCYPY
DVPDYASLRS LVASSGTLEF INEDFNWTGV TQSGGSYACK RGSVNSFFSR
LNWLHESEYK YPALNVTMPN NGKFDKLYIW GVHHPSTDKE QTKLYVRASG
RVTVSTKRSQ QTVIPNIGSR PWVRGLSSGI SIYWTIVKPG DILLINSIGN
LIAPRGYFKI RTGKSSIMRS DAPIGTCSSE CITPNGSIPN DKPFQNVNKI
TYGACPRYVK QNTLKLATGM RNVPEKQT R [HA1]
SEQ ID NO.: 54: Amino acid sequence of Influenza virus
Hemagglutinin HA1, type H3N2, Strain Gui89 full sequence
QKLPGNDNST ATLCLGHHAV PNGTLVKTIT NDQIEVTNAT ELVHSSSTGR
ICDSPHRILD GKNCTLIDAL LGDPHCDGFQ NKEWDLFVER SKAYSNCYPY
DVPDYASLRS LVASSGTLEF INEDFNWTGV AQSGGSYACK RGSINSFFSR
LNWLHESEHK YPALNVTMPN NGKFDKLYIW GVHHPITDRE QTNLYVRASG
RVTVSTKRSQ QTVIPNIGSR PWVRGLSSRI SIYWTIVKPG DILLINSTGN
LIAPRGYFKI RTGKSSIMRS DAPIGTCSSE CITPNGSIPN DKPFQNVNRI
TYGACPRYVK QNTLKLATGM RNVPEKQT R [HA1]
SEQ ID NO.: 55: Amino acid sequence of Influenza virus
Hemagglutinin HA1, type H3N2, Strain Kit93 full sequence
QKLPGNDNST ATLCLGHHAV PNGTLVKTIT NDQIEVTNAT ELVQSSSTGR
ICDSPHRILD GKNCTLIDAL LGDPHCDGFQ NKEWDLFVER SKAYSNCYPY
DVPDYASLRS LVASSGTLEF INEDFNWTGV AQDGGSYACK RGSVNSFFSR
LNWLHKSEYK YPALNVSMPN NGKFDKLYIW GVHHPSTDSD QTSLYVQASG
RVTVSTKRSQ QTVTPNIGSR PWVRGQSSRI SIYWTIVKPG DILLINSTGN
LIAPRGYFKI RNGKSSIMRS DAPIGTCSFE CITPNGSIPN DKPFQNVNRI
TYGACPRYVK QNTLKLATGM RNVPEKQT R [HA1]
SEQ ID NO.: 56: Amino acid sequence of Influenza virus
Hemagglutinin HA1, type H3N2, Strain Syd97 full sequence
QKIPGNDNST ATLCLGHHAV PNGTLVKTIT NDQIEVTNAT ELVQSSSTGR
ICDSPHRILD GENCTLIDAL LGDPHCDGFQ NKEWDLFVER SKAYSNCYPY
DVPDYASLRS LVASSGTLEF NNESFNWTGV AQNGTSYACK RSSIKSFFSR
LNWLHQLKYK YPALNVTMPN NDKFDKLYIW GVHHPSTDSD QTSIYAQASG
RVTVSTKRSQ QTVIPNIGSR PWVRGISSRI SIYWTIVKPG DILLINSTGN
LIAPRGYFKI RSGKSSIMRS DAPIGKCNSE CITPNGSIPN DKPFQNVNRI
TYGACPRYVK QNTLKLATGM RNVPEKQT R [HA1]
SEQ ID NO.: 57: Amino acid sequence of Influenza virus
Hemagglutinin HA1, type H3N2, Strain Pan99 full sequence
QKLPGNDNST ATLCLGHHAV SNGTLVKTIT NDQIEVTNAT ELVQSSSTGR
ICDSPHQILD GENCTLIDAL LGDPHCDGFQ NKEWDLFVER SKAYSNCYPY
DVPDYASLRS LVASSGTLEF NNESFNWTGV AQNGTSSACK RRSNKSFFSR
LNWLHQLKYK YPALNVTMPN NEKFDKLYIW GVHHPSTDSD QISIYAQASG
RVTVSTKRSQ QTVIPNIGSS PWVRGVSSRI SIYWTIVKPG DILLINSTGN
LIAPRGYFKI RSGKSSIMRS DAPIGKCNSE CITPNGSIPN DKPFQNVNRI
TYGACPRYVK QNTLKLATGM RNVPEKQT R [HA1]
SEQ ID NO.: 58: Amino acid sequence of Influenza virus
Hemagglutinin HA1, type H3N2, Strain Wyo03 full sequence
QKLPGNDNST ATLCLGHHAV PNGTIVKTIT NDQIEVTNAT ELVQSSSTGG
ICDSPHQILD GENCTLIDAL LGDPQCDGFQ NKKWDLFVER SKAYSNCYPY
DVPDYASLRS LVASSGTLEF NNESFNWAGV TQNGTSSACK RRSNKSFFSR
LNWLTHLKYK YPALNVTMPN NEKFDKLYIW GVHHPGTDSD QISLYAQASG
RITVSTKRSQ QTVIPNIGSR PRVRDVSSRI SIYWTIVKPG DILLINSTGN
LIAPRGYFKI RSGKSSIMRS DAPIGKCNSE CITPNGSIPN DKPFQNVNRI
TYGACPRYVK QNTLKLATGM RNVPEKQT R [HA1]
SEQ ID NO.: 59: Amino acid sequence of Influenza virus
Hemagglutinin HA1, type H3N2, Strain NY04 full sequence
QKLPGNDNST ATLCLGHHAV PNGTIVKTIT NDQIEVTNAT ELVQSSSTGG
ICDSPHQILD GENCTLIDAL LGDPQCDGFQ NKKWDLFVER SKAYSNCYPY
DVPDYASLRS LVASSGTLEF NNESFNWTGV TQNGTSSSCK RRSNNSFFSR
LNWLTHLKFK YPALNVTMPN NEKFDKLYIW GVHHPVTDND QIRLYAQASG
RITVSTKRSQ QTVIPNIGSR PRVRDIPSRI SIYWTIVKPG DILLINSTGN
LIAPRGYFKI RSGKSSIMRS DAPIGKCNSE CITPNGSIPN DKPFQNVNRI
TYGACPRYVK QNTLKLATGM RNVPEKQT R [HA1]
SEQ ID NO.: 60: Amino acid sequence of Influenza virus
Hemagglutinin HA1, type H1N1, Strain NC99 full sequence
LLCTFTATYA DTICIGYHAN NSTDTVDTVL EKNVTVTHSV NLLEDSHNGK
LCLLKGIAPLQ LGNCSVAGWI LGNPECELLI SKESWSYIVET PNPENGTCYPG
YFADYEELRE QLSSVSSFERFEI FPKESSWPNH TVTGVSASCS HNGKSSFYRN
LLWLTGKNGL YPNLSKSYVN NKEKEVLVLW GVHHPPNIGN QRALYHTENA
YVSVVSSHYS RRFTPEIAKR PKVRDQEGRI NYYWTLLEPG DTIIFEANGN
LIAPWYAFAL SRGFGSGIITS NAPMDECDAK CQTPQGAINS SLPFQNVHPV
TIGECPKYVR SAKLRMVTGL RNIPSIQS R [HA1]
SEQ ID NO.: 61: H3HA forward, 5′-GCAAAAGCAGGGGATAATTCT
SEQ ID NO.: 62: H3HA backward, 5′-GTAGAAACAAGGGTGTTTTTAATTA
SEQ ID NO.: 63: H3HA 568, 5′-TGAACGTGACTATGCCAAACAATG

TABLE 1
(in the table VDW refers to van der Walls force)

| F5126 | | | | HA (acceptor (dist)/No. of vdw) | | | |
|---|---|---|---|---|---|---|---|
| Paratope | H-bond | | VDW | H-bond | | VDW | |
| HCDR1 | Ser31 | O | | Ser91 | OG (3.63) | | |
| | | OG | | | O (3.87) | | |
| | | | vdw | | | Ser91 | 2 |
| | | | vdw | | | Lys92 | 1 |
| HCDR2 | Asp54 | OD1 | | NAG-2 | O7 (2.58) | | |
| | | O | | MAN-2 | O2 (3.78) | | |
| | | | vdw | | | NAG-2 | 7 |
| | Gly55 | O | | BMA | O2 (3.56) | | |
| | Gln56 | OE1 | | BMA | O2 (2.96) | | |
| | | | | NAG-2 | O4 (3.49) | | |
| | | NE2 | | | O4 (3.0) | | |
| | | | vdw | | | NAG-2 | 1 |
| | | | vdw | | | BMA | 3 |
| | Thr57 | OG1 | | MAN-4 | O6 (3.47) | | |
| FR3 | Gly74 | O | | Gly240 | N (3.75) | | |
| | | | vdw | | | Gly240 | 6 |
| | | | vdw | | | Pro239 | 1 |
| HCDR3 | Val98 | O | | Asp271 | OD2 (3.2) | | |
| | | | vdw | | | Ser91 | 1 |
| | Arg99 | N | | Asp271 | OD2 (3.7) | | |
| | | O | | NAG-1 | O6 (2.5) | | |
| | | | vdw | | | NAG-1 | 2 |
| | | | vdw | | | Asp271 | 1 |
| | | | vdw | | | Ser91 | 1 |
| | Gly100 | O | | NAG-1 | O5 (3.1) | | |
| | | | vdw | | | Pro284 | 3 |
| | | | vdw | | | NAG-1 | 1 |
| | | | vdw | | | Ser270 | 1 |
| | | | vdw | | | Asp285 | 1 |

Table 2 (Tables 2A and 2B) The atomic coordinates of H3 which are particularly relevant to the binding related to CDR1, CDR2, FR3, CDR3 of the heavy chain. As used herein, CDR1 sections of Tables 2A and 2B form Table 2-1; CDR2 sections of Tables 2A and 2B form Table 2-2; FR3 sections of Tables 2A and 2B form Table 2-3; CDR3 sections of Tables 2A and 2B form Table 2-4.
Table 2A shows the atomic coordinates of H3 which are particularly relevant to the binding (JKL-90_{—}120608.pdb).
Table 2B shows the atomic coordinates of H3 which are particularly relevant to the binding (JKL-90_{—}0101.pdb).
The effective number of decimal place is first decimal place, in notation of the atomic coordinate.
Numbers of amino acids in F5126 H chain are shown as serial numbers of the amino acids in Table 2. Kabat numbering is applied to the amino acids in Tables 1 and 4, Figures, and documents in the present patent. Asp55, Gly56, Gln57, Thr58, Gly75, Val102, and Arg103 in Table 2 correspond to Asp54, Gly55, Gln56, Thr57, Gly74, Val98, and Arg99, respectively, in Tables 1 and 4, Figures, and the documents. The atoms in CDR1, CDR2, CDR3, and FR3 that are colored form hydrogen bonds with the atoms in HA that are shown in the same colors as described in Table 1.

TABLE 2A

| | | Atom | | | | | | | | temper- | | | | |
|---|---|---|---|---|---|---|---|---|---|---|---|---|---|---|
| | Atom | (α, β, | amino | Chain | residue | Coordinate | Coordinate | Coordinate | occupation | ature | | | | |
| | No. | γ, δ) | acid | ID | No. | (Å) m | (Å) | (Å) | ratio | factor | | | | |
| CDR1 | | | | | | | | | | | | | | |
| ATOM | 84964 | N | SER | 9 | 31 | −5.529 | −2.939 | 96.087 | 1.00 | 138.85 | 9 | N | 31 | F5126-VH |
| ATOM | 84965 | CA | SER | 9 | 31 | −4.649 | −1.971 | 96.736 | 1.00 | 138.85 | 9 | C | 31 | |
| ATOM | 84966 | CB | SER | 9 | 31 | −3.180 | −2.305 | 96.450 | 1.00 | 134.44 | 9 | C | 31 | |
| ATOM | 84967 | OG | SER | 9 | 31 | −2.917 | −2.336 | 95.059 | 1.00 | 134.44 | 9 | O | 31 | Yellow* |
| ATOM | 84968 | C | SER | 9 | 31 | −4.963 | −0.552 | 96.271 | 1.00 | 138.85 | 9 | C | 31 | |
| ATOM | 84969 | O | SER | 9 | 31 | −4.156 | 0.363 | 96.435 | 1.00 | 138.85 | 9 | O | 31 | Blue* |
| ATOM | 37507 | N | SER | L | 91 | −0.281 | 0.916 | 100.728 | 1.00 | 124.24 | L | N | | HA-b |
| ATOM | 37508 | CA | SER | L | 91 | −1.034 | −0.243 | 100.261 | 1.00 | 124.24 | L | C | | |
| ATOM | 37509 | CB | SER | L | 91 | −2.526 | −0.051 | 100.536 | 1.00 | 120.61 | L | C | | |
| ATOM | 37510 | OG | SER | L | 91 | −3.031 | 1.073 | 99.838 | 1.00 | 120.61 | L | O | | Blue* |
| ATOM | 37511 | C | SER | L | 91 | −0.809 | −0.433 | 98.767 | 1.00 | 124.24 | L | C | | |
| ATOM | 37512 | O | SER | L | 91 | −0.805 | −1.557 | 98.265 | 1.00 | 124.24 | L | O | | |
| ATOM | 37513 | N | LYS | L | 92 | −0.622 | 0.678 | 98.062 | 1.00 | 118.88 | L | N | | |
| ATOM | 37514 | CA | LYS | L | 92 | −0.386 | 0.654 | 96.624 | 1.00 | 118.88 | L | C | | |
| ATOM | 37515 | CB | LYS | L | 92 | −0.238 | 2.083 | 96.099 | 1.00 | 115.59 | L | C | | |
| ATOM | 37516 | CG | LYS | L | 92 | 1.078 | 2.737 | 96.500 | 1.00 | 115.59 | L | C | | |
| ATOM | 37517 | CD | LYS | L | 92 | 1.116 | 4.220 | 96.174 | 1.00 | 115.59 | L | C | | |
| ATOM | 37518 | CE | LYS | L | 92 | 0.285 | 5.024 | 97.157 | 1.00 | 115.59 | L | C | | |
| ATOM | 37519 | NZ | LYS | L | 92 | 0.450 | 6.489 | 96.940 | 1.00 | 115.59 | L | N | | |
| ATOM | 37520 | C | LYS | L | 92 | 0.896 | −0.126 | 96.345 | 1.00 | 118.88 | L | C | | |
| ATOM | 37521 | O | LYS | L | 92 | 1.212 | −0.438 | 95.196 | 1.00 | 118.88 | L | O | | |

*same color indicates a hydrogen-bonding pair.

| | | | | | | | | | | | | | | |
|---|---|---|---|---|---|---|---|---|---|---|---|---|---|---|
| CDR2 | | | | | | | | | | | | | Kaba | |
| ATOM | 85161 | N | ASP | 9 | 55 | −11.073 | −2.803 | 102.101 | 1.00 | 150.41 | 9 | N | 54 | F5126-VH |
| ATOM | 85162 | CA | ASP | 9 | 55 | −11.428 | −1.867 | 103.165 | 1.00 | 150.41 | 9 | C | 54 | |
| ATOM | 85163 | CB | ASP | 9 | 55 | −11.052 | −0.445 | 102.751 | 1.00 | 185.11 | 9 | C | 54 | |
| ATOM | 85164 | CG | ASP | 9 | 55 | −11.465 | 0.592 | 103.777 | 1.00 | 185.11 | 9 | C | 54 | |
| ATOM | 85165 | OD1 | ASP | 9 | 55 | −10.956 | 0.541 | 104.915 | 1.00 | 185.11 | 9 | O | 54 | Blue* |
| ATOM | 85166 | OD2 | ASP | 9 | 55 | −12.301 | 1.454 | 103.44 | 1.00 | 185.11 | 9 | O | 54 | |
| ATOM | 85167 | C | ASP | 9 | 55 | −12.913 | −1.921 | 103.515 | 1.00 | 150.41 | 9 | C | 54 | |
| ATOM | 85168 | O | ASP | 9 | 55 | −13.344 | −1.340 | 104.507 | 1.00 | 150.41 | 9 | O | 54 | |
| ATOM | 48969 | C1 | NAG | L | 542 | −12.823 | 1.703 | 109.786 | 1.00 | 154.7 | L | C | NAG | HA-a |
| ATOM | 48970 | C2 | NAG | L | 542 | −13.084 | 0.199 | 109.884 | 1.00 | 154.7 | L | C | | |
| ATOM | 48971 | N2 | NAG | L | 542 | −11.988 | −0.532 | 109.281 | 1.00 | 154.7 | L | N | | |
| ATOM | 48972 | C7 | NAG | L | 542 | −11.748 | −0.421 | 107.979 | 1.00 | 154.7 | L | C | | |
| ATOM | 48973 | O7 | NAG | L | 542 | −11.145 | 0.533 | 107.489 | 1.00 | 154.7 | L | O | | Blue* |
| ATOM | 48974 | C8 | NAG | L | 542 | −12.264 | −1.534 | 107.083 | 1.00 | 154.7 | L | C | | |
| ATOM | 48975 | C3 | NAG | L | 542 | −14.397 | −0.153 | 109.178 | 1.00 | 154.7 | L | C | | |
| ATOM | 48976 | O3 | NAG | L | 542 | −14.749 | −1.498 | 109.474 | 1.00 | 154.7 | L | O | | |
| ATOM | 48977 | C4 | NAG | L | 542 | −15.573 | 0.790 | 109.596 | 1.00 | 154.7 | L | C | | |
| ATOM | 48978 | O4 | NAG | L | 542 | −16.654 | 0.645 | 108.653 | 1.00 | 154.7 | L | O | | |
| ATOM | 48979 | C5 | NAG | L | 542 | −15.126 | 2.259 | 109.606 | 1.00 | 154.7 | L | C | | |
| ATOM | 48980 | O5 | NAG | L | 542 | −13.914 | 2.416 | 110.366 | 1.00 | 154.7 | L | O | | |
| ATOM | 48981 | C6 | NAG | L | 542 | −16.154 | 3.190 | 110.216 | 1.00 | 154.7 | L | C | | |
| ATOM | 48982 | O6 | NAG | L | 542 | −15.783 | 3.587 | 111.529 | 1.00 | 154.7 | L | O | | |
| ATOM | 49005 | C1 | MAN | L | 547 | −16.268 | −3.807 | 108.710 | 1.00 | 194.29 | L | C | MA | |
| ATOM | 49006 | C2 | MAN | L | 547 | −15.550 | −4.531 | 107.548 | 1.00 | 194.29 | L | C | | |
| ATOM | 49007 | O2 | MAN | L | 547 | −14.393 | −3.804 | 107.175 | 1.00 | 194.29 | L | O | | |
| ATOM | 49008 | C3 | MAN | L | 547 | −15.160 | −6.001 | 107.851 | 1.00 | 194.29 | L | C | | |
| ATOM | 49009 | O3 | MAN | L | 547 | −14.129 | −6.406 | 106.916 | 1.00 | 194.29 | L | O | | |
| ATOM | 49010 | C4 | MAN | L | 547 | −14.694 | −6.236 | 109.341 | 1.00 | 194.29 | L | C | | |
| ATOM | 49011 | O4 | MAN | L | 547 | −14.675 | −7.624 | 109.642 | 1.00 | 194.29 | L | O | | |
| ATOM | 49012 | C5 | MAN | L | 547 | −15.669 | −5.527 | 110.273 | 1.00 | 194.29 | L | C | | |
| ATOM | 49013 | O5 | MAN | L | 547 | −15.671 | −4.123 | 109.962 | 1.00 | 194.29 | L | O | | |
| ATOM | 49014 | C6 | MAN | L | 547 | −15.366 | −5.682 | 111.753 | 1.00 | 194.29 | L | C | | |
| ATOM | 49015 | O6 | MAN | L | 547 | −14.847 | −4.449 | 112.297 | 1.00 | 194.29 | L | O | | |
| | | | | | | | | | | | | | Kaba | |
| ATOM | 85169 | N | GLY | 9 | 56 | −13.689 | −2.615 | 102.690 | 1.00 | 140.03 | 9 | N | 55 | F5126-VH |
| ATOM | 85170 | CA | GLY | 9 | 56 | −15.116 | −2.743 | 102.932 | 1.00 | 140.03 | 9 | C | 55 | |
| ATOM | 85171 | C | GLY | 9 | 56 | −15.814 | −1.518 | 103.500 | 1.00 | 140.03 | 9 | C | 55 | |
| ATOM | 85172 | O | GLY | 9 | 56 | −16.633 | −1.636 | 104.411 | 1.00 | 140.03 | 9 | O | 55 | Green* |
| ATOM | 48983 | C1 | BMA | L | 543 | −17.816 | 0.047 | 109.111 | 1.00 | 177.38 | L | C | BM | HA-a |
| ATOM | 48984 | C2 | BMA | L | 543 | −18.974 | 0.394 | 108.171 | 1.00 | 177.38 | L | C | | |
| ATOM | 48985 | O2 | BMA | L | 543 | −18.643 | 0.016 | 106.840 | 1.00 | 1.00177.3 | L | O | | Green* |
| ATOM | 48986 | C3 | BMA | L | 543 | −20.250 | −0.336 | 108.620 | 1.00 | 177.38 | L | C | | |
| ATOM | 48987 | O3 | BMA | L | 543 | −21.333 | −0.043 | 107.703 | 1.00 | 177.38 | L | O | | |
| ATOM | 48988 | C4 | BMA | L | 543 | −19.959 | −1.880 | 108.711 | 1.00 | 177.38 | L | C | | |
| ATOM | 48989 | O4 | BMA | L | 543 | −21.101 | −2.537 | 109.247 | 1.00 | 177.38 | L | O | | |
| ATOM | 48990 | C5 | BMA | L | 543 | −18.753 | −2.096 | 109.637 | 1.00 | 177.38 | L | C | | |
| ATOM | 48991 | O5 | BMA | L | 543 | −17.605 | −1.371 | 109.133 | 1.00 | 177.38 | L | O | | |
| ATOM | 48992 | C6 | BMA | L | 543 | −18.350 | −3.553 | 109.827 | 1.00 | 177.38 | L | C | | |
| ATOM | 48993 | O6 | BMA | L | 543 | −17.645 | −4.068 | 108.673 | 1.00 | 177.38 | L | O | | |
| | | | | | | | | | | | | | Kaba | |
| ATOM | 85173 | N | GLN | 9 | 57 | −15.491 | −0.341 | 102.973 | 1.00 | 136.19 | 9 | N | 56 | F5126-VH |
| ATOM | 85174 | CA | GLN | 9 | 57 | −16.116 | 0.899 | 103.428 | 1.00 | 136.19 | 9 | C | 56 | |
| ATOM | 85175 | CB | GLN | 9 | 57 | −15.051 | 1.903 | 103.875 | 1.00 | 175.84 | 9 | C | 56 | |
| ATOM | 85176 | CG | GLN | 9 | 57 | −15.591 | 3.153 | 104.561 | 1.00 | 175.84 | 9 | C | 56 | |
| ATOM | 85177 | CD | GLN | 9 | 57 | −16.313 | 2.846 | 105.859 | 1.00 | 175.84 | 9 | C | 56 | |
| ATOM | 85178 | OE1 | GLN | 9 | 57 | −17.514 | 2.577 | 105.871 | 1.00 | 175.84 | 9 | O | 56 | |
| ATOM | 85179 | NE2 | GLN | 9 | 57 | −15.576 | 2.875 | 106.961 | 1.00 | 175.84 | 9 | N | 56 | |
| ATOM | 85180 | C | GLN | 9 | 57 | −16.91 | 1.468 | 102.259 | 1.00 | 136.19 | 9 | C | 56 | |
| ATOM | 85181 | O | GLN | 9 | 57 | −16.535 | 1.281 | 101.102 | 1.00 | 136.19 | 9 | O | 56 | |
| | | | | | | | | | 1.00 | | | | | |
| ATOM | 48983 | C1 | BMA | L | 543 | −17.816 | 0.047 | 109.111 | 1.00 | 177.38 | L | C | BM | HA-a |
| ATOM | 48984 | C2 | BMA | L | 543 | −18.974 | 0.394 | 108.171 | 1.00 | 177.38 | L | C | | |
| ATOM | 48985 | O2 | BMA | L | 543 | −18.643 | 0.016 | 106.84 | 1.00 | 1773.8 | L | O | | |
| ATOM | 48986 | C3 | BMA | L | 543 | −20.25 | −0.336 | 108.62 | 1.00 | 177.38 | L | C | | |
| ATOM | 48987 | O3 | BMA | L | 543 | −21.333 | −0.043 | 107.703 | 1.00 | 177.38 | L | O | | |
| ATOM | 48988 | C4 | BMA | L | 543 | −19.959 | −1.880 | 108.711 | 1.00 | 177.38 | L | C | | |
| ATOM | 48989 | O4 | BMA | L | 543 | −21.101 | −2.537 | 109.247 | 1.00 | 177.38 | L | O | | |
| ATOM | 48990 | C5 | BMA | L | 543 | −18.753 | −2.096 | 109.637 | 1.00 | 177.38 | L | C | | |
| ATOM | 48991 | O5 | BMA | L | 543 | −17.605 | −1.371 | 109.133 | 1.00 | 177.38 | L | O | | |
| ATOM | 48992 | C6 | BMA | L | 543 | −18.35 | −3.553 | 109.827 | 1.00 | 177.38 | L | C | | |
| ATOM | 48993 | O6 | BMA | L | 543 | −17.645 | −4.068 | 108.673 | 1.00 | 177.38 | L | O | | |
| ATOM | 48969 | C1 | NAG | L | 542 | −12.823 | 1.703 | 109.786 | 1.00 | 154.7 | L | C | NAG | |
| ATOM | 48970 | C2 | NAG | L | 542 | −13.084 | 0.199 | 109.884 | 1.00 | 154.7 | L | C | | |
| ATOM | 48971 | N2 | NAG | L | 542 | −11.988 | −0.532 | 109.281 | 1.00 | 154.7 | L | N | | |
| ATOM | 48972 | C7 | NAG | L | 542 | −11.748 | −0.421 | 107.979 | 1.00 | 154.7 | L | C | | |
| ATOM | 48973 | O7 | NAG | L | 542 | −11.145 | 0.533 | 107.489 | 1.00 | 154.7 | L | O | | |
| ATOM | 48974 | C8 | NAG | L | 542 | −12.264 | −1.534 | 107.083 | 1.00 | 154.7 | L | C | | |
| ATOM | 48975 | C3 | NAG | L | 542 | −14.397 | −0.153 | 109.178 | 1.00 | 154.7 | L | C | | |
| ATOM | 48976 | O3 | NAG | L | 542 | −14.749 | −1.498 | 109.474 | 1.00 | 154.7 | L | O | | |
| ATOM | 48977 | C4 | NAG | L | 542 | −15.573 | 0.790 | 109.596 | 1.00 | 154.7 | L | C | | |
| ATOM | 48978 | O4 | NAG | L | 542 | −16.654 | 0.645 | 108.653 | 1.00 | 154.7 | L | O | | Orange* |
| ATOM | 48979 | C5 | NAG | L | 542 | −15.126 | 2.259 | 109.606 | 1.00 | 154.7 | L | C | | |
| ATOM | 48980 | O5 | NAG | L | 542 | −13.914 | 2.416 | 110.366 | 1.00 | 154.7 | L | O | | |
| ATOM | 48981 | C6 | NAG | L | 542 | −16.154 | 3.190 | 110.216 | 1.00 | 154.7 | L | C | | |
| ATOM | 48982 | O6 | NAG | L | 542 | −15.783 | 3.587 | 111.529 | 1.00 | 154.7 | L | O | | |
| | | | | | | | | | | | | | Kaba | |
| ATOM | 85173 | N | GLN | 9 | 57 | −15.491 | −0.341 | 102.973 | 1.00 | 136.19 | 9 | N | 56 | F5126-VH |
| ATOM | 85174 | CA | GLN | 9 | 57 | −16.116 | 0.899 | 103.428 | 1.00 | 136.19 | 9 | C | 56 | |
| ATOM | 85175 | CB | GLN | 9 | 57 | −15.051 | 1.903 | 103.875 | 1.00 | 175.84 | 9 | C | 56 | |
| ATOM | 85176 | CG | GLN | 9 | 57 | −15.591 | 3.153 | 104.561 | 1.00 | 175.84 | 9 | C | 56 | |
| ATOM | 85177 | CD | GLN | 9 | 57 | −16.313 | 2.846 | 105.859 | 1.00 | 175.84 | 9 | C | 56 | |
| ATOM | 85178 | OE1 | GLN | 9 | 57 | −17.514 | 2.577 | 105.871 | 1.00 | 175.84 | 9 | O | 56 | |
| ATOM | 85179 | NE2 | GLN | 9 | 57 | −15.576 | 2.875 | 106.961 | 1.00 | 175.84 | 9 | N | 56 | Purple* |
| ATOM | 85180 | C | GLN | 9 | 57 | −16.91 | 1.468 | 102.259 | 1.00 | 136.19 | 9 | C | 56 | |
| ATOM | 85181 | O | GLN | 9 | 57 | −16.535 | 1.281 | 101.102 | 1.00 | 136.19 | 9 | O | 56 | |
| ATOM | 48969 | C1 | NAG | L | 542 | −12.823 | 1.703 | 109.786 | 1.00 | 154.7 | L | C | NAG | HA-a |
| ATOM | 48970 | C2 | NAG | L | 542 | −13.084 | 0.199 | 109.884 | 1.00 | 154.7 | L | C | | |
| ATOM | 48971 | N2 | NAG | L | 542 | −11.988 | −0.532 | 109.281 | 1.00 | 154.7 | L | N | | |
| ATOM | 48972 | C7 | NAG | L | 542 | −11.748 | −0.421 | 107.979 | 1.00 | 154.7 | L | C | | |
| ATOM | 48973 | O7 | NAG | L | 542 | −11.145 | 0.533 | 107.489 | 1.00 | 154.7 | L | O | | |
| ATOM | 48974 | C8 | NAG | L | 542 | −12.264 | −1.534 | 107.083 | 1.00 | 154.7 | L | C | | |
| ATOM | 48975 | C3 | NAG | L | 542 | −14.397 | −0.153 | 109.178 | 1.00 | 154.7 | L | C | | |
| ATOM | 48976 | O3 | NAG | L | 542 | −14.749 | −1.498 | 109.474 | 1.00 | 154.7 | L | O | | |
| ATOM | 48977 | C4 | NAG | L | 542 | −15.573 | 0.790 | 109.596 | 1.00 | 154.7 | L | C | | |
| ATOM | 48978 | O4 | NAG | L | 542 | −16.654 | 0.645 | 108.653 | 1.00 | 154.7 | L | O | | Purple* |
| ATOM | 48979 | C5 | NAG | L | 542 | −15.126 | 2.259 | 109.606 | 1.00 | 154.7 | L | C | | |
| ATOM | 48980 | O5 | NAG | L | 542 | −13.914 | 2.416 | 110.366 | 1.00 | 154.7 | L | O | | |
| ATOM | 48981 | C6 | NAG | L | 542 | −16.154 | 3.190 | 110.216 | 1.00 | 154.7 | L | C | | |
| ATOM | 48982 | O6 | NAG | L | 542 | −15.783 | 3.587 | 111.529 | 1.00 | 154.7 | L | O | | |
| | | | | | | | | | | | | | Kaba | |
| ATOM | 85182 | N | THR | 9 | 58 | −18.004 | 2.162 | 102.557 | 1.00 | 128.31 | 9 | N | 57 | F5126-VH |
| ATOM | 85183 | CA | THR | 9 | 58 | −18.842 | 2.725 | 101.505 | 1.00 | 128.31 | 9 | C | 57 | |
| ATOM | 85184 | CB | THR | 9 | 58 | −19.965 | 1.743 | 101.121 | 1.00 | 125.36 | 9 | C | 57 | |
| ATOM | 85185 | OG1 | THR | 9 | 58 | −20.698 | 1.375 | 102.296 | 1.00 | 125.36 | 9 | O | 57 | Pink* |
| ATOM | 85186 | CG2 | THR | 9 | 58 | −19.386 | 0.493 | 100.478 | 1.00 | 125.36 | 9 | C | 57 | |
| ATOM | 85187 | C | THR | 9 | 58 | −19.482 | 4.062 | 101.862 | 1.00 | 128.31 | 9 | C | 57 | |
| ATOM | 85188 | O | THR | 9 | 58 | −19.995 | 4.244 | 102.966 | 1.00 | 128.31 | 9 | O | 57 | |
| ATOM | 49091 | C1 | MAN | L | 534 | 44.907 | −20.118 | 116.721 | 1.00 | 223.79 | L | C | MA | HA-a |
| ATOM | 49092 | C2 | MAN | L | 534 | 44.312 | −19.386 | 117.954 | 1.00 | 223.79 | L | C | | |
| ATOM | 49093 | O2 | MAN | L | 534 | 45.318 | −19.185 | 118.969 | 1.00 | 223.79 | L | O | | |
| ATOM | 49094 | C3 | MAN | L | 534 | 43.599 | −18.048 | 117.624 | 1.00 | 223.79 | L | C | | |
| ATOM | 49095 | O3 | MAN | L | 534 | 43.491 | −17.26 | 118.802 | 1.00 | 223.79 | L | O | | |
| ATOM | 49096 | C4 | MAN | L | 534 | 44.321 | −17.233 | 116.525 | 1.00 | 223.79 | L | C | | |
| ATOM | 49097 | O4 | MAN | L | 534 | 43.502 | −16.153 | 116.100 | 1.00 | 223.79 | L | O | | |
| ATOM | 49098 | C5 | MAN | L | 534 | 44.599 | −18.159 | 115.352 | 1.00 | 223.79 | L | C | | |
| ATOM | 49099 | O5 | MAN | L | 534 | 45.485 | −19.203 | 115.788 | 1.00 | 223.79 | L | O | | |
| ATOM | 49100 | C6 | MAN | L | 534 | 45.236 | −17.483 | 114.152 | 1.00 | 223.79 | L | C | | |
| ATOM | 49101 | O6 | MAN | L | 534 | 46.354 | −16.695 | 114.530 | 1.00 | 223.79 | L | O | | Pink* |

*same color indicates a hydrogen-bonding pair: for orange, two hydrogen bonding pairs are: Gln57 OE1 to BMA O2, and Gln57 C

| | | | | | | | | | | | | | | |
|---|---|---|---|---|---|---|---|---|---|---|---|---|---|---|
| FR3 | | | | | | | | | | | | | Kaba | |
| ATOM | 85323 | N | GLY | 9 | 75 | −11.940 | −11.373 | 94.122 | 1.00 | 149.41 | 9 | N | 74 | F5126-VH |
| ATOM | 85324 | CA | GLY | 9 | 75 | −11.459 | −12.554 | 93.433 | 1.00 | 149.41 | 9 | C | 74 | |
| ATOM | 85325 | C | GLY | 9 | 75 | −11.261 | −12.421 | 91.934 | 1.00 | 149.41 | 9 | C | 74 | |
| ATOM | 85326 | O | GLY | 9 | 75 | −10.344 | −13.024 | 91.379 | 1.00 | 149.41 | 9 | O | 74 | Blue* |
| | | | | | | | | | 1.00 | | | | | |
| ATOM | 38674 | N | GLY | J | 240 | −9.394 | −15.103 | 94.368 | 1.00 | 147.24 | J | N | | HA-b, Blue |
| ATOM | 38675 | CA | GLY | J | 240 | −8.610 | −14.772 | 93.192 | 1.00 | 147.24 | J | C | | |
| ATOM | 38676 | C | GLY | J | 240 | −7.333 | −15.582 | 93.096 | 1.00 | 147.24 | J | C | | |
| ATOM | 38677 | O | GLY | J | 240 | −6.894 | −15.934 | 92.001 | 1.00 | 147.24 | J | O | | |

*same color indicates a hydrogen-bonding pair.

| | | | | | | | | | | | | | | |
|---|---|---|---|---|---|---|---|---|---|---|---|---|---|---|
| CDR3 | | | | | | | | | | | | | Kaba | |
| ATOM | 85532 | N | VAL | 9 | 102 | −7.031 | 4.781 | 100.707 | 1.00 | 159.63 | 9 | N | 98 | F5126-VH |
| ATOM | 85533 | CA | VAL | 9 | 102 | −7.100 | 3.364 | 101.039 | 1.00 | 159.63 | 9 | C | 98 | |
| ATOM | 85534 | CB | VAL | 9 | 102 | −6.340 | 2.505 | 99.995 | 1.00 | 131.25 | 9 | C | 98 | |
| ATOM | 85535 | CG1 | VAL | 9 | 102 | −6.531 | 1.024 | 100.291 | 1.00 | 131.25 | 9 | C | 98 | |
| ATOM | 85536 | CG2 | VAL | 9 | 102 | −6.834 | 2.828 | 98.595 | 1.00 | 131.25 | 9 | C | 98 | |
| ATOM | 85537 | C | VAL | 9 | 102 | −6.465 | 3.159 | 102.409 | 1.00 | 159.63 | 9 | C | 98 | |
| ATOM | 85538 | O | VAL | 9 | 102 | −5.806 | 4.055 | 102.934 | 1.00 | 159.63 | 9 | O | 98 | Blue* |
| | | | | | | | | | 1.00 | | | | | |
| ATOM | 47105 | N | ASP | L | 271 | −1.987 | 4.738 | 104.257 | 1.00 | 117.63 | L | N | | HA-a |
| ATOM | 47106 | CA | ASP | L | 271 | −1.916 | 5.400 | 102.955 | 1.00 | 117.63 | L | C | | |
| ATOM | 47107 | CB | ASP | L | 271 | −3.008 | 4.864 | 102.026 | 1.00 | 124.59 | L | C | | |
| ATOM | 47108 | CG | ASP | L | 271 | −2.673 | 3.506 | 101.448 | 1.00 | 124.59 | L | C | | |
| ATOM | 47109 | OD1 | ASP | L | 271 | −1.839 | 3.443 | 100.522 | 1.00 | 124.59 | L | O | | |
| ATOM | 47110 | OD2 | ASP | L | 271 | −3.244 | 2.504 | 101.921 | 1.00 | 124.59 | L | O | | Blue* |
| ATOM | 47111 | C | ASP | L | 271 | −2.108 | 6.900 | 103.135 | 1.00 | 117.63 | L | C | | |
| ATOM | 47112 | O | ASP | L | 271 | −2.192 | 7.646 | 102.161 | 1.00 | 117.63 | L | O | | |
| | | | | | | | | | 1.00 | | | | | |
| | | | | | | | | | 1.00 | | | | Kaba | |
| ATOM | 85539 | N | ARG | 9 | 103 | −6.674 | 1.982 | 102.987 | 1.00 | 149.12 | 9 | N | 99 | F5126-VH |
| ATOM | 85540 | CA | ARG | 9 | 103 | −6.114 | 1.661 | 104.293 | 1.00 | 149.12 | 9 | C | 99 | |
| ATOM | 85541 | CB | ARG | 9 | 103 | −4.586 | 1.563 | 104.200 | 1.00 | 133.42 | 9 | C | 99 | |
| ATOM | 85542 | CG | ARG | 9 | 103 | −3.985 | 0.382 | 104.946 | 1.00 | 133.42 | 9 | C | 99 | |
| ATOM | 85543 | CD | ARG | 9 | 103 | −4.366 | −0.922 | 104.269 | 1.00 | 133.42 | 9 | C | 99 | |
| ATOM | 85544 | NE | ARG | 9 | 103 | −4.053 | −2.089 | 105.086 | 1.00 | 133.42 | 9 | N | 99 | |
| ATOM | 85545 | CZ | ARG | 9 | 103 | −4.660 | −3.264 | 104.956 | 1.00 | 133.42 | 9 | C | 99 | |
| ATOM | 85546 | NH1 | ARG | 9 | 103 | −5.609 | −3.419 | 104.043 | 1.00 | 133.42 | 9 | N | 99 | |
| ATOM | 85547 | NH2 | ARG | 9 | 103 | −4.329 | −4.279 | 105.742 | 1.00 | 133.42 | 9 | N | 99 | |
| ATOM | 85548 | C | ARG | 9 | 103 | −6.499 | 2.718 | 105.329 | 1.00 | 149.12 | 9 | C | 99 | |
| ATOM | 85549 | O | ARG | 9 | 103 | −7.652 | 3.141 | 105.404 | 1.00 | 149.12 | 9 | O | 99 | |
| ATOM | 47105 | N | ASP | L | 271 | −1.987 | 4.738 | 104.257 | 1.00 | 117.63 | L | N | | HA-a |
| ATOM | 47106 | CA | ASP | L | 271 | −1.916 | 5.400 | 102.955 | 1.00 | 117.63 | L | C | | |
| ATOM | 47107 | CB | ASP | L | 271 | −3.008 | 4.864 | 102.026 | 1.00 | 124.59 | L | C | | |
| ATOM | 47108 | CG | ASP | L | 271 | −2.673 | 3.506 | 101.448 | 1.00 | 124.59 | L | C | | |
| ATOM | 47109 | OD1 | ASP | L | 271 | −1.839 | 3.443 | 100.522 | 1.00 | 124.59 | L | O | | |
| ATOM | 47110 | OD2 | ASP | L | 271 | −3.244 | 2.504 | 101.921 | 1.00 | 124.59 | L | O | | |
| ATOM | 47111 | C | ASP | L | 271 | −2.108 | 6.900 | 103.135 | 1.00 | 117.63 | L | C | | |
| ATOM | 47112 | O | ASP | L | 271 | −2.192 | 7.646 | 102.161 | 1.00 | 117.63 | L | O | | |
| ATOM | 48955 | C1 | NAG | L | 541 | −9.146 | 5.332 | 110.891 | 1.00 | 131.56 | L | C | NAG | |
| ATOM | 48956 | C2 | NAG | L | 541 | −10.638 | 5.656 | 110.952 | 1.00 | 131.56 | L | C | | |
| ATOM | 48957 | N2 | NAG | L | 541 | −10.891 | 6.628 | 111.996 | 1.00 | 131.56 | L | N | | |
| ATOM | 48958 | C7 | NAG | L | 541 | −11.904 | 7.480 | 111.879 | 1.00 | 131.56 | L | C | | |
| ATOM | 48959 | O7 | NAG | L | 541 | −11.881 | 8.446 | 111.117 | 1.00 | 131.56 | L | O | | |
| ATOM | 48960 | C8 | NAG | L | 541 | −13.132 | 7.222 | 112.737 | 1.00 | 131.56 | L | C | | |
| ATOM | 48961 | C3 | NAG | L | 541 | −11.416 | 4.372 | 111.221 | 1.00 | 131.56 | L | C | | |
| ATOM | 48962 | O3 | NAG | L | 541 | −12.808 | 4.648 | 111.216 | 1.00 | 131.56 | L | O | | |
| ATOM | 48963 | C4 | NAG | L | 541 | −11.063 | 3.295 | 110.142 | 1.00 | 131.56 | L | C | | |
| ATOM | 48964 | O4 | NAG | L | 541 | −11.673 | 2.032 | 110.483 | 1.00 | 131.56 | L | O | | |
| ATOM | 48965 | C5 | NAG | L | 541 | −9.537 | 3.122 | 110.075 | 1.00 | 131.56 | L | C | | |
| ATOM | 48966 | O5 | NAG | L | 541 | −8.897 | 4.397 | 109.838 | 1.00 | 131.56 | L | O | | |
| ATOM | 48967 | C6 | NAG | L | 541 | −9.095 | 2.192 | 108.966 | 1.00 | 131.56 | L | C | | |
| ATOM | 48967 | O6 | NAG | L | 541 | −8.913 | 2.892 | 107.743 | 1.00 | 131.56 | L | O | Oran | |
| | | | | | | | | | | | | | Kaba | |
| ATOM | 85550 | N | GLY | 9 | 104 | −5.513 | 3.145 | 106.113 | 1.00 | 132.74 | 9 | N | 100 | F5126-VH |
| ATOM | 85551 | CA | GLY | 9 | 104 | −5.735 | 4.131 | 107.156 | 1.00 | 132.74 | 9 | C | 100 | |
| ATOM | 85552 | C | GLY | 9 | 104 | −6.503 | 5.393 | 106.802 | 1.00 | 132.74 | 9 | C | 100 | |
| ATOM | 85553 | O | GLY | 9 | 104 | −7.283 | 5.885 | 107.618 | 1.00 | 132.74 | 9 | O | 100 | Pink* |
| ATOM | 48955 | C1 | NAG | L | 541 | −9.146 | 5.332 | 110.891 | 1.00 | 131.56 | L | C | NAG | HA-a |
| ATOM | 48956 | C2 | NAG | L | 541 | −10.638 | 5.656 | 110.952 | 1.00 | 131.56 | L | C | | |
| ATOM | 48957 | N2 | NAG | L | 541 | −10.891 | 6.628 | 111.996 | 1.00 | 131.56 | L | N | | |
| ATOM | 48958 | C7 | NAG | L | 541 | −11.904 | 7.480 | 111.879 | 1.00 | 131.56 | L | C | | |
| ATOM | 48959 | O7 | NAG | L | 541 | −11.881 | 8.446 | 111.117 | 1.00 | 131.56 | L | O | | |
| ATOM | 48960 | C8 | NAG | L | 541 | −13.132 | 7.222 | 112.737 | 1.00 | 131.56 | L | C | | |
| ATOM | 48961 | C3 | NAG | L | 541 | −11.416 | 4.372 | 111.221 | 1.00 | 131.56 | L | C | | |
| ATOM | 48962 | O3 | NAG | L | 541 | −12.808 | 4.648 | 111.216 | 1.00 | 131.56 | L | O | | |
| ATOM | 48963 | C4 | NAG | L | 541 | −11.063 | 3.295 | 110.142 | 1.00 | 131.56 | L | C | | |
| ATOM | 48964 | O4 | NAG | L | 541 | −11.673 | 2.032 | 110.483 | 1.00 | 131.56 | L | O | | |
| ATOM | 48965 | C5 | NAG | L | 541 | −9.537 | 3.122 | 110.075 | 1.00 | 131.56 | L | C | | |
| ATOM | 48966 | O5 | NAG | L | 541 | −8.897 | 4.397 | 109.838 | 1.00 | 131.56 | L | O | | Pink* |
| ATOM | 48967 | C6 | NAG | L | 541 | −9.095 | 2.192 | 108.966 | 1.00 | 131.56 | L | C | | |
| ATOM | 48968 | O6 | NAG | L | 541 | −8.913 | 2.892 | 107.743 | 1.00 | 131.56 | L | O | | |

*same color indicates a hydrogen-bonding pair.
The effective number of decimal place is first decimal place, in notation of the atomic coordinate.

TABLE 2B

| CDR1 | | | | | | | | | | | | | Kabat | |
|---|---|---|---|---|---|---|---|---|---|---|---|---|---|---|
| ATOM | 84988 | N | SER | 9 | 31 | −5.533 | −2.941 | 96.077 | 1.00 | 138.89 | 9 | N | 31 | F5126-VH |
| ATOM | 84989 | CA | SER | 9 | 31 | −4.653 | −1.973 | 96.725 | 1.00 | 138.89 | 9 | C | 31 | |
| ATOM | 84990 | CB | SER | 9 | 31 | −3.185 | −2.306 | 96.440 | 1.00 | 133.94 | 9 | C | 31 | |
| ATOM | 84991 | OG | SER | 9 | 31 | −2.922 | −2.341 | 95.049 | 1.00 | 133.94 | 9 | O | 31 | Yellow* |
| ATOM | 84992 | C | SER | 9 | 31 | −4.968 | −0.553 | 96.259 | 1.00 | 138.89 | 9 | C | 31 | |
| ATOM | 84993 | O | SER | 9 | 31 | −4.16 | 0.361 | 96.422 | 1.00 | 138.89 | 9 | O | 31 | Blue* |
| ATOM | 45725 | N | SER | L | 91 | −0.285 | 0.917 | 100.73 | 1.00 | 124.22 | L | N | | HA-R |
| ATOM | 45726 | CA | SER | L | 91 | −1.037 | −0.241 | 100.264 | 1.00 | 124.22 | L | C | | |
| ATOM | 45727 | CB | SER | L | 91 | −2.529 | −0.050 | 100.54 | 1.00 | 121.35 | L | C | | |
| ATOM | 45728 | OG | SER | L | 91 | −3.036 | 1.072 | 99.839 | 1.00 | 121.35 | L | O | | Blue* |
| ATOM | 45729 | C | SER | L | 91 | −0.813 | −0.431 | 98.769 | 1.00 | 124.22 | L | C | | |
| ATOM | 45730 | O | SER | L | 91 | −0.811 | −1.554 | 98.266 | 1.00 | 124.22 | L | O | | Yellow* |
| ATOM | 45731 | N | LYS | L | 92 | −0.625 | 0.681 | 98.065 | 1.00 | 119.46 | L | N | | |
| ATOM | 45732 | CA | LYS | L | 92 | −0.39 | 0.658 | 96.626 | 1.00 | 119.46 | L | C | | |
| ATOM | 45733 | CB | LYS | L | 92 | −0.244 | 2.088 | 96.103 | 1.00 | 115.22 | L | C | | |
| ATOM | 45734 | CG | LYS | L | 92 | 1.070 | 2.744 | 96.507 | 1.00 | 115.22 | L | C | | |
| ATOM | 45735 | CD | LYS | L | 92 | 1.106 | 4.227 | 96.180 | 1.00 | 115.22 | L | C | | |
| ATOM | 45736 | CE | LYS | L | 92 | 0.308 | 5.037 | 97.186 | 1.00 | 115.22 | L | C | | |
| ATOM | 45737 | NZ | LYS | L | 92 | 0.443 | 6.500 | 96.940 | 1.00 | 115.22 | L | N | | |
| ATOM | 45738 | C | LYS | L | 92 | 0.892 | −0.120 | 96.346 | 1.00 | 119.46 | L | C | | |
| ATOM | 45739 | O | LYS | L | 92 | 1.209 | −0.430 | 95.197 | 1.00 | 119.46 | L | O | | |

*same color indicates a hydrogen-bonding pair.

| | | | | | | | | | | | | | | |
|---|---|---|---|---|---|---|---|---|---|---|---|---|---|---|
| CDR2 | | | | | | | | | | | | | Kabat | |
| ATOM | 85185 | N | ASP | 9 | 55 | −11.077 | −2.804 | 102.089 | 1.00 | 150.24 | 9 | N | 54 | F5126-VH |
| ATOM | 85186 | CA | ASP | 9 | 55 | −11.432 | −1.869 | 103.152 | 1.00 | 150.24 | 9 | C | 54 | |
| ATOM | 85187 | CB | ASP | 9 | 55 | −11.057 | −0.446 | 102.739 | 1.00 | 185.10 | 9 | C | 54 | |
| ATOM | 85188 | CG | ASP | 9 | 55 | −11.47 | 0.590 | 103.765 | 1.00 | 185.10 | 9 | C | 54 | |
| ATOM | 85189 | OD1 | ASP | 9 | 55 | −10.962 | 0.539 | 104.903 | 1.00 | 185.10 | 9 | O | 54 | Blue* |
| ATOM | 85190 | OD2 | ASP | 9 | 55 | −12.307 | 1.453 | 103.428 | 1.00 | 185.10 | 9 | O | 54 | |
| ATOM | 85191 | C | ASP | 9 | 55 | −12.916 | −1.924 | 103.503 | 1.00 | 150.24 | 9 | C | 54 | |
| ATOM | 85192 | O | ASP | 9 | 55 | −13.348 | −1.344 | 104.497 | 1.00 | 150.24 | 9 | O | 54 | Yellow* |
| ATOM | 48993 | C1 | NAG | L | 542 | −12.824 | 1.705 | 109.787 | 1.00 | 155.29 | L | C | NAG-2 | HA-R |
| ATOM | 48994 | C2 | NAG | L | 542 | −13.085 | 0.202 | 109.887 | 1.00 | 155.29 | L | C | | |
| ATOM | 48995 | N2 | NAG | L | 542 | −11.988 | −0.530 | 109.283 | 1.00 | 155.29 | L | N | | |
| ATOM | 48996 | C7 | NAG | L | 542 | −11.747 | −0.418 | 107.981 | 1.00 | 155.29 | L | C | | |
| ATOM | 48997 | O7 | NAG | L | 542 | −11.141 | 0.535 | 107.494 | 1.00 | 155.29 | L | O | | Blue* |
| ATOM | 48998 | C8 | NAG | L | 542 | −12.266 | −1.528 | 107.083 | 1.00 | 155.29 | L | C | | |
| ATOM | 48999 | C3 | NAG | L | 542 | −14.397 | −0.151 | 109.181 | 1.00 | 155.29 | L | C | | |
| ATOM | 49000 | O3 | NAG | L | 542 | −14.749 | −1.496 | 109.478 | 1.00 | 155.29 | L | O | | |
| ATOM | 49001 | C4 | NAG | L | 542 | −15.573 | 0.792 | 109.598 | 1.00 | 155.29 | L | C | | |
| ATOM | 49002 | O4 | NAG | L | 542 | −16.655 | 0.646 | 108.656 | 1.00 | 155.29 | L | O | | |
| ATOM | 49003 | C5 | NAG | L | 542 | −15.127 | 2.262 | 109.606 | 1.00 | 155.29 | L | C | | |
| ATOM | 49004 | O5 | NAG | L | 542 | −13.915 | 2.420 | 110.366 | 1.00 | 155.29 | L | O | | |
| ATOM | 49005 | C6 | NAG | L | 542 | −16.155 | 3.192 | 110.216 | 1.00 | 155.29 | L | C | | |
| ATOM | 49006 | O6 | NAG | L | 542 | −15.785 | 3.589 | 111.530 | 1.00 | 155.29 | L | O | | |
| ATOM | 49029 | C1 | MAN | L | 547 | −16.267 | −3.806 | 108.716 | 1.00 | 194.75 | L | C | MAN-2 | |
| ATOM | 49030 | C2 | MAN | L | 547 | −15.549 | −4.529 | 107.555 | 1.00 | 194.75 | L | C | | |
| ATOM | 49031 | O2 | MAN | L | 547 | −14.392 | −3.802 | 107.181 | 1.00 | 194.75 | L | O | | Yellow* |
| ATOM | 49032 | C3 | MAN | L | 547 | −15.158 | −5.999 | 107.857 | 1.00 | 194.75 | L | C | | |
| ATOM | 49033 | O3 | MAN | L | 547 | −14.128 | −6.404 | 106.922 | 1.00 | 194.75 | L | O | | |
| ATOM | 49034 | C4 | MAN | L | 547 | −14.693 | −6.234 | 109.347 | 1.00 | 194.75 | L | C | | |
| ATOM | 49035 | O4 | MAN | L | 547 | −14.672 | −7.622 | 109.648 | 1.00 | 194.75 | L | O | | |
| ATOM | 49036 | C5 | MAN | L | 547 | −15.669 | −5.526 | 110.278 | 1.00 | 194.75 | L | C | | |
| ATOM | 49037 | O5 | MAN | L | 547 | −15.672 | −4.122 | 109.969 | 1.00 | 194.75 | L | O | | |
| ATOM | 49038 | C6 | MAN | L | 547 | −15.367 | −5.681 | 111.759 | 1.00 | 194.75 | L | C | | |
| ATOM | 49039 | O6 | MAN | L | 547 | −14.85 | −4.448 | 112.304 | 1.00 | 194.75 | L | O | | |
| | | | | | | | | | | | | | Kabat | |
| ATOM | 85193 | N | GLY | 9 | 56 | −13.693 | −2.617 | 102.678 | 1.00 | 140.24 | 9 | N | 55 | F5126-VH |
| ATOM | 85194 | CA | GLY | 9 | 56 | −15.120 | −2.745 | 102.920 | 1.00 | 140.24 | 9 | C | 55 | |
| ATOM | 85195 | C | GLY | 9 | 56 | −15.818 | −1.5210 | 103.489 | 1.00 | 140.24 | 9 | C | 55 | |
| ATOM | 85196 | O | GLY | 9 | 56 | −16.635 | −1.64 | 104.402 | 1.00 | 140.24 | 9 | O | 55 | Green* |
| ATOM | 49007 | C1 | BMA | L | 543 | −17.816 | 0.048 | 109.115 | 1.00 | 177.65 | L | C | BMA | HA-R |
| ATOM | 49008 | C2 | BMA | L | 543 | −18.975 | 0.396 | 108.176 | 1.00 | 177.65 | L | C | | |
| ATOM | 49009 | O2 | BMA | L | 543 | −18.644 | 0.018 | 106.844 | 1.00 | 177.65 | L | O | | Green* |
| ATOM | 49010 | C3 | BMA | L | 543 | −20.250 | −0.335 | 108.624 | 1.00 | 177.65 | L | C | | |
| ATOM | 49011 | O3 | BMA | L | 543 | −21.334 | −0.042 | 107.709 | 1.00 | 177.65 | L | O | | |
| ATOM | 49012 | C4 | BMA | L | 543 | −19.960 | −1.879 | 108.716 | 1.00 | 177.65 | L | C | | |
| ATOM | 49013 | O4 | BMA | L | 543 | −21.101 | −2.536 | 109.251 | 1.00 | 177.65 | L | O | | |
| ATOM | 49014 | C5 | BMA | L | 543 | −18.753 | −2.094 | 109.641 | 1.00 | 177.65 | L | C | | |
| ATOM | 49015 | O5 | BMA | L | 543 | −17.606 | −1.37 | 109.137 | 1.00 | 177.65 | L | O | | |
| ATOM | 49016 | C6 | BMA | L | 543 | −18.350 | −3.551 | 109.832 | 1.00 | 177.65 | L | C | | |
| ATOM | 49017 | O6 | BMA | L | 543 | −17.645 | −4.067 | 108.678 | 1.00 | 177.65 | L | O | | |
| | | | | | | | | | | | | | Kabat | |
| ATOM | 85197 | N | GLN | 9 | 57 | −15.497 | −0.344 | 102.961 | 1.00 | 136.36 | 9 | N | 56 | F5126-VH |
| ATOM | 85198 | CA | GLN | 9 | 57 | −16.123 | 0.896 | 103.416 | 1.00 | 136.36 | 9 | C | 56 | |
| ATOM | 85199 | CB | GLN | 9 | 57 | −15.058 | 1.900 | 103.864 | 1.00 | 176.32 | 9 | C | 56 | |
| ATOM | 85200 | CG | GLN | 9 | 57 | −15.599 | 3.149 | 104.549 | 1.00 | 176.32 | 9 | C | 56 | |
| ATOM | 85201 | CD | GLN | 9 | 57 | −16.324 | 2.844 | 105.846 | 1.00 | 176.32 | 9 | C | 56 | |
| ATOM | 85202 | OE1 | GLN | 9 | 57 | −17.526 | 2.577 | 105.855 | 1.00 | 176.32 | 9 | O | 56 | |
| ATOM | 85203 | NE2 | GLN | 9 | 57 | −15.589 | 2.868 | 106.949 | 1.00 | 176.32 | 9 | N | 56 | |
| ATOM | 85204 | C | GLN | 9 | 57 | −16.917 | 1.465 | 102.247 | 1.00 | 136.36 | 9 | C | 56 | |
| ATOM | 85205 | O | GLN | 9 | 57 | −16.540 | 1.281 | 101.090 | 1.00 | 136.36 | 9 | O | 56 | |
| ATOM | 49007 | C1 | BMA | L | 543 | −17.816 | 0.048 | 109.115 | 1.00 | 177.65 | L | C | BMA | HA-R |
| ATOM | 49008 | C2 | BMA | L | 543 | −18.975 | 0.396 | 108.176 | 1.00 | 177.65 | L | C | | |
| ATOM | 49009 | O2 | BMA | L | 543 | −18.644 | 0.018 | 106.844 | 1.00 | 177.65 | L | O | | Orange* |
| ATOM | 49010 | C3 | BMA | L | 543 | −20.250 | −0.335 | 108.624 | 1.00 | 177.65 | L | C | | |
| ATOM | 49011 | O3 | BMA | L | 543 | −21.334 | −0.042 | 107.709 | 1.00 | 177.65 | L | O | | |
| ATOM | 49012 | C4 | BMA | L | 543 | −19.960 | −1.879 | 108.716 | 1.00 | 177.65 | L | C | | |
| ATOM | 49013 | O4 | BMA | L | 543 | −21.101 | −2.536 | 109.251 | 1.00 | 177.65 | L | O | | |
| ATOM | 49014 | C5 | BMA | L | 543 | −18.753 | −2.094 | 109.641 | 1.00 | 177.65 | L | C | | |
| ATOM | 49015 | O5 | BMA | L | 543 | −17.606 | −1.37 | 109.137 | 1.00 | 177.65 | L | O | | |
| ATOM | 49016 | C6 | BMA | L | 543 | −18.350 | −3.551 | 109.832 | 1.00 | 177.65 | L | C | | |
| ATOM | 49017 | O6 | BMA | L | 543 | −17.645 | −4.067 | 108.678 | 1.00 | 177.65 | L | O | | |
| ATOM | 48993 | C1 | NAG | L | 542 | −12.824 | 1.705 | 109.787 | 1.00 | 155.29 | L | C | NAG-2 | |
| ATOM | 48994 | C2 | NAG | L | 542 | −13.085 | 0.202 | 109.887 | 1.00 | 155.29 | L | C | | |
| ATOM | 48995 | N2 | NAG | L | 542 | −11.988 | −0.530 | 109.283 | 1.00 | 155.29 | L | N | | |
| ATOM | 48996 | C7 | NAG | L | 542 | −11.747 | −0.418 | 107.981 | 1.00 | 155.29 | L | C | | |
| ATOM | 48997 | O7 | NAG | L | 542 | −11.141 | 0.535 | 107.494 | 1.00 | 155.29 | L | O | | |
| ATOM | 48998 | C8 | NAG | L | 542 | −12.266 | −1.528 | 107.083 | 1.00 | 155.29 | L | C | | |
| ATOM | 48999 | C3 | NAG | L | 542 | −14.397 | −0.151 | 109.181 | 1.00 | 155.29 | L | C | | |
| ATOM | 49000 | O3 | NAG | L | 542 | −14.749 | −1.496 | 109.478 | 1.00 | 155.29 | L | O | | |
| ATOM | 49001 | C4 | NAG | L | 542 | −15.573 | 0.792 | 109.598 | 1.00 | 155.29 | L | C | | |
| ATOM | 49002 | O4 | NAG | L | 542 | −16.655 | 0.646 | 108.656 | 1.00 | 155.29 | L | O | | Orange* |
| ATOM | 49003 | C5 | NAG | L | 542 | −15.127 | 2.262 | 109.606 | 1.00 | 155.29 | L | C | | |
| ATOM | 49004 | O5 | NAG | L | 542 | −13.915 | 2.420 | 110.366 | 1.00 | 155.29 | L | O | | |
| ATOM | 49005 | C6 | NAG | L | 542 | −16.155 | 3.192 | 110.216 | 1.00 | 155.29 | L | C | | |
| ATOM | 49006 | O6 | NAG | L | 542 | −15.785 | 3.589 | 111.530 | 1.00 | 155.29 | L | O | | |
| | | | | | | | | | | | | | Kabat | |
| ATOM | 85197 | N | GLN | 9 | 57 | −15.497 | −0.344 | 102.961 | 1.00 | 136.36 | 9 | N | 56 | F5126-VH |
| ATOM | 85198 | CA | GLN | 9 | 57 | −16.123 | 0.896 | 103.416 | 1.00 | 136.36 | 9 | C | 56 | |
| ATOM | 85199 | CB | GLN | 9 | 57 | −15.058 | 1.900 | 103.864 | 1.00 | 176.32 | 9 | C | 56 | |
| ATOM | 85200 | CG | GLN | 9 | 57 | −15.599 | 3.149 | 104.549 | 1.00 | 176.32 | 9 | C | 56 | |
| ATOM | 85201 | CD | GLN | 9 | 57 | −16.324 | 2.844 | 105.846 | 1.00 | 176.32 | 9 | C | 56 | |
| ATOM | 85202 | OE1 | GLN | 9 | 57 | −17.526 | 2.577 | 105.855 | 1.00 | 176.32 | 9 | O | 56 | |
| ATOM | 85203 | NE2 | GLN | 9 | 57 | −15.589 | 2.868 | 106.949 | 1.00 | 176.32 | 9 | N | 56 | Purple* |
| ATOM | 85204 | C | GLN | 9 | 57 | −16.917 | 1.465 | 102.247 | 1.00 | 136.36 | 9 | C | 56 | |
| ATOM | 85205 | O | GLN | 9 | 57 | −16.540 | 1.281 | 101.090 | 1.00 | 136.36 | 9 | O | 56 | |
| ATOM | 48993 | C1 | NAG | L | 542 | −12.824 | 1.705 | 109.787 | 1.00 | 155.29 | L | C | NAG-2 | HA-R |
| ATOM | 48994 | C2 | NAG | L | 542 | −13.085 | 0.202 | 109.887 | 1.00 | 155.29 | L | C | | |
| ATOM | 48995 | N2 | NAG | L | 542 | −11.988 | −0.530 | 109.283 | 1.00 | 155.29 | L | N | | |
| ATOM | 48996 | C7 | NAG | L | 542 | −11.747 | −0.418 | 107.981 | 1.00 | 155.29 | L | C | | |
| ATOM | 48997 | O7 | NAG | L | 542 | −11.141 | 0.535 | 107.494 | 1.00 | 155.29 | L | O | | |
| ATOM | 48998 | C8 | NAG | L | 542 | −12.266 | −1.528 | 107.083 | 1.00 | 155.29 | L | C | | |
| ATOM | 48999 | C3 | NAG | L | 542 | −14.397 | −0.151 | 109.181 | 1.00 | 155.29 | L | C | | |
| ATOM | 49000 | O3 | NAG | L | 542 | −14.749 | −1.496 | 109.478 | 1.00 | 155.29 | L | O | | |
| ATOM | 49001 | C4 | NAG | L | 542 | −15.573 | 0.792 | 109.598 | 1.00 | 155.29 | L | C | | |
| ATOM | 49002 | O4 | NAG | L | 542 | −16.655 | 0.646 | 108.656 | 1.00 | 155.29 | L | O | | Purple* |
| ATOM | 49003 | C5 | NAG | L | 542 | −15.127 | 2.262 | 109.606 | 1.00 | 155.29 | L | C | | |
| ATOM | 49004 | O5 | NAG | L | 542 | −13.915 | 2.420 | 110.366 | 1.00 | 155.29 | L | O | | |
| ATOM | 49005 | C6 | NAG | L | 542 | −16.155 | 3.192 | 110.216 | 1.00 | 155.29 | L | C | | |
| ATOM | 49006 | O6 | NAG | L | 542 | −15.785 | 3.589 | 111.530 | 1.00 | 155.29 | L | O | | |
| | | | | | | | | | | | | | Kabat | |
| ATOM | 85206 | N | THR | 9 | 58 | −18.012 | 2.156 | 102.545 | 1.00 | 128.65 | 9 | N | 57 | F5126-VH |
| ATOM | 85207 | CA | THR | 9 | 58 | −18.849 | 2.720 | 101.493 | 1.00 | 128.65 | 9 | C | 57 | |
| ATOM | 85208 | CB | THR | 9 | 58 | −19.974 | 1.739 | 101.110 | 1.00 | 125.49 | 9 | C | 57 | |
| ATOM | 85209 | OG1 | THR | 9 | 58 | −20.706 | 1.372 | 102.286 | 1.00 | 125.49 | 9 | O | 57 | Pink* |
| ATOM | 85210 | CG2 | THR | 9 | 58 | −19.397 | 0.489 | 100.468 | 1.00 | 125.49 | 9 | C | 57 | |
| ATOM | 85211 | C | THR | 9 | 58 | −19.488 | 4.058 | 101.850 | 1.00 | 128.65 | 9 | C | 57 | |
| ATOM | 85212 | O | THR | 9 | 58 | −20.000 | 4.242 | 102.954 | 1.00 | 128.65 | 9 | O | 57 | |
| ATOM | 49115 | C1 | MAN | L | 534 | 44.909 | −20.120 | 116.726 | 1.00 | 223.86 | L | C | MAN-4 | HA-R |
| ATOM | 49116 | C2 | MAN | L | 534 | 44.314 | −19.389 | 117.959 | 1.00 | 223.86 | L | C | | |
| ATOM | 49117 | O2 | MAN | L | 534 | 45.320 | −19.188 | 118.975 | 1.00 | 223.86 | L | O | | |
| ATOM | 49118 | C3 | MAN | L | 534 | 43.601 | −18.05 | 117.630 | 1.00 | 223.86 | L | C | | |
| ATOM | 49119 | O3 | MAN | L | 534 | 43.494 | −17.262 | 118.808 | 1.00 | 223.86 | L | O | | |
| ATOM | 49120 | C4 | MAN | L | 534 | 44.322 | −17.236 | 116.531 | 1.00 | 223.86 | L | C | | |
| ATOM | 49121 | O4 | MAN | L | 534 | 43.504 | −16.155 | 116.106 | 1.00 | 223.86 | L | O | | |
| ATOM | 49122 | C5 | MAN | L | 534 | 44.601 | −18.161 | 115.358 | 1.00 | 223.86 | L | C | | |
| ATOM | 49123 | O5 | MAN | L | 534 | 45.487 | −19.205 | 115.793 | 1.00 | 223.86 | L | O | | |
| ATOM | 49124 | C6 | MAN | L | 534 | 45.237 | −17.485 | 114.158 | 1.00 | 223.86 | L | C | | |
| ATOM | 49125 | O6 | MAN | L | 534 | 46.358 | −16.700 | 114.535 | 1.00 | 223.86 | L | O | | Pink* |

*same color indicates a hydrogen-bonding pair.

| | | | | | | | | | | | | | | |
|---|---|---|---|---|---|---|---|---|---|---|---|---|---|---|
| FR3 | | | | | | | | | | | | | Kabat | |
| ATOM | 85347 | N | GLY | 9 | 75 | −11.945 | −11.376 | 94.112 | 1.00 | 149.52 | 9 | N | 74 | F5126-VH |
| ATOM | 85348 | CA | GLY | 9 | 75 | −11.463 | −12.557 | 93.422 | 1.00 | 149.52 | 9 | C | 74 | |
| ATOM | 85349 | C | GLY | 9 | 75 | −11.266 | −12.424 | 91.923 | 1.00 | 149.52 | 9 | C | 74 | |
| ATOM | 85350 | O | GLY | 9 | 75 | −10.347 | −13.027 | 91.369 | 1.00 | 149.52 | 9 | O | 74 | Blue* |
| ATOM | 38692 | N | GLY | J | 240 | −9.391 | −15.098 | 94.365 | 1.00 | 147.33 | J | N | | HA-L, Blue* |
| ATOM | 38693 | CA | GLY | J | 240 | −8.606 | −14.767 | 93.190 | 1.00 | 147.33 | J | C | | |
| ATOM | 38694 | C | GLY | J | 240 | −7.329 | −15.577 | 93.095 | 1.00 | 147.33 | J | C | | |
| ATOM | 38695 | O | GLY | J | 240 | −6.888 | −15.929 | 92.000 | 1.00 | 147.33 | J | O | | |

*same color indicates a hydrogen-bonding pair.

| | | | | | | | | | | | | | | |
|---|---|---|---|---|---|---|---|---|---|---|---|---|---|---|
| CDR3 | | | | | | | | | | | | | Kabat | |
| ATOM | 85556 | N | VAL | 9 | 102 | −7.033 | 4.779 | 100.694 | 1.00 | 159.45 | 9 | N | 98 | F5126-VH |
| ATOM | 85557 | CA | VAL | 9 | 102 | −7.103 | 3.362 | 101.026 | 1.00 | 159.45 | 9 | C | 98 | |
| ATOM | 85558 | CB | VAL | 9 | 102 | −6.343 | 2.503 | 99.983 | 1.00 | 132.14 | 9 | C | 98 | |
| ATOM | 85559 | CG1 | VAL | 9 | 102 | −6.535 | 1.022 | 100.278 | 1.00 | 132.14 | 9 | C | 98 | |
| ATOM | 85560 | CG2 | VAL | 9 | 102 | −6.835 | 2.827 | 98.582 | 1.00 | 132.14 | 9 | C | 98 | |
| ATOM | 85561 | C | VAL | 9 | 102 | −6.468 | 3.156 | 102.396 | 1.00 | 159.45 | 9 | C | 98 | |
| ATOM | 85562 | O | VAL | 9 | 102 | −5.809 | 4.052 | 102.921 | 1.00 | 159.45 | 9 | O | 98 | Blue* |
| ATOM | 47127 | N | ASP | L | 271 | −1.988 | 4.738 | 104.261 | 1.00 | 117.47 | L | N | | HA-R |
| ATOM | 47128 | CA | ASP | L | 271 | −1.917 | 5.399 | 102.960 | 1.00 | 117.47 | L | C | | |
| ATOM | 47129 | CB | ASP | L | 271 | −3.009 | 4.862 | 102.031 | 1.00 | 124.59 | L | C | | |
| ATOM | 47130 | CG | ASP | L | 271 | −2.673 | 3.506 | 101.452 | 1.00 | 124.59 | L | C | | |
| ATOM | 47131 | OD1 | ASP | L | 271 | −1.824 | 3.443 | 100.538 | 1.00 | 124.59 | L | O | | |
| ATOM | 47132 | OD2 | ASP | L | 271 | −3.256 | 2.504 | 101.910 | 1.00 | 124.59 | L | O | | Blue* |
| ATOM | 47133 | C | ASP | L | 271 | −2.111 | 6.899 | 103.138 | 1.00 | 117.47 | L | C | | |
| ATOM | 47134 | O | ASP | L | 271 | −2.197 | 7.643 | 102.163 | 1.00 | 117.47 | L | O | | |
| | | | | | | | | | | | | | Kabat | |
| ATOM | 85563 | N | ARG | 9 | 103 | −6.679 | 1.980 | 102.975 | 1.00 | 148.87 | 9 | N | 99 | F5126-VH, |
| | | | | | | | | | | | | | | Yellow* |
| ATOM | 85564 | CA | ARG | 9 | 103 | −6.120 | 1.660 | 104.281 | 1.00 | 148.87 | 9 | C | 99 | |
| ATOM | 85565 | CB | ARG | 9 | 103 | −4.592 | 1.561 | 104.189 | 1.00 | 133.45 | 9 | C | 99 | |
| ATOM | 85566 | CG | ARG | 9 | 103 | −3.991 | 0.379 | 104.934 | 1.00 | 133.45 | 9 | C | 99 | |
| ATOM | 85567 | CD | ARG | 9 | 103 | −4.375 | −0.925 | 104.258 | 1.00 | 133.45 | 9 | C | 99 | |
| ATOM | 85568 | NE | ARG | 9 | 103 | −4.060 | −2.093 | 105.073 | 1.00 | 133.45 | 9 | N | 99 | |
| ATOM | 85569 | CZ | ARG | 9 | 103 | −4.666 | −3.267 | 104.944 | 1.00 | 133.45 | 9 | C | 99 | |
| ATOM | 85570 | NH1 | ARG | 9 | 103 | −5.617 | −3.423 | 104.032 | 1.00 | 133.45 | 9 | N | 99 | |
| ATOM | 85571 | NH2 | ARG | 9 | 103 | −4.333 | −4.283 | 105.728 | 1.00 | 133.45 | 9 | N | 99 | |
| ATOM | 85572 | C | ARG | 9 | 103 | −6.504 | 2.716 | 105.317 | 1.00 | 148.87 | 9 | C | 99 | |
| ATOM | 85573 | O | ARG | 9 | 103 | −7.657 | 3.140 | 105.392 | 1.00 | 148.87 | 9 | O | 99 | Orange* |
| ATOM | 47127 | N | ASP | L | 271 | −1.988 | 4.738 | 104.261 | 1.00 | 117.47 | L | N | | HA-R |
| ATOM | 47128 | CA | ASP | L | 271 | −1.917 | 5.399 | 102.960 | 1.00 | 117.47 | L | C | | |
| ATOM | 47129 | CB | ASP | L | 271 | −3.009 | 4.862 | 102.031 | 1.00 | 124.59 | L | C | | |
| ATOM | 47130 | CG | ASP | L | 271 | −2.673 | 3.506 | 101.452 | 1.00 | 124.59 | L | C | | |
| ATOM | 47131 | OD1 | ASP | L | 271 | −1.824 | 3.443 | 100.538 | 1.00 | 124.59 | L | O | | |
| ATOM | 47132 | OD2 | ASP | L | 271 | −3.256 | 2.504 | 101.910 | 1.00 | 124.59 | L | O | | Yellow* |
| ATOM | 47133 | C | ASP | L | 271 | −2.111 | 6.899 | 103.138 | 1.00 | 117.47 | L | C | | |
| ATOM | 47134 | O | ASP | L | 271 | −2.197 | 7.643 | 102.163 | 1.00 | 117.47 | L | O | | |
| ATOM | 48979 | C1 | NAG | L | 541 | −9.148 | 5.335 | 110.896 | 1.00 | 131.43 | L | C | NAG-1 | |
| ATOM | 48980 | C2 | NAG | L | 541 | −10.640 | 5.659 | 110.957 | 1.00 | 131.43 | L | C | | |
| ATOM | 48981 | N2 | NAG | L | 541 | −10.894 | 6.629 | 112.001 | 1.00 | 131.43 | L | N | | |
| ATOM | 48982 | C7 | NAG | L | 541 | −11.907 | 7.481 | 111.884 | 1.00 | 131.43 | L | C | | |
| ATOM | 48983 | O7 | NAG | L | 541 | −11.883 | 8.448 | 111.123 | 1.00 | 131.43 | L | O | | |
| ATOM | 48984 | C8 | NAG | L | 541 | −13.136 | 7.222 | 112.74 | 1.00 | 131.43 | L | C | | |
| ATOM | 48985 | C3 | NAG | L | 541 | −11.418 | 4.374 | 111.223 | 1.00 | 131.43 | L | C | | |
| ATOM | 48986 | O3 | NAG | L | 541 | −12.810 | 4.650 | 111.218 | 1.00 | 131.43 | L | O | | |
| ATOM | 48987 | C4 | NAG | L | 541 | −11.064 | 3.298 | 110.144 | 1.00 | 131.43 | L | C | | |
| ATOM | 48988 | O4 | NAG | L | 541 | −11.674 | 2.035 | 110.484 | 1.00 | 131.43 | L | O | | |
| ATOM | 48989 | C5 | NAG | L | 541 | −9.538 | 3.126 | 110.078 | 1.00 | 131.43 | L | C | | |
| ATOM | 48990 | O5 | NAG | L | 541 | −8.898 | 4.401 | 109.843 | 1.00 | 131.43 | L | O | | |
| ATOM | 48991 | C6 | NAG | L | 541 | −9.094 | 2.197 | 108.967 | 1.00 | 131.43 | L | C | | |
| ATOM | 48992 | O6 | NAG | L | 541 | −8.917 | 2.898 | 107.744 | 1.00 | 131.43 | L | O | | Orange* |
| | | | | | | | | | | | | | Kabat | |
| ATOM | 85574 | N | GLY | 9 | 104 | −5.519 | 3.144 | 106.100 | 1.00 | 132.49 | 9 | N | 100 | F5126-VH |
| ATOM | 85575 | CA | GLY | 9 | 104 | −5.741 | 4.130 | 107.143 | 1.00 | 132.49 | 9 | C | 100 | |
| ATOM | 85576 | C | GLY | 9 | 104 | −6.508 | 5.392 | 106.789 | 1.00 | 132.49 | 9 | C | 100 | |
| ATOM | 85577 | O | GLY | 9 | 104 | −7.290 | 5.883 | 107.605 | 1.00 | 132.49 | 9 | O | 100 | Pink* |
| ATOM | 48979 | C1 | NAG | L | 541 | −9.148 | 5.335 | 110.896 | 1.00 | 131.43 | L | C | NAG-1 | HA-R |
| ATOM | 48980 | C2 | NAG | L | 541 | −10.640 | 5.659 | 110.957 | 1.00 | 131.43 | L | C | | |
| ATOM | 48981 | N2 | NAG | L | 541 | −10.894 | 6.629 | 112.001 | 1.00 | 131.43 | L | N | | |
| ATOM | 48982 | C7 | NAG | L | 541 | −11.907 | 7.481 | 111.884 | 1.00 | 131.43 | L | C | | |
| ATOM | 48983 | O7 | NAG | L | 541 | −11.883 | 8.448 | 111.123 | 1.00 | 131.43 | L | O | | |
| ATOM | 48984 | C8 | NAG | L | 541 | −13.136 | 7.222 | 112.740 | 1.00 | 131.43 | L | C | | |
| ATOM | 48985 | C3 | NAG | L | 541 | −11.418 | 4.374 | 111.223 | 1.00 | 131.43 | L | C | | |
| ATOM | 48986 | O3 | NAG | L | 541 | −12.810 | 4.650 | 111.218 | 1.00 | 131.43 | L | O | | |
| ATOM | 48987 | C4 | NAG | L | 541 | −11.064 | 3.298 | 110.144 | 1.00 | 131.43 | L | C | | |
| ATOM | 48988 | O4 | NAG | L | 541 | −11.674 | 2.035 | 110.484 | 1.00 | 131.43 | L | O | | |
| ATOM | 48989 | C5 | NAG | L | 541 | −9.538 | 3.126 | 110.078 | 1.00 | 131.43 | L | C | | |
| ATOM | 48990 | O5 | NAG | L | 541 | −8.898 | 4.401 | 109.843 | 1.00 | 131.43 | L | O | | Pink* |
| ATOM | 48991 | C6 | NAG | L | 541 | −9.094 | 2.197 | 108.967 | 1.00 | 131.43 | L | C | | |
| ATOM | 48992 | O6 | NAG | L | 541 | −8.917 | 2.898 | 107.744 | 1.00 | 131.43 | L | O | | |

*same color indicates a hydrogen-bonding pair.
The effective number of decimal place is first decimal place, in notation of the atomic coordinate.

TABLE 3
Data collection and refinement statistics.

| Data collection | F005-126-H3 Complex |
|---|---|
| Beamline | BL41XU |
| Wavelength (Å) | 1.00 |
| Space group | C 2 |
| Unit cell parameters | a = 391.04, b = 241.17, c = 223.21 Å |
| | α = γ = 90.0°, β = 123.62 |
| Resolution (Å) | 40-4.0 (4.25-4.00) |
| Observations | 554,887 |
| Unique reflections | 142,111 (23,528) |
| Redundancy | 3.9 (3.9) |
| Completeness (%) | 98.0 (98.1) |
| <I/σI> | 6.4 (1.26) |
| Rmerge | 0.23 (2.09) |
| Z ac | 12 |
| Refinement | |
| Resolution (Å) | 40-4.0 |
| Rwork (free) | 0.31 (0.33) |
| No. atoms | |
| Protein atoms | 85,260 |
| Carbohydrate atoms | 2,664 |
| Waters | 0 |
| B-factors | |
| Protein | 170 |
| Carbohydrate | 196 |
| r.m.s deviations | |
| Bond length (Å) | 0.004 |
| Bond angles (°) | 1.02 |
| Ramachandran statistics | (%) f |
| Favored | 93.9 |
| Outliers | 0.9 |

TABLE 4
Hydrogen bonds and Van der Waals contacts between F005-126 and HA.
N-acetyl-D-glucosamine, α-D-mannose, and β-D-mannose are
abbreviated as NAG, MAN, and BMA, respectively.
Hydrogen bonds

| | | |
|---|---|---|
| F005-126 | HA | distance |

| | | | | | |
|---|---|---|---|---|---|
| HCDR1 | Ser31 | O | S91 | OG | 3.63 |
| | Ser31 | OG | S91 | O | 3.87 |
| HCDR2 | Asp54 | OD1 | NAG-2 | O7 | 2.58 |
| | Asp54 | O | MAN-2 | O2 | 3.78 |
| | Gly55 | O | BMA | O2 | 3.56 |
| | Gln56 | OE1 | BMA | O2 | 2.96 |
| | Gln56 | OE1 | NAG-2 | O4 | 3.49 |
| | Gln56 | NE2 | NAG-2 | O4 | 3.0 |
| | Thr57 | OG1 | MAN-4 | O6 | 3.47 |
| HFR3 | Gly74 | O | G240 | N | 3.75 |
| HCDR3 | Val98 | O | D271 | OD2 | 3.2 |
| | Arg99 | N | D271 | OD2 | 3.7 |
| | Arg99 | O | NAG-1 | O6 | 2.6 |
| | Gly100 | O | NAG-1 | O5 | 3.1 |

Van der Waals contacts

| | | |
|---|---|---|
| F005-126 | HA | Contacts |

| | | | |
|---|---|---|---|
| HCDR1 | Ser31 | S91 | 2 |
| | Ser31 | K92 | 1 |
| HCDR2 | Asp54 | NAG-2 | 7 |
| | Gln56 | NAG-2 | 1 |
| | Gln56 | BMA | 3 |
| HFR3 | Gly74 | G240 | 6 |
| | Gly74 | P239 | 1 |
| HCDR3 | Val98 | S91 | 1 |
| | Arg99 | NAG-1 | 2 |
| | Arg99 | D271 | 1 |
| | Arg99 | S91 | 1 |
| | Gly100 | P284 | 3 |
| | Gly100 | NAG-1 | 1 |
| | Gly100 | S270 | 1 |
| | Gly100 | D285 | 1 |

TABLE 5
The sequence of the Site L and Site R, estimating variations within subtypes of Influenza A viruses.

| | | Site L | | | | | Site R | | | | | |
|---|---|---|---|---|---|---|---|---|---|---|---|---|
| | | 171-173 | 239-240 | order | appear | frequency(%) | 91-92 | 270-273 | 284-285 | order | appear | frequency (%) |
| H3N2 | Aic68 | NDN | PG | 5 | 259 | 5.7% | SK | SDAP | PN | 1 | 4112 | 90.8% |
| n = 4531 | Fuk70 | NDN | PG | 5 | 259 | 5.7% | SK | SDAP | PN | 1 | 4112 | 90.8% |
| | Tok73 | NDN | PG | 5 | 259 | 5.7% | SK | SDAP | PN | 1 | 4112 | 90.8% |
| | Yam77 | NDN | PG | 5 | 259 | 5.7% | SK | SDAP | PN | 1 | 4112 | 90.8% |
| | Nii81 | NGN | PG | 8 | 20 | 0.4% | SK | SDAP | PN | 1 | 4112 | 90.8% |
| | Fuk85 | NGK | PG | 3 | 332 | 7.3% | SK | SDAP | PN | 1 | 4112 | 90.8% |
| | Gui89 | NGK | PG | 3 | 332 | 7.3% | SK | SDAP | PN | 1 | 4112 | 90.8% |
| | Kit93 | NGK | PG | 3 | 332 | 7.3% | SK | SDAP | PN | 1 | 4112 | 90.8% |
| | Syd97 | NDK | PG | 4 | 329 | 7.3% | SK | SDAP | PN | 1 | 4112 | 90.8% |
| | Pan99 | NEK | PG | 1 | 2010 | 44.4% | SK | SDAP | PN | 1 | 4112 | 90.8% |
| | Wyo03 | NEK | PG | 1 | 2010 | 44.4% | SK | SDAP | PN | 1 | 4112 | 90.8% |
| | NY04 | NEK | PG | 1 | 2010 | 44.4% | SK | SDAP | PN | 1 | 4112 | 90.8% |
| H1N1 | SC1918 | NKG | PG | 3 | 44 | 2.2% | SN | SDAP | PH | 19 | 1 | 0.0% |
| n = 2028 | NC99 | NKE | PG | 1 | 1839 | 90.7% | PN | SNAP | PQ | 1 | 1618 | 79.8% |
| H1N1 | Cal109pdm | DKG | PG | 1 | 7209 | 99.3% | PS | SDTP | PK | 6 | 31 | 0.4% |
| (pdm) | | | | | | | | | | | | |
| n = 7258 | | | | | | | | | | | | |
| H5N1 | Viet04 | TNQ | PN | 1 | 167 | 72.3% | AN | SELE | PM | 1 | 105 | 45.5% |
| n = 231 | | | | | | | | | | | | |

TABLE 6
The ranking out-put corresponding to Docking order (S) from docking
study in Experiment 6. Three Docking Pause Results of hit compound
(Nos. 2, 3 and 6) or possible active agents are shown in FIG. 21, 22
and 23.

| No | Compounds | Weight | mseq | S |
|---|---|---|---|---|
| 1 | Dactinomycin | 1617.6689 | 1696 | −14.162 |
| 2 | Bacitracin | 2554.1008 | 746 | −14.1208 |
| 3 | Colistimethate sodium | 1422.719 | 3109 | −13.695 |
| 4 | Gramicidin | 1462.705 | 3054 | −13.4522 |
| 5 | Nystatin | 1625.905 | 2879 | −12.5476 |
| 6 | Polymyxin B sulfate | 1160.495 | 3040 | −12.3328 |
| 7 | Tyrothricin | 1422.636 | 3518 | −12.2135 |
| 8 | Suramin | 1255.438 | 1519 | −12.161 |
| 9 | Cyclosporine | 1271.46 | 2717 | −11.8786 |
| 10 | Ramoplanin | 1422.719 | 3110 | −11.8438 |
| 11 | Daptomycin (=Cubicin) | 1877.6639 | 687 | −11.3177 |
| 12 | Mepartricin | 997.405 | 1437 | −11.3174 |
| 13 | Bleomycin | 1143.486 | 2251 | −11.182 |
| 14 | Iodixanol | 1665.9249 | 3519 | −10.9615 |
| 15 | Candicidin | 1109.317 | 1473 | −10.9461 |
| 16 | Sirolimus (Rapamycin) | 1160.495 | 3039 | −10.8022 |
| 17 | Ubidecareneone | 1291.2479 | 2881 | −10.7934 |
| 18 | Tannic Acid | 863.365 | 1251 | −10.7785 |
| 19 | Fast green FCF | 1208.5389 | 3026 | −10.7067 |
| 20 | Enoxaparin sodium (1% | 929.1609 | 2887 | −10.5709 |
| | wt/vol in 10% aq DMSO) | | | |
| 21 | Thiostrepton | 1701.2059 | 1766 | −10.5208 |
| 22 | Tilmicosin | 929.1609 | 2888 | −10.4248 |
| 23 | Nonoxynol-9 | 1202.635 | 2176 | −10.421 |
| 24 | Solanesol | 1764.432 | 3048 | −10.2698 |
| 25 | Solanesyl acetate | 1141.359 | 1474 | −9.9841 |
| 26 | Teicoplanin | 926.107 | 1694 | −9.9754 |
| 27 | Atracurium besylate | 871.163 | 1637 | −9.8871 |
| 28 | Sucralfate sodium (10 mM | 1202.635 | 2178 | −9.7955 |
| | 10% aq DMSO) | | | |
| 29 | Colistin sulfate | 914.187 | 1487 | −9.7463 |
| 30 | Filipin | 631.086 | 2536 | −9.7377 |
| 31 | Tylosin | 917.12 | 1701 | −9.4949 |
| 32 | Chicago sky blue 6B | 654.838 | 1483 | −9.3396 |
| 33 | Cyclosporin A | 1550.188 | 1062 | −9.2419 |
| 34 | Colistin sulfate | 762.881 | 2883 | −9.2275 |
| 35 | Atracurium besylate | 900.856 | 2886 | −9.1969 |
| 36 | Beta-Escin | 673.123 | 1128 | −9.1695 |
| 37 | Bacitracin (Prestw-919) | 1131.225 | 1688 | −9.0946 |
| 38 | Tyloxapol | 616.833 | 1433 | −9.0144 |

What is claimed is:

1. An isolated antibody directed to hemagglutinin (HA) trimer of an influenza virus, wherein said antibody comprises:
(i) the sequence of CDR1 (SEQ ID NO: 3) of F005-126 antibody heavy chain (SEQ ID NO: 2), or a functionally equivalent sequence thereof;
(ii) the sequence of CDR2 (SEQ ID NO: 4) of F005-126 antibody heavy chain (SEQ ID NO: 2), or a functionally equivalent sequence thereof;
(iii) the sequence of CDR1 (SEQ ID NO: 5) of F005-126 antibody heavy chain (SEQ ID NO: 2), or a functionally equivalent sequence thereof; and
(iv) the sequence of FR3 (SEQ ID NO: 8) of F005-126 antibody heavy chain (SEQ ID NO: 2), or a functionally equivalent sequence thereof.

2. The antibody according to claim 1, which further comprises
(v) the sequence of FR1 (SEQ ID NO: 6) of F005-126 antibody heavy chain (SEQ ID NO: 2), or a functionally equivalent sequence thereof
(vi) the sequence of FR2 (SEQ ID NO: 7) of F005-126 antibody heavy chain (SEQ ID NO: 2), or a functionally equivalent sequence thereof; and
(vii) the sequence of FR4 (SEQ ID NO: 9) of F005-126 antibody heavy chain (SEQ ID NO: 2), or a functionally equivalent sequence thereof.

3. The antibody according to claim 2, which further comprises:
the sequence of F005-126 antibody light chain (SEQ ID NO: 13), or a functionally equivalent sequence thereof.

4. The antibody according to claim 1, wherein said antibody comprises at least one of the properties selected from the group consisting of:
(1) having broad strain specificity against H3;
(2) binds to HA1 head region but does not inhibit binding to cell;
(3) inhibits structural change of HA;
(4) said CDR1, CDR3 and FR3 bind to HA by van der Waals contact;
(5) said CDR2 binds to N285 (according to the Kabat's numbering shown in FIG. 5-2) sugar chain which is conserved in HA;
(6) binds to the HA trimer across two HA subunits thereof which are adjacent to each other;
(7) intra- and inter-subunit interactions between HA1 and HA2 by salt bridges are located on the amino acid sequence of the molecular surface in the vicinity of the portion which maintains structure of the HA trimer;
(8) comprising hydrogen bonds.

5. The antibody according to claim 4, wherein the antibody has a property of binding to the HA trimer across two HA subunits thereof which are adjacent to each other.

6. The antibody according to claim 1 which is a neutralizing antibody.

7. The antibody according to claim 1 which is an antibody neutralizing H3.

8. The antibody according to claim 1, wherein the antibody comprises (a) the sequence set forth in SEQ ID NO: 2, or (b) a sequence derived from the sequence of (a) comprising one or more amino acid substitution(s), deletion(s) and/or addition(s).

9. The antibody according to claim 1, wherein the antibody comprises:
(a) the sequence set forth in SEQ ID NO: 2, or
(b) a sequence derived from the sequence of (a) comprising one or more amino acid substitution(s), deletion(s) and/or addition(s) at positions other than the binding site with HA of CDR1 sequence of F005-126 antibody (amino acid No. 31 (Ser) of SEQ ID NO. 2), the binding site with HA of CDR2 sequence of F005-126 antibody (SEQ ID NO: 10 (amino acids No. 54-58(Tyr Asn Gly Asn Thr) of SEQ ID NO. 2)), the binding site with HA of CDR3 sequence of F005-126 antibody (amino acids No. 74-76 (Thr Ser Thr) of SEQ ID NO. 2), and the binding site with HA of FR3 sequence of F005-126 antibody (SEQ ID NO: 11 (amino acids No. 102-105 (Val Arg Gly Val) of SEQ ID NO. 2)), wherein the sequence maintains the binding activity with the HA trimer.

10. The antibody according to claim 1, wherein the antibody comprises:
(a) the sequence set forth in SEQ ID NO: 2, or
(b) a sequence derived from the sequence of (a) comprising one or more amino acid substitution(s), deletion(s) and/or addition(s) at the positions other than the CDR1 sequence of F005-126 antibody heavy chain (SEQ ID NO: 3), the CDR2 sequence of F005-126 antibody heavy chain (SEQ ID NO: 4), the CDR3 sequence of F005-126 antibody heavy chain (SEQ ID NO: 5), and the FR3 sequence of F005-126 antibody heavy chain (SEQ ID NO: 8), wherein the sequence maintains the binding activity with the HA trimer.

11. The antibody according to claim 1, which consists of the sequence set forth in SEQ ID NO: 2 and SEQ ID NO: 13.

12. A screening kit for an antibody against hemagglutinin (HA) trimer of an influenza virus, comprising the antibody according to claim 1.

13. The kit according to claim 12, further comprising a protein or protein complex comprising the sequence of concave region of the HA trimer (SEQ ID NOs; 48 and 21).

14. An influenza virus passive immunotherapy agent comprising the antibody according to claim 1.

15. A method of influenza virus passive immunotherapy comprising the step of administering the antibody according to claim 1 to a patient in need thereof.

16. A kit for paratope analysis of an influenza neutralizing antibody comprising a protein or protein complex comprising the sequence of concave region of a HA trimer.

17. The kit according to claim 16, wherein the protein or protein complex comprises
(A) the full length sequence of the HA trimer; or
(B) a sequence derived from the full length sequence of (B) comprising one or more amino acid substitution(s), deletion(s) and/or addition(s) at the positions other than the sequence of concave region, wherein the sequence maintains the binding activity with F005-126 antibody.

18. The kit according to claim 16, wherein the protein or protein complex consists of (A) the full length sequence of the HA trimer.

19. The kit according to claim 16, wherein the paratope is related to an antibody against Group 2 hemagglutinin.

20. The kit according to claim 16 wherein the paratope is related to an antibody against hemagglutinin H3.

21. The kit according to claim 16 wherein the paratope is related to an antibody against hemagglutinin H3, whose strain is selected from the group consisting of Aic68 (SEQ ID NO: 48 and 21), Fuk70 (SEQ ID NO: 48 and 22), Tok73 (SEQ ID NO: 50 and 23), Yam77 (SEQ ID NO: 51 and 24), Nii81 (SEQ ID NO: 52 and 25), Fuk85 (SEQ ID NO: 53 and 26), Gui89 (SEQ ID NO: 54), Kit93 (SEQ ID NO: 55), Syd97 (SEQ ID NO: 56 and 27), Pan99 (SEQ ID NO: 57 and 28), Wyo03 (SEQ ID NO: 58 and 29) and NY04 (SEQ ID NO: 59 and 30).

22. A method for identifying a binding substance to a hemagglutinin (HA) trimer of an influenza virus, the method comprising the steps of:
(A) providing a 3D structural representation of the HA trimer, wherein the 3D structural representation of the HA trimer comprises the atomic co-ordinates relating to a 3D structural representation of the amino acid residues contained in the HA of Table 1 described in the specification;
(B) providing a 3D structural representation of a candidate substance of the binding substance;
(C) using a computer to dock the 3D structural representation of the candidate substance with the 3D structural representation of the HA trimer, wherein a candidate substance that docks with the HA trimer at the site comprising the amino acid residues contained in the HA of the Table 1, is identified as the binding substance of the HA trimer;
(D) contacting the candidate substance identified in step (C) with HA trimer or a fragment thereof containing the 3D structure of the amino acid residues contained in the HA of Table 1; and
(E) assaying the interaction between the candidate substance and the HA trimer or the fragment thereof, to determine whether the binding substance identified in step (C) is a binding substance for the HA trimer.

23. The method according to claim 22, wherein the 3D structural representation comprises at least one interaction selected from the group consisting of van der Waals contacts, electrostatic interactions, and hydrogen bonding.

24. The method according to claim 22, wherein the 3D structural representation comprises van der Waals contacts, electrostatic interactions, and hydrogen bonding.

25. The method according to claim 22, wherein the 3D structural representation of the amino acid residues contained in the HA of the following Table 1 comprises
(A) the atomic co-ordinates set forth in Table 2 consisting of Tables 2-1, 2-2, 2-3 and 2-4 in the specification.
or
(B) variant atomic co-ordinates of (A), in which the r.m.s. deviation of the x, y and z co-ordinates for all heavy atoms is less than 2.5 Angstroms (or 4.0 Angstroms).

26. The method according to claim 22, wherein the 3D structural representation of the amino acid residues contained in the HA of the following Table 1 comprises the entire atomic co-ordinates set forth in PDB1, PDB2, PDB3 and/or PDB4.

27. The method according to claim 22, wherein said step of docking comprises geometric matching or minimizing the energy of interaction between the candidate substance and the amino acid residues of the HA trimer contained in the HA of Table 1.

28. The method according to claim 22 wherein the candidate substance comprises a library of antibodies.

29. The method according to claim 22 wherein the binding substance is a fusogenic conformational change inhibitor for HA trimer.

30. The method according to claim 22, wherein the step of docking comprises referring to the 3D structural representation of the antibody set forth in Table 1.

31. A method for screening an active agent for hemagglutinin comprising:
(a) constructing a 3D structure model of hemagglutinin using any one of PDB1, PDB2, PDB3 and PDB4;
(b) identifying a dock site;
(c) carrying out docking simulations for a first library of compounds as an initial screen;
(d) selecting hits from the initial screen; and
(e) performing a secondary screen using a combined library of the hits from the initial screen and a second library thereby determining the active agents.

32. A method for estimating variations within subtypes of Influenza A viruses, comprising:
(a) providing amino acid sequences of Influenza A virus;
(b) extracting complete Hemagglutinin sequences from the amino acid sequences of step (a);
(c) aligning the sequences extracted in step (b) and identifying the epitope regions according to the positions shown in FIG. 8.; and
(d) estimating the variation of each subtype by computing Shannon index of each site, by counting the number of different kind of sequences and by making sequence logos.

33. A method for screening active agent for regulating influenza virus or influenza virus hemagglutinin comprising:
(a) constructing a 3D structure model of hemagglutinin using any one of PDB1, PDB2, PDB3 and PDB4;
(b) identifying a dock site;
(c) carrying out docking simulations for a first library of compounds as an initial screen;
(d) selecting hits from the initial screen; and
(e) performing a biological assay with the candidate compound to confirm that the compound has the regulating activity.
